(19) United States
(12) Patent Application Publication
(10) Pub. No.: US 2005/0085555 A1
(43) Pub. Date: Apr. 21, 2005

(54) Composition, synthesis and therapeutic applications of polyamines

(76) Inventors: Michael A Murphy, La Jolla, CA (US); Mitchell R Malachowski, San Diego, CA (US)

Correspondence Address: CHARMASSON & BUCHACA & LEACH LLP, 1545 HOTEL CIRCLE SOUTH, SUITE 150, SAN DIEGO, CA, 92108-3412, US

(21) Appl. No.: 10/499,931

(22) PCT Filed: Dec. 18, 2002
(86) PCT No.: PCT/US02/40732

Related U.S. Application Data
(63) Continuation-in-part of application No. 10/017,235, filed on Dec. 18, 2001.
Application No. 10/017,235 is a continuation-in-part of application No. 09/486,310, filed on Feb. 23, 2000, now Pat. No. 6,576,672.
Application No. 10/017,235 is a continuation of application No. 08/915,660, filed on Aug. 21, 1997, now Pat. No. 5,906,996.

(51) Int. Cl. A61K 31/13

(52) U.S. Cl. 514/674

(57) ABSTRACT

This invention relates to a process of synthesis and composition of open chain (ring), closed ring, linear branched and or substituted polyamines, polyamine derived tyrosine phosphatase inhibitors and PPAR partial agonists/partial antagonists via a series of substitution reactions and optimizing the bioavailability and biological activities of the compounds. Polyamines prevent the toxicty of neutoxins and diabetogenic toxins including paraquat, methyphenyl pyridine radical, rotenone, diazoxide, streptozotocin and alloxan. These polyamines can be to treat neurological, cardiovascular, endocrine acquired and inherited mitochondrial DNA damage diseases and other disorders in mammalian subjects, and more specifically to the therapy of Parkinson's disease, Alzheimer's disease, Lou Gehrig's disease, Binswanger's disease, Olivopontine Cerebellar Degeneration, Lewy Body disease, Diabetes, Stroke, Atherosclerosis, Myocardial Ischemia, Cardiomyopathy, Nephropathy, Ischemia, Glaucoma, Presbycussis, Cancer, Osteoporosis, Rheumatoid Arthritis, Inflammatory Bowel Disease, Multiple Sclerosis and as Antidotes to Toxin Exposure.

### FIELD OF INVENTION

This invention relates to a process of synthesis and composition of open chain (ring), closed ring, linear branched and or substituted polyamines and polyamine derived tyrosine phosphatase inhibitors/PPARα and PPARγ partial agonists/partial antagonists for the treatment of neurological, cardiovascular, endocrine and other disorders in mammalian subjects, and more specifically to the therapy of Parkinson's disease, Alzheimer's disease, Lou Gehrig's disease, Binswanger's disease, Olivopontine Cerebellar Degeneration, Lewy Body disease, Diabetes, Stroke, Atherosclerosis, Myocardial Ischemia, Cardiomyopathy, Nephropathy, Ischemia, Glaucoma, Presbycussis, Cancer, Osteoporosis, Rheumatoid Arthritis, Inflammatory Bowel Disease, Multiple Sclerosis and as Antidotes to Toxin Exposure.

### CHEMICAL AND THERAPEUTIC BACKGROUND

Chemistry

There are seven groups of polyamines described herein, (1) predominately linear tetraamines and polyamines linked by 1,3-propylene and/or ethylene groups, those derived from 1,3-bis-[(2′-aminoethyl)amino]propane (2,3,2-tetramine); (2) predominately branched tetraamines and polyamines linked by 1,3-propylene and/or ethylene groups; (3) cyclic polyamines linked by 1,3-propylene and/or ethylene groups, ), those derived from the macrocycle 1,4,8,11-tetraazacyclotetradecane (cyclam); (4) combinations of linear, branched and cyclic polyamines linked by one or more 1,3-propylene and/or ethylene groups, (5) substituted polyamines, (6) polyamines derivatized to form tyrosine phosphatase inhibitor molecules with linear or branched chains attached and (7) derivatives of 2,2′-diaminobiphenyl with linear or branched chains attached. Of the collection of compounds described, most are not presently known but a few have been prepared previously.

Inherited and Acquired Mitochondrial DNA Damage

Individuals carrying mild mitochondrial DNA base substitutions manifest late onset diseases like Parkinson's and Alzheimer's diseases and familial deafness, whereas persons with moderately deleterious base substitutions develop Type II diabetes, Leber's Hereditary Optic Neuropathy, Myclonic Epilepsy and Ragged Red Fiber Disease (MERRF). Individuals with severely deleterious base substitutions develop pediatric onset myopathies, dystonias and Leigh's syndrome. Wallace D. C. (1992 a,b) suggests that aging and common degenerative diseases result from energetic decline caused by inherited oxidative phosphorylation (OXPHOS) gene defects and acquired somatic mutations. Mild mitochondrial deoxyribonucleic acid (DNA) rearrangements and duplications cause maternally inherited adult-onset diabetes and deafness. More severe rearrangements and deletions have been associated with adult-onset Chronic Progressive External Ophthalmoplegia (CPEO) and Kearns-Sayre Syndrome (KSS) and Pearson's Marrow/Pancreas Syndrome. Primary oxidative phosphorylation (OPHOS) diseases frequently have a delayed onset organ selectivity and an episodic, progressive course. For example the A3243G mutation associated with mitochondrial encephalopathy, lactic acidemia, stroke-like episodes (MELAS) can pure a pure cardiomyopathy, pure diabetes and deafness, or pure external ophthalmoplegia (Naviaux R. K. 2000).

The level of oxidative damage to mitochondrial and nuclear DNA, as measured by 8-hydroxy-2′-deoxy guanosine increases with age (Mecocci P. et al 1993) and oxidative damage to mitochondrial DNA occurs in Alzheimer's disease (Mecocci P. et al 1994 and 1998).

Some organs may be more prone to oxidative damage due to lack of protective substances, for example uric acid an antioxidant and transition metal chelator (Ames B. N. et al 1981) is not present in brain that may limit recovery from ischemic reperfusion damage and metal accumulation post stroke.

Examples of Diseases Where Mitochondrial DNA Malfunctions

In Parkinson's disease reduced glutathione is depleted due to loss of endogenous polyamines, thus reducing the activity of glutathione peroxidase and permitting oxidative damage. Oxidative damage disintegrates mitochondrial DNA into hundreds of types of mitochondrial DNA fragments which causes release of apoptotic factors and cell death (Ozawa T. et al 1997).

Mitochondrial DNA deletions in brain tissue also increase with age and the increase varies from one brain region to another (Corral-Debrinski M. et al 1992), deletions being highest in the substantia nigra and striatum (Soong N. W. et al 1992) and is also regionally distributed in Alzheimer's disease (Corral-Debrinski M. et al 1994). Environmental agents and nuclear gene defects may cause mitochondrial diseases by predisposing to multiple mitochondrial DNA deletions or quantitative depletions of mitochondrial DNA content A reversible depletion of mitochondrial DNA occurs during zidovudine (AZT) therapy (Arnaudo E. et al 1991). Adriamycin inhibits mitochondrial cytochrome c oxidase (COX if) gene transcription leading to cardiomyopathy (Papadopoulou L. C. et al 1999). Mendelian traits causing qualitative and quantitative changes in mitochondrial DNA have been observed (Zeviani M et al 1995). Nuclear recessive factors can also affect mitochondrial translation and cause age-related respiration deficiency (Isobe K. et al 1998). Wolfram syndrome can be caused by either a mitochondrial or nuclear gene defect (Bu X. et al 1993).

Mitochondrial disorders with neurologic manifestations include; Ptosis, ophthalmoplegia, exercise intolerance, fatigability, myopathy, ataxia, seizures, myoclonus, stroke, optic neuropathy, sensorineural hearing loss, dementias, peripheral neuropathy, headache, dystonia, myelopathy. Mitochondrial disorders with systemic manifestations include; cardiomyopathy, cardiac conduction defects, short stature, cataract, pigmentary retinopathy, metabolic acidosis, nausea and vomiting, hepatopathy, nephropathy, intestinal pseudo-obstruction, pancytopenia, sideroblastic anemia, diabetes mellitus, exocrine pancreatic dysfunction and hypoparathyroidism.

DNA Damage in Neurodegenerative Disorders

Mitochondrial DNA is not protected by histones and lacks a pyrimidine dimer repair system (Clayton DA et al 1974). Mitochondrial DNA has a relatively short half life of six to ten days compared with an up to one month half life of nuclear DNA. The error insertion frequency of polymerase γ is approximately 1 in 7,000 bases, leading to 2-3 mismatched nucleotides per cycle of replication. Hypoxia induces damage to nuclear DNA and to a greater extent to mitochondrial DNA (Englander E. et al 1999). Nuclear and mitochondrial DNA repair declines during aging in neurons and in cortical glial cells (Schmitz C. et al 1999). 8-hydroxyguanosine (8-OHG) immunoreactivity is increased in the substantia nigra, nucleus raphe dorsalis and occulomotor nucleus of Parkinson's disease patients, and 8-HG immunoreactivity is also increased in the substantia nigra of Olivopontine cerebellar degeneration (OCD or MSA) and Lewy body disease patients. Lewy bodies were proposed to be degenerating mitochondria (Gai W. P. et a] 1977). Mitochondria partially though not completely repair DNA damage caused by bleomycin (Shen C. 1995). Polyamines promote repair of X-ray induced DNA strand breaks (Snyder R. D. 1989). Polyamine depletion caused by α-difluoromethylornithine (DFMO) increases the number of strand breaks caused by 1,3-bis(2chloro-ethyl)-1-nitrourea (BCNU) (Cavanaugh P. F. et al 1984). Physiological concentrations of spermine and spermidine prevent single strand DNA breaks induced by superoxide (102) (Khan A. U. et al 1992). L-DOPA and Cu(II) generate reactive oxygen species, conversion of guanine to 8-hydroxyguanine and cause strand breakage of DNA (Husain S. et al 1995). The metal catalyzed oxidation of dopamine and related amines to quinones and semiquinones occurs during pigment deposition and may precipitate cellular damage in Parkinson's and Lou Gehrig's diseases (Levay G. et al 1997). Melanin in association with Cu(II) is also capable of causing DNA strand breakage (Husain S. et al 1997). Copper concentrations in the cerebrospinal fluid of Alzheimer's patients is increased 2.2 fold and caeruloplasmin concentrations is also increased (Bush ALL et al 1994). Copper concentrations are elevated to 0.4 mM and iron and zinc to 1 mM in the neuropil of Alzheimer's brain (Lovell M. et al 1998, Smith M. A. et al 1997).

Mitochondrial DNA content is depleted in Parkinsonian brain and following MPTP administration in experimental animals due to deficient DNA replication in both instances (Miyako K. et al 1997 and 1999). MPP+destabilizes D-loop structure thereby inhibiting the transition from transcription to replication of mitochondrial DNA (Umeda S. et al 2000).

Alzheimer's disease patients brains have decreased levels of mitochondrial DNA, increased levels of 8-OHdeoxyguanosine and increased DNA fragmentation (de la Monte S. M. et al 2000). Increased levels of point mutations, for example at nucleotide pair 4366 in the tRNA^{GLN} gene was observed (Shoffner J. M. et al 1993). The risk of Alzheimer's disease increases when a maternal relative is afflicted with the disease (Duara R. et al 1993, Edland S. D. et al 1996).

DNA damage was proposed as a cause of Lou Gehrig's disease by Bradley W. G et al and deficiency of cytochrome c oxidase activity and a cytochrome c microdeletion were observed by Borthwick G. M. et al (1999) and Comi G. P. et al (1998).

A decreased activity of mitochondrial complex IV and citrate synthase was observed in Olivopontine Cerebellar Degeneration (OCD or MSA) (Schapira A. H. V. 1994, 1998).

Biological Actions of Polyamines that Maintain Brain Function and Prevent Neurodegeneration

However the pathology of several disease states which are described below involves more than the initial DNA damage and correspondingly the influence of therapeutic agents in these diseases involves control of DNA damage and other cellular injuries simultaneously.

I previously reported the ability of 2,3,2 tetramine in Murphy U.S. Pat. No. 5,906,996 to prevent MPTP induced dopamine loss and the applicability of such compounds in the treatment of neurodegeneration, this being notated herein in its entirety by this reference.

A model of neurodegeneration involving Parkinson's, Alzheimer's, Olivopontine Cerebellar Degeneration, Lewy Body, Binswanger's and Lou Gehrig's diseases involving a similar constellation and cascade of events, whereby the final disease is determined by the duration of damage and the anatomical distribution of damage was described. The principal highlights of this pattern of neurodegeneration and its treatment by polyamines are summarized as follows:

The Neurodegenerative Pathway in Parkinson's, Olivopontine Cerebellar Atrophy (MSA), Alzheimer's, Lewy Body, Binswanger's and Lou Gehrig's Diseases

There are five principal aspects of neuronal damage in this pattern of neurodegeneration all of which are prevented by optimized polyamine molecules; Mitochondrial DNA Damage, Growth Factor Functions, Receptor Activities, Energetics and Redox Homeostasis and Deposition of Amyloid.

Cascade of Events in the Pathogenesis of Neurodegeneration:

Mitochondrial DNA is damaged by dopamine and xenobiotics in the presence of reduced levels of naturally occurring polyamines.

Polyamines competitively block the uptake of xenobiotics which depigment pigment Depigmentation releases organic molecules and free metals which damage mitochondrial DNA bases. Polyamines protect DNA from damage by organic molecules by steric interactions (Baeza L. et al 1992). They sequester the metals directly and induce transcription of metallothionein (Goering P. L. et al 1985), the metals being catalytic in reactions damaging DNA bases. They also induce transcription of growth factors such as nerve growth factor, brain derived neuronotrophic factor (Chu P. et al 1995, Gilad G. et al 1989. Polyamines regulate the activity N-methyl-d-aspartate (NMDA) receptor and affect the level of agonism or antagonism at the MK801 ion channel (Beneviste M. Et al 1993, McGurk J. F. 1990) and the activity of protein kinase C (Mezzetti G et al 1988, Moruzzi M. S. et al 1990, 1995).

Polyamines regulate redox homeostasis by binding glutathione (Dubin D. T. 1959). These primary deficits associated with polyamine deficiency cause the neuronal dedifferentiation processes of these diseases via the changes in growth factor levels or ratios, the rapid entry of calcium via the MK801 ion channel and the metabolic consequences by damaged RNA transcripts causing production of defective cytochromes.

Secondarily defective cytochromes are proteolysed and release enkephalin by products and also release free iron into the mitochondrial matrix The iron is leached from damaged calcium laden mitochondria into the cytosol of the neurons. NMDA receptor activation causes excess calcium entry into cells.

Thirdly gross elevation of the free level of a metal such as iron causes displacement of other metals such as copper, nickel, cobalt and lead from sites where they are bound. One or more of these metals overactivate preasapatate proteases (Abraham 199a, 199b, 1992, Black 1989, Blomgren 1989, Chakrabarti 1989, Dawson 1987, Dawson 1988, Edelstein 1988, Hamakubo 1986, Koistra 1984, Matus 1987, Perlmutter L. S. et al 1988, Press E. M. 1960, Rabbazoni B. L. 1992, Rose C. 1988, Rose C. 1989, Scanu A. M. 1987, Whitaker J. N. 1979) which can produce β-amyloid and tangle associated proteins. In Parkinson's Disease and Alzheimer's Disease there is an increase in free copper levels in the absence of an absolute increase in copper levels or more likely an actual decrease in total tissue copper levels due to its loss in the cerebrospinal fluid. The free copper will activate amine oxidase, tyrosinase, copper zinc superoxide dismutase and monoamine oxidase B. The preaspartate proteases may be activated by several divalent metal ions including such as zinc, iron, calcium, cobalt. The literature on these proteases indicates that zinc and calcium and copper are particularly likely. Given a role for divalent metals in activating preaspartate proteases and amyloid production as a tertiary event in this model, it is in concordance with the clinical situation whereby patients present with Parkinson's Disease and subsequently Alzheimer's Disease rather than the converse. In Guamanian Parkinsonian Dementia the plaque formation likewise follows motor neuron and Parkinsonian pathology after many years or decades.

More specifically, therapeutic polyamine compounds like 2,3,2-tetramine have multiple actions on this cascade of events extending from DNA damage to amyloid production;

a) Competitive inhibition of uptake of xenobiotics at the polyamine transport site, such organic molecules being a cause of depigmentation and DNA damage; b) Steric shielding of DNA from organic molecules by compacting DNA, c) Limitation of mitochondrial DNA damage by removal of free copper, iron, nickel, mercury and lead ions by the presence of a polyamine, d) Induction of metallothionein gene transcription; e) Induction of nerve growth factor, brain derived neuronotrophic factor and neuronotrophin-3 gene transcription; f) Regulation of affinity of NMDA receptors and blockade of the MK801 ion channel; g) Inhibition of protein kinase C; h) Mitochondrial reuptake of calcium; i) Binding and conservation of reduced glutathione; j) Induction of ornithine decarboxylase by glutathione; k) Maintenance of the homeostasis of the redox environment in brain; 1) Non toxic chelation of divalent metals in brain, m) Regulation of activity of preaspartate proteases; n) Inhibition of acetylcholinesterase and butyrylcholinesterase; o) Blockade of muscarinic M₂ receptors; p) Maintenance of ratio of membrane phosphatidylcholine: phosphatidylserine ratio, q) Inhibition of superoxide dismutase, amine oxidase, monoamine oxidase B by binding of free copper, r) Regulation of brain polyamine levels in dementias with maintenance of endogenous polyamine levels; s) Blockade of neuronal n and p type calcium channels.

Success therapy must prevent glutathione loss, prevent mitochondrial DNA damage or cytochrome enzyme malfunction, prevent release of metals including calcium from mitochondria, NMDA receptor blockade, prevent hyperpigmentation and ensuing depigmentation, prevent oxidative enzyme and amyloid producing enzyme activation. Polyamines compounds described herein uniquely have the relevant profile of the above actions and prevent MPTP induced dopamine loss in an animal model.

Because none of the changes in Parkinson's or Alzheimer's diseases are pathognomic and because of the overlapping sets of mitochondrial and cytosolic events in Parkinson's disease, Guamnanian Parkinsonian dementia, Alzheimer's disease, Binswanger's diseases, Lewy body disease, hereditary cerebral hemorrhage—Dutch type, Olivopontine cerebellar atrophy and Batten's Disease it is anticipated that these compounds will be beneficial in controlling dementia development The major pathological difference between Parkinson's and Alzheimer's pathological features being the presence of amyloid in Alzheimer's disease and the diseases being closely interlinked by the evolution of Parkinson's disease into Alzheimer's disease with amyloid deposition as the former progresses. At post mortem forty percent of Parkinson brains have amyloid deposits.

The Neurodegeneration Process—Prevention & Treatment by Polyamines:

The following summarizes the principal concurrent and sequential components of neurodegeneration in Parkinson's, Alzheimer's and Lou Gehrig's diseases, the sites of cellular damage and the pivotal relationship between neurotoxins and polyamines in precipitating and preventing neurodegeneration.

Excessive exposure to xenobiotic molecules that migrate into the cell across the polyamine transport pump initiate depolymerization of pigment During depigmentation more organic molecules and stored heavy metals are released intracellularly. The excessive exogenous (xenobiotics) and endogenous quinones and semiquinones (neurotransmitter by products) organics mutate mitochondrial DNA bases randomly when catalyzed by heavy metals.

When mitochondrial DNA is damaged, the cytochrome proteins produced are dysfunctional. Breakdown of these proteins releases iron intramitochondrially and subsequently intracellularly. The inactive cytochromes fail to produce the energy storage compound adenosine triphosphate (ATP) which operates the cell's various metabolic processes.

The metals released from the pigment and the iron from the mitochondria activates various enzymes including amine oxidase that breaks down polyamines and preaspartate proteases that produce amyloid from its precursor protein. Decreasing polyamine levels below a threshold level by excessive amine oxidase activity results in a positive feedback cycle of further polyamine loss because polyamines bind and conserve the peptide glutathione (GSH) that stimulates the rate limiting enzyme of polyamine production, ornithine decarboxylase.

As well as regulating the inflow and outflow of xenobiotics and binding of toxic free metals, polyamines also compact mitochondrial DNA that is not coiled or supercoiled like nuclear DNA; they promote transcription of several neuronal growth factors; they regulate the activities of several cell surface receptor systems including the n-methyl-d-aspartate (NMDA) receptor. All of these components of neurodegeneration can be controlled using an optimized polyamine.

Peripheral Neuropathy

Peripheral neuropathy occurs in association with mitochondrial encephalomyopathies (Chu C. et al 1997). Vacuolar degeneration of dorsal root ganglia cells may consist of degenerating mitochondria Mitochondrial DNA mutations may be caused by lipid peroxidation α-lipoic acid affected improvement in streptozotocin diabetic neuropathy (Low P. A. et al 1997). Glutathione treats experimental diabetic neuropathy (Brabenboer B. et al 1995).

Probucol and Vitamin E improve nerve blood flow and electrophysiology (Cameron N. E. et al 1994, Karasu C. et al 1995). Hydroxytoluene and carvidilol were also effective in preventing damage in diabetic neuropathy (Cameron N. E. et al 1993 and Cotter M. A. et al 1995).

Optic Neuropathy

Optic neuropathy occurs in multiple sclerosis patients and occasionally these multiple sclerosis patients have LHON associated mitochondrial DNA mutations.

Optic neuropathy also occurs from toxic exposure to tobacco and methanol as in Cuban epidemic optic neuropathy (CEON) (Sadun A. and Johns D. R. et al 1994). Methanol leads to formate production that inhibits cytochrome oxidase and adenosine triphosphate production is diminished. Decrease in ATP results in decreased mitochondrial transportation and shutdown of axonal transportation.

Glaucoma

In glaucoma the M ganglion cells of the retina degenerate and there is defective axoplasmic flow (Quigley H. A. 1995). Glutamate is elevated in the vitreous body of glaucoma patients (Dreyer E. B. et al 1996), glutamate being more toxic to M ganglion cells (Dreyer M. et al 1994).

The excitotoxic cascade caused by NMDA receptor activation in the optic nerve results in excess calcium influx, increased nitric oxide synthesis and production of oxygen free radicals (Sucher N. J. et al 1997).

Diabetes Mellitus

Mitochondrial DNA Damage in Diabetes

Mitochondrial DNA content in peripheral blood was observed to be 35% lower in Non Insulin Dependent diabetics (NIDDM) than in controls Lee H. K. et al 1998) and the decline precedes the onset of diabetes. Reduced oxidative disposal of glucose results in insulin resistance in skeletal muscle and/or defective insulin secretion in pancreatic islets. Decreased mitochondrial DNA content impairs fat oxidation in the presence of increased fatty acid availability, fatty acyl CoA accumulates in the cytosol and thus causes insulin resistance (Park K. S. et al 1999).

Streptozotocin causes oxidant mediated repression of mitochondrial transcription (Kristal B. S. et al 1997) and the quantity of mitochondrial DNA decreases in the islets of diabetes prone GK rats (Serraas P. et al 1995). Forty two different mitochondrial DNA point mutations, deletions and substitutions have been associated with NIDDM (Matthews C. E. et al 1998). Mitochondrial DNA mutations such as the M3243 base substitution can also cause maturity onset diabetes of the young (MODY) and auto antibody positive insulin dependent diabetes mellitus (IDDM) (Oka Y. 1993 and 1994). Free radicals can cause deletions of the mitochondrial genome (Wei Y. H. et al 1996). Nitric oxide and hydroxyl radical production in response to environmental agents were proposed as a means of producing mitochondrial DNA damage, expression of mutated proteins which cause MHC restricted immune responses and P cell death in Type 1 diabetes by Gerbitz K. D. (1992). Reductions in β cell numbers and islet amyloidosis containing islet amyloid polypeptide occurs in a high percentage of NIDDM patients (Clark A. et al 1995).

These defects impair oxidative phosphorylation, such impairment diminishing insulin secretion. Treatment with coenzyme Q10 has been reported to be successful in a patient with the M3243 A to G mutation (Suzuki Y. et al 1995). Glucagon secretion is also decreased in diabetes mellitus associated with mitochondrial DNA defects (Odawara M. et al 1996).

Insulin dependent diabetes, autoantibody positive also occurs in patients carrying the M3243 mutation. (Oka Y. et al 1993). 8-hydroxydeoxyguanosine (80HDG) content and extent of deletion of mitochondrial DNA base 4977 deletion correlates with duration of NIDDM and the frequency of diabetic proliferative and simple retinopathy and nephropathy (Suzuki Y. et al 1999). Hyperglycemia causes oxidative damage to the mitochondrial DNA of vascular smooth muscle and endothelial cells precipitating vasculopathy (Fukagawa N. K et al 1999). High insulin levels are also implicated in damaging smooth muscle and endothelial cells (O'Brien S. F. et al 1997). Monosaturated palmitic acid causes DNA fragmentation of rat islet cells in culture. It also reduces the β cell proliferation caused by hyperglycemia. Palmitic acid also induced release of cytochrome c and apoptois of β cells (Maedler K et al 2001).

Exocytosis of Insulin

The methyl ester of succinnic acid may bypass defects in glucose transport, phosphorylation and further catabolism and stimulate insulin secretion and release (McDonald J. et al 1988 and Malaisse W. J. et al 1994). Succinate esters increase the supply of succinnic acid and acetyl CoA to the Krebs cycle (Malaisse W. J. 1993a), they stimulate insulin synthesis and release Malaise W. J. et al 1993b), they increase insulin output at high concentrations of glucose (Akkan A-G. et al 1993), they maintain insulin secretion when β cells are challenged with streptozotocin (Malaisse W. J. 1994), they enhance the inslinotropic effect of hypoglycemic sulfonylureas (Vicent D. et al 1994), they improve the secretory potential of exocrine pancreas when administered prior to streptozotocin (Akkan A. G. et al 1993), they protect against the cytotoxic effect of interleukin-1 (Eizirik D. L. et al 1994) and they do not show any glucagonotropic effect (Vicent D. etal 1994).

Glutamate also stimulates exocytosis of insulin, primarily by an intracellular mechanism acting downstream of mitochondrial metabolism, as oligomycin that abolishes the insulin release response to succinate does not inhibit the insulin release caused by glutamate (Maechler P. et al 2000). Also glutamate induced insulin release seems to require other factors such as ATP induced closure of potassium channels followed by influx of calcium and exocytosis.

Protein Kinase C and Insulin Resistance

Hyperglycemia increases the activity of protein kinase C (Lee T. S. et al 1989). Activation of protein kinase C increases the tram endothelial permeability of proteins such as albumin (Lynch J. J. et al 1990). Albumin, hyperglycemia, H₂O₂ can cause the 4977 bp mitochondrial DNA deletion associated with diabetes (Egawhary, D. N. et al 1995 and Swoboda, B. E. et al 1995). Circulating endothelial cells containing this deletion are particularly common in patients with nephropathy and peripheral vascular disease. The same deletion is also present during aging and more frequently in patients with impaired glucose tolerance or insulin resistance, hyperglycemia and free radicals being precipitants thereof (Liang P. et al 1997).

Triglyceride hydrolysis generates diacylglycerol which activates protein kinase C which promotes serine/threonin phosphorylation thus reducing tyrosine kiinase activity. Feeding animlas high fat diets increases the ratio of membrane bound to cytosolic protein kinase C sixfold. Protein kinase C α, β, ε and δ is increased in muscle of rats fed a fat rich diet (Schmitz-Pfeiffer C. et al 1997) and in regularly fed Goto-Kakizaki rats, a strain of rats with insulin resistance (Avignon A et al 1996). Inhibition of protein kinase C in rat adipocytes prevented insulin resistance (Muller H. K. et al 1991). Protein kinase C 8 is overexpressed in Psammomys preceding the onset of overt insulin resistance and is a prediabetic stage (Ikeda Y et al 1999). Protein kinase C causes retinopathy, neuropathy and nephropathy in diabetes (Koya D et al 1998).

Polyamines and Insulin Concentration

Erythrocyte spermidine levels are elevated in insulin dependent diabetic patients and patients with microalbuinuria and macroalbuminuria and retinopathy (Seghieri G. et al 1992). Spermine oxidase activity is lower in insulin dependent diabetics though not in patients with proliferative retinopathy (Seghieri G. et al 1990). Polyamines are present in high concentrations in B cells and are concentrated in secretory granules (Houggard D. M. et al 1986). Putresine, spermidine and spermine increase synthesis of (pro)insulin, however spermine increases insulin mRNA levels and promotes insulin release (Welsh N et al 1988). Spermine protects the insulin mRNA from degradation (Welsh N. 1990).

Daibetogenic Toxins

Taurine (Trachtman R et al 1995) and vitamin C (Craven P. A. et al 1997) reduced glomerular hypertrophy, albuminuria, glomerular collagen and TGF-01 accumulation in a streptozotocin induced diabetic rat model.

In streptozotocin induced diabetes metal distribution is altered, there are increases in the quantities of hepatic copper, zinc, manganese, renal copper and zinc and plasma zinc. Insulin administration returns the metal levels to within normal ranges (Failla M. I. et al 1981). In contrast to the elevated hepatic and renal zinc concentrations in diabetic pregnant rats, their fetuses have lower concentrations of hepatic zinc Uriu-Hare J. et al 1988). Higher groundwater zinc concentrations reduces the incidence of insulin dependent diabetes mellitus in childhood (Haglund B. et al 1996). Low serum zinc and hyperzincuria have been reported in the initial stages of Type 1 diabetes (Hagglof B. et al 1983). Hyperzincuria and borderline zinc deficiency also occurs in type II diabetes (Kinlaw W. B. et al 1983). Preloading animals with zinc, which induces metallothionein synthesis, metallothionein being a radical scavenger, partially prevents streptozotocin induced diabetes (Yang Y. et al 1994). Elevated metallothionein increased resistance to DNA damage and to depletion of NAD+, increased resistance to hyperglycemia and reduced β cell degranulation and necrosis (Chen H. et al 2001). Metallothionein is highly inducible and does not seem to have deleterious effects at higher concentrations.

In alloxan induced diabetes diethylenetriamine pentaacetic acid inhibits the hyperglycemic response (Grankvist K. et al 1983). Part of the cytoprotective effect of spirohydantoin-derivative aldose reductase inhibitors in diabetes may relate to their ability to chelate copper ions and thus inhibit ascorbic acid oxidation (Jiang Z. Y. et al 1991).

Iron-catalyzed peroxidative reactions may account for the diabetes found as a common side effect of transfusion siderosis, dietary iron overload and idiopathic hemochromatosis McLaren G. D. et al 1983). Plasma copper levels are higher in diabetic patients and are highest in diabetics with angiopathy and diabetics who have alterations in lipid metabolism (Mateo M. C. M. et al 1978, Noto R. et al 1983). Carboxymethyl lysine (CML) levels are twice as high in the skin collagen of diabetics as compared with age matched controls (Dyer G. D. et al), and correlate positively with the presence of retinopathy and nephropathy (McCance D. R. et al 1993).

Matrix metalloproteinase-9 (MMP-9) concentrations are increased in noninsulin dependent diabetes mellitus (NIDDM) prior to development of microalbuminuria (Ebihara L et al 1998). This proteinase is activated by zinc, calcium and oxidative stress.

Treatment with antioxidants polyethylene glycol-superoxide dismutase and N-acetyl-L-cysteine reduces MMP-9 activity (Uemura S. et al 2001). Increased MMP-9 activity is also observed in myocardial infarction, unstable angina and in atherosclerosis.

Polyamines as blockers of uptake of xenobiotics, as molecules which compact DNA and as chelates of redox metals which, redistribute metals to storage sites and induce metallothionein can prevent the damage caused by organic toxins and metal induced redox damage.

Vanadium and Insulin Sensitivity

Vanadium decrease blood glucose and D-3-hydroxybutyrate levels in diabetes, it also restores fluid intake and body weight of diabetic animals. These metabolic effects occur because vanadium decreases P-enolpyruvate carboxykinase (PEPCK) transcription, thus decreasing gluconeogenesis; secondly it decreases tyrosine aminotransferase gene expression, Thirdly it increases expression of glucokinase gene; fourthly it induces pyruvate kinase; fifthly it decreases mitochondrial 3-hydroxy-3-methylglutaryl-CoA synthase (HMGCoAS) gene expression, sixth it decreases the expression of the liver and pancreas glucose-transporter GLUT-2 gene in diabetic animals to the level seen in controls (Valera A et al 2001); seventh it increases the amount of the insulin-sensitive glucose transporter, GLUT4 by stimulating its transcription (Strout H. V. et al); eighth the metabolic effects of vanadium are mediated by inhibition of protein tyrosine phosphatases (PT). Peroxovanadium compounds irreversibly oxidize the thiol group of the essential cysteine at the PTP catalytic site (Fantus I. G. et al 1998). Vanadium is a structural analog of phosphate. Vanadium does not exhibit the growth effects and mitogenic effects of insulin and thus might avoid the macrovascular diseases consequences of hyperinsulinemia and be clinically useful in disease where insulin resistance is caused by defects in the insulin signaling pathway. Vanadium mimics the effects of insulin in restoring G proteins and adenyl cyclase activity increasing cyclic AMP levels. (Anand-Srivastava M. B. et al 1995); ninth vanadyl ion suppresses nitric oxide production by macrophages (Tsuji A. et al 1996); tenth it has a positive cardiac inotropic effect (Heyliger C. E. et al 1985); eleventh vanadium restores albumin mRNA levels in diabetic animals by increasing hepatic nuclear factor 1 (HNF 1) (Barrera Hernandez G. et al 1998); twelfth it restores triiodothyronine T₃ levels (Moustaid N. et al 1991).

In type I diabetes vanadium appears to reverse defects secondary to chronic insulin deficiency and hyperglycemia and may be useful in newly diagnosed diabetics who still have pancreatic reserve (Cam M. C. et al 2000). Vanadium is also P cell protective in streptozotocin diabetic rats (Cam M. C. et al 1999). In type II diabetes vanadium improves glucose tolerance whilst decreasing plasma insulin levels.

Improvement occurs in fasting plasma glucose, glycosylated hemoglobin levels, insulin stimulated glucose uptake and reduction of hepatic glucose output (Cohen N. et al 1995). Free fatty acid and triglyceride levels are controlled more quickly in diabetic animals than glucose levels (Cam M. C. et al 1993). Type I and Type II diabetic patients treated with vanadium had significantly less need for insulin (Goldfine A. B. et al 1995 & 2000).

The toxicity of vanadate was reduced by administering it in chelate form, sodium 4,5 dihydroxybenzene-1,3 disulfonate (Tiron) (Domingo J. L. et al 1995). The organic forms of vanadium corrected the hyperglycemia and impaired hepatic glycolysis more safely and potently than vanadium sulphate (Reul B. A. et al 1999).

Vanadium completed with the biguanide drug metformin was not more effective in lowering blood glucose in streptozotocin treated rats than bis(maltolato)oxovanadium(IV) salts (Lenny C. Y. et al 1999). Vanadate acts as a phosphate analog and binds to phosphoryl transfer enzymes, where it can assume a trigonal bipyramidal structure. Hydrogen peroxide may complex with vandium, forming pervanadate, which may oxidize the catalytic cysteine of tyrosine phsophatase (Huyer G. et al 1997).

Tyrosine Phosphatase Inhibition and Isulin Sensitivity

Tyrosine phosphatases and tyrosine kinases play crucial roles in cellular growth and differentiation, signal transduction, metabolism, motility, cytoskeletal organization, cell cell interaction, gene transcription and the immune response (Zhang Z. 1998, Li L. 2000, den Hertog J. 1999). It is estimated that there may be five hundred such tyrosine phosphatase proteins coded for in the human genome. The catalytic domains of several hundred has been sequenced and consist of an approximate two hundred and forty amino acids in the amino terminal (Walchli S. et al 2000) which contain the active site sequence (I/V)HCXXGXXR(S/T), referred to as a C(X)₅R motif (Dixon J. E 1995). The carboxyterminal is a regulatory domain.

Of the protein tyrosine phosphatases sequenced there are two groups a) transmembrane proteins of 680-2000 amino acids with extracellular domains which are likely to function as receptors for extracellular signals and b) proteins of 360-930 amino acids which appear to be entirely cytoplasmic (Krueger N. et al 1990). Protein tyrosine phosphatase 1B (PT-1B) which regulates insulin sensitivity is associated with microsomal membranes, with its phosphatase domain oriented towards the cytoplasm. The C-terminal 35 amino acids target the endoplasmic reticulum (Frangioni J. V. et al 1992). It could also regulate Endoplasmic reticulum functions such as protein synthesis, post translational protein modification, lipid synthesis and vesicular trafficking. In addition to dephosphorylating autophosphorylated insulin PTP-1B can dephosphorylate epidermal growth factor receptors (Tappia P. S et al 1991, Milarski K. L. et al 1993).

Autoantigens in Diabetes Mellitus

Insulin dependent diabetes mellitus (1DM) antibodies to glutamic acid decarboxylase (a 64-kDa autoantigen) are present in more than seventy percent of newly diagnosed patients and have been detected up to seven years before the onset of clinical disease (Baekkeskov S. et al 1990). The tyrosine phosphatase IA-2 (a 37/40-kDa antigen) was found in fifty four percent of newly diagnosed IDDM patients (Passini N. et al 1995, Payton M. A. et al 1995). Eighty eight percent of IDDM patients had antibodies to one or both of these antigens (Bonifacio E. et al 1995). A further antigen, insulinoma associated protein IA-2β (phogrin), which is an insulin granule membrane tyrosine phosphatase, has been observed in IDDM patients, it being the 37-kDa antigen and the IA-2 being the 40-kDa antigen (Lu J. et al 1996). Phogrin has a high homology with IA-2 protein. Fifty six percent of new onset IDDM patients had antibodies to phogrin (Kawasaki E. et al 1996).

In monozygotic twins the antibodies to IA-2, IA-2ic, GAD₆₅ and ICA were all predictive of diabetes development (Hawa M et al 1997). IA-2 and GAD antibody measurements when used in combination are as clinically useful as islet cell antibodies (ICA) measurements in predicting onset of diabetes (Borg. H. et al 1997). IA-2 antibodies seem to antedate the occurrence of IA-2β antibodies during the onset of type 1 diabetes (Bonifacio E. et al 1998).

Insulin binding antibodies were observed in eighteen percent of IDDM patients (Palmer J. et al 1983). The monosialoganglioside (GM2-1) is expressed at a one hundred fold higher level in pancreatic islets than in the remainder of the pancreas and it is hyperexpressed in mouse islets in the non obese diabetic mouse model (Dotta F. et al 1995). Antibodies to carboxypeptidase, which is a major protein of insulin secretory granules and helps convert proinsulin to insulin, were observed in a prediabetic patient (Castano L. et al 1991). A 38-kDa mitochondrial autoantigen was overexpressed in a newly diagnosed IDDM patient (Arden S. et al 1996).

At the onset of IDDM there is a dose dependent T cell response to IA-2 as measured form peripheral blood lymphocyte samples. The response does not correlate with age, sex or HLA-DR type (Dotta F. et al 1999). Four of five epitopes recognized by IA-2 human monoclonal antibodies were within the PTP like domain of IA-2, which is the most conserved region of tyrosine phosphatase proteins. The fifth epitope was within the juxtamembrane region of IA-2 (Kolm-Litty V. et al 2000).

IA-2 specific IFN-γ production, which is characteristic of a T cell response occurred in spleen cells of non obese diabetic mice (NOD), with development of diabetes a few weeks after the response peaked (Trembleau S. et al 2000).

Low dose streptozotocin induces an immunological, non antigen specific diabetes mellitus. On low dose streptozotocin administration the ICA 512 protein tyrosine phosphatase was decreased on the third day without induction of ICA-specific cytotoxic T cells. Toxic destruction of B cells stimulates recruitment of macrophages and production of monokines such as IL-1 and TNF-α, which have a cytopathic action on islet, cells (Li Z. et al 2000).

Macrophages stimulate T helper cells to release IFN-γ, the cytokine that is most likely responsible for induction of MHC Class 1 expression in the endocrine cells. IFN-γ was observed to induce islet cell MHC antigens and enhance streptozotocin induced diabetes in the CBA mouse model (Campbell L et al 1988).

Protein tyrosine phosphatase 1B levels were increased in obese non diabetics and further increased in obese diabetics. However PTP-1B activity per unit of PTP-1B protein was markedly reduced in obese non diabetics and in obese diabetics. Body mass index correlates with PTP-1B activity per unit of PTP-1B. Thus impaired PTP-1B activity may be pathogenic for insulin resistance (Cheung A. et al 1999). PTPase activity from subcellular fractions from nondiabetic subjects was increased and PTPase activity from obese non insulin diabetics was decreased (Ahmad F. et al 1997). Insulin increases tyrosine phosphatase activity in rat hepatoma (Hashimoto N. et al 1992) and rat L6 muscle cells (Kenner K. A. et al 1993). In the ob/ob mouse model levels of insulin receptor and tyrosine phosphatase PTP-1B decrease such that in muscle the ratio of PTP-1B to insulin receptors increases six fold compared with ob+control mice (Kennedy B. P. et al 2000).

Protein Tyrosine Phosphatase Catalysis

Peroxovanadium compounds are potent inhibitors of PNP-1B and examples such as mpV(2,6pdc) and mpV(pic) wee selective inhibitors, causing lesser inhibition of epidermal growth factor receptor (EGFR) dephophorylation (Posner B. I. et al 1994). The cysteine residue 215 and its surrounding residues from histidine 214 to arginine 221 reside in a hydrophobic pocket which recruits the phosphorylated tyrosine. The alanine 217 and glutamine 262 residues particularly contribute to the hydrophobicity. The cysteine residue is phosphorylated through a thiophosphate linkage during catalytic turnover and the phosphoenzyme intermediate is subsequently hydrolyzed by a water molecule, which attacks the just vacated leaving site. The cysteine residue (Cys215) forms a covalent cysteinyl phophoenzyme intermediate. The Asp 181 acts as a general acid to donate a proton to the phenolic/alcoholic oxygen and forms a network of hydrogen bonds to the phenolic oxygen of phosphotyrosine and a buried water molecule. The Asp residue is positioned to donate a proton to the tyrosine leaving group during the first hydrolysis step. The Asp residue also plays a role as a general base to activate a nucleophilic water molecule during the dephosphorylation step. An arginine plays a role in substrate recognition and transition state stabilization.

Use of a difluorophosphonate, substitution of a naphthalene ring for a phenyl ring in the phosphotyrosyl and addition of a hydroxyl in the naphthyl 4-position enhanced inhibitory potency of PIP-1B inhibitors (Burke T. R. et al 1996). The fluorines introduce hydrogen bonding interactions with the Asp 181-Phe 182 amide nitrogen, displace a water molecule and reduce the second phosphonate ionization constant (pKₐ₂). The hydroxyl group displaces two water molecules. 2-O-tyrosinyl malonate ethers, particularly when containing the difluoro substitution at the methylene bridge had enhanced efficiency as inhibitors (Burke T. R. et al 1996b). Benzylic and negatively charged substituents para to the hydrolyzable phosphate greatly increase affinity for PTPase Montserat J. et al 1996). A non phosphorous PTP inhibitor (2-(oxalyl-amino)-benzoic acid containing a basic nitrogen substituted in the tetrahydropyridine ring forms a salt bridge with Asp-48 of PTP-1B. Most other PTPases contain an asparagine amino acid at this position. This creates an efficacious selective competitive inhibitor of PTP-1B (Iversen L. G. et al 2000). A second aryl phosphate binding site close to the catalytic site has been identified and binds substrates such as phosphotyrosine and bis-(para-phosphophenyl)methane (BPPM) Puius Y. et al 1997). 11-arylbenzo[b]naphtho[2,3-d]furans and 11-arylbenzo[b]naphtho[2,3-d]thiophenes were observed to act as efficacious inhibitors of PTPase (Wrobel J. et al 1999).

Polycations, including polyamines were observed to increase tyrosine phophatase activity (Tonks et al 1988). Conversely inhibition of polyamine synthesis by DFMO was found to increase tyrosine phosphatase and decrease tyrosine phosphorylation and adding putrescine to the medium diminished tyrosine phosphatase activity and increased tyrosine phophorylation (Oetken C. et al 1992).

Tyrosine Phosphatase Inhibitors/PPARα and PPARγ Partial Agonists/Partial Antagonists

Polyamines covalently bind to glutathione, however they also covalently bind with sterols and a spermine coupled cholesterol metabolite was identified in shark. It had potent central appetite suppressant effects in genetically obese mice (Zasloff M. et al 2001).

Prostaglandin J2 is an endogenous of PPARy and stimulates adipocyte differentiation (Wolf G 1996). The thiazolidinedione drugs are PPARy stimulators and may be useful in the treatment of the insulin resistance syndrome otherwise known as cardiovascular dymetabolic syndrome or syndrome X (Fujiwara T. et al 2000). PPARγ is not readily stimulated by fatty acids whereas PPARα in Liver and muscle is (Forman B. M. et al 1996).

Insulin Resistance Syndrome

The insulin resistance syndrome includes hyperinsulinemia, impaired glucose tolerance, hypertension, dyslipidemia, hyperuricemia, high fibrinogen levels and elevated plasminogen activator inhibitor-1 concentrations Heaven G. M. 1993). All these factors are associated with abdominal adiposity and are risk factors for coronary artery disease (Van Gaal L. F. et al 1999). Mitochondrial DNA damage and quantitative loss of mitochondria in preclinical diabetes overactivity of protein kinase C are key events, which precipitate insulin resistance.

Metabolic Effects of Chromium

As with low zinc consumption predisposing to IDDM, dietary chromium deficiency has been associated with development of atherosclerosis and glucose intolerance. Chromium concentration in human tissues decreases very considerably after the first two decades of life. Further chromium excretion by the kidney is increased following oral glucose loading (Schroeder H. A. 1967). Modern diets containing refined carbohydrates have been depleted of their chromium content Chromium concentrations in the hair of insulin dependent diabetic children were significantly lower than in controls (Hambidge K. M. et al 1968). Hepatic chromium concentrations were significantly decreased in diabetics and non significantly in atherosclerotic patients (Morgan J. M. 1972). Patients who died of cardiovascular diseases had lower aortic chromium concentrations than controls (Schroeder H. A. et al 1970). Human subjects with impaired glucose tolerance had significant improvement in impaired glucose tolerance, reduction of the exaggerated insulin response to a glucose load and reduction of serum cholesterol in response to chromium (Freiberg J. M. et al (1975). In spontaneously hypertensive rats chromium lead to a significant reduction in plasma glucose without significant effect on plasma insulin following intraperitoneal glucose challenge (Yoshimoto S. et al 1992). Chromium supplementation in diabetics improves glucose tolerance, decreases blood cholesterol and triglycerides, and increases high density lipoprotein (HDL) (Abraham A. S. et al 1992).

Plasma chromium levels and insulin levels after oral glucose loading were higher in obese controls than in lean controls, plasma chromium levels were similar in obese and lean insulin dependent diabetics (IDD), plasma chromium levels were higher in lean non insulin dependent diabetics (NIDD) than in controls. Chromium levels correlate with body mass index (BMI) and rise in the obese and in non insulin dependent diabetics (NIDD) in response to insulin resistance. Chromium excretion was significantly increased in lean insulin dependent diabetics (IDD) (Bale K. E. et al 1989).

Thus the major biochemical Components of Diabetes Mellitus include, Mitochondrial Dysfunction and energetics dysfunction, Impairment of Exocytosis of Insulin, Impaired Glucose Tolerance and Diminished Insulin Sensitivity with consequent Altered Carbohydrate and Fat Metabolism, Neuronal, Microvascular and Macrovascular Complications.

Atherosclerosis

In the hearts of patients having coronary artery disease the levels of mitochondrial DNA deletions M4977, M7436, M10,422 increase significantly and especially in left ventricle muscle, this area accumulating twenty seven times as many deletions as the left atrium (Corral-Debrinski M et al 1992). Ischemia causes a decrease in reduced glutathione and superoxide dismutase activity in heart (Ferrari R et al 1985). Hearts that have experienced acute myocardial infarction have elevated levels of mitochondrial DNA over controls though lesser elevation than occurs in coronary artery disease hearts (Ferrari R. et al 1996). Reduced pH and increase Pi result from accumulation of lactate and hydrolysis of ATP. Reduced pH and increased Pi downregulate contractility and cause akinesia of the ischemic zone. GF-109293X protects against hypoxia induced apoptosis in cardiac myocytes (Chen S. J. et al 1998).

The severity of clinical symptoms and survival time correlated with mitochondrial DNA defects in cardiomyopathy patients and hundreds of different DNA minicircles were observed (Ozawa T. et al 1995). Decreased activity levels of Complex I, III, IV and V occur in cardiomyopathy patients inheriting mutations or deletions of mitochondrial NDA (Marin-Garcia J. et al 1999) and depletion of mitochondrial DNA (Marin-Garcia J. et al 1988). Fifty percent of patients with hypertrophic cardiomyopathy were observed to have respiratory chain abnormalities (Zeviani M et al 1995). Alcohol, ischemia and adriamycin also cause cardiomyopathy with mitochondrial DNA deletions. Mitochondrial DNA defects occur less frequently in dilated cardiomyopathy as compared with hypertrophic cardiomyopathy (Arbustni E. 1998 and 2000). Coenzyme Q₁₀ has been found to be an effective therapy in cardiomyopathy and in the treatment of congestive heart failure (Langsjoen P. H. et al 1988).

In vascular smooth muscle PPARY activation inhibits matrix metallprotease-9 (MMP-9) expression and acivity Marx N. et al 1998). PPARY agonists stimulate uptake of oxidized low density lipoprotein by macrophages by increasing activity of the scavenger receptor CD36 (Tontonoz P. et al 1998). Troglizatone, rosiglitazone and 15-deoxy-PGJ-2 inhibited migration of vascular smoth muscle and migration of monocytes (Hsuch W. A. 2001). PPARα agonists such as fibrate drugs lowers the progression of aherosclerotic lesiions and PPARγ agonists such as troglitazone decreases intimal thickness in human carotid arteries (Law R et al 1998).

Stroke

Decreased levels of ATP, low pH, increase levels of intracellular glutamate, intracellular calcium ions and free radicals and protein kinase C activity occur during and post stroke. DNA fragmentation and oxidative damage occur (Chen J. et al 1997 and Cui J. et al 2000). Mitochondrial damage and cell death cause release of large quantities of redox metals locally in the area of the lesion. Endoplasmic reticulum releases calcium and this can be prevented in experimental stroke by dantrolene (Tasker R. C. et al 1998) Uric acid, a scavenger of peroxynitrite and hydroxyl radicals (Yu F. et al 1998), vitamin E (Tagami M. et al 1999) and estrogen (Goodman Y. et al 1996) can prevent apoptosis in stroke models. Putrescine, spermine and spermidine protected neurons in the CA1 layer of hippocampus and in the mediolateral body of striatum from degeneration after global ischemia in a gerbil strokemodel (Gilad G. etal 1991) and a syntetic polyamine N,N-di(4aminobutyl)-1-aminoindian being more protective against neuronal damagte post global forebrain ischemia in the gerbil (Gilad G. m., Gilad V. H. 1999). In a transgenic mouse overexpressing ornithine decarboxylase, the increased ornithine decarboxylase and resultatn induction of transcription factors c-Fos and zif-268 in the hippocampus were not damaging (Lukkarainen J et al 1995).

Presbycussis

Presbycussis results from mitochondrial DNA mutations such as the M3243 point mutation (Bonte C. A. et al 1997). Acetyl-carnitine and α-lipoic acid protected rats from developing hearing loss and diminished the quantity of mitochondrial DNA deletions which accumulated during aging (Seidman M. D. et al 2000). These compounds can be effective in upregulating cochlear mitochondrial function.

Cancer

Cell Division/Growth Factors

During the synthesis phase of cell division methionine is increasingly converted to homocysteine thiolactone, thioretinaco is converted to thioco and cobalamin is removed from binding to mitochondrial and endoplasmic reticulum membranes. Thus increased quantities of oxygen radical species are produced. Homocysteic aid is formed by oxidation of homocysteine thiolactone (McCully K. S. 1971). Homocysteic acid stimulates release of growth factors such as insulin like growth factor (Clopath P. et al 1976).

Depletion of Thioretinaco in Aging and Cancer

Depletion of thioretinaco from mitochondrial and microsomal membranes causes increased formation of oxygen radicals and their release within neoplastic and senescent cells (Olszewski A. J. et al 1993). Depletion of thioretinaco from mitochondrial and microsomal membranes causes; excessive homocysteine thiolactone synthesis; increased conversion of thioretinaco to thioco; inhibition of oxidative phosphorylation; and accumulation of toxic oxygen radical species McCully 1994a). Malignant cells accumulate homocysteine thiolactone. Deficient intracellular methionine and adenosyl methionine in malignant cells may result from excessive conversion of methionine to homocysteine lactone.

Metabolites and Retinoic Acid

Folic acid and riboflavin are required for the conversion of homocysteine to methionine. Reduced folate intake is associated with increased incidence of heart disease and stroke. Also DNA damage from hypomethylation occurs due to deficiency of adenosyl methionine.

Pro Carcinogenic and Anti Carcinogenic Compounds

Thioretinaco and thioretinamide are cytostatic in cultured malignant cells (McCully K. S. 1992). Homocysteine thiolactone causes fibrosis, necrosis, inflammation, squamous metaplasia, dysplasia, neoplasia, calcification and angiogenesis (McCully K. S. et al 1989, 1994a). Homocysteine induces apoptosis (Kruman L et al 2000). Secondary increase in homocysteine thiolactone leads to disulphide bond formation with amino acids. Homocysteic acid is produced by from oxidation of homocysteine thiolactone.

Neovascularization

Oxygen radicals cause tissue damage during neovascularization. Arteriosclerosis is observed in the new vasculature as cancer grows and invades. Atherogenesis is correlated with total homocysteine. Homocysteine is correlated with total cholesterol and low density lipoprotein (LDL)+high density lipoprotein (HDL) cholesterol McCully K. S. 1990) Increased synthesis of homocysteine thiolactone enhances atherogenesis because of thiolation of amino acids of apoB of low density lipoprotein producing aggregation and uptake of LDL by nacrophages.

ATP Formation and Oxygen Species Holding

Under normal circumstances the disulfonium form of thioretinaco, in the presence of ascorbate, is the electrophile that catalyzes reduction of radical oxygen species to water, concomitant with binding of ATP from the F1 complex 1994a,b). Binding of the oxygen anions of the proximal and terminal phosphates of ATP to the disulfonium complex releases ATP from the F1 binding site McCully K. S. 1994a). Adenosyl methionine formation and further formation of thioretinaco result from cleavage of the adenosyl triphosphate bond.

Toxic Models of Disease

Paraquat causes cell death in E. coli, which action is promoted by copper (Kohen R. et al 1985) and iron (Korbashi P. et al 1989). Paraquat causes single strand DNA breaks in mouse lymphoblasts (Ross W. E. et al 1979). Zinc displaced a redox metal and was effective in preventing paraquat toxicity in E. coli (Korbashi P. et. al.).

Histidine was successful in preventing MPP⁺ induced damage in E. coli (Haskel Y. et al). MPDP⁺, the monoamine oxidase metabolite of MPTP is also mutagenic (Cashman J. R. (1986). Poly (ADP-ribose) polymerase (PARP) activity is increased causing depletion of NAD⁺ and ATP. PARP inhibitors prevent MPTP induced damage in rodent substantia nigra (Zhang J et al 1995).

Rotenone induces Parkinosnism in animals and is an inhibitor of NADH dehydrogenase component of the electron transport chain Leach C. K. et al 1970, Erikson S. E. 1982, Phillips M. K. et al 1982). Diazoxide induces diabetes by inhibiting pancreatic glycerol phosphate dehydrogenase (MacDonald M. J. 1981 and thus inhibiting insulin release (Steinke J et al 1968).

Streptozotocin (N-methylnitrosocarbamoyl)Dglucosamine) which induces diabetes in animals, reduces DNA synthesis (Rosenkranz H. S. et al 1970) and induces DNA strand breakage (Reusser F 1971). Streptozotocin by causing DNA strand breaks increases poly (ADP-ribose) polymersase (PARP) activity resulting in NAD+ and ATP depletion (Pieper A A et al 1999, Cardinal J. W. et al 1999).

Oxidative metabolism of glucose is impaired after alloxan exposure (Borg L. A. et al 1979). Alloxan induces DNA strand breaks and poly (ADP-ribose) polymerase (PARP) activity and depletion of NAD (Yamamoto R et al 1981a, 1981b and Uchigata Y et al 1982). Alloxan causes oxidation of mitochondrial pyridine nucleotides (Frei B. et al 1985) with efflux of mitochondrial calcium. Alloxan lowers mitchondrial glutathione content of mitochondria (Boquist L. et al 1983). Alloxan inhibits glucose induced insulin release and activates the ATP sensitive K channel (Carroll P. B. et al 1994).

Contrast Media

Contrast media used in radiologic examinations include complexes of the following metals; trivalent gadolinium, iron, trivalent lanthanide (Aime S. et al 2002, Vilringer k, et al 1988 and Desreux J. F. et al 1988), manganese, technetium. Basic requirements for human use are that compound(s) are non ionic (Parvez Z et al 1991, Lloyd K. 1994), do not have COO groups, have OH groups in various positions around the molecule (Almen T. 1990), and are water-soluble. Secondary composition possibilities are that they may be monomers, dimers, trimers or tetramers (Morris T. 1993), may be incorporated into liposomes, will have low viscosity, will exhibit low osmolality (Matthai W. H. 1994), and have a particle size between 0.6 and 3 microns to avoid capillary embolism.

The toxicity of contrast media is caused by the following characteristics and actions; binding to proteins, enzyme inhibition, histamine release, alterations in electrolyte environment, hyperosmolality, prolonging whole blood clotting time in a dose dependent manner, inhibiting aggregation of platelets, opening of blood brain barrier, release of vasoactive substances from endothelial cells, activation of complement, alteration of Gibbs Donnan equilibrium, reduction of plasma calcium and magnesium, inhibition of cholinesterase, stimulation of prostaglandin release, immune system response, vasovagal response, platelet activation, alteration in secondary messenger systems, inhibition of clotting factors, lipid solubility and membrane alterations. The toxicity of iodine contrast media has caused interest in development of other metal complexes as alternatives for specific and broader uses in human and veterinary medicine.

Chain polyamine (Kim E. E. et al 1981) and polyazomacrocyclic polyamines (Sherry A. D. et al, Kiefer G. E. et al) have been used successfully as contrast media The biphenyl family of polyamines synthesized herein may have broad clinical applications as contrast media

An iron polyamine complex may be used in hepatic MRI imaging Zhang. XL. et al 2002, Chang D. et al 2002).

A manganese polyamine complex may be used as liver and pancreas contrast MRI agent amongst other uses (Gong J. et al 2002, Diehl S. J. et al 1999, Wang C. et al 1998). A liposome preparation of the complex can be used.

A gadolinium polyamine complex may be used for angiography, intraarticular examinations and hepatobiliary MRU It has no renal toxicity as compared with iodine media and can be used in patients who have had previous anaphylactic reactions to iodine media(Spinosa D. J. et al 2002).

A technetium polyamine complex may be used in detection and evaluation of myocardial ischemia patients. The chain polyamine triethylene tetramine has been used as a technetium gastric contrast media (Kim E. E. et al 1981).

### SUMMARY OF THE INVENTION

The invention is a process for synthesizing polyamine compounds via a series of substitution reactions, optimizing the bioavailability and biological activities of the compounds, and their use as therapeutic agents for the treatment of Parkinson's disease, Alzheimer's disease, Lou Gehrig's disease, Binswanger's disease, Olivopontine Cerebellar Degeneration, Lewy Body disease, Diabetes, Stroke, Atherosclerosis, Myocardial Ischemia, Cardiomyopathy, Nephropathy, Ischemia, Glaucoma, Presbycussis, Cancer, Osteoporosis, Rheimatoid Arthritis, Inflammatory Bowel Disease, Multiple Sclerosis and Toxin Exposure. Tetraamines and polyamines produced herein are compounds that act as bases and which can be prepared by the reaction of acyclic and cyclic amines or alkyl halides with a variety of substrates that will add to the amines or displace the halides. These tetraamines fall into a number of structural classes. These classes are: (1) predominately linear tetraamines and polyamines linked by 1,3-propylene and/or ethylene groups; (2) predominately branched tetraamines and polyamines linked by 1,3-propylene and/or ethylene groups; (3) cyclic polyamines linked by 1,3-propylene and/or ethylene groups; (4) combinations of linear, branched and cyclic polyamines linked by one or more 1,3-propylene and/or ethylene groups, (5) substituted polyamines, (6) polyamines derivatized to formtyrosine phosphatase inhibitor molecules and/or PPAR partial agonists—partial antagonists, with linear or branched chains attached and ((7) polyamine derivatives of 2,2′-diaminobiphenyl with linear or branched chains attached Further, the linked tetraamines may have one or more pendant alkyl, aryl cycloalkyl or heterocyclic moieties attached to the nitrogens.

Accordingly, in one aspect the invention is directed to compounds of the formula: 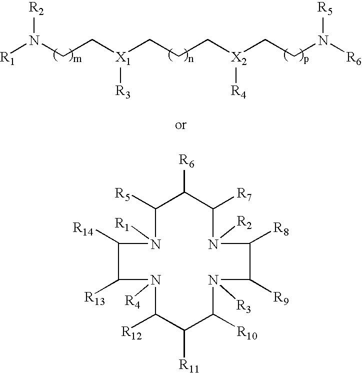
Wherein
R₁ and R₂ may be the same or different and are hydrogen, alkyl, aryl, cycloalkyl, amino acid, glutathione, uric acid, ascorbic acid, taurine, estrogen, dehydroepiandrosterone, probucol, vitamin E, hydroxytoluene, carvidilol, α-lipoic acid, α-tocopherol, ubiquinone, phylloquinone, β-carotene, meanadione, glutamate, succinate, acetyl-L-carnitine, co-enzyme Q, lazeroids, polyphenolic flavonoids, homocysteine, menaquinone, idebenone, dantrolene, —(CH₂)ₙ[XCH₂)ₙ]NH₂— wherein n=3-6 and X=nitrogen, sulfur, phosphorous or carbon, or heterocycle wherein R₁ and R₂ taken together are —(CH₂XCH₂)ₙ— wherein n=36 and X=nitrogen, sulfur, phosporous or carbon. R₃ and R₄ may be the same or different and are hydrogen, alkyl, aryl, cycloalkyl, amino acid, glutathione, uric acid, ascorbic acid, taurine, estrogen, dehydroepiandrosterone, probucol, vitamin E, hydroxytoluene, carvidilol, α-lipoic acid, α-tocopherol, ubiquinone, phylloquinone, β-carotene, meanadione, glutamate, succinate, acetyl-L-carnitine, coenzyme Q, lazeroids, polyphenolic flavonoids, homocysteine, menaquinone, idebenone, dantrolene or heterocycle wherein R₃ and R₄ taken together are —(CH₂XCH₂)ₙ— wherein n=36 and X=nitrogen, sulfur, phosporous or carbon R₅ and R₆ may be the same or different and are hydrogen, alkyl, aryl, cycloalkyl, amino acid, glutathione, uric acid, ascorbic acid, taurine, estrogen, dehydroepiandrosterone, probucol vitamin E, hydroxytoluene, carvidilol, α-lipoic acid, α-tocopherol, ubiquinone, phylloquinone, β-carotene, meanadione, glutamate, succinate, acetyl-L-carnitine, co-enzyme Q, lazeroids, polyphenolic flavonoids, homocysteine, menaquinone, idebenone, dantrolene —(CH₂)ₙ[XCH₂)NH₂— wherein n=36 and X=nitrogen, sulfur, phosporous or carbon, or heterocycle wherein R₅ and R₆ taken together are —(CH₂XCH₂)ₙ— wherein n=3-6 and X=nitrogen, sulfur, phosporous or carbon. R₇, R₈, R₉, R₁₀, R₁₁, R₁₂, R₁₃, and R₁₄, may be the same or different and are hydrogen, alkyl, aryl, cycloalkyl, amino acid, glutathione, uric acid, ascorbic acid, taurine, estrogen, dehydroepiandrosterone, probucol, vitamin E, hydroxytoluene, carvidilol, α-lipoic acid, α-tocopherol, ubiquinone, phylloquinone, β-carotene, meanadione, glutamate, succinate, acetyl-L-carnitine, co-enzyme Q, lazeroids, polyphenolic flavonoids, homocysteine, menaquinone, idebenone, dantrolene —(CH₂)ₙ[XCH₂)ₙ]NH₂— wherein n=3-6 and X=nitrogen, sulfur, phosporous or carbon, or heterocycle wherein R₅ and Rr taken together are —(CH₂XCH₂)ₙ— wherein n=3-6 and X=nitrogen, sulfur, phosporous or carbon.

M, n, and p may be the same or different and are bridging groups of variable length from 3-12 carbons.

X₁ and X₂ may be the same or different and are nitrogen, sulfur, phosporous or carbon.

As used herein, “alkyl” has its conventional meaning as a straight chain or branched chain saturated hydrocarbyl residue such as methyl, ethyl, propyl, isopropyl, isobutyl, t-butyl, octyl, decyl and the like. The alkyl substituents of the invention are of 1 to 12 carbons which may be substituted with 1 to 2 substitutents.

“Cycloalkyl” refers to a cyclic allyl structure containing 3 to 25 carbon atoms. The cyclic structure may have alkyl substituents at any position. Representative groups include cyclopropyl, cyclopentyl, cyclohexyl, 4-methylcyclohexyl, cyclooctyl and the like.

“Aryl” refers to aromatic ring systems such as phenyl, naphthyl, pyridyl, quinolyl, indolyl and the like; aryl alkyl refers to aryl residues linked to the position indicated through an alkyl residue.

“Heterocycle” refers to ringed moieties with rings of 3-12 atoms and which contain nitrogen, sulfur, phosphorus or oxygen.

As shown from the above structures, examples include derivatives of 1,3-bis-[(2′-aminoethyl)-amino]propane (referred to hereafter as 2,3,2-tetraamine); 1,4-bis-[(3′-aminopropyl) amino]butane (referred to as 3,3,3-tetraamine); and 1,4,8,11-Tetraazacyclotetradecane (cyclam). Specific examples include N, N′,N″,N′″-tetramethyl 2,3,2-tetramine; N,N′″-diethyl 2,3,2-tetramine; N,N′″-Dipiperidyl-2,3,2-tetramine, N,N′,N″,N′″-tetramethylcyclam and N,N′,N″,N′″-tetraadamantylcyclam.

Particularly preferred embodiments of R₁ and R₄ are piperidine, piperizine, or adamantane. In this embodiment, N₁ and N₄ are part of the piperidine or piperazine rings while in the adamantane case, N₁ and N₄ are appended from the rings.

It will be understood that the compounds of 1 and 2, since they contain a basic amine group, form salts with non-toxic acids and such salts are included within the scope of this invention. These salts may enhance the pharmaceutical application of the compounds. Representative of such salts are the hydrochloride, hydrobromide, sulfate, phosphate, acetate, lactate, glutamate, succinate, propionate, tartrate, salicylate, citrate and bicarbonate.

There are three structural motifs that are being exploited in this invention. 1,3-bis-[(2′-aminoethyl)-amino]propane (2,3,2-tetramine) and its derivatives are tetramines that are known to have a large number of physiological actions. They are well known binders of metal ions and form very stable complexes with a variety of transition metals. Secondly, polyazamacrocycles such as 1,4,8,11-tetramethyl-1,4,8,11-tetrazacyclotetradecane (cyclam) are of considerable interest due to their ability to form strong complexes with transition metals such as copper, cobalt, iron, zinc, cadmium, manganese and chromium.

Accordingly, in a second aspect the invention is directed to compounds of the formula: 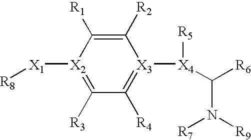
Wherein
R₁-R₄ may be the same or different and are hydrogen, alkyl, aryl, cycloalkyl, hydroxyl, thiol, amino acid, glutathione, phosphate, phosphonates, uric acid, ascorbic acid, taurine, estrogen, dehydroepiandrosterone, probucol, vitamin E, hydroxytoluene, carvidilol, α-lipoic acid, α-tocopherol, ubiquinone, phylloquinone, β-carotene, meanadione, succinate, acetyl-L-carnitine, coenzyme Q, lazeroids, polyphenolic flavonoids, —(CH₂)ₙ[XCH₂)ₙ]NH₂— wherin n=3-12 and X=nitrogen, sulfur, phosporous or carbon, or heterocycle wherein R₁ and R₂ taken together are —(CH₂XCH₂)ₙ— wherein n=3-12 and X=nitrogen, sulfur, phosphorous or carbon. R₅ and R₅ may be the same or different and are hydrogen, alkyl, aryl, cycloalkyl, hydroxyl, thiol, amino acid, glutathione, phosphate, phosphontes, uric acid, ascorbic acid, taurine, estrogen, dehydroepiandrosterone, probucol, vitamin E, hydroxytoluene, carvidilol, α-lipoic acid, α-tocopherol, ubiquinone, phylloquinone, β-carotene, meanadione, succinate, acetyl-L-carnitine, co-enzyme Q, lazeroids, polyphenolic flavonoids, or heterocycle wherein R₃ and R₄ taken together are —(CH₂XCH₂)ₙ— wherein n=3-12 and X=nitrogen, sulfur, phosphorous or carbon. R₇ and R₈ may be the same or different and are hydrogen, alkyl, aryl, cycloalkyl, hydroxyl, thiol, amino acid, glutathione, phosphate, phosphonate, uric acid, ascorbic acid, taurine, estrogen, dehydroepiandrosterone, probucol, vitamin E, hydroxytoluene, carvidilol, α-lipoic acid, α-tocopherol, ubiquinone, phylloquinone, β-carotene, meanadione, succinate, acetyl-L-carnitine, co-enzyme Q, lazeroids, polyphenolic flavonoids, —(CH₂)ₙ[XCH₂)ₙ]NH₂— wherin n=3-12 and X=nitrogen, sulfur, phosporous or carbon, or heterocycle wherin R₅ and R₆ taken together are —(CH₂XCH₂)ₙ— wherin n=3-12 and X=nitrogen, sulfur, phosphorous or carbon. R₉ is hydrogen, alkyl, aryl, cycloalkyl, hydroxyl, thiol, amino acid, glutathione, phosphate, phosphonate, uric acid, ascorbic acid, taurine, estrogen, dehydroepiandrosterone, probucol, vitamin E, hydroxytoluene, carvidilol, α-lipoic acid, α-tocopherol, ubiquinone, phylloquinone, β-carotene, meanadione, succinate, acetyl-L-carnitine, co-enzyme Q, lazeroids, polyphenolic flavonoids, —(CH₂)ₙ[XCH₂)ₙ]NH₂— wherin n=3-12 and X=nitrogen, sulfur, phosporous or carbon, or heterocycle wherein R₅ and R₆ taken together are {CH₂XCH₂)ₙ— wherein n=3-12 and X=nitrogen, sulfur, phosphorous or carbon.

X₁-X₄ may be the same or different and are nitrogen, sulfur, phosphorous or carbon. 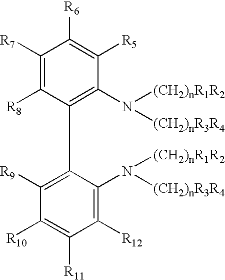
wherein
R₁-R₄ may be the same or different and are hydrogen, alkyl, aryl, cycloalkyl, hydroxyl, thiol, amino acid, pyridine, pyrazole, 3,5-dimethylpyrazole, imidazole, quinoline, phenol, 4-X-phenol and 5-X-phenol where X=chloro, bromo, nitro, methyl, ethyl, methoxy, amino, hydroxy; glutathione, phosphate, phosphonates, uric acid, ascorbic acid, taurine, estrogen, dehydroepiandrosterone, probucol, vitamin E, hydroxytoluene, carvidilol, α-lipoic acid, α-tocopherol, ubiquinone, phylloquinone, β-carotene, meanadione, succinate, acetyl-L-carnitine, co-enzyme Q, lazeroids, polyphenolic flavonoids, —(CH₂)ₙ[XCH₂)ₙ]NH₂— wherin n=3-6 and X=nitrogen, sulfur, phosporous or carbon, or heterocycle wherin R₁ and R₂ taken together are —(CH₂XCH₂)ₙ— wherin n=3-6 and X=nitrogen, sulfur, phosphorous or carbon. R₅ and R₅ may be the same or different and are hydrogen, alkyl, aryl, cycloalkyl, hydroxyl, thiol, amino acid, pyridine, pyrazole, 3,5 dimethylpyrazole, imidazole, quinoline, phenol, 4-X-phenol and 5-X-phenol where X=chloro, bromo, nitro, methyl, ethyl, methoxy, amino, hydroxy; glutathione, phosphate, phosphontes, uric acid, ascorbic acid, taurine, estrogen, dehydroepiandrosterone, probucol, vitamin E, hydroxytoluene, carvidilol, α-lipoic acid, α-tocopherol, ubiquinone, phylloquinone, β-carotene, meanadione, succinate, acetyl-L-carnitine, co-enzyme Q, lazeroids, polyphenolic flavonoids, or heterocycle wherin R₃ and R₄ taken together are —(CH₂XCH₂)ₙ— wherin n=3-6 and X=nitrogen, sulfur, phosphorous or carbon.
R₅-R₁₂ may be the same or different and are hydrogen, alkyl, aryl, cycloalkyl, hydroxyl, thiol, amino acid, pyridine, pyrazole, imidazole, quinoline, phenol, 4-X-phenol and 5-X-phenol where X=chloro, bromo, nitro, methyl, ethyl, methoxy, amino, hydroxy; glutathione, phosphate, phosphonate, uric acid, ascorbic acid, taurine, estrogen, dehydroepiandrosterone, probucol, vitamin E, hydroxytoluene, carvidilol, α-lipoic acid, α-tocopherol, ubiquinone, phylloquinone, 1-carotene, meanadione, succinate, acetyl-L-carnitine, co-enzyme Q, lazeroids, polyphenolic flavonoids, —(CH₂)ₙ[XCH₂)ₙ]NH₂— wherin n=3-6 and X=nitrogen, sulfur, phosporous or coon, or heterocycle wherin R₅ and & taken together are —(CH₂XCH₂)ₙ— wherin n=3-6 and X=nitrogen, sulfur, phosphorous or carbon.
N is an integer with values from 0-10.

### BRIEF DESCRIPTION OF THE DRAWINGS

FIGS. 1-41 depict reaction schemes for the preparation of a variety of intermediates and the subsequent polyamines described in the invention and FIGS. 42-46 depict the effect of polyamines on toxin induced bacterial inactivation as follows:

FIG. 1 Route of Synthesis of 1,3-bis-[(2′-aminoethyl)-amino]propane and analogous compounds

FIG. 2 Route of Synthesis of [2-methylethylamino)ethyl](3-{[2-(methylamino)ethyl]amino}propyl)amine and analogous compounds

FIG. 3 Route of Synthesis of (2-piperidylethyl)-{3-[(2-piperidylethyl)amino]propyl}amine and analogous compounds

FIG. 4 Route of Synthesis of (2-piperazinylethyl){3-[(2-piperazinylethyl)amino]propyl}amine and analogous compounds

FIG. 5 (2-aminoethyl)(3-[(2-aminoethyl)methylamino]propyl}methylamine and analogous compounds

FIG. 6 [2-(bicyclo[3.3.1]non-3-ylamino)ethyl](3-{2-(bicyclo[3.3.1]non-3-ylamino)ethyl]amino}propyl)amine and analogous compounds

FIG. 7 (2-aminoethyl){3-[(2-aminoethyl)amino]-1-methylbutyl}amine and analogous compounds

FIG. 8 (2-pyridylmethyl){3-[(2-pyridylmethyl)amino]propyl}amine and analogous compounds

FIG. 9 methyl(3-[methyl(2-pyridylmethyl)amino]propyl}(2-pyridylmethyl)amine and analogous compounds

FIG. 10 [2-dimethylamino)ethyl](3-{[2-(dimethylamino)ethyl]methylamino}propyl)methylamine and analogous compounds

FIG. 11 2-[3-2-aminoethylthio)propylthio]ethylamine and analogous compounds

FIG. 12 1,4,8,11-tetraaza-1,4,8,11-tetramethylcyclotetradecane and analogous compounds

FIG. 13 1,4,8,11-tetraaza-1,4,8,11-tetra(2-piperidylethyl)cyclotetradecane and analogous compounds

FIG. 14 1,4,8,11-tetraaza-1,4,8,11-tetrabicyclo[3.3.1]non-3-ylcyclotetradecane and analogous compounds

FIG. 15 1,4,8,11-tetraaza-1,4,8,11-tetraethylcyclotetradecane and analogous compounds

FIG. 16 N,N′-(2′-dimethylphosphinoethyl)-propylenediamine and analagous compounds

FIG. 17 3-(3-(2-aminoethoxy)propoxy)propylamine and analagous compounds

FIG. 18 Vanadyl 2,3,2-Tetramine and analagous compounds

FIG. 19 Chromium 2,3,2-Tetramine and analagous compounds

FIG. 20 Vanadyl (2-piperidylethyl)-{3-[(2-piperidylethyl)amino]propyl}amine)(Cl)₂ and analagous compounds

FIG. 21 Chromium (2-piperidylethyl)-{3-[(2-piperidylethyl)amino]propyl}amine (Cl)₂]Cl and analagous compounds

FIG. 22 Vanadyl (1,4,8,11-tetraaza-1,4,8,11-tetrabicyclo[3.3.1]non-3-ylcyclotetra decane) (Cl)₂ and analagous compounds

FIG. 23 (Chromium 1,4,8,11-tetraaza-1,4,8,11-tetrabicyclo[3.3.1]non-3-ylcyclotetradecane(Cl)₂]Cl and analagous compounds

FIG. 24 p-(Phosphonomethyl)-DL-phenylalanine-Butylamine salt and analagous compounds

FIG. 25 2-amino-N(2-{[3-(2-amino-3-4-phosphonomethylphenyl)propanolylamino]ethyl)amino)propyl]amino}ethyl-3-(4-phosphonomethylphenyl)propamide and analagous compounds

FIG. 26 2,2′-diamino (bis-N,N′-pyridylmethyl)biphenyl and analagous compounds

FIG. 27 2,2′-diamino(bis-N,N-pyridylmethyl)-6,6′-dimethylbiphenyl and analagous compounds

FIG. 28 2,2′-diamino (bis-N,N′-quinilylmethyl)biphenyl and analagous compounds

FIG. 29 [(3,5-dimethylpyrazolyl)methyl][2-2-{[(3,5dimethylpyrazolyl) methyl]amino}phenyl)phenyl]amine and analagous compounds

FIG. 30 2-{[(2-{2-[(2-pyridylmethylamino]phenyl}-phenyl)amino]methyl}phenol and analagous compounds

FIG. 31 2-({[2-2-{[2hydroxyphenyl)methyl]amino}phenyl)phenyl]amino}methyl)phenol and analagous compounds

FIG. 32 4-methyl-2-{[(2-{2-[(2-pyridylmethylamino]phenyl}-phenyl)amino]methyl}phenol and analagous compounds

FIG. 33 3-nitro-2-{([(2-{2-[(2-pyridylmethylamino]phenyl}-phenyl)amino]methyl}phenol and analagous compounds

FIG. 34 4-chloro-2-{[(2-{2-[(2-pyridylmethylamino]phenyl}-phenyl)amino]methyl}phenol and analagous compounds

FIG. 35 2,amino-3-(-(4phosphonomethylphenyl)-N-2-{-2-[benzylamio]phenyl}phenyl)propamide and analagous compounds

FIG. 36 Manganese (2,2diamino (bis-N,N′-quinilylmethyl)biphenyl)(Cl)₂ and analagous compounds

FIG. 37 Iron (4-chloro-2-{[(2-{2-[(2-pyridylmethylamino]phenyl}-phenyl)amino]methyl}phenol)(Cl)₂]Cl and analagous compounds

FIG. 38 Vanadium (2,2′-diamino(bis-N,N′-pyridylmethyl)biphenyl)Cl₂ and analagous compounds

FIG. 39 Gadolinium (2,2′-diamino(bis-N,N′-pyridylmethyl)biphenyl)Cl₂]Cl and analagous compounds

FIG. 40 Chromium (2-({[2-(2-{[2-hydroxyphenyl)methyl]amino}phenyl) phenyl]amino}methyl)phenol)Cl)₂]Cl and analagous compounds

FIG. 41 Schematic of 2,3,2, tetramine structure, 1,3-bis-[(2′-aminoethyl)-amino]propane

FIG. 42 Effect of Spermidine on Diazoxide-induced Bacterial Inactivation

FIG. 43 Effect of 2,3,2-piperidine on Diazoxide-induced Bacterial Inactivation

FIG. 44 Effect of 2,3,2-pyridine on Diazoxide-Induced Bacterial inactivation

FIG. 45 Effect of 2,3,2diCH₃ on Diazoxide-Induced Bacterial Inactivation

FIG. 46 Effect of Cyclam Adamantane on Diazoxide-Induced Bacterial Inactivation

### DESCRIPTION OF PREFERRED EMBODIMENTS

### Heats of Formation

Among the reasons for the selection of compounds to be used for these formulations are the results of a series of calculations using heats of formations of the molecules. The relative stables of the compounds were determined in order to predict which would lead to the most stable metal complexes when they react with metals such as copper, cobalt, iron, zinc, cadmium, manganese and chromium These metals are of particular interest due to their importance in neurological and other diseases.

Heats of formation (ΔH°) are calculated by looking at the formation of a compound from its constituent atoms. The lower the heat of formation, the more stable is the compound. The assumption in this computational work is that the calculated heats of formation for the complexes will correlate with the ability of the organic compound to complex with metal ions in biological systems. The more strongly the binding occurs, the more likely it is that the organic molecule will interact with the metal ion of choice. There are other factors that enter into the actual binding ability of the organic molecules, but heats of formation help suggest how different organic molecules might behave. By varying the organic molecules, the heats of formation for the complexes can be compared and correlations between the stability of the complexes and the structure of the complexes can be made. The relative stabilities of a representative survey of organic compounds is shown in Table I while the heats of formation for the metal complexes are shown in Tables II-VIII.

TABLE I
Heats of Formation of Organic Compounds

| Compound | ΔH° (Kcal/mol) |
|---|---|
| 2,3,2-tetramine | −18.24 |
| 2,2,2-tetramine | −17.09 |
| 3,3,3-tetramine | −32.70 |
| 2,3,2-methylated on N1/N4 | −13.81 |
| 2,3,2-methylated on N2/N3 | −10.35 |
| 2,3,2-piperidine | −32.47 |
| 2,3,2-piperizine | 4.33 |
| 2,3,2-tetra sulfur | −26.25 |
| cyclam | −15.65 |
| cyclam-methylated | 18.73 |
| cyclam-adamantane | −40.02 |

TABLE II
Heats of Formation of Copper Complexes

| Compound | ΔH° (Kcal/mol) |
|---|---|
| Cu 2,3,2-tetramine | 244.10 |
| Cu 2,2,2-tetramine | 252.36 |
| Cu 3,3,3-tetramine | 224.16 |
| Cu 2,3,2-methylated on N1/N4 | 243.98 |
| Cu 2,3,2-methylated on N2/N3 | 241.42 |
| Cu 2,3,2-isopropyl on N1/N4 | 207.69 |
| Cu 2,3,2-isopropyl on N2/N3 | 250.17 |
| Cu 2,3,2-dibenzyl on N2/N3 | 314.08 |
| Cu 2,3,2-tetramethyl | 273.85 |
| Cu 2,3,2-tetraisopropyl | 229.83 |
| Cu 2,3,2-benzylated | 380.10 |
| Cu 2,3,2-piperidine | 255.10 |
| Cu 2,3,2-piperizine | 288.68 |
| Cu 2,3,2-adamantane | 269.53 |
| Cu 2,3,2-methyls on carbons 5/7 | 227.45 |
| Cu 2,3,2-tetra sulfur | 210.42 |
| Cu cyclam | 260.20 |
| Cu cyclam-methylated | 298.97 |
| Cu cyclam-benzylated | 405.60 |
| Cu cyclam-adamantane | 254.55 |
| Cu cyclam-isopropyl | 271.59 |
| Cu cyclam-S4 | 207.15 |
| Cu cyclen | 285.10 |
| Cu cyclam 3,3,3 | 245.28 |

TABLE III
Heats of Formation of Iron Complexes

| Compound | ΔH° (Kcal/mol) |
|---|---|
| Fe 2,3,2 | 12.16 |
| Fe 2,2,2 | 37.16 |
| Fe 3,3,3 | −1.39 |
| Fe 2,3,2-methylated on N1/N4 | −8.19 |
| Fe 2,3,2-piperidine | −54.23 |
| Fe 2,3,2-piperizine | −18.51 |
| Fe 2,3,2-adamantane | −19.16 |
| Fe 2,3,2-methyls on carbons 5/7 | 7.99 |
| Fe 2,3,2-tetra sulfur | 87.39 |
| Fe cyclam | −5.75 |
| Fe cyclam-methylated | −69.53 |
| Fe cyclam-adamantane | −92.82 |
| Fe cyclam-isopropyl | −83.02 |
| Fe cyclam-S4 | 137.13 |
| Fe cyclen | 17.76 |
| Fe cyclam 3,3,3 | −31.73 |

TABLE IV
Heats of Formation of Zinc Complexes

| Compound | ΔH° (Kcal/mol) |
|---|---|
| Zn 2,3,2 | 355.75 |
| Zn 2,2,2 | 352.45 |
| Zn 3,3,3 | 328.73 |
| Zn 2,3,2-methylated on N1/N4 | 336.55 |
| Zn 2,3,2-isopropyl on N1/N4 | 316.18 |
| Zn 2,3,2-isopropyl on N2/N3 | 330.81 |
| Zn 2,3,2-tetramethyl | 351.00 |
| Zn 2,3,2-benzlyated | 478.96 |
| Zn 2,3,2-piperizine | 351.70 |
| Zn 2,3,2-methyls on carbons 5/7 | 342.21 |
| Zn 2,3,2-tetra sulfur | 329.15 |
| Zn cyclam | 358.25 |
| Zn cyclam-methylated | 388.64 |
| Zn cyclam-benzylated | 485.39 |
| Zn cyclam-adamantane | 347.52 |
| Zn cyclam-isopropyl | 330.81 |
| Zn cyclam-S4 | 339.04 |
| Zn cyclam 3,3,3 | 351.89 |

TABLE V
Heats of Formation of Manganese Complexes

| Compound | ΔH° (Kcal/mol) |
|---|---|
| Mn 2,3,2 | 266.79 |
| Mn 2,2,2 | 235.44 |
| Mn 3,3,3 | 194.42 |
| Mh 2,3,2-tetra sulfur | 264.50 |
| Mn cyclam | 215.97 |
| Mn cyclam-methylated | 198.40 |
| Mn cyclam-S4 | 248.57 |

TABLE VI
Heats of Formation of Cobalt Complexes

| Compound | ΔH° (Kcal/mol) |
|---|---|
| Co 2,3,2 | −1250.81 |
| Co 2,2,2 | −1236.41 |
| Co 3,3,3 | −1265.92 |
| Co 2,3,2-methylated on N1/N4 | −1269.13 |
| Co 2,3,2-piperidine | −1300.69 |
| Co 2,3,2-adamantane | −1250.92 |
| Co 2,3,2-methyls on carbons 5/7 | −1268.45 |
| Co 2,3,2-tetra sulfur | −1258.52 |
| Co cyclam | −1187.9 |
| Co cyclam-methylated | −1265.64 |
| Co cyclam-isopropyl | |
| Co cyclam-S4 | −1265.56 |

TABLE VII
Heats of Formation of Cadmium Complexes

| Compound | ΔH° (Kcal/mol) |
|---|---|
| Cd 2,3,2 | 393.21 |
| Cd 2,2,2 | 401.00 |
| Cd 3,3,3 | 382.04 |
| Cd 2,3,2-isopropyl on N1/N4 | 366.86 |
| Cd 2,3,2-isopropyl on N2/N3 | 376.40 |
| Cd 2,3,2-piperidine | 374.06 |
| Cd 2,3,2-adamantane | 354.51 |
| Cd 2,3,2-tetra sulfur | 357.79 |
| Cd cyclam | 411.95 |
| Cd cyclam-isopropyl | 376.40 |
| Cd cyclam-S4 | 356.13 |

TABLE VIII
Heats of Formation of Chromium Complexes

| Compound | ΔH° (Kcal/mol) |
|---|---|
| Cr 2,3,2 | 398.73 |
| Cr 2,3,2-isopropyl on N1/N4 | 379.87 |
| Cr 2,3,2-piperidine | 403.22 |
| Cr cyclam | 399.99 |
| Cr cyclam-isopropyl | 430.05 |

This tabular data can be analyzed by comparing the various structural features of the molecules as in examples 19 to 24 below.

### Preparation of the Invention Compounds

There are numerous compounds described in the invention but in general, the invention compounds are obtained by converting the starting di- or tetramine of the formula:

A variety of reactions were used to prepare the compounds. Compound 1 was prepared via a nucleophilic substitution reaction followed by conversion of the free amine to its HCl salt. The amine acts as the nucleophile in displacing the di-alkyl halide, a reaction of general utility. Compound 2 also involved a nucleophilic substitution reaction, this time done in basic solution with a protection/deprotection sequence also involved in the synthesis. The use of acetyl groups to protect the amines could be exploited to alkylate tetramines.

Compounds 3 and 4 were synthesized by having 1,3-diaminopropane serve as the nucleophile with displacement occurring on the α carbon to the piperidine or piperizine. The β position is particularly susceptible to nucleophilic attack in molecules of this type. Other heterocyclic moieties could be added in similar fashion starting from the appropriate Methyl heterocycle in this fashion.

The theme of using amines to attack alkyl halides in nucleophilic substitution reactions was also exploited in the formation of 6 and 14. The 1-position in the bromoadamantane is much more reactive than expected and so the adamantane moiety could be added to numerous amines in this fashion Compound 7 involves a novel preparation of an existing compound as we reversed the nature of the nucleophile and the electrophile to lead to high yields of the product. In the case described, the 1,3-substituted portion is the alkyl halide while the amine is used to form the terminal nitrogens.

Compounds 8 and 9 were prepared using substitution reactions rather than the previously reported (for 8) imine formation reaction followed by a reduction. The α-carbon on the pyridine ring is extremely reactive due to resonance stabilization of any intermediate formed. This is a general approach and numerous other aromatic heterocycles could be added in this fashion.

We have continued the take advantage of nucleophilic substitution reactions to prepare 11 with the electrophilic 2-chloroethylamine. Once again, this scheme illustrates the extreme reactivity of the carbon on amines when used to do substitution reactions. 2-chloroethylamine could be added to many amines to form other tetraamines including many that are not symmetrical.

Compound 13 was prepared in a fashion similar to that used to synthesize 3. The staring amine here is the macrocyclic cyclam This reaction illustrates the power of using macrocycles in these schemes as the substitution led cleanly to the tetramine. Compound 15 was prepared under strongly basic conditions using the anion of the cyclam as the nucleophile attacking an alkyl halide. Certainly any primary alkyl halide could be substituted in this sequence. Phosphine also can be incorporated into these molecules as been done for Compound 16. This molecule was prepared via the use of an addition/reduction sequence starting with an amine. This reaction could be used on any number of amines covered in this patent. This was done for the preparation of compound 17 where oxygens were incorporated into the internal positions of the molecule.

Compounds 1-17 can be used to make metal complexes. Examples include the preparation of the vanadium complexes 18, 20 and 22 where 2,3,2-tetramine is converted into their vanadium complexes by treatment with a vanadium precursor. Compounds 19, 21 and 23 were prepared in similar fashion starting with a chromium precursor. Any number of metal complexes such as copper, cobalt, iron, manganese could be prepared from any of the compounds 1-17 by treating these compounds with the appropriate metal salt followed by isolation of the metal complex.

Compound 24 is a tyrosine phosphatase inhibitor molecule that was prepared in this work. It has also been attached to polyamines via a protection-substitution-deprotection sequence resulting in islation of 25 and 35. These novel compounds include both the polyamine backbone portion along with the tyrosine-phosphate portion.

Compound 26 incorporates the biphenyl moiety into a polyamine compound This compound is prepared by a nucleophilic substitution reaction of the biphenyl precursor with the chloromethylated pyridine. The a position of the heterocyclic pyridine is particularly reactive and we take advantage of this fact in the synthesis of 26.

The related compound 27 was prepared in a two step process by first forming the imine that is isolated and purified followed by reduction. This two step reaction sequence is also used to prepare 28, 30, 31, 32, 33 and 34 from the appropriate substituted heterocycle and the substituted biphenyl. Large numbers of other imines could be formed and converted to the desired amines using a similar sequence of steps.

Compound 29 was synthesized by an unusual nucleophilic substitution using a hydroxy group as the leaving group from a hydroxymethylpyrazole in its reaction a substituted biphenyl.

Compounds 36-40 are metal complexes prepared from the compounds described above. These Mn, Fe, V, Gd and Cr complexes are representative of the utility of compounds 24-35 as electron donors to metal ions. Numerous other metal complexes could be prepared. 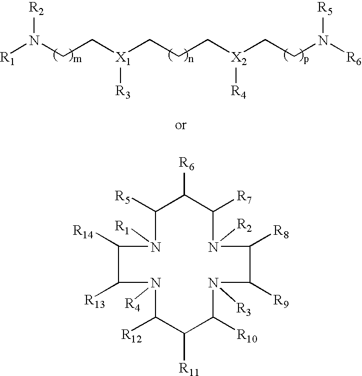
(where A and B equal hydrogen or alkyl and m, n, and p may be the same or different) to the corresponding N-substituted compound by treating these compounds with an alkyl halide under conditions that affect the conversion.
R₁, R₂, R₃, R₄, R₅, R₆, R₇, R₉, R₉, R₁₀, R₁₁, R₁₂, R₁₃, and R₁₄, may be different and are hydrogen, alkyl, aryl, cycloalkyl, amino acid, glutathione, uric acid, ascorbic acid, taurine, estrogen, dehydroepiandrosterone, probucol, vitamin E, hydroxytoluene, carvidilol, α-lipoic acid, α-tocopherol, ubiquinone, phylloquinone, β-carotene, meanadione, glutamate, succinate, acetyl-L-carnitine, co-enzyme Q, lazeroids, polyphenolic flavonoids, homocysteine, menaquinone, idebenone, dantrolene —(CH₂)ₙ[XCH₂)ₙ]NH₂— wherein n=3-6 and X=nitrogen, sulfur, phosphorous or carbon, or heterocycle wherein R₅ and R₆ taken together are —(CH₂XCH₂)ₙ— wherein n=3-6 and X=nitrogen, sulfur, phosporous or carbon.
M, n, and p may be the same or different and are bridging groups of variable length from 3-12 carbons.
X₁ and X₂ may be the same or different and are nitrogen, sulfur, phosporous or carbon

Accordingly, in a second aspect the invention is directed to compounds of the formula: 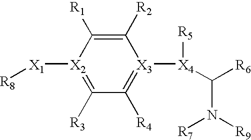
Wherein
R₁-R₄ may be the same or different and are hydrogen, alkyl aryl, cycloalkyl, hydroxyl, thiol, amino acid, glutathione, phosphate, phosphonates, uric acid, ascorbic acid, taurine, estrogen, dehydroepiandrosterone, probucol, vitamin E, hydroxytoluene, carvidilol, α-lipoic acid, α-tocopherol, ubiquinone, phylloquinone, β-carotene, meanadione, succinate, acetyl-L-carnitine, co-enzyme Q, lazeroids, polyphenolic flavonoids, —(CH₂)ₙ[CH₂)ₙ]NH₂— wherin n=3-12 and X=nitrogen, sulfur, phosporous or carbon, or heterocycle wherin R₁ and R₂ taken together are —(CH₂XCH₂)ₙ— wherin n=3-12 and X=nitrogen, sulfur, phosphorous or carbon. R₅ and R₅ may be the same or different and are hydrogen, alkyl, aryl, cycloalkyl, hydroxyl, thiol, amino acid, glutathione, phosphate, phosphontes, uric acid, ascorbic acid, taurine, estrogen, dehydroepiandrosterone, probucol, vitamin E, hydroxytoluene, carvidilol, α-lipoic acid, α-tocopherol, ubiquinone, phylloquinone, β-carotene, meanadione, succinate, acetyl-L-carnitine, enzyme Q, lazeroids, polyphenolic flavonoids, or heterocycle wherin R₃ and R₄ taken together are —(CH₂XCH₂)ₙ— wherin n=3-12 and X=nitrogen, sulfur, phosphorous or carbon.
R₇ and R₈ may be the same or different and are hydrogen, alkyl, aryl, cycloalkyl, hydroxyl, thiol, amino acid, glutathione, phosphate, phosphonate, uric acid, ascorbic acid, taurine, estrogen, dehydroepiandrosterone, probucol, vitamin E, hydroxytoluene, carvidilol, α-lipoic acid, α-tocopherol, ubiquinone, phylloquinone, β-carotene, meanadione, succinate, acetyl-L-carnitine, co-enzyme Q, lazeroids, polyphenolic flavonoids, —(CH₂)ₙ[XCH₂)ₙ]NH₂— wherin n=3-12 and X=nitrogen, sulfur, phosporous or carbon, or heterocycle wherin R₅ and R₆ taken together are —(CH₂XCH₂)ₙ— wherin n=3-12 and X=nitrogen, sulfur, phosphorous or carbon. R₉ is hydrogen, alkyl, aryl, cycloakyl, hydroxyl, thiol, amino acid, glutathione, phosphate, phosphonate, uric acid, ascorbic acid, taurine, estrogen, dehydroepiandrosterone, probucol, vitamin E, hydroxytoluene, carvidilol, α-lipoic acid, α-tocopherol, ubiquinone, phylloquinone, β-carotene, meanadione, succinate, acetyl-L-carnitine, co-enzyme Q, lazeroids, polyphenolic flavonoids, —(CH₂)ₙ[XCH₂)ₙ]NH₂— wherin n=3-12 and X=nitrogen, sulfur, phosporous or carbon, or heterocycle wherin R₅ and R₆ taken together are —(CH₂XCH₂)ₙ— wherin n=3-12 and X=nitrogen, sulfur, phosphorous or carbon. X₁-X₄ may be the same or different and are nitrogen, sulfur, phosphorous or carbon. or 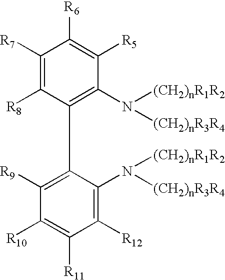
   Wherein
R₁-R₄ may be the same or different and are hydrogen, alkyl, aryl, cycloalkyl, hydroxyl, thiol, amino acid, pyridine, pyrazole, 3,5-dimethylpyrazole, imidazole, quinoline, phenol, 4-X-phenol and 5-X-phenol where X=chloro, bromo, nitro, methyl, ethyl, methoxy, amino, hydroxy, glutathione, phosphate, phosphonates, uric acid, ascorbic acid, taurine, estrogen, dehydroepiandrosterone, probucol, vitamin E, hydroxytoluene, carvidilol, α-lipoic acid, α-tocopherol, ubiquinone, phylloquinone, β-carotene, meanadione, succinate, acetyl-L-carnitine, co-enzyme Q, lazeroids, polyphenolic flavonoids, —(CH₂)ₙ[XCH₂)ₙ]NH₂— wherin n=3-6 and X=nitrogen, sulfur, phosporous or carbon, or heterocycle wherin R₁ and R₂ taken together are —(CH₂XCH₂)ₙ— wherin n=3-6 and X=nitrogen, sulfur, phosphorous or carbon. R₅ and R₅ may be the same or different and are hydrogen, alkyl, aryl, cycloalkyl, hydroxyl, thiol, amino acid, pyridine, pyrazole, 3,5-dimethylpyrazole, imidazole, quinoline, phenol, 4-X-phenol and 5-X-phenol where X=chloro, bromo, nitro, methyl, ethyl, methoxy, amino, hydroxy; glutathione, phosphate, phosphontes, uric acid, ascorbic acid, taurine, estrogen, dehydroepiandrosterone, probucol, vitamin E, hydroxytoluene, carvidilol, α-lipoic acid, α-tocopherol, ubiquinone, phylloquinone, β-carotene, meanadione, succinate, acetyl-L-carnitine, co-enzyme Q, lazeroids, polyphenolic flavonoids, or heterocycle wherin R₃ and R₄ taken together are —(CH₂XCH₂)ₙ— wherin n=3-6 and X=nitrogen, sulfur, phosphorous or carbon. R₅-R₁₂ may be the same or different and are hydrogen, alkyl, aryl, cycloalkyl, hydroxyl, thiol, amino acid, pyridine, pyrazole, imidazole, quinoline, phenol, 4-X-phenol and 5-X-phenol where X=chloro, bromo, nitro, methyl, ethyl, methoxy, amino, hydroxy; glutathione, phosphate, phosphonate, uric acid, ascorbic acid, taurine, estrogen, dehydroepiandrosterone, probucol, vitamin E, hydroxytoluene, carvidilol, α-lipoic acid, α-tocopherol, ubiquinone, phylloquinone, β-carotene, meanadione, succinate, acetyl-L-carnitine, co-enzyme Q, lazeroids, polyphenolic flavonoids, —(CH₂)ₙ[XCH₂)ₙ]NH₂— wherin n=3-6 and X=nitrogen, sulfur, phosporous or carbon, or heterocycle wherin R₅ and R₆ taken together are —(CH₂XCH₂)ₙ— wherin n=3-6 and X=nitrogen, sulfur, phosphorous or carbon.
N is an integer with values from 0-10.

There are also instances where some forms of 2,3,2-tetramine need to be protected prior to adding on the various groups as is true for 2 and 6. For the cyclam type molecules, nucleophilic substitution reactions were generally used to prepare the compounds (compounds 10-15).

Compounds 2, 3, 4, 6, 9, 13, 14, 16, 17, 18, 19, 20, 21, 22, 23, 24, 25, 26, 27, 28, 29, 32, 33, 34, 35, 36, 37, 38, 39, 40 are prepared in this invention for the first time. Of the known compounds described here, most (5, 7, 8, 10, 11, 12, 15, 26, 30, 31) have been prepared in a fashion significantly different than that found in the literature. In addition, many of the compounds covered in the invention but not used as examples have not been prepared elsewhere and will be prepared as part of this invention for the first time.

The base compound 1,3-bis-[(2′-aminoethyl)-amino]propane, 1, was prepared in a fashion similar to that found in the literature (Van Alphen J. 1936). However, in the original literature preparation, an impurity was found that significantly reduced the purity of the product. Subsequent preparations have taken a number of tacks to lead to a pure product. We have eliminated this problem by developing a purification strategy that works through the hydrochloride salt that leads to a single product of very high purity.

In order to prepare the novel compound 2, ([2{methylethylamino)ethyl](3-{[2-(methylamino)ethyl]amino}propyl)amine), protection of the more reactive terminal nitrogens 1 and 4 as their acetyl derivatives was performed prior to methylation of nitrogens 2 and 3. Deprotection of the acetyl groups with KOH led to the desired compound.

Compounds 3 ((2-piperidylethyl)-{3-[(2-piperidylethyl)amino]propyl}amine) and 4 ((2-piperazinylethyl)-{3-[(2-piperazinylethyl)amino]propyl}amine) were made in similar fashion through the nucleophilic substitution reaction of 1,3-diaminopropane with 1-(2-chloroethyl)piperidine (to give 3) or 1-(2-chloroethyl)piperizine (to give 4). Numerous other tetramines are accessible through similar reactions where the nature of the amine is varied.

(2-aminoethyl){3-[(2-aminoethyl)methylamino]propyl}methylamine, 5, is a known compound (Barefield E. K et al 1976) but was prepared in a novel way here. The physical properties of our compound do not match those found in the literature but the NMR data in the literature in no way fits the structure of the compound while our NMR and mass spectral data are consistent with the formulation.

Compound 6, [2-bicyclo[3.3.1]non-3-ylamino)ethyl](3-{2-bicyclo[3.3.1]non-3-ylamino)ethyl]amino}propyl)amine and compound 14, 1,4,8,11-tetraaza-1,4,8,11-tetrabicyclo[3.3.1]non-3-ylcyclotetradecane were prepared in a similar way. Direct amination (Krumkalns E. V. et al 1968) of 1-bromoadamantane with the appropriate amine led to the pure products.

Compound 7, (2-aminoethyl){3-[(2-aminoethyl)amino]-1-methylbutyl}amine, has been prepared previously through the reaction of N,N′-bis(chloroacetyl)-2,4-pentanediamine with methylamine (Mikukami F. 1975). We have prepared the compound in a completely different way by following a similar procedure as that used for compound 1.

(2-pyridylmethyl){3-[(2-pyridylmethyl)amino]propyl}amine, 8, is a known compound but was prepared by a completely different procedure than that found in the literature. Instead of making this compound via the two step process of a Schiff base condensation of pyridine-2-carboxaldehyde with 1,3-propanediamine followed by a reduction reaction (Fischer H. R. et al 1984), we prepared it directly through a nucleophilic substitution of picolyl chloride with 1,3-propanediamine. This results in higher overall yields since we employ a one step process.

The preparation of the novel compound 9, methyl(3-[methyl(2-pyridylmethyl)amino]propyl)(2-pyridylmethyl)amine, was performed in a fashion similar to that used to synthesize 8. The product was of high purity and its analytical data matched the desired structure.

Compound 10, [2-(dimethylamino)ethyl](3-{[2-(dimethylamino)ethyl]methylamino}propyl)methylamin, was prepared by the literature procedure (Golub G. et al 1992.) and the synthesis resulted in a high yield of a pure product. Although the literature did not supply physical data for the compound, our results are consistent with the structure of the compound.

2-[3-2-aminoethylthio)propylthio]ethylamine, 11, is a known compound (Hay R. W. et al 1975) but was prepared by a novel procedure here. Nucleophilic substitution of 1,3-dimercaptopropane with 2-chloroethyamine resulted in formation of 11 that had physical properties similar to those reported.

The preparation of 1,4,8,11-tetraaza-1,4,8,11-tetramethylcyclotetradecane, 12, was performed in a manner similar to that found in the literature (Barefield K et al 1973). The analytical data for this compound matches that found previously.

Compound 13, 1,4,8,11-tetraaza-1,4,8,11-tetra(2-piperidylethyl)cyclotetradecane, was prepared from cyclam through a nucleophilic substitution in a fashion similar to the one we use to prepare compound 4. Many other derivatives of cyclam could be prepared using this type of reaction.

1,4,8,11-tetraaza-1,4,8,11-tetraethylcyclotetradecane, 15, is a known compound (Oberholzer M. R. et al 1995) but was prepared here by a modified procedure using similar reagents but with different reactions conditions and purification steps.

Compound 16 is a novel compound that incorporates phosphorous into the molecule in the place of the two nitrogens. This internal substitution is done via addition/reduction process and could be changed to include oxygen or other donors if desired.

Compound 17, 3-(3-(2-aminoethoxy)propoxy)propylamine, is a novel compound that incorporates oxygen into the molecule in place of two of the nitrogens of 2,3,2-tetramine. This internal substitution is done via Williamson-type chemistry starting with a di-alkoxide and a di-alkyl halide.

The preparation of the novel vanadium complexes 18, 20 and 22 occurs in straight-forward fashion by mixing a vanadium precursor with the appropriate starting material. The novel chromium complexes 19, 21, and 23 are prepared in similar fashion using a chromium precursor.

Compound 24, p-Phosphonomethyl)-DL-phenylalanine, is a known compound (Marseigne, L, et al 1988) that was prepared in six steps starting from p-cyanobenzylbromide. This compound was converted into its butylamine salt by treatment of 24 with an aqueous solution of butylamine followed by precipitation. Numerous other salts of this compound could be prepared, all of which would have substantially modified properties.

Compound 24 was used as one of the precursors for the preparation of 25, 2-amino-N-(2-{[3-(2-amino-3-4-phosphonomethylphenyl)propanolylamino]ethyl}amino)propyl]amino}ethyl-3-(4-phosphonomethylphenyl)propamide, by reacting Boc-protected 24 with 2,3,2-tetramine, 1, after activation of the carboxylic acid group. This novel compound 25 incorporates the tyrosine phosphate inhibitor group into the tetramine backbone. Compound 24 could be added to any number of the amines described here to form novel polyamine compounds.

The preparation of 2,2′ diamino (bis-N,N-pyridylmethyl)biphenyl, 26, has been described previously (Malachowski M. R. et al 1999). The novel compound 27, 2,2′-diamino(bis-N,N′-pyridylmethyl)-6,6-dimethylbiphenyl illustrates an example of incorporating additional substituents onto the biphenyl rings, in this case methyl groups in the 6,6′-positions. This will force the rings further out of plane. Compound 27 was made by an Ullman coupling of substituted benzenes followed by a two-step process involving catalytic hydrogenation and then reaction of the amine with 2-pyridinecarboxaldehyde and NaBH₄.

The new compound 28, 2,2′-diamino (bis-N,N-quinilylmethyl)biphenyl was prepared by the formation of the intermediate imine via a substitution-elimination pathway starting with 2,2′-diaminobiphenyl and 2quinoline carboxaldehyde. This reaction was followed by a reduction of the imine using NaBH₄. Compound 28 is a novel compound related to compound 26 where the pyridine rings are replaced with the bulkier quinoline rigs.

Compound 29, [(3,5-dimethylpyrazolyl)methyl][2-2-{[(3,5-dimethylpyrazolyl)methyl]amino}phenyl)phenyl]amine is prepared for the first time and incorporates pyrazole rings via a nucleophilic substitution pathway, using 2,2′-diaminobiphenyl and 3,5-dimethyl-N-hydroxymethylpyrazole as the stating materials. Compound 30, 2-{[(2-{2-[(2-pyridylmethylamino]phenyl}-phenyl)amino]methyl}phenol, is a known compound that is formed via the preparation of the Schiff-base followed by reduction with sodium borohydride (Goodwin k et al 1960).

It is also of value to generate polyamine compounds with less symmetry so the two rings of the biphenyls are substituted with different groups. Compound 31, 2-({[2-(2-{[2-hydroxyphenyl)methyl]amino}phenyl)phenyl]amino}methyl)phenol is a known compound that contains three nitrogens and one oxygen (Melby L. R et al 1975). Derivatives of 31 where the phenol ring is substituted with a CH₃ in the 4position led to the new compound 32, 4-methyl-2-{[(2-{2-[(2-pyridylmethylamino]phenyl}-phenyl)amino]methyl}phenol. This compound was formed by the reaction of N-2-pyridylmethyl)-2,2′-diamino biphenyl with the substituted salicylaldehyde.

Compound 33, 3-nitro-2-{[(2-{2-[(2-pyridylmethylamino]phenyl}-phenyl)amino]methyl}phenol was synthesized in similar fashion by treating the nitro-substituted salicylaldehyde with the same polyamine as used for 32 while 34, 4-chloro-2-{[(2-{2-[(2-pyridylmethylamino]phenyl}-phenyl)amino]methyl}phenol is prepared with the suitable chloro-substituted precursor compound. This addition-elimination-reduction sequence could be exploited for a large number of substituted salicylaldehydes.

Compound 35, 2, amino-3-(-(4-phosphonomethylphenyl)-N-(2-{-2-[benzylamino]phenyl)phenyl)propamide, incorporates components of the tyrosine phosphate inhibitor and the biphenyl rings to form the polyamine. 35 was prepared by reacting N-(2-pyridylmethyl)-2,2′diamino biphenyl with the Boc-protected amino acid molecule 24 followed by deprotection with acid. Numerous related polyamines that incorporate the biphenyl backbone can be prepared in this fashion.

Various metal complexes of the examples described above were prepared Compound 36 resulted from the reaction of MICl₂ with 2,2′diamino (bis-N,N′-quinilylmethyl)biphenyl (28) in a substitution reaction. Compound 37 incorporates iron into a complex with 34 via the reaction of FeCl₃ while 38 is formed by reacting VCl₂ with Compound 26. The gadolinium complex 39 was prepared via the reaction of 26 with GdCl₃. Compound 40 was prepared by the reaction of CrCl₃ with 30 resulting in the chromium complex Numerous other metals such as copper, cobalt, technetium and other transition metals reacting with compounds 1-17 and 24-35 should lead smoothly to novel metal complexes.

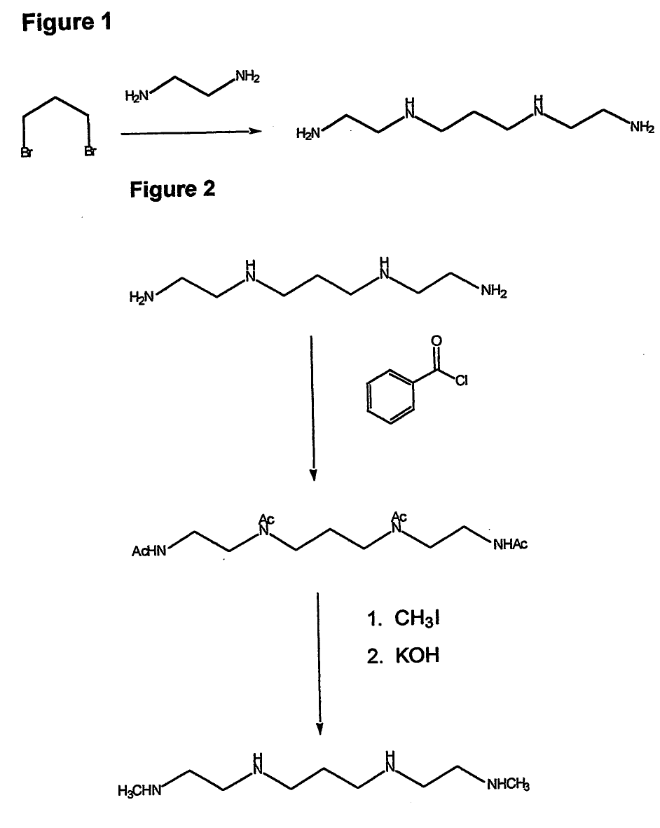

Compounds 1 to 40 correspond with FIGS. 1 to 40 and Examples 1 to 40.

The following examples are intended to illustrate but not to limit the number of compounds within the scope of the invention.

### EXAMPLE 1

### 1,3bis-[(2′-aminoethyl)-amino]propane [FIG. 1]

A mixture of 15 g of 1,3-dibromopropane and 50 mL of absolute EtOH was added slowly to 25 g of 1,2 diaminoethane hydrate. The mixture immediately became warm. It was then heated to 500C for 1 hour, 20 g of KCl added and the heating continued for 30 minutes. The mixture was filtered from the KBr and distilled at reduced pressure. The residue formed two layers that were separated. The top layer was distilled and the product had a b.p. of 115-116° C., (1 mm). The compound was further purified by converting the free amine to its tetrahydrochloride salt by addition of 6M. HCl. The melting point of the salt was 278-283° C. It was converted back to its free amine by treatment with NH₄OH. Mass spectral analysis showed a m/e=160. ¹H NMR (CDCl₃): δ 1.26 (6H, s), 1.60 (2H, quin), 2.60 (4H, t), 2.71 (8H, t).

### EXAMPLE 2

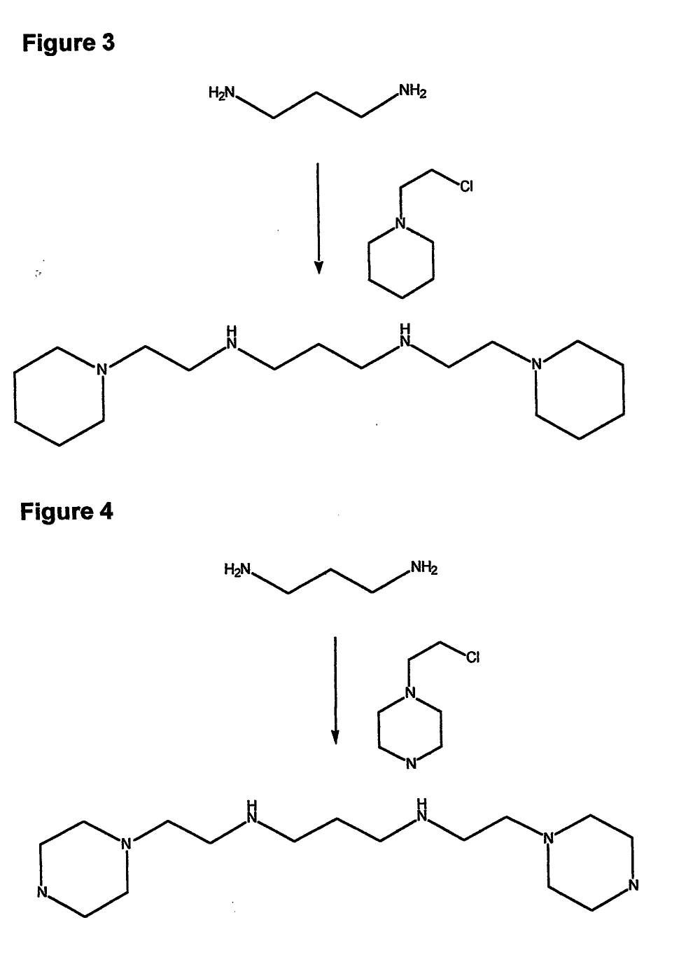

### [12(methylethylamino)ethyl](3-[(methylamino)ethyl]amino)propyl)amine [FIG. 2]

A mixture of 0.37 g (0.0155 mol) of magnesium turnings, 5.0 g (0.031 mol) of 1,3-bis-[(2′-aminoethyl)-amino]propane, 50 mL of benzene and 3.76 g (0.047 mol) of acetyl chloride is heated under reflux for 2 h. The reaction mixture is cooled in an ice bath and the liquid portion is decanted into a separatory funnel. The residue in the flask is washed twice with 50 mL portions of ether, and the ethereal solution is poured over ice. The ether-water mixture is then added to the benzene solution in the separatory funnel and separated. The organic phase is washed once with 50 mL of 5% sodium bicarbonate and once with water and dried over CaCl₂. The solution is filtered and used without further purification.

A magnetically stirred mixture of 5.0 g (8.67 mmol) of the acetylated 2,3,2-tetramine prepared above and 2.0 g (80.7 mmol) of sodium hydride in 75 mL of N,N-dimethylformamide was heated at 60° C. under N₂ for 3 h. The resultant mixture was treated with 19.8 g (0.164 mol) of iodomethane and stirred at 50° C. After 24 h at 50° C., the reaction was quenched by the addition of 95% EtOR Volatiles were removed at reduced pressure and 50 mL of water was added to the residue. The product was extracted with three 50 mL portions of chloroform. The combined organic extracts were successively washed with water and NaCl, dried over anhydrous sodium sulfate, and concentrated to give 6.3 g of yellowish oil. The oil was purified by flash chromatography with 1:4 hexanes-ethyl acetate as the eluent to give acetylated [2-(methylethylamino)ethyl](3-{[2-(methylamino)ethyl]amino}propyl)amine as an oil.

A stirred solution of 3.0 g (4.54 mmol) of acetylated [2-(methylethylamino)ethyl](3-{[2-(methylamino)ethyl]amino)propyl)amine, 10.0 g (0.178 mol) of potassium hydroxide, 70 mL of methanol and 15 mL of water was heated under reflux for 24 h. The methanol was removed at reduced pressure and the product was extracted into 2×50 mL of ether. The combined extracts were washed with NaCl, dried over sodium sulfate and concentrated under vacuum. The crude mixture was purified by flash chromatography with 5:1 hexanes-ethyl acetate as the eluent. After evaporation of the solvents, 0.79 g (71%) of the product was obtained as a colorless oil. Mass spectral analysis showed a m/e=244. ¹H NMR (CDCl₃): δ1.03 (12H, d), 1.26 (6H, s), 1.60 (2H, quin), 2.60 (4H, t), 2.71 (8H, t), 3.23 (2H, m).

### EXAMPLE 3

### (2-piperidylethyl)-(3-[(2-piperidylethyl)amino]propyl}amine [FIG. 3]

To a mixture of 0.5 g (6.75 mmol) of 1,3-diaminopropane and 50 mL of absolute EtOH was added 1.62 g (40.5 mmol) of NaOH To this solution was added dropwise 2.48 g (13.45 mmol) of 1-(2-chloroethyl)piperidine in 50 mL of EtOH over 30 min. The solution was allowed to stir for 24 h The solvent was evaporated and the residue was extracted with 2×50 mL of CH₂Cl₂, dried over Na₂SO₄, and evaporated to dryness. The compound was purified by converting it to its hydrochloride salt by addition of HCl. The melting point of the salt was >300° C. It was converted back to its free amine by treatment with NH₄OH. The resultant oil (1.04 g, 52%) was analyzed. Mass spectral analysis showed a m/e=297 (M+1). ¹H NMR (CDCl₃): δ 1.40-1.82 (14H, m), 2.40-2.58 (14H, quin), 2.60-2.72 (10H, m).

### EXAMPLE 4

### (2-piperazinylethyl)-3-[(2piperazinylethyl)amino]propyl)amine [FIG. 4]

To a mixture of 0.5 g (6.75 mmol) of 1,3-diaminopropane and 50 mL of absolute EtOH was added 1.62 g (40.5 mmol) of NaOH. To this solution was added dropwise 2.48 g (13.45 mmol) of 1-2-chloroethyl)piperizine in 50 mL of EtOH over 30 min. The solution was allowed to stir for 24 h. The solvent was evaporated and the residue was extracted with 2×50 mL of CH₂Cl₂, dried over Na₂SO₄, and evaporated to dryness. The compound was purified by converting it to its hydrochloride salt by addition of HCl. The melting point of the salt was >300° C. It was converted back to its free amine by treatment with NH₄OH. The resultant oil (0.82 g, 41%) was analyzed. Mass spectral analysis showed a m/e=299 (M^{+·}+1). ¹H NMR (CDCl₃): δ1.40-1.82 (10H, m), 2.42-2.55 (14H, quin), 2.58-2.77 (10H, m).

### EXAMPLE 5

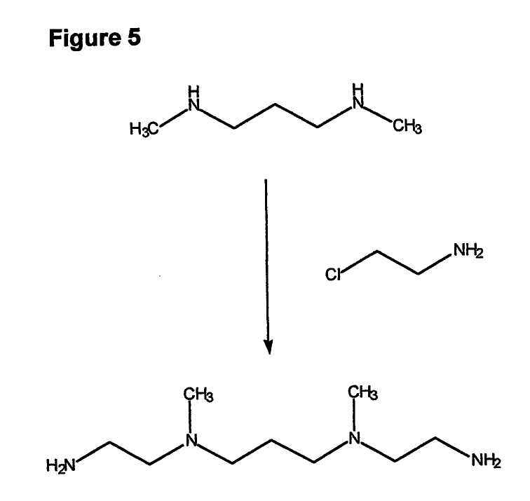

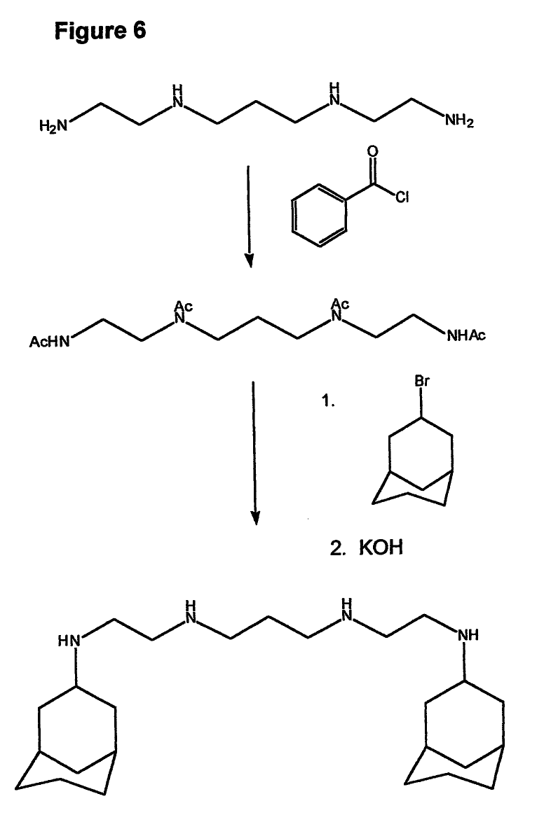

### (2-aminoethyl)(3-[(2-aminoethyl)methylaminopropyl}methylamine [FIG. 5]

To a solution of 1.0 g (0.0128 mol) of N,N-dimethyl-1,3-propanediamine in 50 mL of EtOH was added a solution of 2.96 g (25.6 mmol) of 2-chloroethylamine in 50 mL of EtOH dropwise over 40 min The solution was stirred at room temperature for 20 h. The solvent was evaporated and the residue was extracted with 2×50 mL of CH₂Cl₂, dried over Na₂SO₄, and evaporated to dryness. The resultant oil (1.52 g, 63%) was distilled (bp 145-148, 1 mm). Mass spectral analysis showed a m/e=189 (M^{+·}+1). ¹H NMR (CDCl₃): δ 1.20 (4H, s), 1.60 (2H, quin), 2.29 (6H, s) 2.57 (4H, t), 2.73 (8H, t).

### EXAMPLE 6

### [2-(bicyclo[3.3.1]non-3-ylamino)ethyl](3-{2-(bicyclo[3.3.1]non-3-ylamino)ethyl]amino}propyl)amine [FIG. 6]

A mixture of 0.06 mol of 1-bromoadamantane and 0.30 mol of acetylated 2,3,2-tetramine were heated in a stainless steel bomb at 215° C. for 6 h The product was poured into a mixture of 250 mL of 2 N HCl and 200 mL of ether. The aqueous layer was separated and made alkaline with 200 mL of 50% aqueous NaOHR The mixture was extracted with ether and the extract dried over K₂CO₃ and evaporated to give an oil (1.32 g, 33%). Mass spectral analysis showed a m/e=406. ¹H NMR (CDCl₃): δ 1.24-1.30 (4H, s), 1.50-2.12 (32H, m), 2.62 (4H, t), 2.75 (8H, t).

### EXAMPLE 7

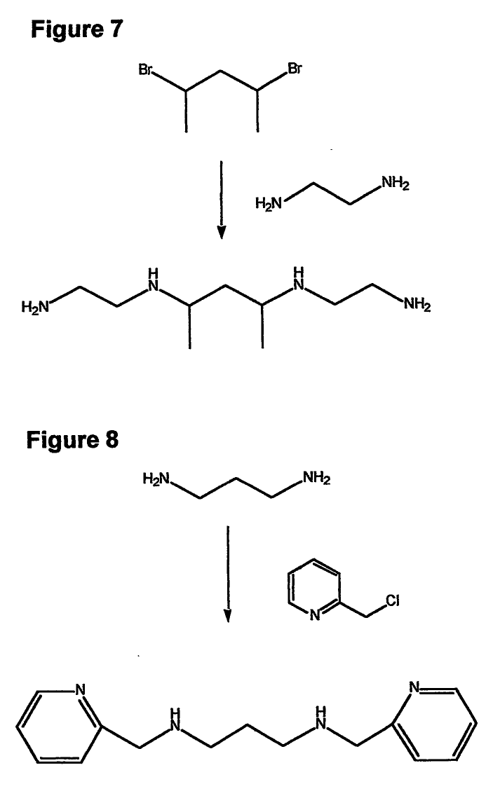

### (2aminoethyl){3-[(2aminoethyl)amino]-1-methylbutyl}amine [FIG. 7]

A mixture of 2.34 g (10 mmol) of 2,4-dibromopentane and 50 mL of absolute EtOH was added slowly to 1.2 g (20 mmol) of 1,2-diaminoethane hydrate. The mixture immediately became warm. It was then heated to 50° C. for 1 hour, 10 g of KCl added and the heating continued for 30 minutes. The mixture was filtered from the KBr and distilled at reduced pressure. The compound was purified by converting it to its hydrochloride salt by addition of HCl. The melting point of the salt was >300° C. It was converted back to its free amine by treatment with NH₄OH. Mass spectral analysis of the oil ((1.28 g, 68%) showed a m/e=188. ¹H NMR (CDCl₃): δ 1.12 (6H, d), 1.30-1.37 (6H, s), 1.60 (2H, t), 2.60 (2H, m), 2.74 (8H, t).

### EXAMPLE 8

### (2-pyridylmethyl){3-[(2pyridylmethyl)amino]propyl}amine [FIG. 8]

To a solution of 1.0 g (0.0135 mol) of 1,3-diaminopropane in 50 mL of EtOH was added a solution of 4.43 g (27.0 mmol) of 2-chloromethylpyridine in 25 mL of water. 10% NaOH was added until the pH reached 9. The solution was stirred at room temperature and NaOH was added to keep the pH at 8-9 over 3 days. The solvent was evaporated and the residue was extracted with 3×30 mL of CH₂Cl₂, dried over Na₂SO₄, and evaporated to dryness. The resultant oil (2.63 g, 76%) was analyzed. Mass spectral analysis showed a m/e=257 (M^{+·}+1). ¹H NMR (CDCl₃): δ 1.60 (2H, quin), 2.62 (4H, t), 4.06 (4H, s), 7.15-7.80 (6H, m), 8.44-8.63 (2H, d).

### EXAMPLE 9

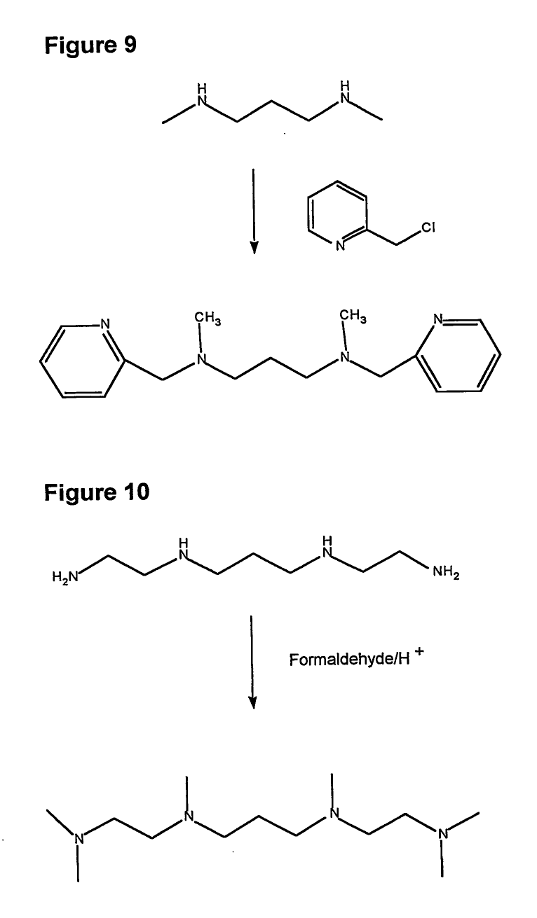

### methyl(3[methyl(2-pyridylmethyl)amino]propyl)(2-pyridylmethyl)amine [FIG. 9]

To a solution of 1.0 g (0.0128 mol) of N,N-dimethyl-1,3-propanediamine in 50 mL of EtOH was added a solution of 4.19 g (25.6 mmol) of 2-chloromethylpyridine in 25 mL of water. 10% NaOH was added until the pH reached 9. The solution was stirred at room temperature and NaOH was added to keep the pH at 8-9 over 3 days. The solvent was evaporated and the residue was extracted with 3×30 mL of CH₂Cl₂, dried over Na₂SO₄, and evaporated to dryness. The resultant oil (2.69 g, 74%) was analyzed Mass spectral analysis showed a m/e=285 (M^{+·}+1). ¹H NMR (CDCl₃): δ 1.55 (2H, quin), 2.30 (6H, s), 2.58 (4H, t), 3.75 (4H, s), 7.07-7.85 (6H, m), (2H, d).

### EXAMPLE 10

### [2-(dimethylamino)ethyl](3-{[2-(dimethylamino)ethyl]methylamino}propyl)methylamine [FIG. 10]

A solution of 1.0 g (6.23 mmol) of 2,3,2-tetramine, 10 mL of formic acid, 10 mL of 37% formaldehyde and 1 mL of water was refluxed for 20 h The solvent was evaporated, the solution was made basic with 3 M NaOH, and was extracted with 3×30 mL of CH₂Cl₂, dried over Na₂SO₄, and evaporated to dryness. The resultant oil (0.88 g, 58%) was analyzed. Mass spectral analysis showed a m/e=244 (M^{+·}+1). ¹H NMR (CDCl₃): δ 1.62 (2H, quin), 2.24-2.30 (18H, s), 2.60 (4H, t), 2.71-2.75 (8H, t).

### EXAMPLE 11

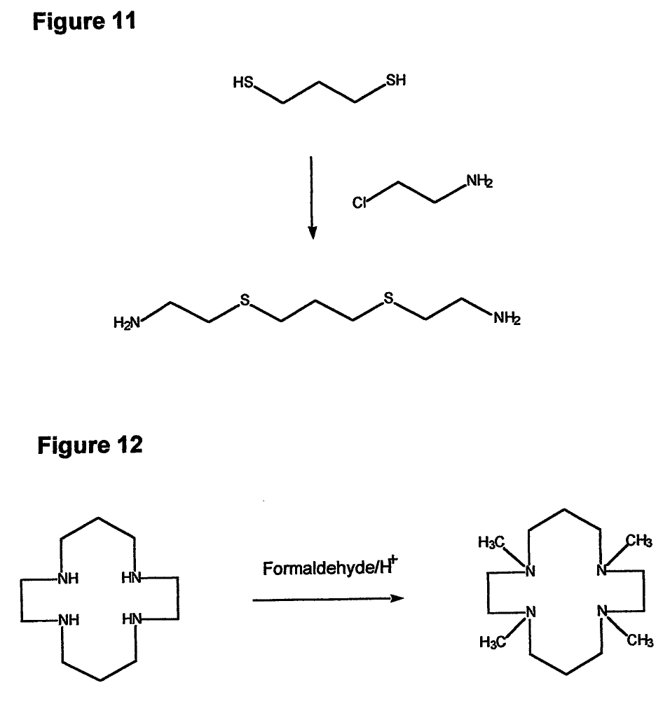

### 2-3-(2-aminoethylthio)propylthio]ethylamine [FIG. 11

To a solution of 1.0 g (0.0128 mol) of 1,3-dimercaptopropane in 50 mL of EtOH was added a solution of 1.48 g of NaOH in 10 mL of water. To the solution was added 214 g (18.48 mmol) of 2-chloroethylamine in 25 mL of EtOH. The solution was refluxed for 8 h. The solvent was evaporated and the residue was extracted with 3×25 mL of CH₂Cl₂, dried over Na₂SO₄, and evaporated to dryness. The resultant oil was distilled at 165-173 (1 mm) to give 1.81 g, 73%. Mass spectral analysis showed a m/e=194. ¹H NMR (CDCl₃): δ 1.48 (4H, s), 2.34-2.86 (14H, m).

### EXAMPLE 12

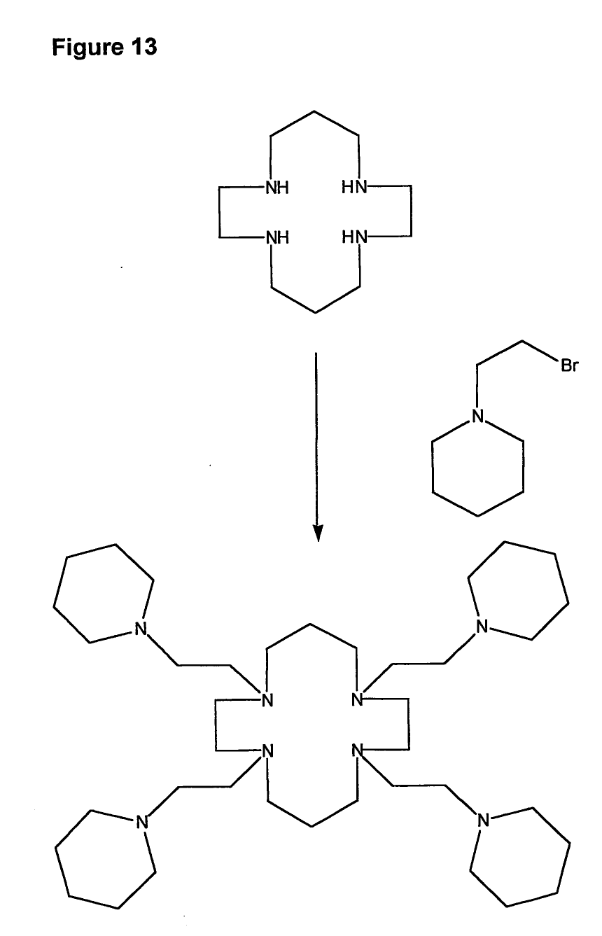

### 1,4,8,11-tetraaza-1,4,8,11-tetramethylcyclotetradecane [FIG. 12]

A solution consisting of 1.0 g (0.005 mol) of cyclam, 5.3 mL of formic acid, 4.5 mL of 37% formaldehyde and 1 mL of water was refluxed for 18 h. The reaction mixture was transferred with 6 mL of water to a beaker and cooled to 5° C. in an ice bath While stirring, a concenrated solution of NaOH was slowly added to pH>12, The temperature was kept below 25° C. during the addition and then extracted with 3×30 mL of CH₂Cl₂., dried over Na₂SO₄, and evaporated to dryness. The resultant oil (0.98 g, 71%) was analyzed Mass spectral analysis showed a m/e=256. ¹H NMR (CDCl₃): δ 1.68 (4H, quin), 2.22 (12H, s), 2.64 (8H, t), 2.75 (8H, t).

### EXAMPLE 13

### 1,4,8,11-tetraaza-1,4,8,11-tetra(2-piperidylethyl)cyclotetradecane FIG. [13]

To a solution of 0.5 g (2.5 mmol) of cyclam in 25 mL of CH₂Cl₂ was added a solution of 0.8 g of NaOH in 25 mL of water. A solution of 1.83 g (9.98 mmol) of 1-(2-chloroethyl)piperidine in 25 mL of CH₂Cl₂ was added dropwise at room temperature. The stirring was continued for 24 h. The solvent was evaporated and the residue was extracted with 3×50 mL of CH₂Cl₂, dried over Na₂SO₄, and evaporated to dryness. The resultant oil (0.725 g, 45%) was analyzed Mass spectral analysis showed a m/e=646 (W-+1). ¹H NMR (CDCl₃): δ 1.28 (8H, q), 1.46-1.72 (24H, m), 1.72 (4H, m), 2.42-2.80 (24H, m), 2.64 (8H, t), 2.75 (8H, t).

### EXAMPLE 14

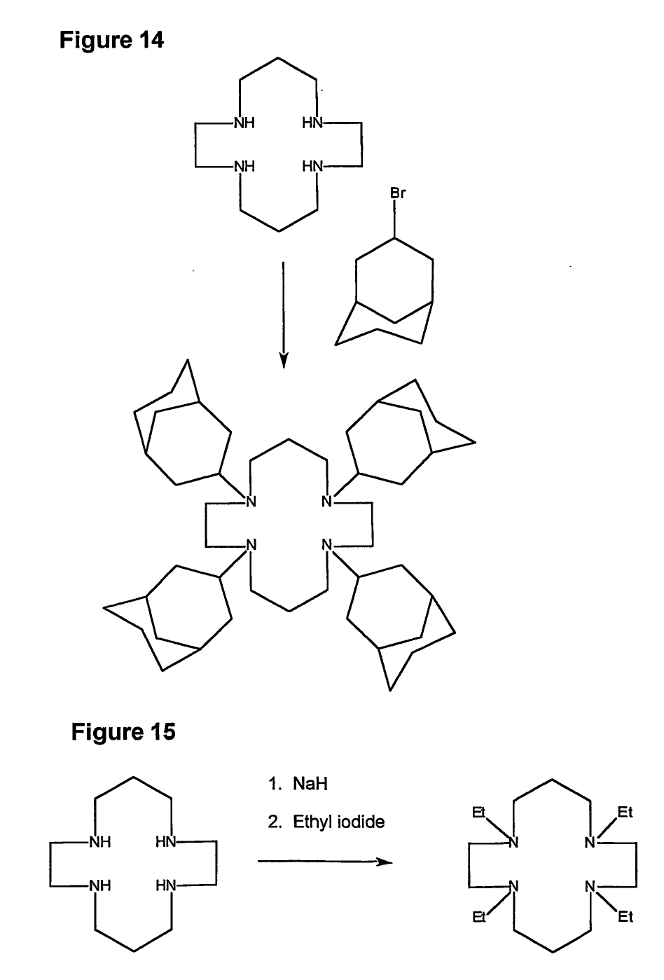

### 1,4,8,11-tetraaza-1,4,8,11-tetrabicyclo[3.3.1]non-3-ylcyclotetradecane [FIG. 14]

To 0.5 g (2.5 mmol) of cyclam in 50 mL of EtOH was added 2.15 g (10.0 mmol) of 1-bromoadamantane in 50 mL of EtOH dropwise over 30 min The solution was heated to reflux and heated for 20 h. The solution was evaporated under reduced pressure, extracted with 3×35 mL of CH₂Cl₂., dried over Na₂SO₄, and evaporated to dryness. The resultant oil (0.53 g, 31%) was analyzed. Mass spectral analysis showed a m/e=690 (M+1). ¹H NMR (CDCl₃): δ 1.24-1.58 (56H, m), 1.66 (4H, quin), 2.62 (8H, t), 2.70 (8H, t).

### EXAMPLE 15

### 1,4,8,11-tetraaza-1,4,8,11-tetraethylcyclotetradecane [FIG. 15]

To a stirred solution of 1.0 g (5.0 mmol) of cyclam in 50 mL of DMF was added 4.0 g (0.1 mol) of NaH in small portions. The solution was heated under nitrogen at 60° C. for 3 h. 3.12 g (20 mmol) of iodoethane was added in one portion. The solution was heated at 60° C. for 18 h. The reaction was quenched with 95% EtOH, extracted with 3×35 mL of CH₂Cl₂., dried over Na₂SO₄, and evaporated to dryness. The resultant oil was purified by flash chromatography using ethyl acetate/MeOH. Mass spectral analysis of the oil (0.72 g, 46%) showed a m/e=312. ¹H NMR (CDCl₃): δ 1.38 (1214, t), 2.16 (8H, q), 3.38 (4H, quin), 3.54 (8H, t), 3.80 (8H, t).

### EXAMPLE 16

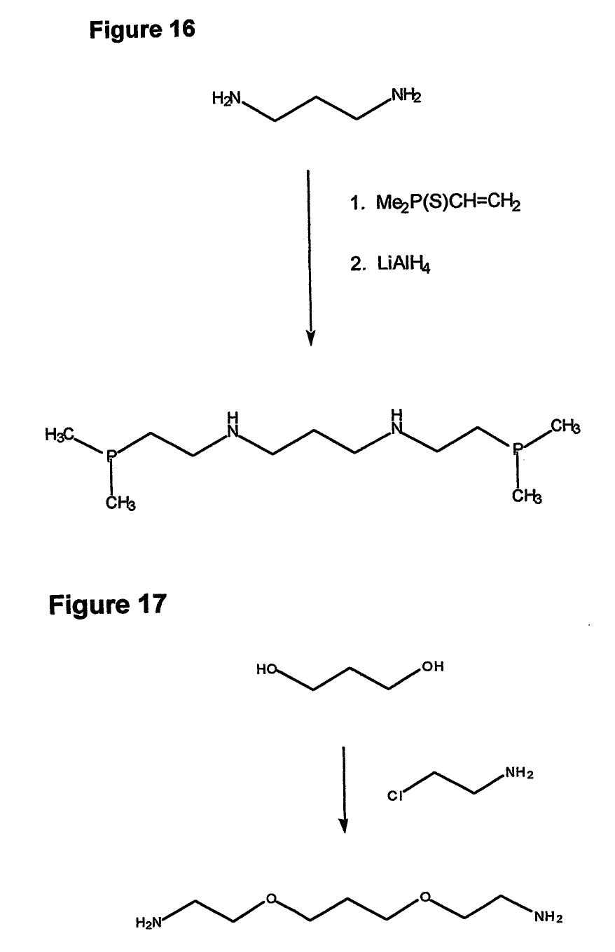

### N,N′-(2′-dimethylphosphinoethyl)propylenediamine [FIG. 16]

Propylenediamine (4.0 g) was dissolved in 200 mL of ethanol. To the solution was added 9.4 g of dimethylvinylphosphine sulfide and the mixture was heated at reflux for 72 h. The solvent was evaporated under reduced pressure and the residue dissolved in 400 mL of chloroform and washed with 50 mL of 2 M NaOH and dried over MgSO₄. The solvent was removed under reduced pressure to give an oil that was crystallized from ethyl acetate to give 6.8 g (51%) of the pure product. To a suspension of LiAlH4 (1.2 g) in 125 mL of dry dioxane was added N,N′-(2′-dimethylphosphinothioethyl)-propylenediamine (prepared as above). The mixture was refluxed for 36 h. The mixture was cooled, dioxane/water added, 3 mL of 2 M NaOH added and then the solution was filtered to give the pure phosphine. ¹H NMR (CDCl₃): δ 1.64(2H, quin), 2.10 (12H, s), 2.57 (4H, t), 2.55-2.80 (8H, m).

### EXAMPLE 17

### 3-(3-(2-aminoethoxy)propoxy)propylamine [FIG. 17]

Sodium 0.20 g, (8.6 mmol) was added in small portions to 50 mL of ethanol. After evolution of hydrogen ceased, 0.33 g (4.3 mmol) of 1,3 propanediol was added and stirred for 1 h 2-chloroethylamine (1.0 g, 8.6 mmol) in 50 mL of ethanol was added dropwise over 30 min. The solution was refluxed for 8 h and the solvent was evaporated. The resultant oil was dissolved in 50 mL of water and extracted with 2×50 mL of CH₂Cl₂ dried over Na₂SO₄, filtered and evaporated. The oil was flash chromatographed using 1:1 ethyl acetate/hexane to give 0.32 g (46%) of 3-3-(2-aminoethoxy)propoxy)propylamine. ¹H NMR (CDCl₃): δ 1.32(4H, s), 1.68 (2H, q), 2.71 (2H, t), 2.96 (8H, t). MS m/z 162 (calcd 162).

### EXAMPLE 18

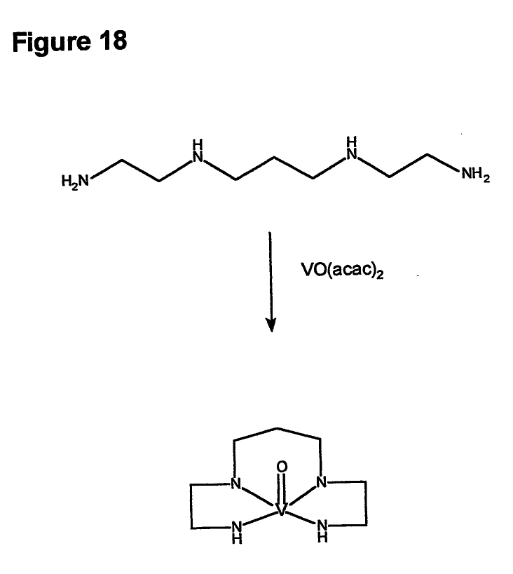

### Vanadyl 2,3,2-Tetramine [FIG. 18]

To 1.0 g (0.624 mol) of 2,3,2 tete in 20 ml of EtOH was added 0.073 g (0.0624 mol) of vanadylacetylacetonate in 20 ml of EtOH. The solution was refluxed for 30 min and cooled to room temperature. Overnight a red-brown solid precipitated. The complex is formulated as being [VO(2,3,2-tetramine)acac].

### EXAMPLE 19

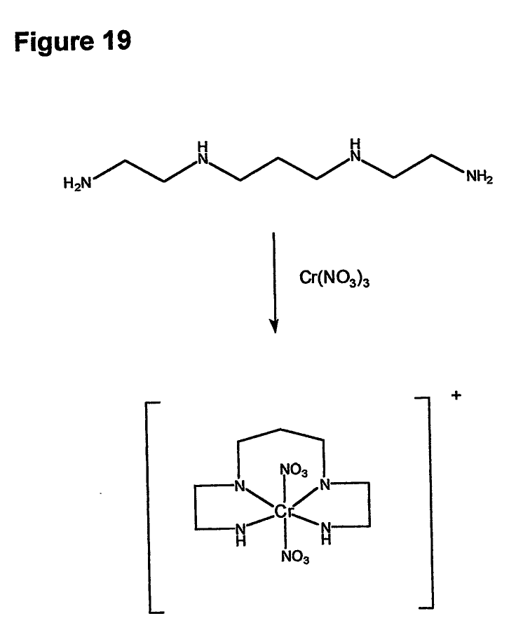

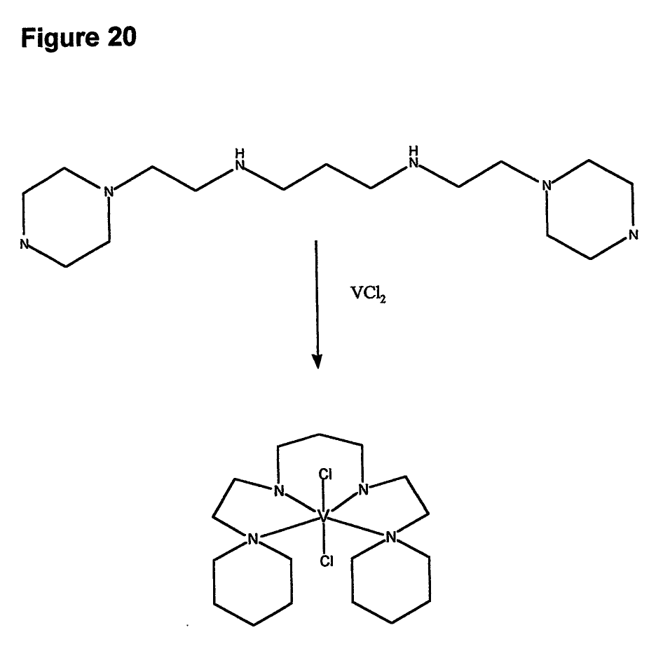

### Chromium 2,3,2-Tetramine [FIG. 19]

To 1.0 g (0.0624 mol) of 2,3,2-tetramine in 20 ml of EtOH was added 0.245 g (0.0624 mol) of chromium (E) nitrate in 20 ml. Of EtOH. The solution was refluxed for 30 min and cooled to room temperature. Overnight a solid precipitated. The complex is formulated as being [Cr(2,3,2-tetraamine)(NO₃)₂]NO₃.

### EXAMPLE 20

### Vanadyl ((2-piperidylethyl)-{3-[(2-piperidylethyl)amino]propyl}amine)(Cl)₂ [FIG. 20]

To 200 mg (0.67 mmol) of 2,3,2-pip in 25 mL of methanol was added a solution of 82 mg (0.67 mmol) of V(II)Cl₂ in 15 mL of methanol. The solution was heated for 30 min and cooled to room temperature. Crystals of Vanadyl ((2-piperidylethyl)-{3-[(2-piperidylethyl)amino]propyl}amine)(Cl)₂ formed overnight which were collected and dried. Anal. Calcd for VC₁₅Cl₂H₃₄N₆: C, 42.86; H. 8.17; N, 19.98. Found: C, 42.33; H. 8.24; N, 20.03.

### EXAMPLE 21

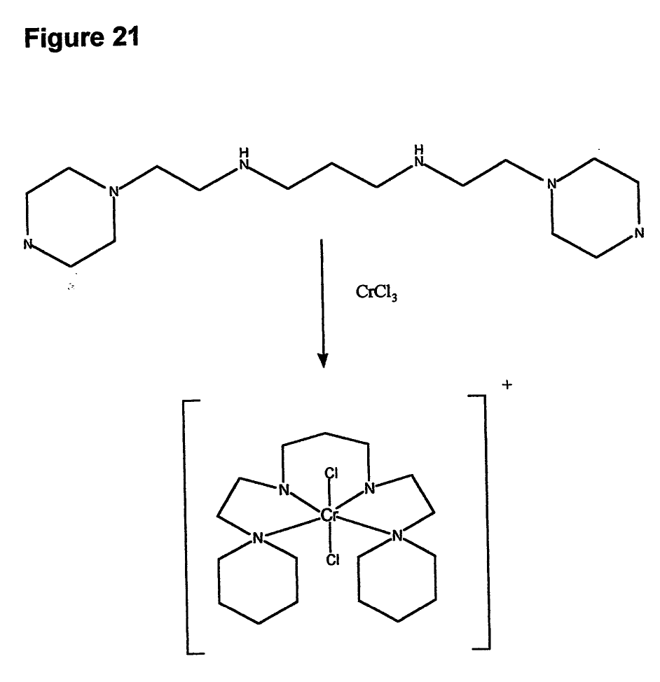

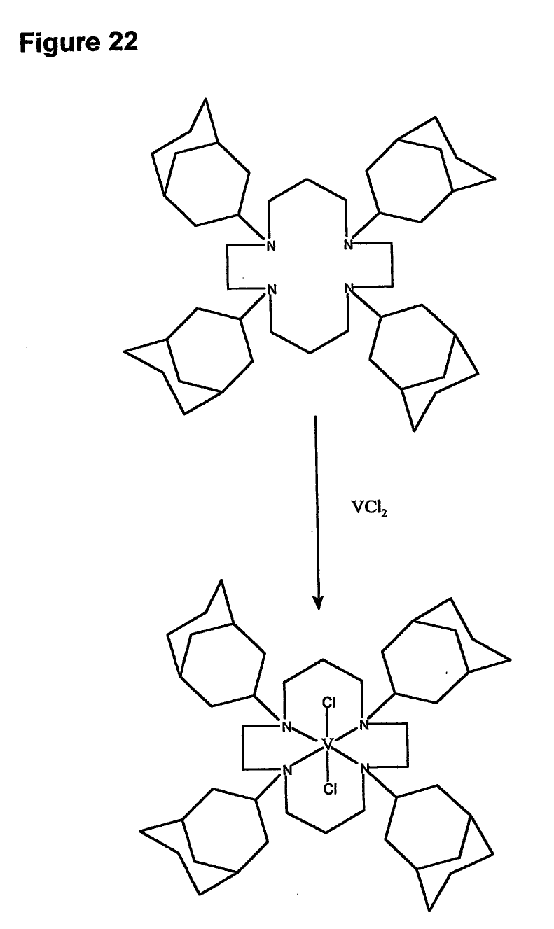

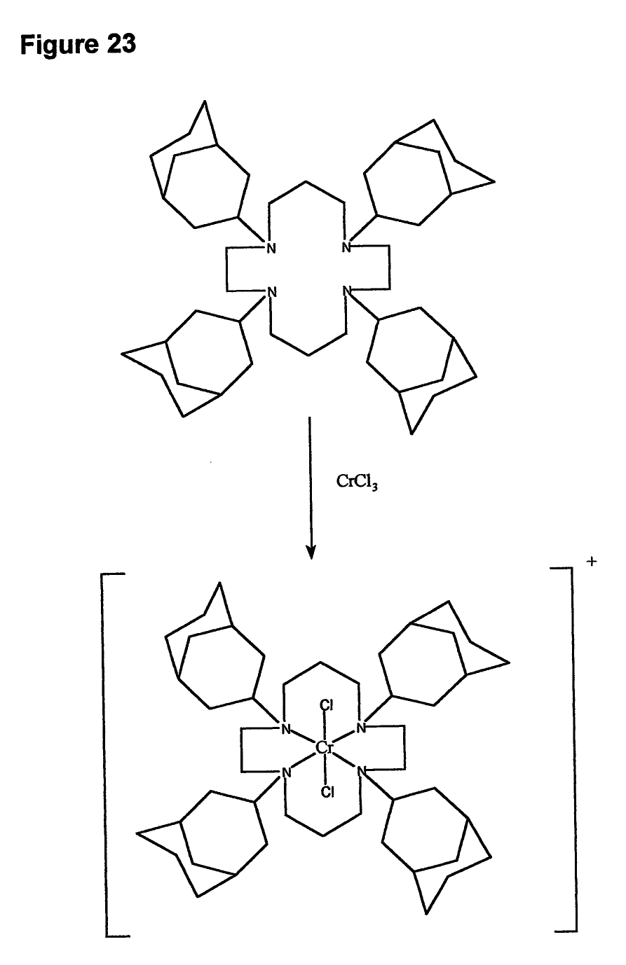

### Chromium ((2-piperidylethyl)-{3-[(2-piperidylethyl)amino]propyl}amine)(Cl)₂]Cl [FIG. 21]

To 200 mg (0.67 mmol) of 2,3,2-pip in 25 mL of methanol was added a solution of 178 mg (0.67 mmol) of Cr(III)Cl₃ in 25 mL of methanol. The solution was heated for 30 min, cooled to room temperature and the solvent was evaporated to 20 mL. Crystals of [Chromium ((2-piperidylethyl)-{3-[(2-piperidylethyl)amino]propyl}amine)(Cl)₂]Cl formed overnight which were collected and dried. Anal. Calcd for CrCl₅C₁₃H₃₄N₆: C, 39.43; H, 7.52; N, 18.39. Found: C, 39.05; H, 7.19; N, 18.54.

### EXAMPLE 22

### Vanadyl (1,4,8,11-tetraaza-1,4,8,11-tetrabicyclo[3.3.1]non-3-ylcyclotetradecane)(Cl)₂ [FIG. 22]

To 300 mg (0.41 mmol) of cyclam-ad in 25 mL of methanol was added a solution of 50 mg (0.41 mmol) of V(II)Cl₂ in 10 mL of methanol. The solution was heated for 30 min, cooled to room temperature and the solvent evaporated to 10 mL. Crystals of Vanadyl (1,4,8,11-tetraaza-1,4,8,11-tetrabicyclo[3.3.1]non-3-ylcyclotetradecane)(Cl)₂ formed in 72 h which were collected and dried. Anal. Calcd for VC₅₀Cl₂H₈₀N₄: C, 69.24; H, 9.32; N, 6.45. Found: C, 69.01; Et 9.45; N, 6.88.

### EXAMPLE 23

### Chromium (1,4,8,11-tetraaza-1,4,8,11-tetrabicyclo[3.3.1]non-3-ylcyclotetradecane)(Cl)₂]Cl [FIG. 23]

To 300 mg (0.41 mmol) of cyclam-ad in 25 mL of methanol was added a solution of 108 mg (0.41 mmol) of Cr(III)Cl₃ in 20 mL of methanol. The solution was heated for 30 min, cooled to room temperature and the solvent evaporated to 10 mL. Crystals of Chromium (1,4,8,11-tetraaza-1,4,8,11-tetrabicyclo[3.3.1]non-3-ylcyclotetradecane)(Cl)₂]Cl formed overnight which were collected and dried. Anal. Calcd for CrC₅₀Cl₃H₈₀N₄: C, 67.04; H. 9.02; N, 6.25. Found: C, 67.11; H. 8.89; N, 6.41.

### EXAMPLE 24

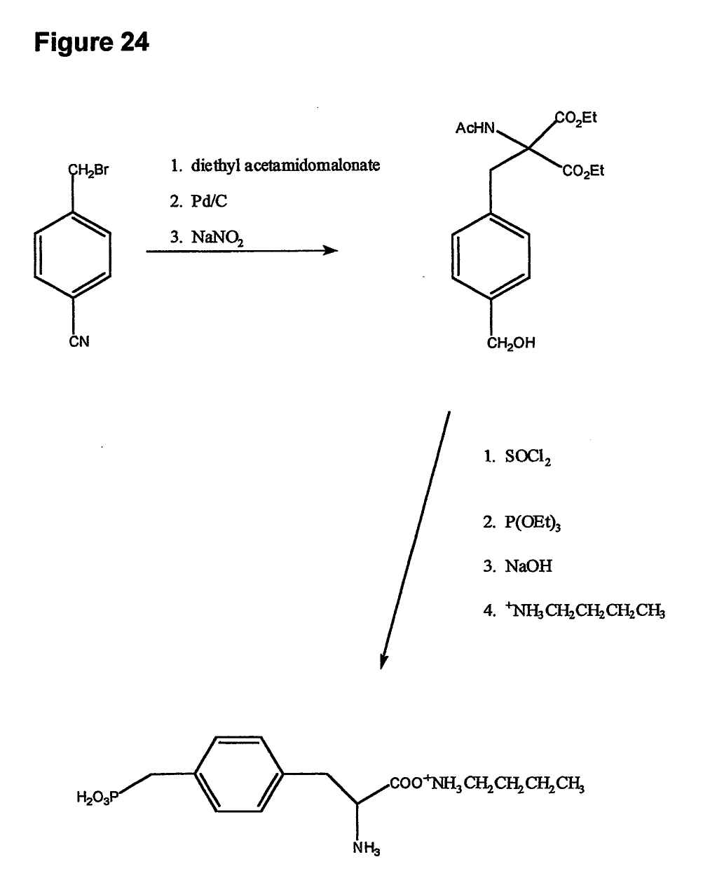

### p-(Phosphonomethyl)Diphenylalanine-Butylamine Salt [FIG. 24]

A solution of Na (0.26 g, 0.011 mol), diethyl acetamidomalonate (2.17 g, 0.01 mol), and p-cyanobenzyl bromide (1) (1.96 g, 0.01 mol) in 25 mL of ethanol was refluxed and stirred for 17 h at 110° C. After cooling and addition of water (50 mL), the crystalline material was collected by filtration and washed twice with water, to yield 2.89 g (87%) of the white solid Diethyl (4-Cyanobenzyl)acetamidomalonate. ¹H NMR (DMSOd) δ 1.12 (t, 6H), 1.93 (s, 3H), 3.42 (s, 2H), 4.14 (q, 4H,), 7.16-7.80 (2 d, 4H), 8.15 (s, 1H).

Diethyl (4-Cyanobenzyl)acetamidomalonate (996 mg, 3 mmol) was hydrogenated at atmospheric pressure and room temperature for 22 h in ethanol (25 mL) and concentrated HCl (1.5 mL) with Pd/C 10% as catalyst (200 mg). After filtration, the solution was taken to dryness. Water (60 mL) was added to the residue and unreacted material was removed by filtration. The filtrate was again concentrated to dryness, giving 956 mg (85%) of the white solid Diethyl [4-(Aminomethyl)benzyl]acetamidomalonate. ¹H NMR (DMSO d) δ 1.12 (t, 6H), 1.92 (s, 3H), 3.40 (s, 2H), 3.88 (s, 2H), 4.11 (q, 4H), 7.03-7.38 (2 d, 4H), 8.21 (s, 3H).

A solution of Diethyl [4-Aminomethyl)benzyl]acetamidomalonate (2.06 g, 5.5 mmol), NaNO₂ (535 mg), and water (100 mL) was heated for 3 h at 110° C., cooled, and extracted with ethyl acetate. The extract was washed with 1 M HCl, water, 5% NaHCO₃, water and brine, dried over Na₂SO₄, filtrated, and taken to dryness, giving a white crystallized solid Diethyl [4-(Hydroxymethyl)benzyl]acetamidomalonate. 1.55 g (83%); ¹HNMR (DMSO-d₆) δ 1.10 (t, 6H), 1.90 (s, 3H), 3.38 (s, 2H), 4.12 (q, 4H), 4.48 (d, 2H), 5.04 (t, 1H), 6.82-7.10 (2 d, 4H), 7.92 (s, 1H).

A solution of Diethyl [4-(Hydroxymethyl)benzyl]acetamidomalonate (210 mg, 0.6 mmol) and thionyl chloride (1.4 mL, 30 equiv) in dichloromethane (15 mL) was refluxed for 18 h and concentrated to dryness. The residue was washed twice with diethyl ether, to give the white solid Diethyl [4-(Chloromethyl)benzyl]acetamidomalonate. 152 mg (68%); ¹H NMR (DMSO-d₄) 1.10 (t, 6H), 1.90 (s, 3H), 3.40 (s, 2H), 4.17 (q, 4H), 4.69 (s, 2H), 6.90-7.38 (2 d, 4H), 8.09 (s, 1H).

Diethyl [4-(Chloromethyl)benzyl]acetamidomalonate (50 mg, 0.14 mmol) was dissolved in triethyl phosphite (4 mL) and refluxed for 22 h. After removal of triethyl phosphite, the oily residue was purified by flash chromatography on silica gel with CH₂Cl₂—CH₃OH (90:10) as eluent, to yield 45.6 mg (71%) of the white solid Diethyl [4-[(Diethoxyphosphinyl)methyl]benzyl]acetamidomalonate. ¹H NMR (DMSO-d₆) δ 1.11 (t, 12H) 1.88 (s, 3H), 3.12 and 3.15 (s, 2H), 3.38 (s, 2H), 3.92 (m, 4H), 4.09 (q, 4H), 6.88-7.18 (d, 4H), 7.92 (s, 1H).

A solution of Diethyl [4-[(Diethoxyphosphinyl)methyl]benzyl]acetamidomalonate (55 mg, 0.06 mmol), 1 N NaOH (0.5 mL, 4 equiv) in water (2 mL), and methanol (2 mL) was stirred for 3 h at room temperature. After addition of 8 mL of water and 5 mL of concentrated HCl, the reaction mixture was refluxed for 4 h (110° C.), cooled, and, after addition of 20 mL of water, adjusted to pH 4 with 10% NaOH. After lyophilization and purification by chromatography on silica gel with 2-propanol-NH₄OH (28%) (60:40) as eluent, the white product (14.2 mg, 40% yield) p-(Phosphonomethyl)-DL-phenylalanine was obtained: ¹H NMR (D₂O) (TMS as external reference) δ 2.70 and 2.83 (s, 2H,), 2.88 and 3.12 (dd, 2H), 3.74 (m, 1H), 7.10 and 7.22 (d, 4H); mass spectrum (FAB), m/e (MH)⁺ calcd 296, found 296. The compound was converted into its butylamine salt by treatment with butylamine followed by precipitation from an aqueous solution.

### EXAMPLE 25

### 2-amino-N-(2-{[3-(2-[2-amino-3-(4-phosphonomethylphenyl) propanolylaminolethyl)amino)propyl]amino)ethyl)-3-(4-phosphonomethylphenyl)propanamide [FIG. 25]

To 5 mL of dioxane was added 2.0 g (7.7 mmol) of p-(phosphonomethyl)-DL-phenylalanine, 0.82 g (8.1 mmol) of triethylamine and 1.68 g (7.7 mmol) of di-tert-butyl dicarbonate. The solution was stirred at room temperature for 3 hours. The solution was evaporated at reduced pressure to dryness. The resultant oil was purified by column chromatography using methanol/chloroform to yield 2.1 g (77%) of the Boc-p-(phosphonomethyl)-DL-phenylalanine.

To a solution of 0.5 g (3.1 mmol) of 2,3,2-tetra ine in 10 mL of DMF was added 2.24 g (6.2 mmol) of Boc-p-phosphonomethyl)-DL-phenylalanine at 0° C. under an atmosphere of nitrogen. To this solution was added a solution of DPPA (1 mL, 3.2 mmol) in 10 mL of DMF and 0.35 g (3.2 mmol) of powered NaHCO₃. The solution was vigorously stirred for 24 hours at 0° C. The solution was diluted with ethyl acetate, washed with 1 N HCl, followed by saturated NaHCO₃. The solution was dried over MgSO₄, filtered, and evaporated under reduced pressure. The oil was purified by flash chromatography to give 1.2 g (46%) of Boc-2-amino-N-(2-{[3-(2-[2-amino-3-4-phosphonomethylphenyl) propanolylamino]ethyl)amino)propyl]amino}ethyl)-3-(4-phosphonomethylphenyl)propanamide as a colorless oil.

A solution of Boc-2-amino-N-2-([3-2-[2-amino-3-(4-phosphonomethylphenyl) propanolylamino]ethyl]amino)propyl]amino}ethyl)-3-(4-phosphonomethylphenyl)propanamide (0.5 g, 0.59 mmol) in 10 mL of methylene chloride and 2 mL of trifluoroacetic acid was stirred at room temperature for 30 min. The solvent was evaporated at reduced pressure. 2-amino-N-(2-{[3-(2-[2-amino-3-(4-phosphonomethylphenyl) propanolylamino]ethyl}amino)propyl]amino}ethyl)-3-(4-phosphonomethylphenyl)propanamide was isolated as the trifluroacetic acid salt which was treated with ammonia to give 0.20 g (52%) 2-amino-N-(2-{[3-(2-[2-amino-3-(4-phosphonomethylphenyl) propanolylamino]ethyl}aminopropyl]amino}ethyl)-3-4-phosphonomethylphenyl)propanamide. ¹H NMR (D₂O) (TMS as external reference) δ 1.32 (8H, s), 1.62 (2H, quin), 2.57 (4H, t), 2.66 (8H, t), 2.75 (4H, s), 2.82 and 3.10 (4H, dd), 3.76 (2H, m), 7.12-7.28 (8H, m).

### EXAMPLE 26

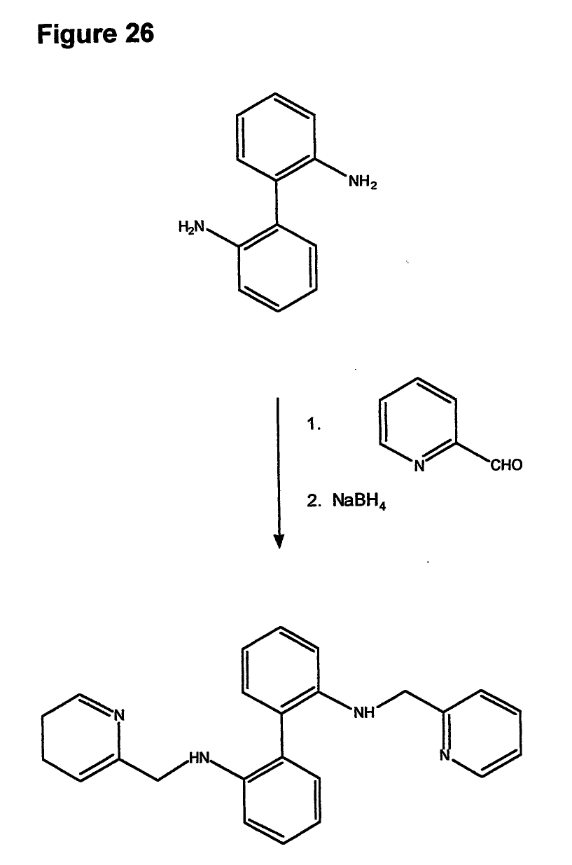

### 2,2′-diamino (bis-NN′-pyridylmethyl)biphenyl [FIG. 26]

To a solution of 1.0 g (5.43 mmol) of 2,2′-diaminobiphenyl in 50 mL of EtOH is added a solution of 3.56 g (21.7 mmol) of 2-picolylchloride hydrochloride in 15 mL of H₂O. A 10% solution of NaOH was added dropwise to the stirring solution until the pH reaches 8-9. A color change from a light yellow to a red-orange is observed at pH 8. The solution is stirred at room temperature and NaOH is added over 5 days to maintain the pH at 8. During this time, precipitation of an off-white solid occurs. The reaction is complete when the pH no longer drops below 8. The precipitate is collected by filtration and recrystallized from EtOH resulting in 1.51 g (75.9% yield) of white crystals of 2,2′-(bis-NN′-pyridylmethyl)biphenyl, mp 135-136° C., lit. mp 1370C.^{8b} Mass spectrum (AB MS), m/z (relative intensity); 367 (100), 274 (35), 195 (23), 180 (29). HRMS (FAB, MNBA) calcd for C₂₄H₂₃N₄ ([M+H]⁺): 366.1922, found 366.1923. ¹H NMR (CDCl₃): 01.62 (2K broad s), 4.48 (4H, s), 6.61-6.83 (8H, m), 7.04-8.50 (6H, m), 8.53 (2H, d). MS m/z 367 (calcd 367). Anal. Calcd for C₂₄H₂₂N₄: C, 78.65; It 6.06; N, 15.28. Found: C, 79.00; H 5.84; N, 15.62.

### EXAMPLE 27

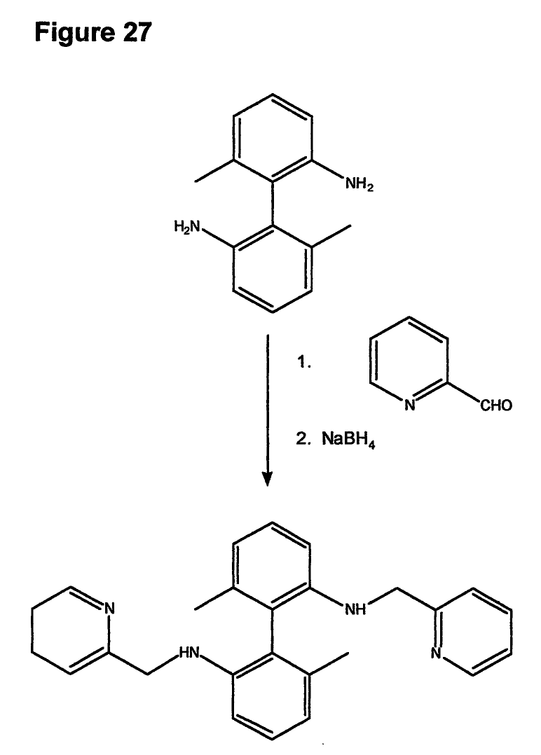

### 2,2′-diamino(bis-N,N′-pyridylmethyl)-6,6′-dimethylbiphenyl [FIG. 27]

To 6.0 g (8.2 mmol) of 6,6′-dimethyl-2,2′-dinitrobiphenyl in 75 mL of EtOH was added 1.0 g of palladium on carbon. The solution was hydrogenated on a Parr system for 4 hours at 60 psi. The catalyst was filtered and the solution was reduced to an oil under reduced pressure. The oil was crystallized from EtOH to yield 5.6 g (75.0%) of the off-white product (mp 66-67° C.) 2,2′-diamino-6,6′-dimethylbiphenyl.

To a refluxing solution of 1.5 g (7.06 mmol) of 2,2′-diamino-6,6′-dimethylbiphenyl in 40 mL of EtOH was added 1.48 g (14.12 mmol) of 2-pyridinecarboxaldehyde in 25 mL of EtOH. The solution was refluxed for 8 h and the solution turns yellow. The solution was cooled and 1.1 g of NaBH₄ was added in one portion. The solution was stirred for 2 days and the solution turned colorless. The solution was evaporated at reduced pressure, the residue was dissolved in water and extracted with 2×50 mL of ether. The ether was evaporated and the residue was crystallized from ethyl acetate/hexane to give white crystals of 2,2′-diamino(bis-N,N′-pyridylmethyl)-6,6′-dimethylbiphenyl. H NMR (CDCl₃): □2.21 (6H, s), 4.23 (4H, s), 7.02-8.13 (14H, m).

### EXAMPLE 28

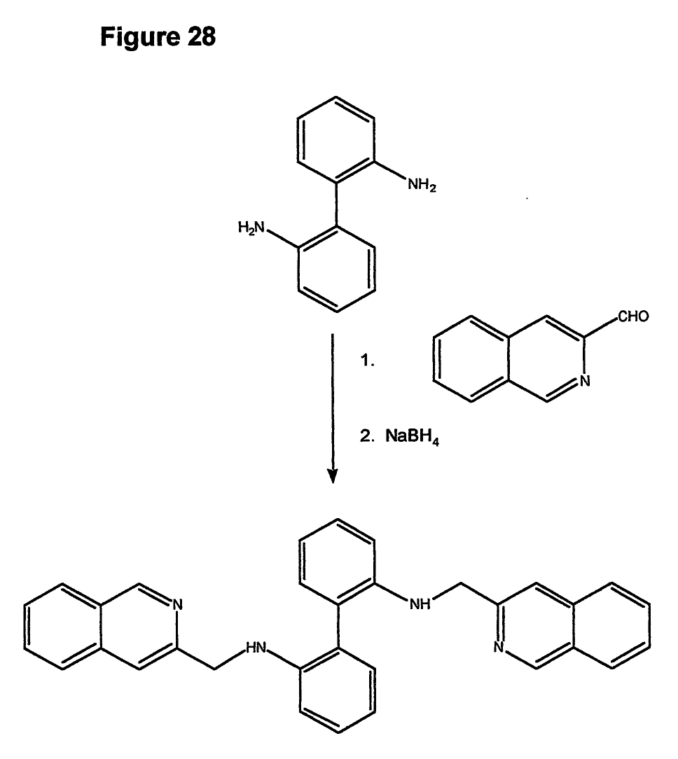

### 2,2′-diamino (bis-N,N′-quinilylmethyl)biphenyl [FIG. 28]

To 1.0 g (5.43 mmol) of 2,2′-diaminobiphenyl in 50 mL of ethanol was added 1.71 g (10.9 mmol) of 2-quinolinecarboxaldehyde. The solution was refluxed for 30 min, cooled to room temperature and the cooled to 0° C. Crystals of 1-aza-1-(2-(2-(1-aza-2-(3-isoquinolyl)vinyl)phenyl)phenyl)-2-(3-isoquinolyl)ethene were collected, mp 138-140° C.

To 1.0 g (3.62 mmol) of 1-aza-1-(2-(2-(1-aza-2-(3-isoquinolyl)vinyl)phenyl)phenyl)-2-3-isoquinolyl)ethene in 50 mL of ethanol was added 0.2 g of NaBH₄. The solution was refluxed for 30 min and then stirred at room temperature for 22 h. The solution was treated with concentrated HCl until acidic, extracted with 2×25 mL of CH₂Cl₂, dried over NaSO₄ and evaporated under reduced pressure. The resultant oil was crystallized from ethanol to yield 1.36 g (64%) of 2,2′-diamino(bis-NN′-quinilylmethyl)biphenyl, mp 156-158° C. ¹H NMR (CDCl₃): □4.60 (4H, d), 5.25 (2H, t), 7.10-8.15 (20H, m).

### EXAMPLE 29

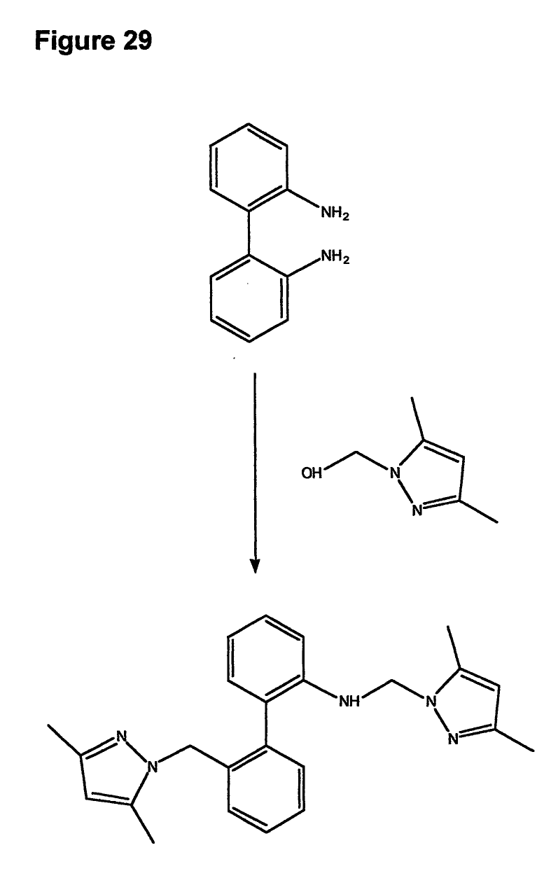

### [(3,5-dimethylpyrazolyl)methyl][2-(2-([(3,5 dimethylpyrazolyl)methyl]amino)phenyl)phenyl]amine [FIG. 29]

To 1.49 g (8.09 mmol) of 2,2′-diaminobiphenyl in 100 mL of acetonitrile was added 2.0 g (16.2 mmol) of N-hydroxymethyl(3, 5 diethyl)pyrazole. The solution was stirred at room temperature for 3 days. The solution was dried over MgSO₄, filtered and evaporated to dryness under reduced pressure. The resultant oil was crystallized from ethyl acetate to give [(3,5dimethylpyrazolyl)methyl][2-(2-{[(3,5-dimethylpyrazolyl)methyl]amino}phenyl)phenyl]amine. ¹H NMR (CDCl₃): □2.18 (6H, s), 2.26 (6E, s), 3.81 (2H, broad s), 4.52 (4H, s), 5.30 (2H, s), 6.63-8.13 (8H, m).

### EXAMPLE 30

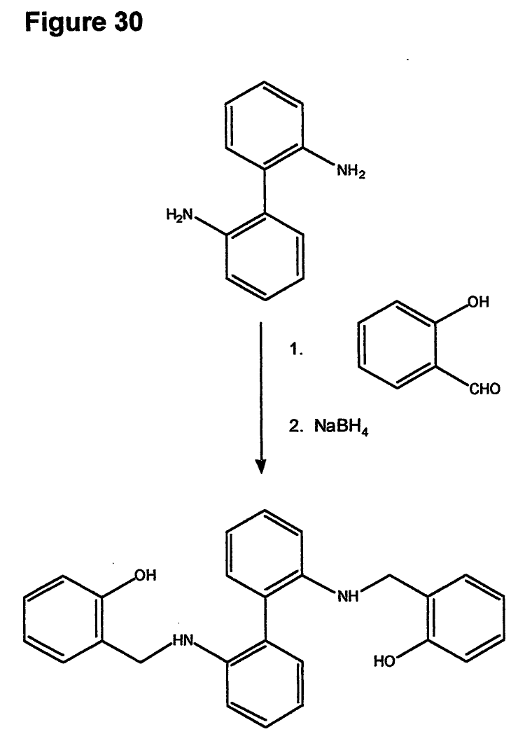

### 2-{[(2-{2-[(2-pyridylmethylamino]phenyl}-phenyl)amino]methyl}phenol [FIG. 30]

To 1.0 g (5.68 mmol) of 2,2′-diaminobiphenyl in 20 mL of ethanol was added 1.38 g (11.3 mmol) of salicylaldehyde. The solution was refluxed for 30 min and cooled in an ice bath. Yellow crystals (1.76 g) of 2-(2-aza-2-(2-(1-aza-2-(2-hydroxyphenyl)vinyl)phenyl)phenyl)vinyl)phenol mp 145-146° C.

To 1.0 g (2.55 mmol) of 2-(2-aza-2-(2-(1-aza-2-(2-hydroxyphenyl)vinyl)phenyl) phenyl)vinyl)phenol in 25 mL of ethanol was added 0.3 g (7.93 mmol) of NaBH. The solution was refluxed for 30 min and stirred at room temperature for 24 h. The solution was treated with concentrated HCl until acidic, extracted with 2×25 mL of CH₂Cl₂, dried over NaSO₄ and evaporated under reduced pressure. The resultant oil was cystallizd from ethyl acetate/hexane to yield 0.72 g (73%) of 2-({[2-2-{[2-hydroxyphenyl)methyl]amino}phenyl)phenyl]amino}methyl)phenol. ¹H NMR (CDCl₃): □1.72 (2H, broad s), 4.25 (4H, s), 7.12-7.70 (16H, m).

### EXAMPLE 31

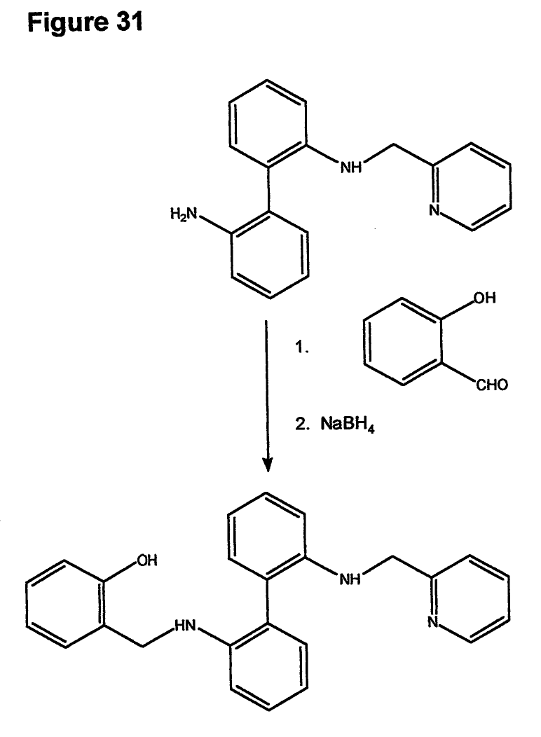

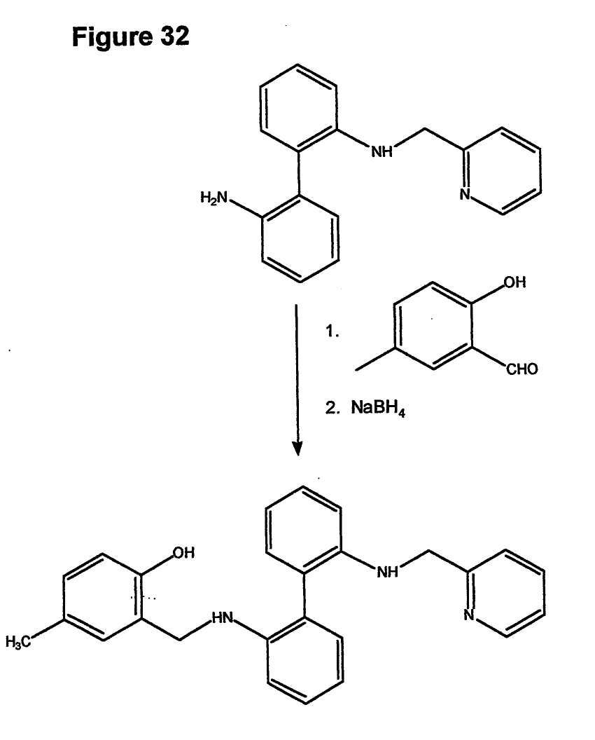

### 2-({[2-(2{[-hydroxyphenyl)methyl]amino}phenyl)phenyl]amino}methyl)phenol [FIG. 31]

To 1.00 g (3.63 mmol) of N-(2-pyridylmethyl)-2,2′-diaminobiphenyl in 25 mL of ethanol was added 0.48 g (3.63 mmol) of salicylaldehyde. The solution was refluxed for 30 min and cooled to room temperature. The solution was evaporated to 10 mL and cooled at °0. Yellow crystals were collected by suction filtration to yield 0.90 g (72%) of 2-(2-aza-2-(2-(2-pyridylmethyl)amino)phenylphenyl)vinyl)phenol, mp 110-112° C.

To 2.0 g (7.26 mmol) of 2-(2-aza-2-(2-(2-pyridylmethyl)amino)phenyl)phenyl) vinyl)phenol in 50 mL of ethanol was added 0.3 g of NaBH. The solution was sired at room temperature for 24 h. The solution was treated with concentrated HCl until acidic, extracted with 2×25 mL of CH₂Cl₂, dried over NaSO₄ and evaporated under reduced pressure. The resultant oil was crystallized from methanol to yield 1.36 g (64%) of 2-{[(2-{2-[(2-pyridylmethylamino]phenyl}-phenyl)amino]methyl}phenol. mp 154-156° C. H NR (CDCl₃): 01.60 (2H, broad s), 4.42 (4H, s), 6.81-8.23 (16H, m). MS m/z 426 (calcd 426).

### EXAMPLE 32

### 4-methyl-2-{[(2-{2-[(2-pyridylmethylamino]phenyl}-phenyl)amino]methyl}phenol [FIG. 32]

To 0.60 g (2.18 mmol) of N-(2-pyridylmethyl)-2,2′-diaminobiphenyl in 25 mL of ethanol was added 0.30 g (2.18 mmol) of 2-hydroxy-5-methylbenzaldehyde. The solution was refluxed for 30 min and cooled to room temperature. The solution was evaporated to 10 mL and cooled at °0. Yellow crystals were collected by suction filtration to yield 0.90 g (72%) of 2-(2-aza-2-(2-(2-pyridylmethyl)amino)phenyl)phenyl)vinyl)-4-methylphenol, mp 158-160° C.

To 2.0 g (7.26 mmol) of 2-2-aza-2-(2-(2-pyridylmethyl)amino)phenyl)phenyl)vinyl)-4-methylphenol in 50 mL of ethanol was added 0.3 g of NaBH₄. The solution was stirred at room temperature for 24 h. The solution was treated with concentrated HCl until acidic, extracted with 2×25 mL of CH₂Cl₂, dried over NaSO₄ and evaporated under reduced pressure. The resultant oil was crystallized from methanol to yield 1.36 g (64%) of 4-methyl-2-{[(2-{2-[(2-pyridylmethylamino]phenyl}-phenyl)amino]methyl}phenol. mp 135-137° C. ¹H NMR (CDCl₃): □1.68 (1H, broad s), 2.03 (3H, s), 4.40 (4H, s), 6.82-8.20 (15H, m).

### EXAMPLE 33

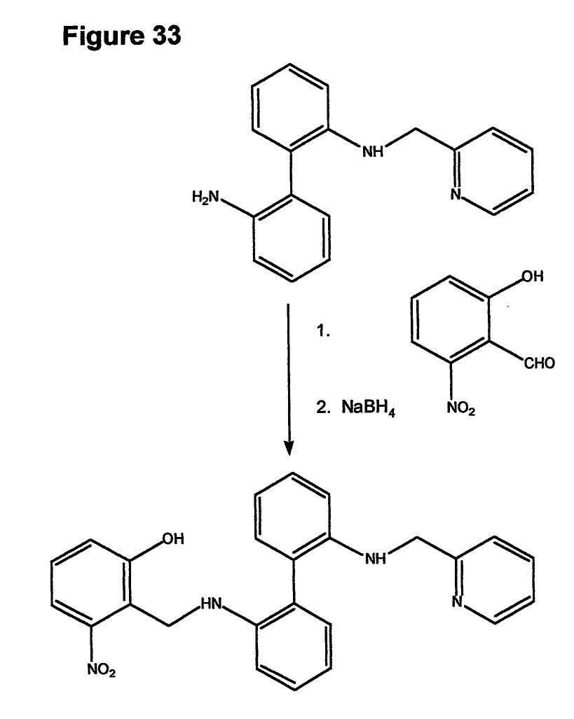

### 3-nitro-2-{[(2-{2-[(2-pyridylmethylamino]phenyl}-phenylamino]methyl}phenol [FIG. 33]

To 0.60 g (2.18 mmol) of N-(2-pyridylmethyl)-2,2′-diaminobiphenyl in 10 mL of ethanol was added 0.36 g (2.18 mmol) of 2-hydroxy-6-nitrobenzaldehyde. The solution was refluxed for 30 min and cooled to room temperature. Yellow crystals were collected by suction filtration to yield 0.36 g (41%) of 2-(2-aza-2-(2-(2-pyridylmethyl)amino)phenyl)phenyl)vinyl)-5-nitrophenol, mp 114-115° C.

To 0.35 g (1.59 mmol) of 2-(2-aza-2-(2-(2-pyridylmethyl)amino)phenyl)phenyl)vinyl)-5-nitrophenol in 50 mL of ethanol was added 0.72 g (19.0 mmol) of NaBH₄. The solution was stirred at room temperature for 24 h. The solution was treated with concentrated HCl until acidic, extracted with 2×25 mL of CH₂Cl₂, dried over NaSO₄ and evaporated under reduced pressure. The resultant oil was crystallized from methanol to yield 0.24 g (61%) of 3-nitro-2-{[(2-{2-[(2-pyridylmethylamino]phenyl}-phenyl)amino]methyl}phenol. mp 178-180° C. ¹H NMR (CDCl₃): □1.53 (1H, broad s), 4.16 (4H, s), 6.92-8.01 (15H, m).

### EXAMPLE 34

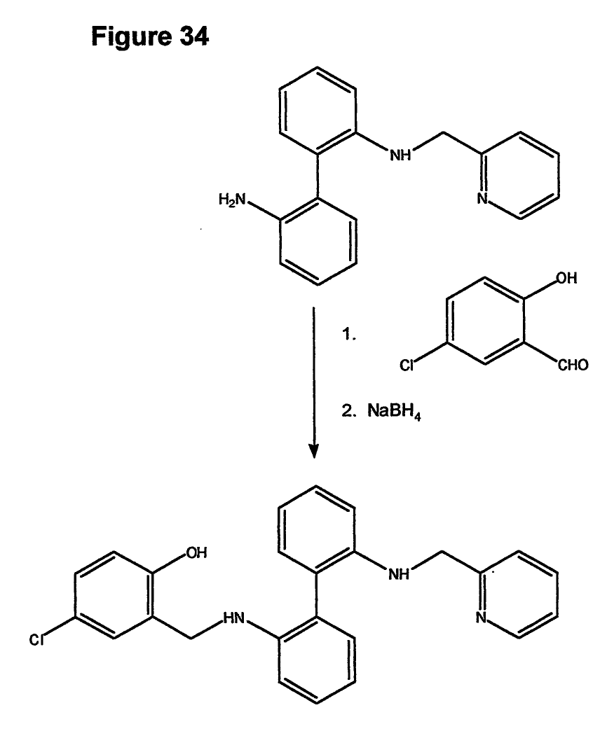

### 4-chloro-2-{[(2-{2-[(2pyridylmethylamino]phenyl}-phenyl)amino]methyl}phenol [FIG. 34]

To 0.60 g (2.18 mmol) of N-(2-pyridylmethyl)-2,2′-diaminobiphenyl in 10 mL of ethanol was added 0.34 g (2.18 mmol) of 5-chlorosalicylaldehyde. The solution was refluxed for 30 min and cooled to room temperate. Yellow crystals were collected by suction filtration to yield 1.06 g (77%) of 2-(2-aza-2-(2-(2-pyridylmethyl)amino)phenyl)phenyl)vinyl)₄chlorophenol, mp 115-116° C.

To 0.35 g (0.91 mmol) of 242-aza-242-(2-pyridylmethyl)amino)phenyl)phenyl)vinyl)₄-chlorophenol in 50 mL of ethanol was added 0.19 g of NaBH₄. The solution was stirred at room temperature for 24 h. The solution was treated with concentrated HCl until acidic, extracted with 2×25 mL of CH₂Cl₂, dried over NaSO₄ and evaporated under reduced pressure. The resultant oil was crystallized from ethyl acetate to yield 0.20 g (57%) of 4-chloro-2-{([(2-{2-[(2-pyridylmethylamino]phenyl}-phenyl)amino]methyl}phenol mp 172-174° C. ¹H NMR (CDCl₃): □1.62 (1H, broad s), 4.52 (4H, s), 7.02-8.31 (15H, m).

### EXAMPLE 35

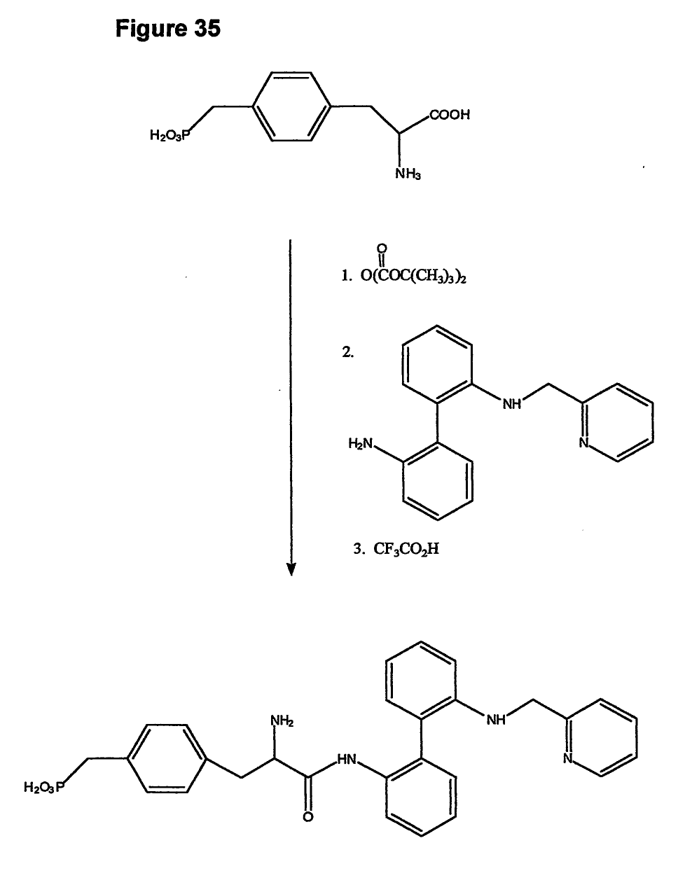

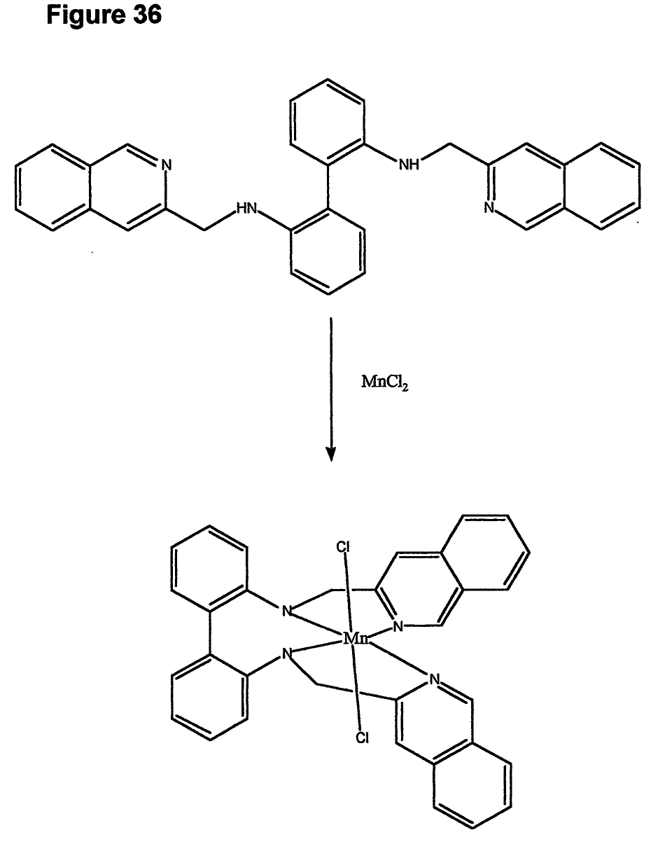

### 2-amino-3-(-(4-phosphonomethylphenyl)-N-(2-{2-[benzylamino]phenyl}phenyl)propanamide [FIG. 35]

To 5 mL of dioxane was added 2.0 g (7.7 mmol) of p-(phosphonomethyl)-DL-phenylalanine, 0.82 g (8.1 mmol) of triethylamine and 1.68 g (7.7 mmol) of di-tert-butyl dicarbonate. The solution was stirred at room temperature for 3 hours. The solution was evaporated at reduced pressure to dryness. The resultant oil was purified by column chromatography using methanol/chloroform to yield 2.1 g (77%) of the Boc-p-(phosphonomethyl)-DL-phenylalanine.

To a solution of 0.77 g (2.78 mmol) of N-(2-pyridylmethyl)-2,2′-diaminobiphenyl in 10 mL of DMF was added 1.0 g (2.78 mmol) of Boc-(phosphonomethyl)-DL-phenylalanine at 0° C. under an atmosphere of nitrogen. To this solution was added a solution of DPPA (1 mL, 2.88 mmol) in 10 mL of DMF and 0.3 g (2.88 mmol) of powered NaHCO₃. The solution was vigorously stirred for 30 hours at 0° C. The solution was diluted with ethyl acetate, washed with 1 N HCl, followed by saturated NaHCO₃. The solution was dried over MgSO₄, filtered, and evaporated under reduced pressure. The oil was purified by flash chromatography to give 1.0 g (62%) of Boc-2-amino-3-(−4-phosphonomethylphenyl)-N-2-{2-[benzylamino]phenyl}phenyl) propanamide as a colorless oil.

A solution of Boc-2-amino-3-(-(4-phosphonomethylphenyl)-N-(2-{2-[benzylamino]phenyl}phenyl) propanamide (0.5 g, 0.81 mmol) in 10 mL of methylene chloride and 2 mL of trifluoroacetic acid was stirred at room temperature for 20 min. The solvent was evaporated at reduced pressure. 2-amino-3-(-(4-phosphonomethylphenyl)-N-2-{2-[benzylamino]phenyl}phenyl) propanamide was isolated as the trifluroacetic acid salt which was treated with ammonia to give 0.27 g (65%) 2amino-3-(-(4-phosphonomethylphenyl)-N-(2-{2-[benzylamino]phenyl}phenyl) propanamide. ¹HNMR (CDCl₃): δ 1.72 (2H, broad s), 2.78 (2H, s), 2.86 and 3.10 (2H, dd), 3.70 (1H, m), 4.48 (2H, s), 6.65-6.85 (8H, m), 7.00-8.50 (7H, m), 8.44 (1H, d).

### EXAMPLE 36

### Manganese (2,2′-diamino(bis-N,N′-quinilylmethyl)biphenyl)(Cl)₂ [FIG. 36]

To 100 mg (0.27 mmol) of 2,2′-diamino (bis-N,N′-quinilylmethyl)biphenyl in 15 mL of methanol was added a solution of 44 mg (0.27 mmol) of Mn(II)Cl₂ in 10 mL of methanol. The solution was heated for 30 min, cooled to room temperature and the solvent was evaporated to 10 mL. Crystals of Manganese (2,2′-diamino (bis-N,N′-quinilylmethyl)biphenyl)(Cl)₂ formed over 7 days which were collected and dried. Anal. Calcd for MnC₃₂C₁₂H₂₆N₄: C, 64.87; H, 4.43; N, 9.45. Found. C, 64.78; H. 4.40; N, 9.94.

### EXAMPLE 37

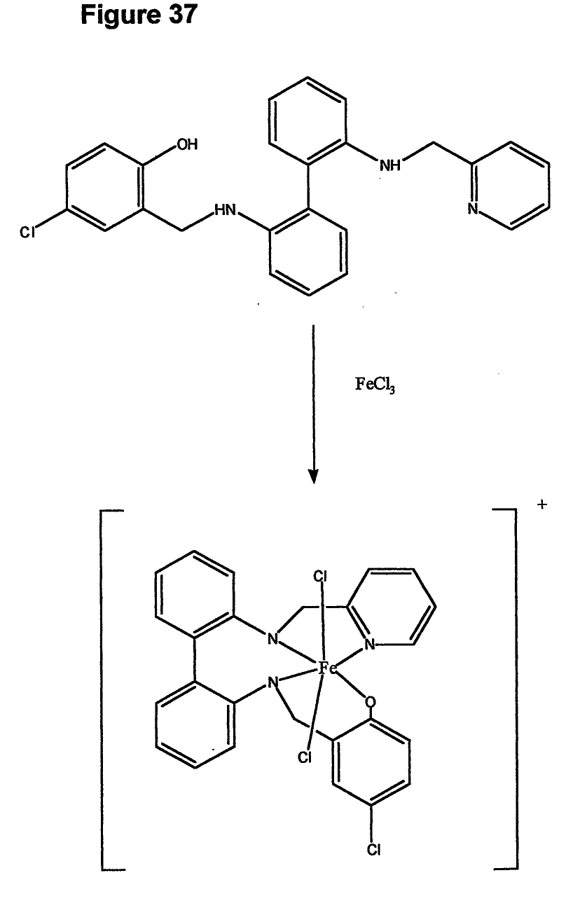

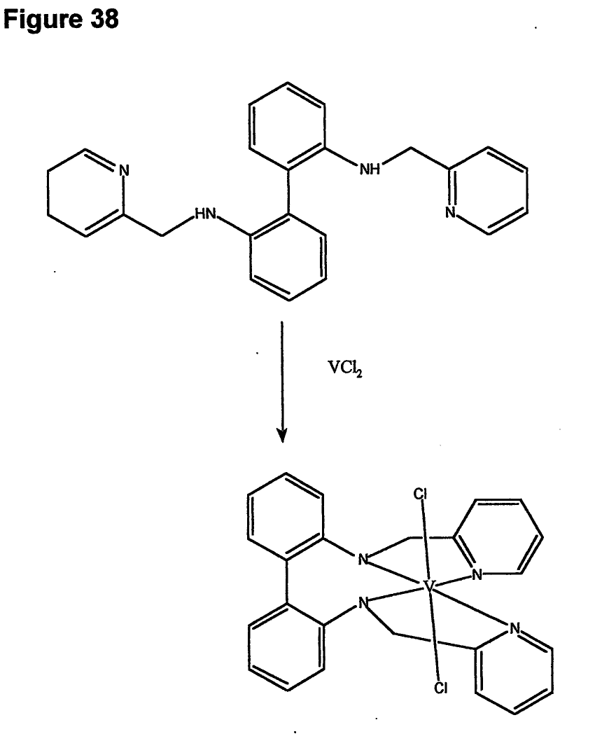

### [Iron (4chloro-2-{[(2-{2-[(2-pyridylmethylamino]phenyl}-phenyl)amino]methyl}phenol)(Cl)₂]Cl [FIG. 37]

To 100 mg (0.26 mmol) of 4-chloro-2-{[(2-{2-[(2-pyridylmethylamino]phenyl}-phenyl)amino]methyl}phenol in 15 mL of methanol was added a solution of 70 mg (0.27 mmol) of Fe(M)Cl₂ in 10 mL of methanol. The solution was heated for 30 min. cooled to room temperature and the solvent was evaporated to 10 mL. Crystals of Iron (4-chloro-2-{[(2-{2-[(2-pyridylmethylamino]phenyl}-phenyl)amino]methyl}phenol)(Cl)₂]Cl formed over 2 days which were collected and dried. Anal. Calcd for FeC₂₅C₄H₂₂N₃O: C, 51.94; H, 3.84; N, 7.26. Found, C, 52.33; H, 3.88; N, 7.41.

### EXAMPLE 38

### [Vanadium (2,2′-diamino(bis-N,N′-pyridylmethyl)biphenyl)Cl₂] [FIG. 38]

To 100 mg (0.27 mmol) of 2,2-diamino (bis-N,N′-pyridylmethyl)biphenyl in 15 mL of methanol was added a solution of 33 mg (0.27 mmol) of V(II)Cl₂ in 10 mL of methanol. The solution was heated for 30 min, cooled to room temperature and the solvent was evaporated to 10 mL. Crystals of [Vanadium (2,2′-diamino(bis-NN′-pyridylmethyl)biphenyl)Cl₂] formed overnight which were collected and dried (78 mg, 69%). Anal. Calcd for VC₂₄C₁₂H₂₂N₄: C, 59.00; 1,4.55; N, 11.47. Found: C, 59.43; H, 4.12; N, 11.37.

### EXAMPLE 39

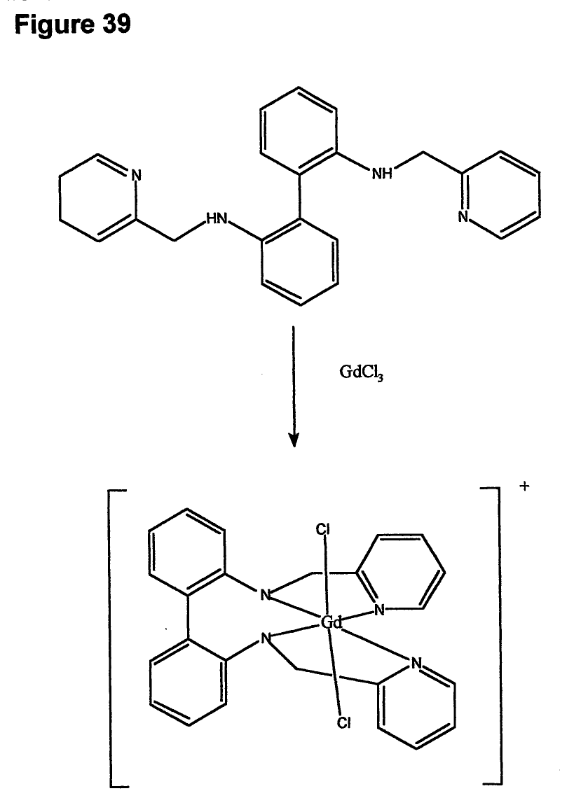

### [Gadolinium (2,2′-diamino(bis N,N′-pyridylmethyl)biphenyl)Cl₂]Cl [FIG. 39]

To 100 mg (0.27 mmol) of 2,2′ diamino (bis-N,N-pyridylmethyl)biphenyl in 15 mL of methanol was added a solution of 100 mg (0.27 mmol) of Gd(III)Cl₃ in 20 mL of methanol. The solution was heated for 30 min, cooled to room temperature and the solvent was evaporated to 10 mL. Crystals of [Gadolinium (2,2′-diamino(bis-N,N′-pyridylmethyl)biphenyl)Cl₂]Cl formed overnight which were collected and dried (78 mg, 69%). Anal. Calcd for GdC₂₋₄C₁₃H₂₂N₄: C, 45.74; 11, 3.53; N, 8.89. Found: C, 45.33; H. 3.78; N, 8.82.

### EXAMPLE 40

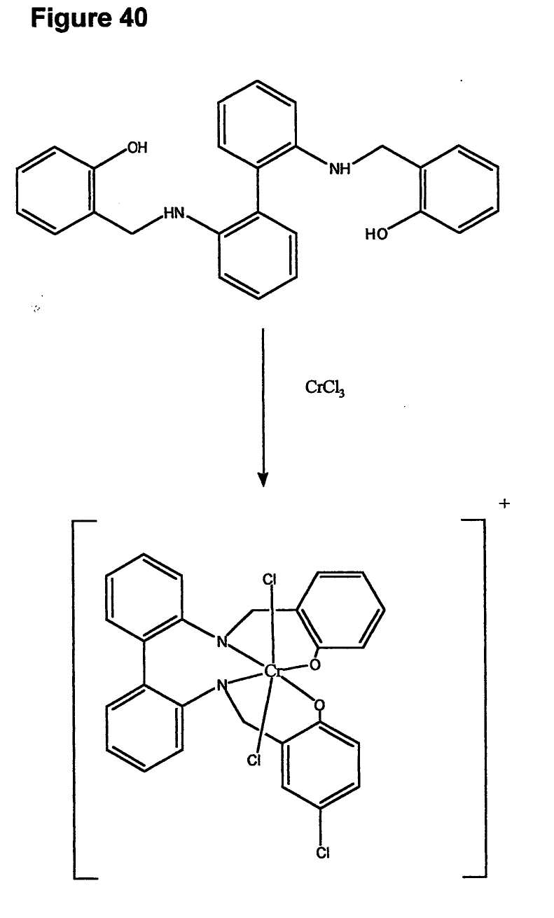

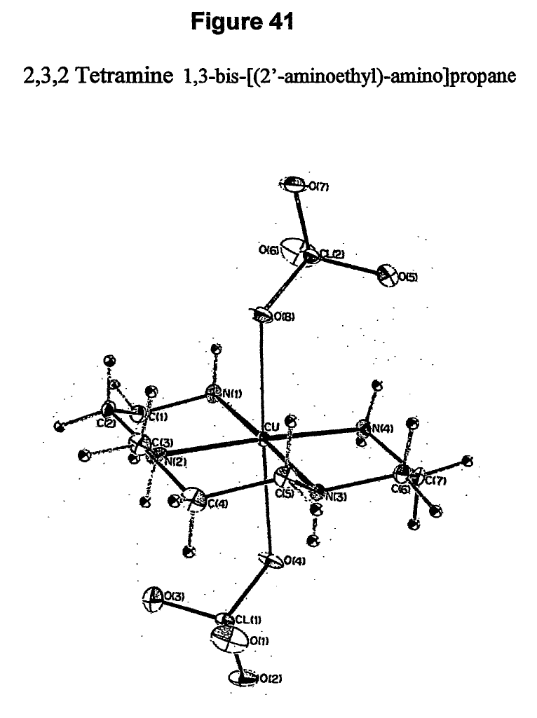

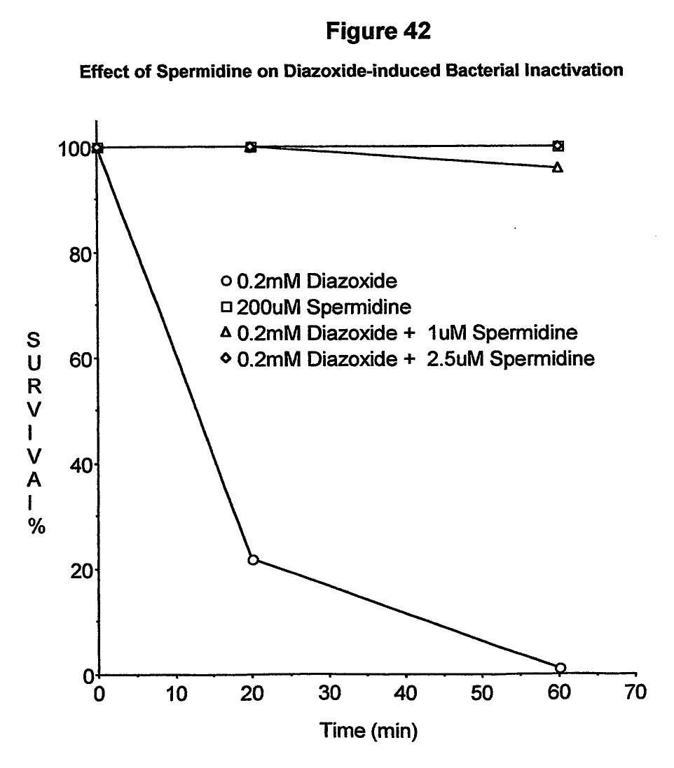

### [Chromium (2-({[2(2-{[2-hydroxyphenyl)methyl]amino}phenyl)phenyl]amino}methyl)phenol)Cl)₂]Cl [FIG. 40]

To 200 mg (0.50 mmol) of 2-({[2-(2-{[2-hydroxyphenyl)methyl]anmino}phenyl) phenyl]amino}methyl)phenol in 25 mL of methanol was added a solution of 133 mg (0.50 mmol) of Cr(III)C₃ in 20 mL of methanol. The solution was heated for 30 min. cooled to room temperature and the solvent was evaporated to 20 mL. Crystals of [Chromium (2-({[2-(2-{[2-hydroxyphenyl)methyl]amino}phenyl)phenyl]amino}methyl)phenol)Cl)₂]Cl formed over 2 weeks which were collected and dried (174 mg, 63%). Anal. Calcd for CrC₂₆C₁₃H₂₄N₂O₂: C, 56.29; H, 4.37; N, 5.05. Found: C, 56.78; H, 4.42; N, 5.01.

### EXAMPLE 41

### Comparison of Stabilities of Metal Ion Complexes

Referring to the heats of formation, the first modification to consider is how the heats of formation are affected by changing the metal ion. The data is quite clear here with the relative stabilities following the pattern: Co>Fe>Mn>Cu>Zn>Cd. Occasionally the Cu complexes are more stable than the Mn but otherwise the trend holds consistently from one set of complexes to another. The trend in changes in stability due to changes in the metal may be exploited by recognizing the affinity that the organic compounds have for various metal ions in the body.

In Table III it is apparent that Fe 2,3,2-piperidine and Fe 2,3,2-adamantane have a low heat of formation which would be attractive to address the excess iron pools in neurodegenerative disease and the excess iron released into brain tissue following lysis of dead neurons post stroke, the adamantane having additional effect on the NMDA receptor.

Similarly from Tables II and IV it is apparent that Fe cyclam methylated and Fe cyclam adamanatane are very stable and Zn cyclam methylated and Zn cyclam adamantine not unduly stable. This behavior could be useful in treating ischemic damage post myocardial infarction where iron exerts toxic redox effects and tissue zinc stores are rapidly depleted.

From Tables II and V it is apparent that there are chain (open ring) molecules binding copper and manganese, Cu 2,3,2-isopropyl on N1/N4 and Mn 3,3,3 respectively are as stable as closed ring molecules. Thus chain (open ring) molecules are comparable with closed ring molecules in their capacity to address free metal excess in neurodegenerative diseases and stroke.

### EXAMPLE 42

### Ring Size

For the 2,3,2-tetramine compounds, formation of 6-membered rings when binding to metal ions increases the stability of the metal complexes. This can be seen when comparing the 3,3,3-tetramine metal complexes to the corresponding 2,3,2-tetramine and the 2,2,2-tetramine compounds. In all cases, the 3,3,3-tetramine complexes are more stable than their 2,3,2-tetramine counterparts. Also, it is generally true that the 2,3,2-tetramine complexes are more stable than the 2,2,2-tetramine complexes. This suggests that the 3,3,3-tetramine compounds may be of considerable interest as companions to the 2,3,2-tetramine compounds. Schugar R and coworkers (Inorg. Chem., 19, 940, 1980) have shown through stability constants that changing the size of the chelate ring has an effect on the resultant stability of the metal complex.

Modification of the cyclam rings so that the rings are smaller or larger also impacts the heats of formation The cyclen complexes are less stable than the cyclam rings, a result that has been documented elsewhere. Increasing the size of the ring as was done for the cyclam 3,3,3-tetramine complexes also leads to enhanced stability compared to cyclam.

These size related changes in stability influence the design of compounds for treatment of neurodegenerative disorders, stroke, glaucoma, Atherosclerosis, cardiomyopathy, ischemia, optic neuropathy, peripheral neuropathy, Presbycussis and cancer.

### EXAMPLE 43

### Addition of Side Groups on Chain (Open Ring) Molecules

Along with changing the size of the rings, various alkyl groups were put on the nitrogens or carbons to see how these modifications affected the stability of the complexes. A number of generalizations can be extracted from the data First, putting small alkyl groups on the nitrogens generally enhances their stability. This can be seen when comparing the 2,3,2-tetramine compounds to the ones where either N1/N4 or N2/N3 are substituted with methyl groups. This result also holds for isopropyl groups substituted on N1/N4 and generally for isopropyls on N2/N3. There is a limit to adding large groups on the nitrogens as seen by the compounds with benzyl substituents. These complexes are very much less stable than the unsubstituted 2,3,2-tetramine complexes.

The placement of alkyl groups on the carbons was only studied in a few cases, but for all of them the addition of methyls led to enhanced stability at a level comparable to that found when the methyls were placed on the nitrogens.

### Addition of Side Groups on Closed Ring Molecules

The trends for the heats of formation of the cyclam complexes are not as consistent as those found for the 2,3,2-tetramine complexes. For example, putting methyl groups on the nitrogens increases the stability in some cases but decreases it in others. The same is true for the complexes where isopropyl is added to the nitrogens. Once again though, benzyl groups greatly decrease the stability of the complexes showing that there is an upper limit as to how bulky the substituents can be before the stability of the complexes is greatly diminished. Surprisingly, the addition of adamantane to the cyclams leads to enhanced stability in all cases. Adamantane is a very large group but it is able to find ways to exist so that the structure is actually quite stable. This stability of the cyclam adamantane compounds may be useful in situations such as stroke and glaucoma where NMDA receptor antagonism is required.

From a biopassaging standpoint the stability of the 2,3,2-isopropyl complexes and molecules with carbon side chains attached to the nag nitrogens or carbons is valuable toward developing compounds which are more lipophilic and thus have better passage across the gastrointestinal tract, blood brain barrier and blood retinal barrier, this being important in the treatment of Parkinson's, Alzheimer's, Lou Gehrig's, Binswanger's, Lewy Body diseases, Olivopontine Cerebellar Degeneration, Stroke, Glaucoma and Optic Neuropathy described herein.

### EXAMPLE 44

### Modifications of Terminal Nitrogens

Another important result is the one shown by changing N1/N4 into piperidine or piperizine nitrogens. It should be noted that these compounds are somewhat different than the ones described above in that the piperidine groups are not added to N1/N4 but rather N1/N4 are replaced by the piperidine or piperizine. With the exception of the copper complexes, these complexes are more stable than the base 2,3,2-tetramine complexes. No generalizations can be made regarding the adamantane compounds but it is noteworthy that they are not excessively unstable compared to the 2,3,2-tetramine compounds (indeed, the Fe complex is more stable while the Co one is equal in stability) even though they are quite large and bulky. This suggests that even large, bulky alkyl groups placed on the nitrogens may not adversely affect their properties and they should be pursued.

The piperidine, piperizine and adamantane derivative molecules are attractive because the terminal groups can substantially alter basicity, lipophilicity and passage through membranes, in addition to altering receptor binding properties. These derivatives may also be attractive where a selective bias towards iron removal versus stored copper removal is sought. This could be applicable to therapeutics for ischemia post myocardial infarction, atherosclerosis and neurodegenerative diseases.

Further the stability of terminally substituted derivatives provides opportunity for substitution with glutathione, uric acid, ascorbic acid, taurine, estrogen, dehydroepiandrosterone, probucol, vitamin B, hydroxytoluene, carvidilol, α-lipoic acid, α-tocopherol, ubiquinone, phylloquinone, β-carotene, meanadione, glutamate, succinate, acetyl-L-carnitine, co-enzyme Q, lazeroids, and polyphenolic flavonoids or homocysteine, menaquinone, idebenone, dantrolene.

These specific derivatives of polyamines may be used as compounds in the treatment of, though not limited to, the following diseases:
glutathione polyamine in peripheral neuropathy and ischemia
uric acid polyamine in stroke
ascorbic acid polyamine in diabetic neuropathy and ischemia
taurine polyamine in diabetic neuropathy
estrogen polyamine in stroke
dehydroepiandrosterone polyamine in stroke
probucol polyamine in peripheral neuropathy
vitamin E polyamine in peripheral neuropathy, Alzheimer's disease, stroke and ischemia,
hydroxytoluene polyamine in peripheral neuropathy
carvidilol polyamine in peripheral neuropathy
α-lipoic acid polyamine in presbycussis, peripheral neuropathy and diabetic neuropathy and Alzheimer's disease
α-tocopherol polyamine in atherosclerosis and ischemia
menaquinone polyamine in diabetes
ubiquinone polyamine in ischemia
phylloquinone (Vitamin K₁) polyamine in atherosclerosis and cardiomyopathy
β-carotene polyamine in ischemia
glutamate polyamine in diabetes
succinate polyamine in diabetes
acetyl-L-carnitine polyamine in Alzheimer's disease and presbycussis
co-enzyme Q polyamine in diabetes, cardiomyoapthy and congestive heart failure
lazeroid (21 aminoquinone) polyamine in stroke
polyphenolic flavonoid (quercetin, catechin, epicatechin) polyamine as antioxidants
homocysteine polyamine in cancer
meanadione (Vitamin K₃) polyamine in cardiomyoapthy
idebenone polyamine in cardiomyoapthy, MELAS and stroke
dantrolene polyamine in stroke
Memantine polyamine, rimantadine polyamine in glaucoma

### EXAMPLE 45

### Internal Substitutions in the Open Chain (Ring) Molecules

It is also possible to replace the nitrogens with other donors such as sulfur. As shown, these complexes excepting the iron ones are considerably more stable than the nitrogen ones. Sulphur containing polyamines terminally derivatized with homocysteine could be used as anti-cancer agents.

Internal Substitutions in the Closed Ring Molecules

Replacing the nitrogens with sulfur enhances the stability of some complexes (Cu, Zn, Co) but not in others (Fe, Mn). This result shows that it is possible to build into the organic compounds selectivity for some metal ions over others. Again a sulphur containing closed ring polyamine derivatized with homocysteine could be used as an anti-cancer agent.

### EXAMPLE 46

### Pharmacokinetic Advantages Versus Stability of Derivatives

Terminal modifications and side chain additions alter pKa, lipophilicity and also the metabolism of these compounds, thus changing half life in vivo. 2,2,2-tetramine is rapidly metabolized to acetyl 2,2,2-tetramine and rapidly excreted with a half life in vivo of only a few hours (Kodama H et al 1997). This metabolism will obviously be altered considerably in terminally derivatized compounds and to some extent in molecules with side chains attached and in internally derivatized molecules. In the treatment of the diseases mentioned above a longer half life and less frent dosing such as once daily dosing will be highly advantageous for therapeutic effect and patient compliance.

Additional Comments

The results in Tables I to VIII shed light on the stability of these molecules and helps direct which ones are appropriate for particular disease situations based upon metal ion selectivity and pharmacological actions and how to enhance the bioavailability of orally or parenterally delivered drugs, and drugs crossing particular membranes such as the blood brain barrier and blood retinal barrier.

### EXAMPLE 47

### Oil Water Partition Coefficients

Partition coefficients were determined by dissolving the compound in a 1:1 mixture of octanol/water and shaking the solution for 12 hours. HPLC was used to determine the partition coefficient The reported values are the log of the octanol/water partition

TABLE IX
Oil Water Partition Coefficients

| | Log Partition Coefficient |
|---|---|
| Compound | Octanol:Water |
| 2,2,2-tetramine | 1.6 |
| 2,3,2-tetramine | 2.1 |
| 2,3,2-pyridine | 2.7 |
| 2,3,2-CH₃ on N1/N4 | 0.4 |
| cyclam-piperidine | 0.7 |

Octanol: water partition log partition coefficients of 2 are optimal for passage through lipid membranes and tissue barriers. Molecules within a range from 0.5 to 4.0 are potential candidates for in vivo use. Thus 2,2,2-tetramine, 2,3,2-tetramine and 2,3,2-pyridine have optimal lipid water partitioning to facilitate their passage through the gastrointestinal barrier and the blood brain barrier.

### EXAMPLE 48

### PKa's

PKa's were determined by standard potentiometric titration methods in aqueous solution with an ionic strength of 0.10 at 25° C. Values are reported as log K values of the equilibrium constant

TABLE X
pKa's

| | pKa(1) | pKa(2) | pKa(3) | pKa(4) |
|---|---|---|---|---|
| 2,2,2-tetramine | 9.7 | 9.1 | 6.6 | 3.3 |
| 2,3,2-tetramine | 10.3 | 9.5 | 7.3 | 6.0 |
| 2,3,2-pyridine | 8.3 | 7.4 | | |
| 2,3,2-piperidine | 9.9 | 9.3 | 6.4 | 3.6 |
| 2,3,2-tetramethyl | 10.2 | 9.4 | 6.1 | 2.9 |
| tetramethylcyclam | 9.7 | 9.3 | 3.1 | 2.6 |

2,3,2-pyridine is less basic and thus more soluble at neutral pH than some of the other amines.

Selection of compounds with appropriate pKa's for use in various diseases where low pKa's would be useful. Selection of compounds with appropriate pKa's for use in various diseases where higher pKas would be useful such as in diabetes and post myocardial infarction.

Experimental Method of Measuring Bacterial Survival in Examples 49-54.

Bacteria were innoculated and cultured in nutrient agar for eighteen hours, using a shaking incubator at 140 r.p.m and 35° C. Medium contained 10 mM H[PES buffer at pH 7. Cells were centrifuged twice in Sorval RC-5 centrifuge 12,000 r.pm.×15 minutes at 4° C. and washed twice in 10 uM HEPES buffer. The resuspended cells were counted using a Hemocrit and diluted to a level of 5×10¹ cells per ml in 10 μM HEPES. The cells, the following toxins; methyl viologen (paraquat), methyl viologen (MPP⁺), rotenone, daizoxide, streptozotocin and alloxan, and antidotes were added, reaching final volumes of 1 ml in Epppendorf tubes and placed on a rotator at room temperature. 10 μM diethylenetriaminepentaacetic acid was added to the samples to terminate the reactions at the specified times of either twenty or sixty minutes. 300 μL of cells were plated on Petri plates containing nutrient agar and incubated at 35° C. overnight. Colonies were counted after 20 hours. Percentage survival as compared with culture controls is calculated from the means of triplicates in each experimental group. Bacteria utilized were E. coli, S. aureus, M. luteus ATCC strains and GM 7359 alkA tag E. coli mutant (Minus ILG. et al 1988 and 1989). Histidine was used for comparison purposes because it ahd previously been reported efficacious in counteracting MPP⁺ and paraquat toxicity in E. Coli (Haskel Y. et al 1991).

### EXAMPLE 49

TABLE XI
Toxins and Antidotes PARAQUAT

Organism: E. coli (ATCC 35150)
Toxin: Paraquat
Antidotes: Histidine, Spermine and 2,3,2-tetramine
Incubation Time: 60 minutes

Analysis of Variance Table

| | | Sum | Mean | |
|---|---|---|---|---|
| Source: | DF: | Squares: | Square: | F-test: |
| Between groups | 11 | 3.546 | .322 | 35.864 |
| Within groups | 24 | .216 | .009 | p = .0001 |
| Total | 35 | 3.762 | | |

Model II estimate of between component variance = .104

| Samples | Mean % | |
|---|---|---|
| PLSD | Survival Fisher | |
| Culture | 100 | |
| 0.5 mM Histidine | 100 | |
| 1 mM Histidine | 100 | |
| 0.5 mM Spermine | 100 | |
| 1 mM Spermine | 96 | |
| 1 mM 2,3,2-tetramine | 100 | |
| 0.5 mM Paraquat | 16 | |
| 1 mM Paraquat | 10 | |
| 0.5 mM Paraquat + 0.5 mM Histidine | 30 | 0.217 |
| 0.5 mM Paraquat + 1.0 mM Histidine | 37 | 0.217 |
| 0.5 mM Paraquat + 0.5 mM Spermine | 60 | 0.217 Sig. at 99% |
| 1.0 mM Paraquat + 0.5 mM Spermine | 100 | 0.217 Sig. at 99% |
| 1.0 mM Paraquat + 1.0 mM 2,3,2-tetramine | 100 | 0.217 Sig. at 99% |

Spermine and 2,3,2-tetramine were more efficacious in preventing cell loss than histidine at comparable doses.

TABLE XII
Toxins and Antidotes PARAQUAT

Organism: S. aureus (ATCC 29213)
Toxin: Paraquat
Antidotes: 2,3,2-tetramine and Cyclam
Incubation Time: 60 minutes

Analysis of Variance Table

| | | Sum | Mean | |
|---|---|---|---|---|
| Source: | DF: | Squares: | Square: | F-test: |
| Between groups | 6 | .884 | .147 | 66.512 |
| Within groups | 14 | .031 | .002 | p = .0001 |
| Total | 20 | .915 | | |

Model II estimate of between component variance = .048

| Samples | Mean % | |
|---|---|---|
| PLSD | Survival Fisher | |
| Culture | 100 | |
| 0.5 mM 2,3,2-tetramine | 100 | |
| 1 mM 2.3,2-tetramine | 100 | |
| 30 uM Cyclam | 87 | |
| 0.5 mM 2,3,2-tetramine | 39 | |
| 0.5 mM Paraquat | 39 | |
| 0.5 mM Paraquat + 0.5 mM 2,3,2-tetramine | 78 | 0.114 Sig. at 99% |
| 0.5 mM Paraquat + 1 mM 2,3,2-tetramine | 80 | 0.114 Sig. at 99% |
| 0.5 mM Paraquat + 30 uM Cyclam | 71 | 0.114 Sig. at 99% |

2,3,2-tetramine and cyclam protected against paraquat induced cell killing, paraquat being effective at lower doses than 2,3,2-tetramine.

### EXAMPLE 50

TABLE XIII
Toxins and Antidotes MPP⁺

Organism: S. aureus (ATCC 29213)
Toxin: MPP⁺
Antidotes: Histidine, 2,3,2-piperidine, Cyclam and Cyclam Adamantane
Incubation Time: 60 minutes

Analysis of Variance Table

| | | Sum | Mean | |
|---|---|---|---|---|
| Source: | DF: | Squares: | Square: | F-test: |
| Between groups | 19 | 5.847 | .308 | 29.918 |
| Within groups | 58 | .597 | .01 | p = .0001 |
| Total | 77 | 6.444 | | |

Model II estimate of between component variance = .078

| Samples | Mean % | |
|---|---|---|
| PLSD | Survival Fisher | |
| Culture | 100 | |
| 0.5 mM Histidine | 90 | |
| 7.5 uM 2,3,2-piperidine | 100 | |
| 30 uM Cyclam | 81 | |
| 30 uM Cyclam Adamantane | 100 | |
| 0.1 mM MPP⁺ | 31 | |
| 0.15 mM MPP⁺ | 15 | |
| 0.2 mM MPP⁺ | 6 | |
| 0.25 mM MPP⁺ | 3 | |
| 0.15 mM MPP⁺ 0.5 mM Histidine | 64 | 0.242 Sig. at 99.5% |
| 0.25 mM MPP⁺ + 1 uM 2,3,2- | 54 | 0.242 Sig. at 99.5% |
| piperidine | | |
| 0.25 mM MPP⁺ + 2 uM 2,3,2- | 54 | 0.242 Sig. at 99.5% |
| piperidine | | |
| 0.25 mM MPP⁺ + 5 uM 2,3,2- | 69 | 0.242 Sig. at 99.5% |
| piperidine | | |
| 0.25 mM MPP⁺ + 7 uM 2,3,2- | 100 | 0.191 Sig. at 99.5% |
| piperidine | | |
| 0.15 mM MPP⁺ + 30 uM Cyclam | 79 | 0.242 Sig. at 99.5% |
| 0.2 mM MPP⁺ + 1 uM Cyclam | 100 | 0.242 Sig. at 99.5% |
| Adamantane | | |
| 0.2 mM MPP⁺ + 2.5 uM Cyclam | 100 | 0.242 Sig. at 99.5% |
| Adamantane | | |
| 0.2 mM MPP⁺ + 5.0 uM Cyclam | 100 | 0.242 Sig. at 99.5% |
| Adamantane | | |
| 0.2 mM MPP⁺ + 7.5 uM Cyclam | 100 | 0.242 Sig. at 99.5% |
| Adamantane | | |

Micromolar doses of 2,3,2-piperidine and cyclam adamantane protected against 200 uM doses of MPP⁺ whereas histidine was only effective in substantially higher dose.

### EXAMPLE 51

TABLE XIV
Toxins and Antidotes ROTENONE

Organism: E. coli (GM 7359) alkA tag E. coli mutant
Toxin: Rotenone
Antidotes: 2,3,2-piperidine, 2,3,2-pyridine, Chromium 2,3,2-pyridine,
2,2,2-tetramine, 2,3,2-diCH₃ and Cyclam Adamantane
Incubatiion Time: 60 minutes

Analysis of Variance Table

| | | Sum | Mean | |
|---|---|---|---|---|
| Source: | DF: | Squares: | Square: | F-test: |
| Between groups | 31 | .911 | .029 | 5.955 |
| Within groups | 103 | .508 | .005 | p = .0001 |
| Total | 134 | 1.419 | | |

Model II estimate of between component variance = .006

| Samples | Mean % | |
|---|---|---|
| PLSD | Survival Fisher | |
| Culture | 100 | |
| 50 uM 2,3,2-piperidine | 100 | |
| 50 uM 2,3,2-pyridine | 100 | |
| 1 uM Chromium 2,3,2-pyridine | 100 | |
| 2 mM 2,2,2-tetramine | 100 | |
| 25 uM 2,3,2-diCH3 | 100 | |
| 25 uM Cyclam Adamantane | 100 | |
| 100 uM Rotenone | 60 | |
| 100 uM Rotenone + 2.5 uM 2,3,2-piperidine | 100 | 0.182 Sig. |
| | | at 99.8% |
| 100 uM Rotenone + 5 uM 2,3,2-piperidine | 100 | 0.182 Sig. |
| | | at 99.8% |
| 100 uM Rotenone + 20 uM 2,3,2-piperidine | 100 | 0.182 Sig. |
| | | at 99.8% |
| 100 uM Rotenone + 50 uM 2,3,2-piperidine | 100 | 0.182 Sig. |
| | | at 99.8% |
| 100 uM Rotenone + 2.5 uM 2,3,2-pyridyine | 100 | 0.182 Sig. |
| | | at 99.8% |
| 100 uM Rotenone + 5 uM 2,3,2-pyridyine | 100 | 0.182 Sig. |
| | | at 99.8% |
| 100 uM Rotenone + 20 uM 2,3,2-pyridyine | 100 | 0.182 Sig. |
| | | at 99.8% |
| 100 uM Rotenone + 50 uM 2,3,2-pyridyine | 100 | 0.182 Sig. |
| | | at 99.8% |
| 100 uM Rotenone + 1 um Chromium 2,3,2-pyridine | 100 | 0.182 Sig. |
| | | at 99.8% |
| 100 mM Rotenone + 0.5 mM 2,2,2-tetramine | 67 | 0.182 |
| 100 mM Rotenone + 2 mM 2,2,2-tetramine | 100 | 0.182 Sig. |
| | | at 99.8% |
| 100 uM Rotenone + 2.5 uM 2,3,2-diCH₃ | 100 | 0.182 Sig. |
| | | at 99.8% |
| 100 uM Rotenone + 5 uM 2,3,2-diCH₃ | 89 | 0.182 Sig. |
| | | at 99.8% |
| 100 uM Rotenone + 12.5 uM 2,3,2-diCH₃ | 100 | 0.182 Sig. |
| | | at 99.8% |
| 100 uM Rotenone + 25 uM 2,3,2-diCH₃ | 100 | 0.182 Sig. |
| | | at 99.8% |
| 100 uM Rotenone + 2.5 uM Cyclam Adamantane | 97 | 0.182 Sig. |
| | | at 99.8% |
| 100 uM Rotenone + 5 uM Cyclam Adamantane | 89 | 0.182 Sig. |
| | | at 99.8% |
| 100 uM Rotenone + 12.5 uM Cyclam Adamantane | 100 | 0.182 Sig. |
| | | at 99.8% |
| 100 uM Rotenone + 25 uM Cyclam Adamantanane | 100 | 0.182 Sig. |
| | | at 99.8% |

Low micromolar doses of 2,3,2-pyridine, 2,3,2-diCH₃ and cyclam adamantane protected against rotenone induced cell killing.

### EXAMPLE 52

TABLE XV
Toxins and Antidotes DIAZOXIDE

Organism S. aureus (ATCC 29213)
Toxin: Diazoxide
Antidotes: Histidine, Spermine, 2,3,2-tetramine, 2,3,2-piperidine,
2,3,2-pyridine and Cyclam
Incubation Time: 60 minutes

Analysis of Variance Table

| | | Sum | Mean | |
|---|---|---|---|---|
| Source: | DF: | Squares: | Square: | F-test: |
| Between groups | 25 | 2.688 | .108 | 5.668 |
| Within groups | 76 | 1.442 | .019 | p = .0001 |
| Total | 101 | 4.13 | | |

Model II estimate of between component variance = .023

| Samples | Mean % | |
|---|---|---|
| PLSD | Survival Fisher | |
| Culture | 100 | |
| 0.5 mM Histidine | 100 | |
| 5 uM Spermine | 99 | |
| 20 uM 2,3,2-tetramine | 99 | |
| 10 uM 2,3,2-piperidine | 100 | |
| 5 uM 2,3,2-pyridine | 99 | |
| 10 uM Cyclam | 100 | |
| 0.15 mM Diazoxide | 25 | |
| 0.15 mM Diazoxide + 500 uM Histidine | 58 | 0.36 |
| 0.15 mM Diazoxide + 5 uM Spermine | 71 | 0.36 Sig. |
| | | at 99.8% |
| 0.15 mM Diazoxide + 2.5 uM 2,3,2-tetramine | 65 | 0.36 Sig. |
| | | at 99.8% |
| 0.15 mM Diazoxide + 5 uM 2,3,2-tetramine | 78 | 0.36 Sig. |
| | | at 99.8% |
| 0.15 MM Diazoxide + 10 uM 2,3,2-tetramine | 78 | 0.36 Sig. |
| | | at 99.8% |
| 0.15 mM Diazoxide + 20 uM 2,3,2-tetramine | 75 | 0.36 Sig. |
| | | at 99.8% |
| 0.15 mM Diazoxide + 2.5 ,2,3,2-piperidine | 100 | 0.36 Sig. |
| | | at 99.8% |
| 0.15 mM Diazoxide + 5 uM 2,3,2-piperidine | 91 | 0.36 Sig. |
| | | at 99.8% |
| 0.15 mM Diazoxide + 10 uM 2,3,2-piperidine | 92 | 0.36 Sig. |
| | | at 99.8% |
| 0.15 mM Diazoxide + 2.5 uM 2,3,2-pyridine | 61 | 0.36 Sig. |
| | | at 99.8% |
| 0.15 mM Diazoxide + 5 uM 2,3,2-pyridine | 84 | 0.36 Sig. |
| | | at 99.8% |
| 0.15 mM Diazoxide + 2.5 uM Cyclam | 99 | 0.36 Sig. |
| | | at 99.8% |
| 0.15 mM Diazoxide + 5 uM Cyclam | 85 | 0.36 Sig. |
| | | at 99.8% |
| 0.15 mM Diazoxide + 10 uM Cyclam | 98 | 0.36 Sig. |
| | | at 99.8% |

2,3,2-tetramine, 2,3,2-piperidine, 2,3,2-pyridine and cyclam prevented diazoxide induced cell killing at low micromolar doses whereas histidine was partially protectiveat substantially higher doses.

TABLE XVI
Toxins and Antidotes DIAZOXIDE

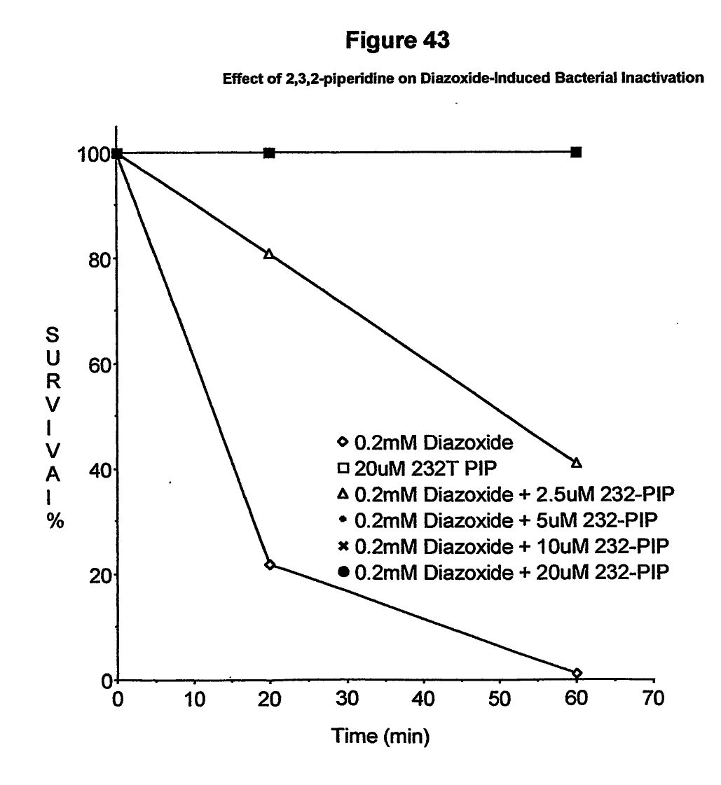

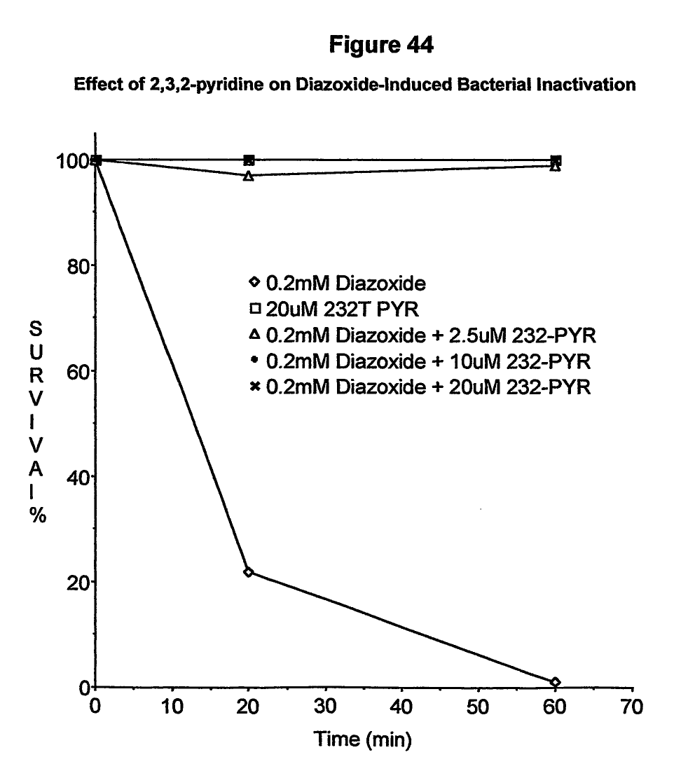

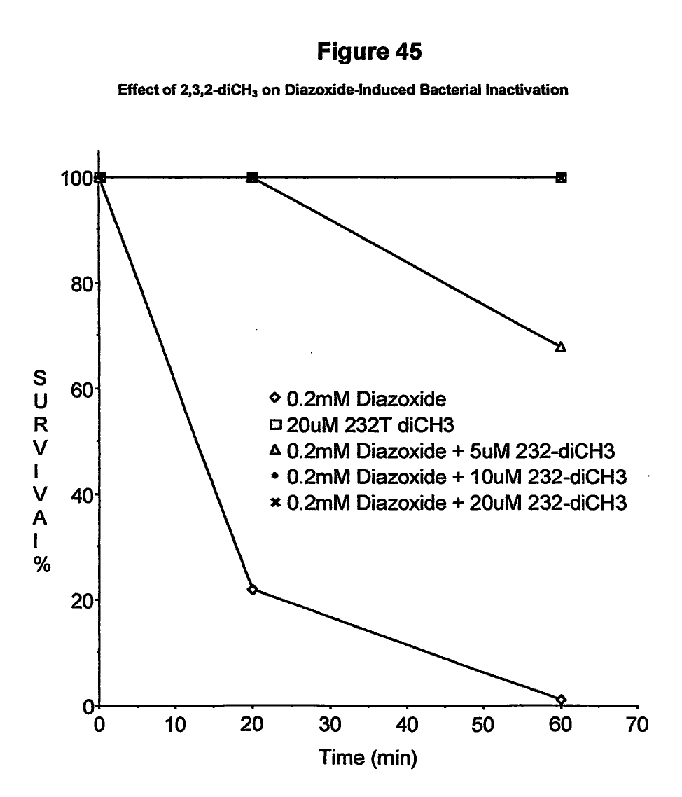

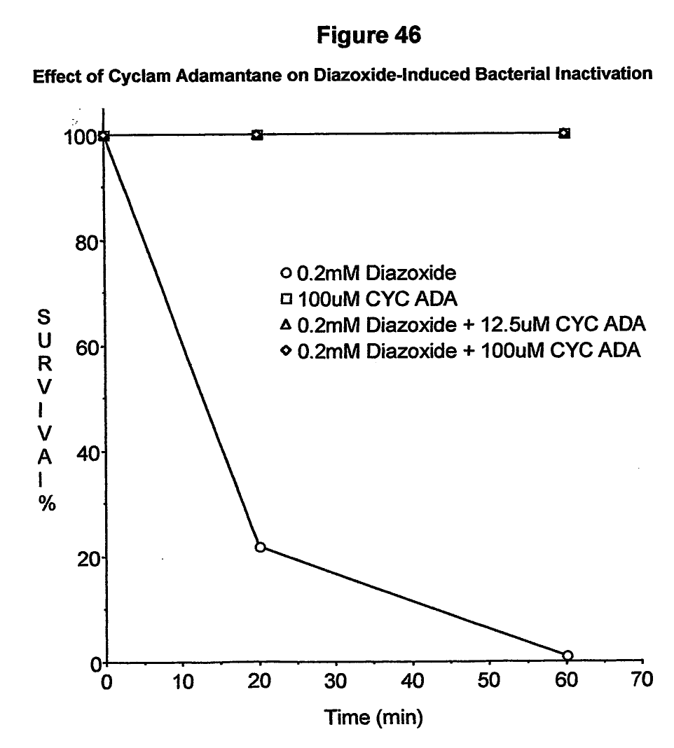

Organism: M. Luteus (ATCC 499732)
Toxin: Diazoxide
Antidotes: Histidine, Spermidine, 2,3,2-piperidine, 2,3,2-pyridine,
Chromium 2,3,2-pyridine, 2,3,2-diCH₃, 2,3,2-sulfur, Cyclam
Adamantane, Vanadium Cyclam Adamantane and Cyclam Piperidine
Incubation Times: 20 and 60 minutes
T20 = Incubation time of 20 minutes
T650 = Incubation time of 60 minutes
See FIGS. 43-47.

| Samples | Mean % |
|---|---|
| PLSD | Survival Fisher |
| T20 | |
| Culture | 100 |
| 200 uM Histidine | 100 |
| 200 uM Spermidine | 100 |
| 50 uM 2,3,2-piperidine | 100 |
| 40 uM 2,3,2 pyridine1 | 100 |
| 5 uM Chromium 2,3,2-pyridine | 100 |
| 5 uM Vanadium 2,3,2-pyridine | 100 |
| 40 uM 2,3,2-diCH₃ | 100 |
| 100 uM 2,3,2-sulfur | 100 |
| 100 uM Cyclam Adamantane | 100 |
| 200 uM Vanadium Cyclam Adamantane | 100 |
| 500 nM Cyclam piperidine | 100 |
| 0.2 mM Diazoxide | 22 |
| T60 | |
| Culture | 100 |
| 200 uM Histidine | 100 |
| 200 uM Spermidine | 100 |
| 50 uM 2,3,2-piperidine | 100 |
| 40 uM 2,3,2-pyridine | 100 |
| 5 uM Chromium 2,3,2-pyridine | 100 |
| 5 uM Vanadium 2,3,2-pyridine | 100 |
| 40 uM 2,3,2-diCH₃ | 100 |
| 100 uM 2,3,2-sulfur | 100 |
| 100 uM Cyclam Adamantane | 100 |
| 200 uM Vanadium Cyclam Adamantane | 100 |
| 500 nM Cyclam Piperidine | 100 |
| 0.2 mM Diazoxide | 1 |

Analysis of Variance Table

| | | Sum | Mean | |
|---|---|---|---|---|
| Source: | DF: | Squares: | Square: | F-test: |
| Between groups | 2 | 1.478 | .739 | 86.793 |
| Within groups | 6 | .051 | .009 | p = .0001 |
| Total | 8 | 1.53 | | |

Model II estimate of between component variance = .244

| T60 | | |
|---|---|---|
| Histidine | | |
| 0.2 mM Diazoxide + 200 uM Histidine | 54 | 0.184 Sig. at 95% |

Analysis of Variance Table

| | | Sum | Mean | |
|---|---|---|---|---|
| Source: | DF: | Squares: | Square: | F-test: |
| Between groups | 4 | 1.496 | .374 | 2467.867 |
| Within groups | 11 | .002 | 1.515E−4 | p = .0001 |
| Total | 15 | 1.497 | | |

Model II estimate of between component variance = .117
T20

| | | |
|---|---|---|
| Spermidine | | |
| 0.2 mM Diazoxide + 1 uM Spermidine | 100 | 0.031 Sig. at 99% |
| 0.2 mM Diazoxide + 2.5 uM Spermidine | 100 | 0.031 Sig. at 99% |
| 0.2 mM Diazoxide + 200 uM Spermidine | 100 | 0.031 Sig. at 99% |

Analysis of Variance Table

| | | Sum | Mean | |
|---|---|---|---|---|
| Source: | DF: | Squares: | Square: | F-test: |
| Between groups | 3 | 2.141 | .714 | 189.358 |
| Within groups | 8 | .03 | .004 | p = .0001 |
| Total | 11 | 2.171 | | |

Model II estimate of between component variance = .237
T60

| | | |
|---|---|---|
| Spermidine | | |
| 0.2 mM Diazoxide + 1 uM Spermidine | 96 | 0.168 Sig. at 99% |
| 0.2 mM Diazoxide + 2.5 uM Spermidine | 100 | 0.168 Sig. at 99% |

Analysis of Variance Table

| | | Sum | Mean | |
|---|---|---|---|---|
| Source: | DF: | Squares: | Square: | F-test: |
| Between groups | 7 | 1.1 | .183 | 16.948 |
| Within groups | 16 | .151 | .011 | p = .0001 |
| Total | 23 | 1.251 | | |

Model II estimate of between component variance = .058
T20

| | | |
|---|---|---|
| 2,3,2-piperidine | | |
| 0.2 mM Diazoxide + 1 uM 2,3,2-piperidine | 81 | 0.253 Sig. at 99% |
| 0.2 mM Diazoxide + 2.5 uM 2,3,2-piperidine | 81 | 0.253 Sig. at 99% |
| 0.2 mM Diazoxideu + 5 uM 2,3,2iperidine | 100 | 0.253 Sig. at 99% |
| 0.2 mM Diazoxide + 10 uM 2,3,2-piperidine | 100 | 0.253 Sig. at 99% |
| 0.2 mM Diazoxide + 20 uM 2,3,2-piperidine | 100 | 0.253 Sig. at 99% |
| 0.2 mM Diazoxide + 50 uM 2,3,2-piperidine | 100 | 0.253 Sig. at 99% |

Analysis of Variance Table

| | | Sum | Mean | |
|---|---|---|---|---|
| Source: | DF: | Squares: | Square: | F-test: |
| Between groups | 5 | 2.736 | .547 | 22.431 |
| Within groups | 12 | .293 | .024 | p = .0001 |
| Total | 17 | 3.029 | | |

Model II estimate of between component variance = .174
T60

| | | |
|---|---|---|
| 2,3,2-piperidine | | |
| 0.2 mM Diazoxide + 2.5 uM 2,2-piperidine | 41 | 0.39 Sig. at 99% |
| 0.2 mM Diazoxide + 5 uM 2,3,2-piperidine | 100 | 0.39 Sig. at 99% |
| 0.2 mM Diazoxide + 10 uM 2,3 -piperidine | 100 | 0.39 Sig. at 99% |
| 0.2 mM diazoxide + 20 uM 2,3 -piperidine | 100 | 0.39 Sig. at 99% |

Analysis of Variance Table

| | | Sum | Mean | |
|---|---|---|---|---|
| Source: | DF: | Squares: | Square: | F-test: |
| Between groups | 6 | 1.067 | .178 | 21.751 |
| Within groups | 14 | .114 | .008 | p = .0001 |
| Total | 20 | 1.182 | | |

Model II estimate of between component variance = .057
T20

| | | |
|---|---|---|
| 2,3,2-pyridine | | |
| 0.2 mM Diazoxide + 2.5 uM 2,3,2-pyridine | 96 | 0.22 Sig. at 99% |
| 0.2 mM Diazoxide + 5 uM 2,3,2-pyridine | 93 | 0.22 Sig. at 99% |
| 0.2 mM Diazoxide + 10 uM 2,3,2-pyridine | 100 | 0.22 Sig. at 99% |
| 0.2 mM Diazoxide + 20 uM 2,3,2-pyridine | 100 | 0.22 Sig. at 99% |
| 0.2 mM Diazoxide + 40 uM 2,3,2-pyridine | 100 | 0.22 Sig. at 99% |

Analysis of Variance Table

| | | Sum | Mean | |
|---|---|---|---|---|
| Source: | DF: | Squares: | Square: | F-test: |
| Between groups | 6 | 2.481 | .413 | 10.74 |
| Within groups | 14 | .539 | .038 | p = .0001 |
| Total | 20 | 3.02 | | |

Model II estimate of between component variance = .125
T60

| | | |
|---|---|---|
| 2,3,2-pyridine | | |
| 0.2 mM Diazoxide + 2.5 uM 2,3,2-pyridine | 99 | 0.477 Sig. at 99% |
| 0.2 mM Diazoxide + 5 uM 2,3,2-pyridine | 100 | 0.477 Sig. at 99% |
| 0.2 mM Diazoxide + 10 uM 2,3,2-pyridine | 100 | 0.477 Sig. at 99% |
| 0.2 mM Diazoxide + 20 uM 2,3,2-pyridine | 100 | 0.477 Sig. at 99% |
| 0.2 mM Diazoxide + 40 uM 2,3,2-pyridine | 70 | 0.477 Sig. at 99% |

Analysis of Variance Table

| | | Sum | Mean | |
|---|---|---|---|---|
| Source: | DF: | Squares: | Square: | F-test: |
| Between groups | 3 | 2.217 | .739 | 354.556 |
| Within groups | 8 | .017 | .002 | p = .0001 |
| Total | 11 | 2.234 | | |

Model II estimate of between component variance = .246

| T60 | | |
|---|---|---|
| Chromium 2,3,2-pyridine | | |
| 0.2 mM Diazoxide + 1 uM chromium | 100 | 0.125 Sig. at 99% |
| 2,3,2-pyridine | | |
| 0.2 mM Diazoxide + 5 uM chromium | 100 | 0.125 Sig. at 99% |
| 2,3,2-pyridine | | |

Analysis of Variance Table

| | | Sum | Mean | |
|---|---|---|---|---|
| Source: | DF: | Squares: | Square: | F-test: |
| Between groups | 2 | 1.63 | .815 | 81.248 |
| Within groups | 6 | .06 | .01 | p = .0001 |
| Total | 8 | 1.691 | | |

Model II estimate of between component variance = .268

| | | |
|---|---|---|
| Vanadium 2,3,2-pyridine | | |
| 0.2 mM Diazoxide + 5 uM Vanadium | 80 | 0.303 Sig. at 99% |
| 2,3,2-pyridine | | |

Analysis of Variance Table

| | | Sum | Mean | |
|---|---|---|---|---|
| Source: | DF: | Squares: | Square: | F-test: |
| Between groups | 5 | 1.113 | .223 | 40.179 |
| Within groups | 15 | .083 | .006 | p = .0001 |
| Total | 20 | 1.196 | | |

Model II estimate of between component variance = .063
T20

| | | |
|---|---|---|
| 2,3,2-diCH₃ | | |
| 0.2 mM Diazoxide + 5 uM 2,3,2-diCH₃ | 100 | 0.155 Sig. at 99% |
| 0.2 mM Diazoxide + 10 uM 2,3,2-diCH₃ | 100 | 0.179 Sig. at 99% |
| 0.2 mM Diazoxide + 20 uM 2,3,2-diCH₃ | 100 | 0.179 Sig. at 99% |
| 0.2 mM Diazoxide + 40 uM 2,3,2-diCH₃ | 100 | 0.179 Sig. at 99% |

Analysis of Variance Table

| | | Sum | Mean | |
|---|---|---|---|---|
| Source: | DF: | Squares: | Square: | F-test: |
| Between groups | 5 | 2.353 | .471 | 8.407 |
| Within groups | 12 | .672 | .056 | p = .0013 |
| Total | 17 | 3.025 | | |

Model II estimate of between component variance = .138
T60

| | | |
|---|---|---|
| 2,3,2-diCH₃ | | |
| 0.2 mM Diazoxide + 5 uM 2,3,2-diCH₃ | 68 | 0.59 Sig. at 99% |
| 0.2 mM Diazoxide + 10 uM 2,3,2-diCH₃ | 100 | 0.59 Sig. at 99% |
| 0.2 mM Diazoxide + 20 uM 2,3,2-diCH₃ | 100 | 0.59 Sig. at 99% |
| 0.2 mM Diazoxide + 40 uM 2,3,2-diCH₃ | 95 | 0.59 Sig. at 99% |

Analysis of Variance Table

| | | Sum | Mean | |
|---|---|---|---|---|
| Source: | DF: | Squares: | Square: | F-test: |
| Between groups | 4 | 1.664 | .416 | 9.477 |
| Within groups | 13 | .57 | .044 | p = .0008 |
| Total | 17 | 2.234 | | |

Model II estimate of between component variance = .106
T60

| | | |
|---|---|---|
| 2,3,2-Sulfur | | |
| 0.2 mM Diazoxide + 12.5 uM 2,3,2-sulfur | 32 | 0.446 |
| 0.2 mM Diazoxide + 100 uM 2,3,2-sulfur | 67 | 0.515 Sig. at 99% |

Analysis of Variance Table

| | | Sum | Mean | |
|---|---|---|---|---|
| Source: | DF: | Squares: | Square: | F-test: |
| Between groups | 6 | 1.349 | .337 | 351 |
| Within groups | 17 | .012 | .001 | p = .0001 |
| Total | 23 | 1.362 | | |

Model II estimate of between component variance = .096
T20

| | | |
|---|---|---|
| Cyclam Adamantane | | |
| 0.2 mM Diazoxide + 12.5 uM Cyclam | 100 | 0.076 Sig. at 99% |
| Adamantane | | |
| 0.2 mM Diazoxide + 50 uM Cyclam | 100 | 0.076 Sig. at 99% |
| Adamantane | | |
| 0.2 mM Diazoxide + 100 uM Cyclam | 100 | 0.076 Sig. at 99% |
| Adamantane | | |

Analysis of Variance Table

| | | Sum | Mean | |
|---|---|---|---|---|
| Source: | DF: | Squares: | Square: | F-test: |
| Between groups | 6 | 2.478 | .413 | 4.892 |
| Within groups | 23 | 1.942 | .084 | p = .0023 |
| Total | 29 | 4.42 | | |

Model II estimate of between component variance = .084
T60

| | | |
|---|---|---|
| Cyclam Adamantane | | |
| 0.2 mM Diazoxide + 500 nM Cyclam | 47 | 0.666 |
| Adamantane | | |
| 0.2 mM Diazoxide + 12.5 uM Cyclam | 100 | 0.666 Sig. at 99% |
| Adamantane | | |
| 0.2 mM Diazoxide + 100 uM Cyclam | 100 | 0.666 Sig. at 99% |
| Adamantane | | |

Analysis of Variance Table

| | | Sum | Mean | |
|---|---|---|---|---|
| Source: | DF: | Squares: | Square: | F-test: |
| Between groups | 7 | 2.71 | .387 | 650.019 |
| Within groups | 28 | .017 | .001 | p = .0001 |
| Total | 35 | 2.727 | | |

Model II estimate of between component variance = .097

| T60 | | |
|---|---|---|
| Vanadium Cyclam Adamantane | | |
| 0.2 mM Diazoxide + 1 uM | 100 | 0.055 Sig. at 99% |
| Vanadium Cyclam Adamantane | | |
| 0.2 mM Diazoxide + 5 uM | 100 | 0.055 Sig. at 99% |
| Vanadium Cyclam Adamantane | | |
| 0.2 mM Diazoxide + 10 uM | 100 | 0.055 Sig. at 99% |
| Vanadium Cyclam Adamantane | | |
| 0.2 mM Diazoxide + 50 uM | 100 | 0.055 Sig. at 99% |
| Vanadium Cyclam Adamantane | | |
| 0.2 mM Diazoxide + 200 uM | 100 | 0.055 Sig. at 99% |
| Vanadium Cyclam Adamantane | | |

Analysis of Variance Table

| | | Sum | Mean | |
|---|---|---|---|---|
| Source: | DF: | Squares: | Square: | F-test: |
| Between groups | 2 | 1.481 | .741 | 661.484 |
| Within groups | 6 | .007 | .001 | p = .0001 |
| Total | 8 | 1.488 | | |

Model II estimate of between component variance = .246
T60

| | | |
|---|---|---|
| Cyclam Piperidine | | |
| 0.2 mM Diazoxide + 500 nM Cyclam Piperidine | 53 | 0.101 Sig. at 99% |

Spermidine, 2,3,2-piperidine, 2,3,2-pyridine, vanadium 2,3,2-pyridine, chromium 2,3,2-pyridine, 2,3,2-diCH₃, 2,3,2-sulfur, cyclam adamantane, vanadium cyclam adamantane and cyclam piperidine prevented diazoxide induced cell killing at low micromolar doses whereas histidine required higher doses to prevent cell killing.

### EXAMPLE 53

TABLE XVII
Toxins and Antidotes STREPTOZOTOCIN

Organism: E. Coli (GM 7359) alkA tag E. coli mutant
Toxin: Streptozotocin
Antidotes: Spermidine, 2,3,2-piperidine, 2,3,2-pyridine, 2,3,2-diCH₃and
Cyclam Adamantane
Incubation Time: 60 minutes

| | |
|---|---|
| Samples | Mean % |
| PLSD | Survival Fisher |

Analysis of Variance Table

| | | Sum | Mean | |
|---|---|---|---|---|
| Source: | DF: | Squares: | Square: | F-test: |
| Between groups | 23 | .803 | .035 | 1.806 |
| Within groups | 72 | 1.393 | .019 | p = .0305 |
| Total | 95 | 2.196 | | |

Model II estimate of between component variance = .004

| | | |
|---|---|---|
| Culture | 100 | |
| 5 uM Spermidine | 97 | |
| 20 uM 2,3,2-piperidine | 100 | |
| 20 uM 2,3,2-pyridine | 100 | |
| 12.5 uM 2,3,2-diCH₃ | 100 | |
| 12.5 uM Cyclam Adamantane | 100 | |
| 100 uM Streptozotocin | 57 | |
| 100 uM Streptozotocin + | 87 | 0.297 Sig. at 99.8% |
| 5 uM Spermidine | | |
| 100 uM Streptozotocin + | 70 | 0.297 |
| 2.5 uM 2,3,2-piperidine | | |
| 100 uM Streptozotocin + | 100 | 0.364 Sig. at 99.8% |
| 5 uM 2,3,2-piperidine | | |
| 100 uM Streptozotocin + | 100 | 0.364 Sig. at 99.8% |
| 20 uM 2,3,2-piperidine | | |
| 100 uM Streptozotocin + | 100 | 0.364 Sig. at 99.8% |
| 2.5 uM 2,3,2-pyridine | | |
| 100 uM Streptozotocin + | 100 | 0.364 Sig. at 99.8% |
| 5 uM 2,3,2-pyridine | | |
| 100 uM Streptozotocin + | 100 | 0.364 Sig. at 99.8% |
| 20 uM 2,3,2-pyridine | | |
| 100 uM Streptozotocin + | 100 | 0.364 Sig. at 99.8% |
| 1 uM 2,3,2-diCH₃ | | |
| 100 uM Streptozotocin + | 100 | 0.364 Sig. at 99.8% |
| 5 uM 2,3,2-diCH₃ | | |
| 100 uM Streptozotocin + | 100 | 0.364 Sig. at 99.8% |
| 12.5 uM 2,3,2-diCH₃ | | |
| 100 uM Streptozotocin + | 93 | 0.297 Sig. at 99.8% |
| 1 uM Cyclam Adamantane | | |
| 100 uM Streptozotocin + | 100 | 0.364 Sig. at 99.8% |
| 5 uM Cyclam Adamantane | | |
| 100 uM Streptozotocin + | 100 | 0.364 Sig. at 99.8% |
| 12.5 uM Cyclam Adamantane | | |

Spermidine, 2,3,2-piperidine, 2,3,2-pyridine, 2,3,2-diCH₃, and cyclam adamantane prevented streptozotocin induced cell killing at low micromolar doses.

### EXAMPLE 54

TABLE XVIII
Toxins and Antidotes ALLOXAN

Organism: E. Coli (GM 7359) alkA tag E. coli mutant
Toxin: Alloxan
Antidotes: 2,3,2-adamantane, 2,3,2-pyridine, Chromium 2,3,2-pyridine,
2,3,2-diCH₃ and Cyclam Adamantane
Incubation Time: 60 minutes

Analysis of Variance Table

| | | Sum | Mean | |
|---|---|---|---|---|
| Source: | DF: | Squares: | Square: | F-test |
| Between groups | 20 | 4.243 | .212 | 22.912 |
| Within groups | 60 | .556 | .009 | p = .0001 |
| Total | 80 | 4.798 | | |

Model II estimate of between component variance = .053

| | Mean % | |
|---|---|---|
| Samples | Survival Fisher PLSD | |
| Culture | 100 | |
| 100 uM 2,3,2-tetramine adamantane | 100 | |
| 10 uM 2,3,2-pyridine | 100 | |
| 100 uM chromium 2,3,2-pyridine | 100 | |
| 10 uM 2,2-diCH3 | 100 | |
| 10 uM Cyclam Adamantane | 100 | |
| 2 mM Alloxan | 20 | |
| 2 mM Alloxan + 1 uM | 51 | 0.229 Sig. at 99.5% |
| 2,3,2-tetramine adamantane | | |
| 2 mM Alloxan + 100 | 69 | 0.229 Sig. at 99.5% |
| uM 2,3,2 tetramine adamantane | | |
| 2 mM Alloxan + 10 | 100 | 0.229 Sig. at 99.5% |
| uM 2,3,2-pyridine | | |
| 2 mM Alloxan + 5 | 57 | 0.229 Sig. at 99.5% |
| uM Chromium 2,3,2-pyridine | | |
| 2 mM Alloxan + 25 | 52 | 0.229 Sig. at 99.5% |
| uM Chromium 2,3,2-pyridine | | |
| 2 mM Alloxan + 100 | 70 | 0.229 Sig. at 99.5% |
| uM Chromium 2,3,2-pyriidne | | |
| 2 mM Alloxan + 2.5 | 100 | 0.229 Sig. at 99.5% |
| uM 2,3,2-diCH₃ | | |
| 2 mM Alloxan + 10 | 100 | 0.229 Sig. at 99.5% |
| uM 2,3,2-diCH₃ | | |
| 2 mM Alloxan + 2.5 | 100 | 0.229 Sig. at 99.5% |
| uM Cyclam Adamantane | | |
| 2 mM Alloxan + 10 | 100 | 0.229 Sig. at 99.5% |
| uM Cyclam Adamantane | | |

2,3,2-tetramine adamantane, 2,3,2-pyridine, chromium 2,3,2-pyriidne, 2,3,2-diCH₃ and cyclam adamantane prevented alloxan induced cell killing at low micromolar concentrations.

### Diseases and Individual Mechanisms of Action

Following are examples of therapeutic actions of polyamines in various diseases:

### EXAMPLE 55

Neurodegenerative Diseases—Parkinson's Disease, Alzheimer's Disease, Lou Gehrig's Disease, Binswanger's Disease, Olivopontine Cerebellar Degeneration, Lewy Body Disease.

Polyamines treat these diseases by,

a) Competitive inhibition of uptake of xenobiotics at the polyamine transport site, such organic molecules being a cause of depigmentation and DNA damage; b) Steric shielding of DNA from organic molecules by compacting DNA; c) Limitation of mitochondrial DNA damage by removal of free copper, iron, nickel, mercury and lead ions by the presence of a polyamine; d) Induction of metallothionein gene transcription; e) Induction of nerve growth factor, brain derived neuronotrophic factor and neuronotrophin-3 gene transcription; f) Regulation of affinity of NMDA receptors and blockade of the MK801 ion channel; g) Inhibition of protein kinase C; h) Mitochondrial reuptake of calcium; i) Binding and conservation of reduced glutathione; j) Induction of ornithine decarboxylase by glutathione; k) Maintenance of the homeostasis of the redox environment in brain; I) Non toxic chelation of divalent metals in brain; m) Regulation of activity of preaspartate proteases; n) Inhibition of acetylcholinesterase and butyrylcholinesterase; o) Blockade of muscarinic M₂ receptors; p) Manteance of ratio of membrane phosphatidylcholine: phosphatidylserine ratio; q) Inhibition of superoxide dismutase, amine oxidase, monoamine oxidase B by binding of free copper, r) Regulation of brain polyamine levels in dementias with maintenance of endogenous polyamine levels; s) Blockade of neuronal n and p type calcium channels. To treat neurodegenerative diseases require prevention of mitochondrial DNA damage, maintenance of the oxidative phosphorylation activity of cells, induction of cellular repair mechanisms, regulation of receptor and enzymatic activities.

### EXAMPLE 56

### Stroke

Polyamines Treat the Consequences of Stroke in the Following Manner,

a) Induction of metallothionein gene transcription; b) Induction of nerve growth factor, brain derived neuronotrophic factor and neuronotrophin-3 gene transcription; c) Regulation of affinity of NMDA receptors and blockade of the MK801 ion channel; d) Inhibition of protein kinase C; e) Mitochondrial reuptake of calcium; f) Binding and conservation of reduced glutathione; g) induction of ornithine decarboxylase by glutathione; h) Maintenance of the homeostasis of the redox environment in brain; i) Non toxic chelation of divalent metals in brain; j) Inhibition of superoxide dismutase and amine oxidase k) Regulation of brain polyamine levels in dementias with maintenance of endogenous polyamine levels; 1) Blockade of neuronal n and p type calcium channels.

Prevention of oxidative damage during the reperfusion post ischemia and removal of redox metals released from dead cells, trapped in the tissue are important objectives.

### EXAMPLE 57

Diabetes Mellitus

Age, growth and metabolic requirements, weight and body mass, predisposition to atherosclerotic and vascular complications influence the treatment selection for diabetes mellitus patents. Several drugs may be developed to treat Type I and Type II diabetes mellitus and its vascular and neuronal complications, treatment choices being related to age, weight, body mass and clinical stage of disease; compositions which provide mitochondrial protection; compositions which additionally increase insulin output, compositions which enhance glucose tolerance, compositions which reduce insulin requirements and compositions which prevent diabetic nephropathy, microvascular damage, macrovascular damage and neuropathy:

Mitochondrial Protection

a) Competitive inhibition of uptake of xenobiotics at the polyamine transport site, such organic molecules being a cause of mitochondrial DNA damage; b) Steric shielding of DNA from organic molecules by compacting DNA; c) Limitation of mitochondrial DNA damage by removal of free copper, iron, nickel, mercury and lead ions by the presence of a polyamine; d) Induction of metallothionein gene transcription; e) Inhibition of protein kinase C; f) Mitochondrial reuptake of calcium, g) Binding and conservation of reduced glutathione; h) Induction of omitnine decarboxylase by glutathione; i) Maintenance of the homeostasis of the redox environment j) Inhibition of superoxide dismutase, amine oxidase by binding of free copper. Succinate and glutamate derivatized polyamines can stimulate insulin release. Prevention of mitochondrial DNA damage, maintaining oxidative phosphorylation, maintaining mitochondrial membrane integrity from free radical induced damage and stimulating insulin secretion via exocytosis or reducing insulin secretion in states of hyperinsulinism are important objectives in the treatment of diabetes.

Enhancing Insulin Release

Succinate polyamines increase the supply of succinic acid and acetyl CoA to the Krebs cycle they stimulate insulin synthesis and release they increase insulin output at high concentrations of glucose. Glutamate polyamines stimulate release of insulin by promoting exocytosis.

However in forms of diabetes associated with hyperinsulinism further insulin secretion is not desired because it may further damage β islet cells thus causing islet amyloid deposition and it contributes to macrovascular damage. Agents which increase glucose tolerance whilst not increasing insulin output can be helpful in managing the disease. Chromium and vanadium polyamine complexes are useful in that regard.

Obesity and Hyperinsulinism and Lipid Balance

A chromium polyamine complex can deliver trivalent chromium to its target sites where it promotes glucose tolerance in instances where body mass index is greater than average. A trivalent chromium polyamine complex can enhance glucose tolerance and decrease blood cholesterol and triglycerides, and increase high density lipoprotein in diabetics with above average body mass index and in obese patients having incipient diabetes. Polyamine tyrosine phosphatase inhibitors and chromium polyamine combines mitochondrial protection with enhanced glucose tolerance and metabolic regulation of lipid and carbohydrate metabolism.

Redwing Insulin Requirement, Carbohydrate Absorption and Maintaining Lipid Balance

Tetravalent vanadium polyamine complexes may be used in Type I and Type II diabetes to achieve metabolic control and diminish insulin requirement A vanadyl polyamine complex delivers vanadium in its cationic vanadyl V(IV) form to the tissues and a smaller dose of vanadium is required than when administered in other salt forms. Vanadium decrease blood glucose and D-3-hydroxybutyrate levels in diabetes, it also restores fluid intake and body weight of diabetic animals. These metabolic effects occur because vanadium a) decreases P-enolpyruvate carboxykinase (PEPCK) transcription, thus decreasing gluconeogenesis; b) it decreases tyrosine aminotransferase gene expression; c) it increases expression of glucokinase gene; d) it induces pyruvate kinase; e) it decreases mitochondrial 3-hydroxy-3-methylglutaryl-CoA synthase (HMGCoAS) gene expression; f) it decreases the expression of the liver and pancreas glucose-transporter GLUT-2 gene in diabetic animals to the level seen in controls; g) it increases the amount of the insulin-sensitive glucose transporter, GLUT4 by stimulating its transcription; h) the insulin like metabolic effects of vanadium are mediated by inhibition of protein tyrosine phosphatases (PIP). Peroxovanadium compounds irreversibly oxidize the thiol group of the essential cysteine at the PTP catalytic site. Vanadium is a structural analog of phosphate. Vanadium does not exhibit the growth effects and mitogenic effects of insulin and thus might avoid the macrovascular diseases consequences of hyperinsulinemia and be clinically useful in disease where insulin resistance is caused by defects in the insulin signaling pathway. Vanadium mimics the effects of insulin in restoring G proteins and adenyl cyclase activity increasing cyclic AMP levels; I) vanadyl ion suppresses nitric oxide production by macrophages; j) it has a positive cardiac inotropic effect; k) vanadium restores albumin mRNA levels in diabetic animals by increasing hepatic nuclear factor 1 (HNF 1); l) it restores triiodothyronine T₃ levels. Vanadyl polyamine has the advantages of mitochondrial protection combined with the ability to regulate the insulin signaling pathways, with effects on glucose, carbohydrate and fat metabolism. It can lower insulin requirements, thus overcoming the vascular consequences of hyperinsulinism, permit viable P cells to continue functioning and will exert these functions irrespective of body mass index.

Diabetic Nephropathy

Polyamines which more potently decrease protein kinase C activity than others may be used in the treatment of diabetic nephropathy. Protein kinase C causes apoptosis in diabetic nephropathy and polyamines reduce protein kinase C activation. Protein kinase C is overactivated due to excess diacylglycerol (DAG) formation from glucose.

The major Components of diabetes mellitus include, mitochondrial dysfunction and energetics dysfunction, impairment of exocytosis of insulin, impaired glucose tolerance and diminished insulin sensitivity with consequent altered carbohydrate and fit metabolism, neuropathy, microvascular and macrovascular complications are treatable with these classes of compounds, particularly by optimizing mitochondrial protection, protein kinase C inhibition, tyrosine phophatase 1B inhibition and PPARa and PPARy partial agonist/partial antagonist activities in a therapeutic compound.

### EXAMPLE 58

Atherosclerosis, Myocardial Ischemia, Cardiomyopathy, Ischemia

Polyamines treat atherosclerosis onset and progression by the following mechanisms;

a) Steric shielding of DNA from organic molecules by compacting DNA; b) Limitation of mitochondrial DNA damage by removal of free copper, iron and cadmium ions by the presence of a polyamine; c) Induction of metallothionein gene transcription; d) Inhibition of protein kinase C; e) Mitochondrial reuptake of calcium; f) Binding and conservation of reduced glutathione; g) Induction of ornithine decarboxylase by glutathione; h) Maintenance of the homeostasis of the redox environment; i) Inhibition of superoxide dismutase and amine oxidase by binding of free copper. Prevention of mitochondrial DNA damage, maintaining oxidative phosphorylation, maintaining normal LDL: HDL lipid ratios and preserving mitochondrial membrane integrity from free radical damage are major objectives in these diseases. In atherosclerosis prevention of oxidation of low density lipoprotein is also important.

The tyrosine phosphatase inhibitor polyamines and chromium polyamines mentioned above in relation to diabetic treatment are useful with regards to improving lipoprotein ratios and preventing atherosclerotic plaque formation. PPARα stimulates fatty acid catabolism in liver, heart, brown adipsoe tissue and PPARγ stimulates fatty acid anabolism or storage as triglycerides in adipose tissue. Free faty acids can cause insulin resistance in liver and muscle with increased hepatic gluconeogenesis. PPARa may act reciprocally with insulin. Thus tyrosine phosphatase inhibitors which are partial agonists/partial antagonists of PPARα and PPARγ can be synthesized from the polyamine tyrosine phosphatase inhibitors described herein and utilized to treat diabetes and atherosclerosis.

### EXAMPLE 59

Glaucoma

Polyamines Treat Glaucoma by;

a) Limitation of mitochondrial DNA damage by removal of free metals by the presence of a polyamine; b) Induction of metallothionein gene transcription; c) Regulation of affinity of NMDA receptors and blockade of the MK801 ion channel; d) Mitochondrial reuptake of calcium; e) Binding and conservation of reduced glutathione; f) Induction of ornithine decarboxylase by glutathione; g) Maintenance of the homeostasis of the redox environment; h) Non toxic chelation of divalent metals; i) Inhibition of superoxide dismutase and amine oxidase by binding of free copper, j) Regulation of polyamine levels in M ganglion cells with maintenance of endogenous polyamine levels. The M ganglion cells are pigment and metal rich and very prone to glutamate toxicity.

### EXAMPLE 60

Presbycussis

Polyamines Treat Presbycussis by,

a) Steric shielding of DNA from organic molecules by compacting DNA; b) Limitation of mitochondrial DNA damage by removal of free copper and iron ions by the presence of a polyamine; c) Induction of metallothionein gene transcription; d) Inhibition of protein kinase C; e) Mitochondrial reuptake of calcium; f) Binding and conservation of reduced glutathione; g) Induction of ornithine decarboxylase by glutathione; h) Maintenance of the homeostasis of the redox environment; i) Inhibition of superoxide dismutase and amine oxidase by binding of free copper. a, b) and c) prevent mitochondrial DNA damage which increases in the cochlea during aging and causes deafness.

### EXAMPLE 61

Optic Neuropathy

Polyamines Treat Optic Neuropathy by;

a) Steric shielding of DNA from organic molecules by compacting DNA; b) Limitation of mitochondrial DNA damage by removal of free copper and iron ions by the presence of a polyamine; c) Induction of metallothionein gene transcription; d) Inhibition of protein kinase C; e) Mitochondrial reuptake of calcium; f) Binding and conservation of reduced glutathione; g) Induction of ornithine decarboxylase by glutathione; h) Maintenance of the homeostasis of the redox environment; i) Inhibition of superoxide dismutase and amine oxidase by binding of free copper, j) counteracting agents which are toxic to mitochondria a, b) and c) prevent mitochondrial DNA damage.

### EXAMPLE 62

Peripheral Neuropathy

Polyamines Treat Peripheral Neuropathy by,

a) Steric shielding of DNA from organic molecules by compacting DNA, b) Limitation of mitochondrial DNA damage by removal of free copper and iron ions by the presence of a polyamine; c) Induction of metallothionein gene transcription; d) Inhibition of protein kinase C; e) Mitochondrial reuptake of calcium; t) Binding and conservation of reduced glutathione; g) Induction of ornithine decarboxylase by glutathione; h) Maintenance of the homeostasis of the redox environment; i) Inhibition of superoxide dismutase and amine oxidase by binding of free copper, j) counteracting agents which are toxic to mitochondria a, b) and c) prevent mitochondrial DNA damage.

### EXAMPLE 63

Cancer

Polyamines form extremely stable complexes with cobalt as indicated by their heats of formation. A cobalt dihomocysteine polyamine complex can behave like thioretinaco. As a non toxic, intracellular electrophile it will promote ATP formation and protect against free oxygen species produced by toxins, radiation and cancer cells. Further it would diminish homocysteic acid formation, which promotes growth factor activity, and thus prevent the invasiveness and neovascularization caused by cancer cells.

### EXAMPLE 64

Treatment of Inherited Mitochondrial Diseases

Mitochondrial deletions, substitutions, mutations caused the following diseases. The expression of the defects is variable from patient to patient Polyamines limit damage to mitochondrial macromolecules as demonstrated herein with cell viability studies using six mitochondrial toxins. Polyamines can be used to treat the consequences of inherited mitochondrial defects. These inherited diseases include; Alpers Syndrome, Alzheimer's Disease, Atherosclerosis, Barth's Disease, Batten's Disease, Beta-Oxidation Disorders, Carnitine Deficiency, Cardiomyopathy, COX (Cytochrome C Oxidase Deficiency), Diabetes, Glaucoma, Glutaric Aciduria, ‘Huntington’s Disease, Kearns-Sayre/CPEO, Leigh's Disease, Leber's Optic Neuropathy/LHON, MELAS, Mitochondrial Cardiomyopathies, Mitochondrial Cytopathies, Mitochondrial Encephalomyopathies, Mitochondrial Myopathies, Optic Neuropathy, Parkinson's Disease, Peripheral Neuropathy, Presbycussis, Respiratory Chain disorders Complexes L I, a, IV and/or V, Seizures and Stroke.

### EXAMPLE 65

Treatment of Osteoporosis, Multiple Sclerosis, Rheumatoid Arthritis and Inflammatory Bowel Disease

Tyrosine phosphatase inhibitors such as orthovanadate prevent glucocorticoid induced osteoporosis (Hulley P. A. et al 2002). Vanadate stimulates osteoblast formation without affecting osteoclast formation PAPRy agonists decreased experimental autoimmune encephalomyelitis in mice, with decreased lymphocyte infiltration, reduced demyelination, decreased chemokine and cytokine expression (Feinstein D. L. et al 2002). Polyamine based PPARγ partial agonists/partial antagonists will treat T cell mediated immune diseases.

### EXAMPLE 66

Antidotes to Organics Toxins and Heavy Metals

The bacterial experiments herein demonstrate broad efficacy of polyamine classes against mitochondrial toxins. Paraquat causes lung, liver and brain damage in man, MPTP/MPP+ and Rotenone cause Parkinsonism, diazoxide, streptozotocin and alloxan cause diabetes mellitus. The toxicity of paraquat and MPTP is exacerbated by heavy metals. Heavy metals are epidemiologically linked to diseases such a Parkinson's disease and some cancers. Polyamines can be used to treat single and cumulative exposure to mitochondrially damaging organics and heavy metals.

### EXAMPLE 67

### Radiologic Uses

Contrast media used in radiologic examinations including complexes of the following metals;

trivalent gadolinium, iron, trivalent lanthanide, manganese, technetium are described in Examples 36, 37 and 39. Basic requirements in synthesizing derivatives of these polyamines for human use are that the compound(s) are non ionic, do not have COO groups, have OH groups in various positions around the molecule, and are water-soluble. Secondary composition possibilities are that they can be prepared as monomers, dimers, trimers or tetramers, they may be incorporated into liposomes, they have low viscosity, they exhibit low osmolality, and have a particle size between 0.6 and 3 microns to avoid capillaly embolism Manganese polyamine may be used as liver and pancreas contrast MRI agent amongst other uses. A liposome preparation of the complex can be used. An iron polyamine may be used in hepatic MRI imaging. Gadolinium polyamine may be used for angiography, intraarticular examinations and hepatobiliary MRI. It has low renal toxicity.

Manganese (2,2′-diamino (bis-N,N-quinilylmethyl)biphenyl)(Cl)₂ (Example 36) May be used as liver and pancreas contrast MRI agent amongst other uses. A liposome preparation of the complex can be used. [Iron (4chloro-2-{[(2-{2-[(2-pyridylmethylamino]phenyl}-phenyl)amimo]methyl}phenol)(Cl)₂]Cl (Example 37) May be used in hepatic MRI imaging Gadolinium (2,2-diamino(bis-N,N′-pyridylmethyl)biphenyl)Cl₂]Cl (Example 39) May be used for angiography, intraarticular examinations and hepatobiliary MRI. It has low renal toxicity.

### REFERENCES CITED

Abraham A. S. Brooks B. A., Eylath U. The effects of chromium supplementation on serum glucose and lipids in patients with and without non-insulin-dependent diabetes. Metabolism: Clin. Exper. (1992) July, 41(7):768-71.
Abraham C. R., Driscoll 3., Potter H., Van Nostrand W. E., Tempst P. A calcium-activated protease from Alzheimer's disease brain cleaves at the N-terminus of the amyloid B-protein. Biochem Biophys. Res. Commun (1991), 174,790-96.
Abraham Ck, Razzaboni B. L., Sisodia S. S., Koo E. H., Price D. L., Van Nostrand W. E., Papastoitsis G. Studies on the proteolytic degradation of the B-protein precursor by proteases purified from Alzheimer's brain Ann New York Acad. Sci. (1991), 640, 161-65.
Abraham C. R., Razzaboni B. I., Papastoitsis G., Picard E., Kanemaru K., Meckelein B., Mucke L. Purification and cloning of brain proteases capable of degrading the B-amyloid precursor protein. Ann New York Acad Sci (1992) 674, 174-179.
Ahmad F., Azevedo J. L., Cortright R., Dobm G. L., Goldstein B. J. Alterations in skeletal muscle protein-tyrosine phosphatase activity and expression in insulin-resistant human obesity and diabetes. Jour. Clin Invest (1997), 100(2), 449-58.
Aime S., Barge A, Dell Castelli D., Fedeli F., Mortillaro A, Nielsen F. U., Terreno E. Paramagnetic lanthanide(III) complexes as pH-sensitive chemical exchange saturation transfer (CEST) contrast agents for MRI applications. Magn. Reson. Med. (2002), 47(4), 639-48.
Akkan A. G., Malaisse W. J. Iterative pulse administration of succinic acid monomethyl ester to streptozotocin diabetic rats. Diabetes Res. (1993), 23(2):55-63.
Aldrich, P., Hermann, E. C., Meier, W. E., Antiviral Agents. Structure-Activity Relationships of Compounds Related to I-Adamantanamine, Jour. Med. Chem., (1971), 14,535-543.
Almen T. Contrast media: the relation of chemical structure, animal toxicity and adverse clinical effects. Am J Cardiol. (1990), 66(14), 2F-8F.
Anand-Srivastava M. B., McNeill J. H. Yang XP. Reversal of defective G-proteins and adenyl cyclase/cAMP signal transduction in diabetic rats by vanadyl sulphate therapy. Molec. Cell. Biochem. (1995), December 6-20,153(1-2):113-9.
Arbustini, E; Diegoli, M; Fasani, R; Grasso, M; Morbini, P; Banchieri, N; Bellini, 0; Dal Bello, B; Pilotto, A; Magrini, G; Campana, C; Fortina, P; Gavazzi, A; Narula, J; Vigano, M Mitochondrial DNA mutations and mitochondrial abnormalities in dilated cardiomyopathy. Amer. Jour. Path (1998), November, 153(5), 1501-10.
Arbustini, E; Morbini, P. Pilotto, A; Gavazzi, A; Tavazzi, L. Genetics of idiopathic dilated cardiomyopathy. Herz (2000), May, 25(3), 15660.
Arden S. D., Roep B. O., Neophytou P. L., Usac E. F., Duinkekrken G., deVries R. P. Hutton J. C. Imogen 38: A novel 38-kD islet mitochondrial autoantigen recognized by T cells from a newly diagnosed type 1 diabetic patient Jour. Clin. Invest (1996), 97(2), 555-61.
Avignon A, Yamada K, Zhou X, Spencer B., Cardona O., Saba-Siddique S., Galloway L., Standaert M. L., Farese R. V. Chronic activation of protein kinase C in soleus muscles and other tissues of insulin-resistant type U diabetic Goto-Kakizaki (GK), obese/aged, and obese/Zucker rats. A mechanism for inhibiting glycogen synthesis. Diabetes (1996), 45(10), 1396-404.
Baekkeskov S., Aanstoot H. J., Christgau S., Reetz A., Solimena M., Cascalho, Folli F., Richter-Olesen H. DeCamilli P. Identification of the 64K autoantigen in insulin-dependent diabetes as the GABA-synthesizing enzyme glutamic acid decarboxylase. Nature (1990), 347, 151-56.
Baeza L, Ibanez M, Wong C., Chavez P., Gariglio P. Possible prebiotic significance of polyamines in the condensation, protection, encapsulation, and biological properties of DNA. Orig. Life Evol. Spec. (1992), 21,225-42.
Barefield, K, Wagner, F., Metal Complexes of 1,4,8,11-Tetramethyl-1,4,8,11-tetraazacyclotetradecane, N-Tetramethylcyclam, Inorg. Chem., (1973), 12, 2435-2436.
Barefield, E. K., Wagner, F., Hodges, K. D., Synthesis of Macrocyclic Tetramines by Metal Ion Assisted Cyclization Reactions. Inorg. Chem., (1976), 15, 1370-1377.
Barrera-Hernandez G., Wanke I. E., Wong N. C. Phlorizin or vanadate treatment reverses impaired expression of albumin and hepatocyte nuclear factor 1 in diabetic rats. Diabetes (1996), September, 45(9):1217-22.
Barrett, G. M., et al., Dissolving Metal Reduction of Esters to Alkanes. Jour. Chem. Soc., Perkin 1, (1981), 1501-1509.
Beneviste M, Mayer M. L. Multiple effects of spermine on n-methyl-D-aspartic acid receptor responses of rat cultured hippocampal neurons. Jour. Physiol. (1993), 464, 131-63.
Black R. A., Kronheim S., Merriam J., March C., Hopp T. A. Preaspartate protease from human leukocytes that cleaves pro-interleukin-1B. Jour. Biol. Chem (1989), 264, 5323-26.
Blomgren K, Nilsson E., Karlsson J. Calpain and calpasatin levels in different organs of the rabbit. Compar. Biochem. Physiol. B. (1989), 93,403-07.
Bonifacio E., Lampasona V., Genovese S., Ferrari M, Bosi E. Identification of protein tyrosine phosphatase-like IA2 (Islet cell antigen 512) as the insulin-dependent diabetes-related 37/40K autoantigen and a target of islet-cell antibodies. Jour. Immunol (1995), 155,5419-26.
Bonifacio E., Lampasona V., Bingley P. J. IA-2 (islet cell antigen 512) is the primary target of humoral autoimmunity against type 1 diabetes-associated tyrosine phosphatase autoantigens. Jour. Immunol. (1998), 161(5), 2648-54.
Bonte, C A; Matthijs, G L; Cassiman, J J; Leys, A M. Macular pattern dystrophy in patients with deafness and diabetes. Retina (1997), 17(3):216-21.
Boquist L., Ponten E., Sandstroem E. Study of isolated mitochondria incubated with labelled and non-labelled alloxan, with regard to intramitochondrial concentrations of reduced glutathione and inorganic phosphate. Diab. Metab. (1983), 9, 106-11.
Borg L. A., eide S. J., Andersson A, Hellerstrom C. Effects in vitro of alloxan on glucose metabolism of mouse pancreatic B-cells. Biochem. Jour. (1979), 182, 797-802.
Borg H., Fernlund P., Sundkvist G. Protein tyrosine phosphatase-like protein IA2-antibodies plus glutamic acid decarboxylase 65 antibodies (GADA) indicates autoimmunity as frequently as islet cell antibodies assay in children with recently diagnosed diabetes mellitus. Clin. Chem. (1997), 43(12), 2358-63.
Bradley W. G., Krasin F. A new hypothesis of the etiology of amyotrophic lateral sclerosis. Arch Neurol. (1982), 39,677-80.
Borthwick, G M; Johnson, M A; Ince, P G; Shaw, P J; Turnbull, D M. Mitochondrial enzyme activity in amyotrophic lateral sclerosis: implications for the role of mitochondria in neuronal cell death Ann. Neurol. (1999), November, 46(5):787-90.
Brabenboer B., Kappelle A. C. Harmers F P T, van Buren T., Erkelens D. W., Gispen W. H. Potential use of glutathione for the prevention and treatment of diabetic neuropathy in the streptozotocin-induced diabetic rat Diabetologia (1992), 35, 813-17.
Brantl V., Gramsch C., Lottspeich F., Henschen A., Jager R. A., Herz A. Novel opioid peptides derived from mitochondrial cytochrome b: cytocrophins. Eur. Jour. Pharmacol. (1985), III, 293-94.
Bu, X, Rotter, J. I. Wolfram syndrome: a mitochondrial-mediated disorder? Lancet, (1993) September 4, 342(8871):598-600.
Burke T. R Jr, Ye B., Yan X., Wang S., Jia Z., Chen L., Zhang Z. Y., Barford D. Small molecule interactions with protein-tyrosine phosphatase PTP1B and their use in inhibitor design. Biochemistry (1996a), 35(50), 15989-96.
Burke T. R. Jr, Ye B., Akamatsu M., Ford H. Jr, Yan X, Kole H. K., Wolf G., Shoelson S. E., Roller P. P. 4′-O-[2-2-fluoromalonyl)]-L-tyrosine: a phosphotyrosyl mimic for the preparation of signal transduction inhibitory peptides. Jour. Med. Chem. (1996b) 39(5), 1021-7.
Bush, A I; Pettingell, W H; Multhaup, G; d Paradis, M; Vonsattel, J P; Gusella, J F; Beyreuther, K; Masters, C L; Tanzi, R E. Rapid induction of Alzheimer A β-amyloid formation by zinc [see comments] Science (1994), September 2, 265(5177):1464-7.
Cam M. C., Rodrigues B., McNeill J. H. Distinct glucose lowering and beta cell protective effects of vanadium and food restriction in streptozotocin-diabetes. Eur. Jour. Endocrin (1999), November, 141(5):546-54.
Cam M. C., Pederson R. A., Brownsey R. W., McNeill J. H., Long-term effectiveness of oral vanadyl sulphate in streptozotocin-diabetic rats. Diabetologia (1993), 36, 218-24.
Car M. C. Brownsey, R. W., McNeill J H. Mechanisms of vanadium action: insulin-mimetic or insulin-enhancing agent? Can. Jour. Physiol. Pharmacol. (2000) October, 78(10):82947.
Cameron N. E., Cotter M. A., Maxfield E. K. Anti-oxidant treatment prevents the development of peripheral nerve dysfunction in streptozotocin-diabetic rats. Diabetologia (1993), 36, 299-304.
Cameron N. E., Cotter M. A., Archoibald V., Dines K. C., Maxfield E. K., Anti-oxidant and Pro-oxidant effects on nerve conduction velocity, endoneurial blood flow and oxygen tension in non-diabetic and streptozotocin-diabetic rats. Diabetologia (1994), 37, 449-59.
Campbell L., Oxbrow L., Koulmanda M., Hrrison L. C. IFN-gamma induces islet cell MHC antigens and enhances autoimmune, streptozotocin-induced diabetes in the mouse. Jour. Immunol. (1988), 140(4), 1111-6.
Cardinal J. W., Allan D. J., Cameron D. P. Poly (ADP-ribose) polymerase activation determines strain sensitivity to streptozotocin-induced B cell death in inbred mice. Jour. Molec. Endocr. (1999), 22, 65-70.
Carroll P. B., Moura A. S., Rojas E., Atwater I. The diabetogenic agent alloxan increases K⁺ permeability by a mechanism involving activation of ATP-sensitive K(+)-channels in mouse pancreatic beta-cells. Mol Cell Biochem. 1994 Nov. 23;140(2):127-36.
Cashman J. R. Mutagenicity of MPTP (1-methyl-4 phenyl-1,2,3,6-tetrahydropyridine) and its metabolites. Toxicology (1987), 173-82.
Castano L., Russo E., Zhou L., Lipes M. A., Eisenbarth G. S. Identification and cloning of a granule autoantigen (carboxypeptidase-H) associated with Type 1 diabetes. Jour. Clin Endocrin Metab. (1991), 73(6), 1197-1201.
Cavanaugh P. F., Pavelic Z. P., Porter C. W. enhancement of 1,3-Bis(2-chloroethyl)-1-nitrosurea-induced cytotoxicity and DNA damage by α-difluoromethylornithine in L1210 leukemia cells. Cancer Res. (1984), 44,3856-61.
Chakrabarti A. K, Banik N., Powers J., Hogan E. The regional and subcellular distribution of calcium activated neutral proteinase (CANP) in the bovine central nervous system. Neurochem. Res. (1989), 14,259-66.
Chang D., Kim B., Yun Y., Hur Y., Lee Y., Choi M., Yoon J., Seong J. Superparamagetic iron oxide-enhanced magnetic resonance imaging of the liver in beagle dogs. Vet Radiol. Ultras. (2002), 43(1), 37-42.
Chen H, Carlson E C., Pellet L., Moritz, J. T., Epstein P. N. Overexpression of metallothionein in pancreatic beta-cells reduces streptozotocin-induced DNA damage and diabetes. Diabetes 2001 September, 50(9):2040-6.
Chen J., Jin K, Chen M., Pei w., Kawaguchi K., Greenberg D. A., Simon R. P. early detection of DNA strand breaks in the brain after focal transient ischemia: implications for the role of DNA damage in apoptosis and neuronal cell death. Jour. Neurochem. (1997), 69,232-45.
Chen, S J; Bradley, M E; Lee, T C. Chemical hypoxia triggers apoptosis of cultured neonatal rat cardiac myocytes: modulation by calcium-regulated proteases and protein kinases. Molec. Cellul. Biochem. (1998), January, 178(1-2), 141-9.
Cheung A, Kusari J., Jansen D., Bandyopadhyay D., Kusari A, Bryer-Ash K Marked impairment of protein tyrosine phosphatase 1B activity in adipose tissue of obese subjects with and without type 2 diabetes mellitus. Jour. Lab. Clin. Med. (1999), 134(2), 115-23.
Chu P., C C; Huang, C C; Fang, W; Chu, N S; Pang, C Y; Wei, Y H. Peripheral neuropathy in mitochondrial encephalomyopathies. Eur. Neurol. (1997), 37(2):110-15.
Chu P., Saito H., Abe K. Polyamines promote regeneration of injured axons of cultured rat hippocampal neurons. Brain Res. (1995), 673,233-41.
Clark, A; de Koning, E J; Hattersley, A T; Hansen, B C; Yajnik, C S; Poulton, J. Pancreatic pathology in non-insulin dependent diabetes (NIDDM). Diabetes Res. Clin. Prac. (1995), August, 28 Suppl., S3947.
Clayton D. A., Doda J. N., Friedberg E. C. The absence of a pyrimidine dimer repair mechanism in mammalian mitochondria Proc. Natl. Acad. Sci. (1974), 71,2777-81.
Clopath P., Smith V. C., McCully K. S. Growth promotion by homocysteic acid. Science (1976), 373-74.
Cohen N., Halberstam M., Shlimovich P., Chang C. J., Shamoon H., Rossetti L. Oral vanadyl sulfate improves hepatic and peripheral insulin sensitivity in patients with non-insulin-dependent diabetes mellitus. Jour. Clin. Invest. (1995), June, 95(6):2501-9.
Comi, G P; Bordoni, A; Salani, S; Franceschina, L; Sciacco, M; Prelle, A; Fortunato, F; Zeviani, M, Napoli, L; Bresolin, N; Moggio, M, Ausenda, C D; Taanman, J W; Scarlato, G. Cytochrome c oxidase subunit 1 microdeletion in a patient with motor neuron disease. Ann. Neurol. (1998), January, 43(1): 110-6.
Corral-Debrinski, M, Shoffner, J M; Lott, M T; Wallace, D C. Association of mitochondrial DNA damage with aging and coronary atherosclerotic heart disease. Mutat Res. (1992), September, 275(3-6), 169-80.
Corral-Debrinski U Mitochondrial DNA deletions in human brain: regional variability and increase with advanced age. Nat Genet. (1992), December; 2(4):324-9.
Corral-Debrinski, M; Horton, T; Loft, M T; Shoffner, J M, McKee, A C; Beal, M F; Graham, B H, Wallace, D C. Marked changes in mitochondrial DNA deletion levels in Alzheimer brains. Genomics (1994), September 15, 23(2):471-6.
Cotter M. A., Cameron N. E. Neuroprotective effects of carvidilol in diabetic rats, prevention of defective peripheral nerve perfusion and conduction velocity. Nauyn Schmiedbergs Arch. Pharmacol. (1995), 351, 630-35.
Craven P. A., DeRubertis F. R., Kagan V. E., Melhem M, Studer R. K., Effects of supplementation with vitamin C or E on albuminuria, glomerular TGF-01 and glomerular size in diabetes. Jour. Amer. Soc. Nephrol. (1997), 8, 1405-11.
Cui, 3; Holmes, E. H.; Greene, T. G.; Liu, P. K. Oxidative DNA damage precedes DNA fragmentation after experimental stroke in rat brain Faseb Journal (2000) May, 14(7):955-67.
Dawson G., Glaser P. Apparent cathepsin B deficiency in neuronal ceroid lipofuscinosis can be explained by peroxide inhibition. Biochem. Biophys. Res. Commun. (1987), 147, 267-74.
Dawson G., Glaser P. Abnormal cathepsin B activity in Batten's disease. Amer. Jour. Med. Genet Suppl. (1988), 5,209-20.
den Hertog J. Protein-tyrosine phosphatases in development Mech. Devel. (1999), 95, 3-14.
Desreux J. F., Barthelemy P. P. Highly stable lanthanide macrocyclic complexes: in search of new contrast agents for NMR imaging. Int. Jour. Rad. Appl. Instrum B. (1988), 15(1), 9-15.
Diehl S. J., Lehmann K. J., Gaa J., McGill S., Hoffmann V., Georgi M. MR imaging of pancreatic lesions. Comparison of manganese-DPDP and gadolinium chelate. Invest Radiol. (1999), 34(9), 589-95.
Dixon J. E Structure and catalytic properties of protein tyrosine phosphatases. Ann. N.Y. Acad. Sci. (1995), 766, 18-0.22.
Domingo J. L., Gomez M, Sanchez D. J., Liobet J M, Keen, C. L. Toxicology of vanadium compounds in diabetic rats: the action of chelating agents on vanadium accumulation Molec. Cell. Biochem. (1995), December 620, 153(1-2):23340.
Dotta F. Previti M., Lenti L., Dionisi S., Casetta B., D'Erme M. D., Eisenbarth G. S., DiMario U. GM2-1 pancreatic islet ganglioside: identification and characterization of a novel islet-specific molecule. Diabetologia (1995), 38, 1117-21.
Dotta F., Dionisi S., Viglietta V., Tiberti C., Matteoli M. C., Cervoni M., Bizzarri C., Marietti G., Testi M, Multari G., Lucentini L., Di Mario U. T-cell mediated autoimmunity to the insulinoma-associated protein 2 islet tyrosine phosphatase in type 1 diabetes mellitus. Eur. Jour. Endocrinol. (1999), 141(3), 272-8.
Dreyer E. B., Pan Z., TORM s., Lipton S. A. Greater sensitivity of larger retinal ganglion cells to NMDA-mediated cell death. NeuroReport (1994), 5, 629-31.
Dreyer E. B., Zurakowski D., Schumer R. A, Podos S. M., Lipton S. A. Elevated glutamate levels in the vitreous body of humans and monkeys with glaucoma Arch. Ophthalmol. (1996), 114, 299-305.
Duara R, Lopez-Alberola R. F., Barker W. W. A comparison of familial and sporadic Alzheimer's disease. Neurology. (1993), Ju1;43(7):1377-84.
Dubin D. T. Evidence for conjugation between glutathione and polyamines in E. Coli Biochem. Biophys. Res. Commun (1959), 1(5), 262-65.
Dyer G. D., Dunn J. A., Thorpe S. R., Accumulation of Maillard reaction products in skin collagen I diabetics and aging. Jour. Clin Invest. (1993), June, 91(6):2463-9.
Earle K. E., Archer A. G., Baillie, J. E. Circulating and excreted levels of chromium after an oral glucose challenge: influence of body mass index, hypoglycemic drugs, and presence and absence of diabetes mellitus. Amer. Jour. Clin. Nutr. (1989), April, 49(4):685-9.
Ebihara L, Nakamura T., Shim N., Koide, H Increased plasma metalloproteinase-9 concentrations precede development of microalbuminuria in non-insulin-dependent diabetes mellitus. [Comment In: Am J Kidney Dis. (1998), October; 32(4):669-71] Amer. Jour. Kidney Dis. (1998) October, 32(4):544-50.
Edelstein C., Kaiser M., Piras G., Scanu A. Demonstration that the enzyme that converts precursor of apolipoprotein A-1 is secreted by the hepatocarcinoma cell line hep G2.
Arch Biochem. Biophys. (1988), 267,23-30.
Edland S. D., Silverman J. Peskind E. R., Tsuan G. D., Wijsman E., Morris J. C. Increased risk of dementia in mothers of Alzheimer's disease cases: evidence for material inheritance. Neurology (1996), July; 47(1):254-6.
Egawhary, D N; Swoboda, B E; Chen, J; Easton, A J; Vince, F P. Diabetic complications and the mechanism of the hyperglycaemia-induced damage to the mt DNA of cultured vascular endothelial cells: (I) Characterization of the 4977 base pair deletion and 13 bp flanking repeats. Biochemical Soc. Trans. (1995), November, 23(4), 518S.
Eizirik D. L., Welsh N., Niemann A., Velloso L. A., Malaisse W. J. Succinic acid monomethyl ester protects rat pancreatic islet secretory potential against interleukin-1β (IL-1β) without affecting glutamate decarboxylase expression or nitric oxide production. FEBS Lett (1994), 337, 298-302.
Englander, E W; Greeley, G H Jr; Wang, G; Perez-Polo, J R, Lee, If Hypoxia-induced mitochondrial and nuclear DNA damage in the rat bri Jour. Neurosci. Res. (1999), October 15, 58(2):262-9.
Erickson S. E. The Respiratory system of the anaerobic nitrogen-fixing gram positive bacterium, Mycobacterium falvum 301. Biochim. Biophys. Acta (1971), 245,63-70.
Failla, M. L., Kiser R. A. Altered tissue content and cytosol distribution of trace metals in experimental diabetes. Jour. Nutr. (1981), November, 111(11):1900-9.
Fantus I. G., Tsiani E. Multifunctional actions of vanadium compounds on insulin signaling pathways: evidence for preferential enhancement of metabolic versus mitogenic effects. Molec. Cell Biochem. (1998), May 182(1-2):109-19.
Fawcett, T. G., Rudich, S. M, Toby, B. R, LaLancette, R. A., Potenza, J. A., Schugar, H. J. Studies of Chelation Therapy. Inorg. Chem. (I 980), 19, 940.
Feinstein D. L., Galea E., Gavrilyuk V., Brosnan C. F., Whitacre C. C., Dumitrescu-Ozimek L., Landreth G. E., Pershadsingh H. A., Weinberg G., Heneka M. T. Peroxisome proliferator-activated receptor-gamma agonists prevent experimental autoimmune encephalomyelitis. Ann. Neurol. (2002), 51(6), 694702.
Ferrari, R. The role of mitochondria in ischemic heart disease. Jour. Cardiovasc. Pharmacol. (1996), 28 Suppl 1, S1-10.
Fischer, H. R., Hodgson, D. J., Michelsen, K., Pedersen, E., Synthesis and Characterization of the Dimeric Cr(III) Complex Di-hydroxobis[{N,N′-bis(2-pyridylmethyl)-1,3-propanediamine}chromium (III)] Perchlorate, Inorg. Chim. Acta, (1984), 88; 143-150.
Forman B. M., Chen J., Evans R. M. The peroxisome proliferator-activated receptors: ligands and activators. Ann. N.Y. Acad. Sci. (1996), 804, 266-75.
Fram R. J, Mack S. L, George M, Marinus M. G. DNA repair mechanisms affecting cytotoxicity by streptozotocin in E. coli. Mutat Res. (1989), 218(2), 125-33.
Fram R. J., Marinus M. G., Volkert M. R Gene expression in E. coli after treatment with streptozotocin. Mutat Res. (1988), 198(1), 45-51.
Frangioni J. V., Beahm P. H., Shifiin V., Jost C. A., Neel B. G. The nontansmembrane tyrosine phosphatase PTP-1B localizes to the endoplasmic reticulum via its 35 amino acid C-terminal sequence. Cell. (1992), 68(3), 545-60.
Frei B., Winterhalter K. H., Richter C. Mechanism of alloxan-induced calcium release from rat liver mitochondria J. Biol. Chem. (1985), 260(12), 7394-401
Freiberg J. M., Schneider T., Streeten D. H. A Effect of Brewer's yeast on glucose tolerance.
Ann Meeting Amer. Diab. Assoc. (1975) Abstract
Fujiwara T., Horikoshi H. Troglitazone and related compounds: therapeutic potential beyond diabetes. Life Sci. (2000) 67(20, :2405-16.
Gai W. P., Blumbergs P. C., Blesing W. W. The ultrastructure of Lewy neurites. Mov. Disord. (1977), 12, Supl. 1:5.
Gerbitz K D. Does the mitochondrial DNA play a role in the pathogenesis of diabetes? Diabetologia (1992), December, 35(12), 1181-6.
Gilad G., Domay M., Gilad V. Polyamines induce precocious development in rats. Possible interaction with growth factors. Int Jour. Devl. Neurosci. (1989), 7(6), 641-53.
Gilad G., Gilad V. H. Polyamines can protect against ischemia-induced nerve cell death in gerbil forebrain. Exper. Neurol. (1991), 111,349-55.
Gilad G. M, Gilad V. H. Novel polyamine derivatives as neuroprotective agents. Jour. Pharm Exper. Therap. (1999), 291(1), 39-43.
Goering P. L., Tandon S. K, Klaassen C. D. Induction of hepatic metallothionein in mouse liver following administration of chelating agents. Toxicol. Appl. Pharmacol. (1985), 80, 467-72.
Gong J., Xu J., Zhou K., Shen K. Role of exocrine cells in pancreatic enhancement using Mn-DPDP-enhanced MR imaging Chin Med. Jour. (Engl). (2002), 115(9), 13636.
Goldfine A. B., Simonson D. C., Folli F., Patti, M E., Kahn C. R. In vivo and in vitro studies of vanadate in human and rodent diabetes mellitus. Molec. Cell Biochem (1995) December 6-20, 153(1-2):217-31.
Goldfine A. B., Patti M. E., Zuberi L., Goldstein B. J., LeBlanc R, Landaker E. J., Jiang Z. Y., Willsky G. R, Kahn C. R. Metabolic effects of vanadyl sulfate in humans with non-insulin-dependent diabetes mellitus: in vivo and in vitro studies. Metabolism: Clin. Experim. (2000) March, 49(3):400-10.
Golub, G., Cohen, H, Meyerstein, D., The Stabilization of Monovalent Copper Ions by Complexation with Saturated Tertiary Amine Ligands in Aqueous Solutions. Jour. Chem. Soc., Chem. Commun., (1992), 397-398.
Goodwin, A., Lions, F. Quadridentate Chelate Compounds. Jour. Am. Chem. Soc., (1960), 82,5013-23.
Goodman Y., Bruce A. J., Cheng B., Mattson M. P. Estrogens attenuate and corticosterone exacerbates excitotoxicity, oxidative injury and amyloid P-peptide toxicity in hippocampal neurons. Jour. Neurochem (1996), 66, 1836-44.
Gralkvist, K; Marklund, S L. Opposite effects of two metal-chelators on alloxan-induced diabetes in mice. Life Sci. (1983), December 19, 33(25):2535-40.
Hagglof B., Hallmans G., Holmgren G., Ludvigsson J., Falkmer S. Prospective and retrospective studies of zinc concentraion in serum, blood clots, hair and urine in young patients with insulin-dependent diabetes mellitus. Acta Endocrin. (Copenh) (1983), 102, 88-95.
Haglund, B; Ryckenberg, K; Selinus, O; Dahlquist, G. Evidence of a relationship between childhood-onset type I diabetes and low groundwater concentration of zinc. Diabetes Care 1996 August, 19(8):873-5.
Hamakubo T., Kannagi R, Murachi T., Matus A. Distribution of calpain I and II in rat brain. Jour. Neurosci. (1986), 6, 3103-11.
Hambidge, K M: Rodgerson, D O; O'Brien, D. Concentration of chromium in the hair of normal children and children with juvenile diabetes mellitus. Diabetes 1968 August, 17(8):517-9.
Hashimoto N., Goldstein B. J. Differential regulation of mRNAs encoding three protein-tyrosine phosphatases by insulin and activation of protein kinase C. Biochem. Biophys. Res. Commun. (1992), 188(3), 13.05-11.
Haskel Y., Udassin R., Chevion M Mechanistic aspects of MPP+toxicity in E. Coli: The role of oxygen and hydrogen peroxide. Israel Jour. Med. Sci. (1991), 27 (4), 207-12.
Hawa M., Rowe R., Lan M. S., Notkins A. L., Pozzilli P., Christie M. R., Leslie R. D. Value of antibodies to islet protein tyrosine phosphatase-like molecule in predicting type 1 diabetes. Diabetes (1997), 46(8), 1270-5.
Hay, R. W., Gidney, P. M., Lawrance, G. A., Cobalt Complexes of 3,7-Dithianonane-1,9-diamine. Jour. Chem Soc., Dalton, (1975), 779-784.
Heyliger C. E., Tahiliani A. G., McNeill J. H. Effect of vanadate on elevated blood glucose and depressed cardiac performance of diabetic rats. Science (1985), March 22, 227(4693):1474-7.
Houggard D. M, Larsson L. I. Localization and possible function of polyamines in protein and peptide secreting cells. Med. Bio. (1986), 64, 89-94.
Hsuch W A. PPAR-gamma effects on the vasculature. Jour. Investig Med. (2001), 49(1), 127-9.
Hulley P. A, Conradie M. M., Langeveldt C. R, Hough F. S., Glucocorticoid-induced osteoporosis in the rat is prevented by the tyrosine phosphatase inhibitor, sodium orthovanadate. Bone. (2002), 31(1), 220-9.
Husain S., Hadi S. M. Strand scission in DNA induced by L-DOPA in the presence of Cu(II). FEBS Lett. (1995), 364, 75-78.
Husain S., Hadi S. M. DNA breakage by L-DOPA and Cu(II): breakage by melanin and bacteriophage inactivation. Mutat Res. (1998), 397, 161-68.
Huyer G., Liu S., Kelly J., Moffat J., Payette P., Kennedy B., Tsaprailis G., Gresser M. J., Ramachandran C. Mechanism of inhibition of protein-tyrosine phosphatases by vanadate and pervanadate. Jour. Biol. Chem. (1997), 272(2), 843-51.
Ikeda Y., Ziv E., Hanse L. L., Busch A. K., Hansen B. F., Shafrir E., Mosthaf L. Cellular mechanism of nutritionally induced insulin resistance in Psammomys obesus: overexpresion of protein kinase C epsilon in skeletal muscle precedes the onset of hyperinsulinemia and hyperglycemia. Diabetes (1999), 48, A76.
Isobe, K; Ito, S; Hosaka, H; Iwamura, Y; Kondo, H; Kagawa, Y; Hayashi, J I. Nuclear-recessive mutations of factors involved in mitochondrial translation are responsible for age-related respiration deficiency of human skin fibroblasts. Jour. Biol. Chen (1998), February 20, 273(8):4601-6.
Iversen L. F., Andersen H. S., Branner S., Mortensen S. B., Peters G. H., Norris K., Olsen O. H., Jeppesen C. B., Lundt B. F., Ripka W., Moller K. B., Moller N. P. Structure-based design of a low molecular weight, nonphosphorus, nonpeptide, and highly selective inhibitor of protein-tyrosine phosphatase 1B. Jour. Biol, Chem. (2000), 275(14), 10300-7.
Jiang Z. Y., Zhou Q. L., Eaton J. W., Koppenol W. H., Hunt J. V., Wolff S. P. Spirohydantoin inhibitors of aldose reductase inhibit iron- and copper-catalysed ascorbate oxidation in vitro. Biochem. Pharmacol. (1991), August 22, 42(6):1273-8.
Johns, D R, Sadun, A A, Cuban epidemic optic neuropathy. Mitochondrial DNA analysis. Jour. Neuro-Ophthalmol., (1994), September, 14(3):130-4.
Karasu C., Dewhurst M, Stevens E. J., Tomlinson D. R., Effects of anti-oxidant treatment on sciatic nerve dysfunction in streptozotocin-diabetic rats; comparison with essential fatty acids. Diabetologia (1995), 38, 129-34.
Kawasaki E., Hutton J. C., Eisenbarth G. S. Molecular cloning and characterization of the human transmembrane protein tyrosine phosphatase homologue, phogrin, an autoantigen of type 1 diabetes. Biochem Biophys. Res. Commun. (1996), 227(2), 440-7.
Kelner M. J., Bagnell R., Hale B., Alexander N. M. Inactivation of intracellular copper-zinc superoxide dismutase by chelating agents without glutathione depletion and methemoglobin formation. Free Radical Biol. Med. (1989), 6 (4), 355-60.
Kennedy B. P., Ramachandran C. Protein tyrosine phosphatase-1B in diabetes. Biochem. Pharmacol. (2000), 60(7), 877-83.
Kenner K. A., Hil D. E., Olefsky J. M., Kusari J. Regulation of protein tyrosine phosphatases by insulin and insulin-like growth factor L. Jour. Biol. Chem. (1993), 268(34), 2545562.
Khan A. U., DiMascio P., Medeiros M. H., Wilson T. Spermine and spermidine protect ion of plasmid DNA against single-strand breaks induced by singlet oxygen. Proc. Nat. Acad. Sci. (1992), 89, 11428-30.
Kiefer G. E. U.S. Pat. No. 5,385,893 Tricyclopolyazamacrocyclophosphonic Acids, Complexes and Derivatives thereof for use as Contrast Agents.
Kiefer G. E. U.S. Pat. No. 5,462,725. 2-pyridylmethylenepolyazamacrocyclophosphonic Acids, Complexes and Derivatives Thereof for use as Contrast Agents.
Kim E. E., Choy Y. C., Domstad P. A., Beihn R., Coupal J., Yonts S., Deland F. H. Biologic gastric emptying time using Tc-99m TETA polystyrene resin in various clinical conditions. Eur. Jour. Nucl. Med. (1981), 6(4), 155-8.
Kinlaw W. B., Levine A. S., Morley J. E., Silvs S. E., McClain C. J. Abnormal zinc metabolism in type II diabetes mellius. Amer. Jour. Med. (1983), 75, 273-7.
Kodama, H; Murata, Y; Iitsuka, T; Abe, T. Metabolism of administered triethylene tetramine dihydrochloride in humans. Life Sciences, (1997), 61(9):899-907.
Kohen R, Chevion M. Transition metals potentiate paraquat toxicity. Free Rad. Res. Commun (1985), 1, 79-88.
Kooistra T., Van Hinsbergh V., Havekes L., Jan Kempen H In vitro studies on origin and site of action of enzyme activity responsible for conversion of human proapoprotein A-1 into apoprotein A-1. FEBS Lett (1984), 170, 109-13.
Kolm-Litty V, Berlo S, Bonifacio E, Bearzatto M, Engel A M, Christie M, Ziegler A G, Wild T, Endl J. Human monoclonal antibodies isolated from type I diabetes patients define multiple epitopes in the protein tyrosine phosphatase-like IA-2 antigen. Jour. Immunol. (2000), 165(8), 4676-84.
Koya D., King G. L. Protein kinase C activation and the development of diabetic complications. Diabetes. (1998), 47(6), 85966.
Korbashi P., Kohen R., Katzhendler J., Chevion M Iron Mediates paraquatt toxicity in Escherichia coli Jour. Biol. Chem (1986), 261 (27), 12472-476.
Korbashi P., Katzhendler J., Saltman P., Chevion M Zinc protects Escherichia coli against copper-mediated paraquat-induced damage. Jour. Biol. Chem (1989), 264 (15), 8479-82.
Kristal B. S., Koopmans S. J., Jackson Y., Ikeno B. J., Park B. J., Yu B. P. Oxidant-mediated repression of mitochondrial transcription in diabetic rats. Free Radical Biol. Med. (1997), 813-22.
Krueger N. X., Streuli M., Saito H. Structural diversity and evolution of human receptor-like protein tyrosine phosphatases. EMBO Jour. (1990), 9(10), 3241-52.
Kruman, II; Culmsee, C; Chan, S L; Kruman, Y; Guo, Z; Penix, L; Mattson, M. P. Homocysteine elicits a DNA damage response in neurons that promotes apoptosis and hypersensitivity to excitotoxicity. Journal of Neuroscience (2000), September 15, 20(18):6920-26.
Krumkalns, E. V., Pfeifer, W., Adamantylamines by Direct Amination of 1-Bromoadamantane, Jour. Med. Chem., (1968), 11, 1103.
Langsjoen, P H; Folkers, K; Lyson, K; Muratsu, K; Lyson, T; Langsjoen, P. Effective and safe therapy with coenzyme Q10 for cardiomyopathy. Klinische Wochenschrift (1988), July 1, 66(13):583-90.
Law R., Meehan W., Xi X, Graf K, Wuthrich D., Coats W., Faxon D., Hsuch W. Troglitazone inhibits vascular smooth muscle cell growth and intimal hyperplasia Jour Clin. Invest (1998), 98, 1897-1905.
Leach C K, Carr N G. Electron transport and oxidative phosphorylation in the blue-green alga Anabaena variabilis. Jour. Gen. Microbiol. (1970), 64(1), 55-70.
Leclerq-Meyer V., Malaisse W. J., Enhancement by succinic acid dimethylester of insulin release evoked by D-glucose and glimepiride in the perfused pancreas of normoglycemic and hyperglycemic rats. Biochem Pharmacol. (1993), 47, 1519-24.
Lee T. S., Saltsman K. A., Ohashi I Activation of protein kinase C by elevation of glucose concentration: proposal for a mechanism in the development of diabetic vascular complications. Proc. Natl. Acad. Sci. (1989), July; 86(13):5141-5.
Lee, H K; Song, J H; Shin, C S; Park, D J; Park, K S; Lee, K U; Koh, C S. Decreased mitochondrial DNA content in peripheral blood precedes the development of non-insulin-dependent diabetes mellitus. Diabetes Res. Clin. Prac. (1998) December, 42(3):161-7.
Levay G., Ye Q., Bodell W. J. Formation of DNA adducts and oxidative base damage by copper mediated oxidation of dopamine and 6-hydroxydopamine. Exper. Neurol. (1997), 146,570-74.
Li L., Dixon J. E. Form, function, and the regulation of protein tyrosine phosphatases and their involvement in human diseases. Semin. Immunol. (2000), 12, 75-84.
Li Z., Zhao L., Sandler S., Karlsson F. A. Expression of pancreatic islet MHC class I, insulin, and ICA 512 tyrosine phosphatase in low-dose streptozotocin-induced diabetes in mice. Jour. Histochem. Cytochem. (2000), 48(6), 761-7.
Lloyd K. Ionic vs. nonionic contrast media Radiol Technol. (1994), 66(1), 57-9.
Lovell M. A., Robertson J. D., Teesdale W. J., Campbell J. L., Markesbery W. R. Copper, iron, and zinc in Alzheimer's disease senile plaques. Jour. Neur. Sci. (1998), 158,47-52.
Lu J., Li Q., Xie H., Chen Z. J., Borovitskaya A. E., Maclaren N. K., Notkins A. L., Lan M. S. Identification of a second transmembrane protein tyrosine phosphatase, IA-2beta, as an autoantigen in insulin-dependent diabetes mellitus: precursor of the 37-kDa tryptic fragment Proc. Natl. Acad. Sci. (1996), 93(6), 2307-11.
Lukkarainen J., Kauppinen R. A., Koistinaho J., Halmekyto M., Alhonen L. Janne J. Cerebral energy metabolism and immediate early gene induction following severe incomplete ischaemia in transgenic mice overexpressing the human ornithine decarboxylase gene: evidence that putrescine is not neurotoxic in vivo. Eur. Jour. Neurosci. (1995), 7(9), 1840-49.
Lynch J. J. Ferro T. J., Blumenstock F. A. Increased endothelial albumin permeability mediated by protein kinase C activation. Jour. Clin Invest. (1990), June;85(6):1991-98.
Low, P A; Nickander, K K; Tritschler, H J. The roles of oxidative stress and antioxidant treatment in experimental diabetic neuropathy. Diabetes (1997), September, 46 Suppl 2:S38-42.
MacDonald M. J., Fahien L. A. Glyceraldehyde phosphate and methyl esters of succinic acid Two “new” potent insulin secretagogues. Diabetes. (1988), July; 37(7):997-9.
Maechler, P; Wollheim, C B. Mitochondrial signals in glucose-stimulated insulin secretion in the β cell. Jour. Physiol. (2000) November 15, 529 Pt 1:49-56.
Maedler, K; Spinas, G A, Dyntar, D; Moritz, W. Kaiser, N; Donath, M Y. Distinct effects of saturated and monounsaturated fatty acids on β-cell turnover and function. Diabetes (2001), January, 50(1):69-76.
Malachowski, M. R., Adams, N, Elia, E., Rheingold, A. L., Kelly, RS., Enforcing Geometrical Constraints On Metal Complexes using Biphenyl-based Ligands. Jour. Chem. Soc., Dalton Trans., (1999), 2177.
Malaisse W. J, Sener A. Metabolic effects and fate of succinate esters in pancreatic islets. Amer. Jour. Physiol. (1993), March, 264(3 Pt I)-E434-40.
Malaisse W. J., Rassachaert J., Villaneuva-Penacanillo M. I., Valverde I. Respiratory, ionic, and functional effects of succinate esters in pancreatic islets. Amer. Jour. Physiol. (1993), March, 264(3 Pt 1):E428-33.
Malaisse, W. J. The β cell in NIDDM: giving light to the blind. Diabetologia (1994), September, 37 Suppl 2:S3642.
Marin-Garcia, J; Goldenthal, M J; Ananthalrishnan, R; Pierpont, M E. The complete sequence of mtDNA genes in idiopathic dilated cardiomyopathy shows novel missense and tRNA mutations. Jour. Card. Fail. (2000), December, 6(4), 321-9.
Marx N, Schonbeck U, Lazar M A, Libby P, Plutzky J. Peroxisome proliferator-activated receptor gamma activators inhibit gene expression and migration in human vascular smooth muscle cells. Circ Res. (1998), 83(11), 1097-103.
Marseigne, I., Roques, B. P.,. Synthesis of New Amino Acids Mimicking Sulfated and Phosphorylated Tyrosine Residues. Jour. Org. Chem., (1988), 53, 3621.
Mateo M. C. M., Bustamante J. B., Cantalapiedra M. A. G. Serum zinc, copper and insulin in diabetes mellitus. Biomed (1978), 29, 56-58.
Matthai W. H. Clinical and economic factors in the selection of low-osmolality contrast media Pharmacoeconomics. (1994), 5(3), 188-97.
Mattson, M P; Culmsee, C; Yu, Z F. Apoptotic and antiapoptotic mechanisms in stroke. Cell Tissue Res. (2000), July, 301(1):173-87.
Matus A., Green G. Age-related increase in a cathepsin D like protease that degrades brain microtubule-associated proteins. Biochemistry (1987), 26, 8083-86.
McCance D. R., Dyer D. G., Dunn J. A., Bailie K. E., Thorpe S. R., Baynes J. W., Lyons T. J. Maillard reaction products and their relation to complications in insulin-dependent diabetes mellitus. Jour. Clin Invest. (1993), June 91(6):2470-8.
McCully K. S. Homocysteine metabolism in scurvy, growth and arteriosclerosis. Nature (1971), 23, 391-92.
McCully K. S., Vezeridis M. P., Histopathological effects of homocysteine thiolactone on epithelial and stromal tissues. Exp. Molec. Pathol. (1989), 51, 159-70.
McCully K. S., Olszewski A. J., Vezeridis M. P. Homocysteine and lipid metabolism in atherogenesis: effect of the homocysteine thiolactonyl derivatives, thioretinaco and thioretinamide. Atherosclerosis (1990), August, 83(2-3): 197-206.
McCully, K. S. Tzanakakis G. N., Vezeridis M. P. Effect of the synthetic N-homocysteine thiolactonyl derivatives, thioretinaco, thioretinamide, and thioco on growth and lactate production by malignant cells. Res. Commun Chem. Pathol. Pharmacol. (1992), July, 77(1):125-8.
McCully K. S. Chemical pathology of homocysteine. III. Cellular function and aging Ann. Clin. Lab. Sci. (1994a), March-April, 24(2):134-52.
McCully, K S. Chemical pathology of homocysteine. II. Carcinogenesis and homocysteine thiolactone metabolism. Ann. Clin Lab. Sci (1994b), January-February, 24(1):27-59.
McGurk J. F., Bennett M. V., Zukin R. S. Polyamines potentiate responses of N-methyl-D-aspartate receptors expressed in Xenopus oocytes. Proc. Natl. Acad. Sci. (1990), 87, 9971-74.
McLaren G. D. Muir W A. Kellermeyer R. W. Iron overload disorders: natural history, pathogenesis, diagnosis and therapy. Crit Rev. Clin. Lab. Sci. (1983), 19, 205-266.
MacDonald M. J. High content of mitochondrial glycerol-3-phosphate dehydrogenase in pancreatic islets and its inhibition by diazoxide. Jour. Biol. Chem. (1981), 256(16), 8287-90.
Mecocci, P; Polidori, M C; Ingegni, T; Cherubini, A; Chionne, F; Cecchetti, R; Senin, U. Oxidative damage to DNA in lymphocytes from AD patients. Neurology (1998), October, 51(4):1014-7.
Mecocci P., MacGarvey U., Kaufman A. E. Oxidative damage to mitochondrial DNA shows marked age dependent changes increases in human brain Ann. Neurol (1993), 34,609-16.
Mecocci, P; MacGarvey, U; Beal, M F. Oxidative damage to mitochondrial DNA is increased in Alzheimer's disease. Annals Neurol. (1994), November, 36(5):747-51.
Melby, L. R., Polymers for Selective Chelation of Transition Metals. Jour. Am. Chem. Soc., (1975), 97, 4044.
Mezzetti G., Monti M. G., Moruzzi M. S. Polyamines and the catalytic domain of protein kinase C. Life Sci (1988), 42,2293-98.
Milarski K. L., Zhu G., Pearl C. G., McNamara D. J., Dobrusin E. M., MacLean D., Thieme-Sefler A., Zhang Z. Y., Sawyer T., Decker S. J. Sequence specificity in recognition of the epidermal growth factor receptor by protein tyrosine phosphatase 1B. Jour. Biol. Chem. (1993), 268(31), 23634-9.
Mizukami, F., Metal Complexes Containing Six-Membered Chelate Rings. The Preparation and Structure of Dichlorocobalt(III) Complexes with Tetramines Derived from 2,4-Pentanedimaine, Bull. Chem. Soc. Jpn., (1975), 48, 1205-1212.
Mikukami, F., Metal Complexes Containing Six-Membered Chelate Rings. IV. The Preparation and Structure of Dichlorocobalt(III) Complexes with Tetramines Derived from 2,4-Pentanediamine. Bull. Chem. Soc, Japn., (1975), 48, 1205-1212.
Miyako, K; Kai, Y; Irie, T;. Takeshige, K; Kang, D. The content of intracellular mitochondrial DNA is decreased by 1-methyl-4-phenylpyridinium ion (MPP+). Jour. Biol. Chem (1997), April 11, 272(15):9605-8.
Miyako, K; Irie, T; Muta, T; Umeda, S; Kai, Y; Fujiwara, T; Takeshige, K; Kang, D.1-Methylphenylpyridinium ion (MPP+) selectively inhibits the replication of mitochondrial DNA Eur. Jour. Biochem. (1999), January, 259(1-2):412-8.
de la Monte, S M; Luong, T; Neely, T R; Robinson, D; Wands, J R. Mitochondrial DNA damage as a mechanism of cell loss in Alzheimer's disease. Lab. Invest (2000), August, 80(8):1323-35.
Montserrat J., Chen L., Lawrence D. S., Zhang Z. Potent low molecular weight substrates for protein-tyrosine phosphatase. Jour. Biol. Chem. (1996), 271(13), 7868-72.
Morgan J. M. Hepatic chromium content in diabetic subjects. Metabolism: Clin. Exper. (1972), April, 21(4):3134.
Morris T. W., X-ray contrast media: where are we now, and where are we going? Radiology. 1993,188(1), 11-6
Moruzzi M. S., Monti M. G., Piccinini G., Marverti G., Tadolini B. Effect of spermine on association of protein kinase C with phospholipid vesicles. Life Sci. (1990), 47(16), 1475-82.
Moruzzi M. S., Marverti G., Piccinini G., Frassineti C., Monti M. G. The effect of spermine on calcium requirement for protein kinase C association with phospholipid vesicles. International Jour. Biochem. Cell Biol. (1995), 27(8), 783-8.
Moruzzi M. S., Piccinini G., Tadolini B., Monti M. G., Barbiroli B., Mezzetti G. In: Progress in polyamine research Effect of polyamines on protein kinase C activation process. Adv. Exp. Med. Biol. (1988), 250:469-80.
Moustaid N., Sul H. S., Regulation of expression of the fatty acid synthase gene in 3T3-L1 cells by differentiation and triiodothyronine. Jour. Biol. Chem. (1991), 18550-554.
Muller H. K., Kellerer M, Ermel B., Muhlhofer A, Obermaier-Kusser B., Vogt B., Haring H. U. Prevention by protein kinase C inhibitors of glucose-induced insulin receptor tyrosine kinase resistance in rat fat cells. Daibetes (1991), 1440-48.
Naviaux, R K. Mitochondrial DNA disorders. Eur. Jour. Ped (2000), December, 159 Suppl 3:S219-26.
Noto R, Alicata R., Sfogliano L. A study of cupremia in a group of elderly diabetics. Acta Diabetol. Latina (1983), 20, 81-85.
Oberholzer, M. R., Neuburger, M, Zehnder, M, Kaden, T. A., Steric Effects in the Cu(II) and Ni(II) Complexes with Tetra-N-alkylated 1,4,8,11-Tetrazacyclotetadecanes. Helv. Chim Acta, (1995), 78, 505.
Odawara, M; Yamashita, K. Mitochondrial gene abnormalities and α- and β-cell dysfunction. Diabetes Care (1996), October, 19(10):1166-7.
Oetken C, Pessa-Morikawa T, Autero M Andersson L C, Mustelin T. Reduced tyrosine phosphorylation in polyamine-starved cells. Exp Cell Res. (1992), 202(2), 370-5.
Oka Y., Katagiri H, Yazaki Y., Murase T., Kobayashi T. Mitochondrial gene mutation in islet-autoantibody-positive patients who were initially non-insulin-dependent diabetics. Lancet (1993), 342, 527-28.
Oka, Y.NIDDM—genetic marker, glucose transporter, glucokinase, and mitochondria gene. Diabetes Res. Clin. Prac. (1994), October, 24 Suppl., S117-21.
Olszewski A. J., McCully K. S. Homocysteine metabolism and the oxidative modification of proteins and lipids. Free Rad. Biol. Med. (1993), June, 14(6):683-93.
Ozawa, T. Mitochondrial DNA mutations in myocardial diseases. European Heart Journal (1995), December, 16 Suppl O, 10-14.
Ozawa, T; Hayakawa, M; Katsumata, K; Yoneda, M; Ikebe, S; Mizuno, Y. Fragile mitochondrial DNA: the missing link in the apoptotic neuronal cell death in Parkinson's disease. Biochem. Biophys. Res. Commun. (1997), June 9, 235(1):158-61.
Palmer J. P., Clemons P., Lyen K., Tatpati O. Insulin antibodies in insulin-dependent diabetics before insulin treatment. Science (1983), 222, 1337-39.
Papadopoulou, L C; Theophilidis, G; Thomopoulos, G N; Tsiftsoglou, A S. Structural and functional impairment of mitochondria in adriamycin-induced cardiomyopathy in mice: suppression of cytochrome c oxidase H gene expression. Biochem. Pharmacol. (1999), March 1, 57(5):481-9.
Park, K S; Lee, K U; Song, J H; Choi C S; Shin, C S; Park, D J; Kim, S K; Koh, J J; Lee, EIK Peripheral blood mitochondrial DNA content is inversely correlated with insulin secretion during hyperglycemic clamp studies in healthy young men. Diabetes Res. Clin. Prac. (2001), May, 52(2):97-102.
Parvez Z., Vik H Nonionic contrast media and blood clotting. A critical review. Invest Radiol. (1991), Suppl 1, S103-6; discussion S107-9.
Passini N., Larigan J. D., Genovese S., Appella E., Sinigaglia F., Rogge L. The 37/40-kilodalton autoantigen in insulin-dependent diabetes mellitus is the putative tyrosine phosphatase IA-2. Proc. Natl. Acad. Sci. (1995), 92(20), 9412-6.
Payton M. A., Hawkes C. J., Christie M. R. Relationship of the 37,000- and 40,000-M(r) tryptic fragments of islet antigens in insulin-dependent diabetes to the protein tyrosine phosphatase-like molecule IA-2 (ICA512). Jour. Clin. Invest, (1995), 96(3), 1506-11.
Perlmutter L. S., Siman R, Gall C., Seubert P., Lynch G. The ultrastructural localization of calcium-activated protease “calpain” in rat brain. Synapse (1988), 2,78-88.
Pieper A. A., Brat D. J., Krug D. K., Watkins C. C., Gupta A, Blackshaw S., Verma A., Wang Z., Snyder S. H., Poly(ADP-ribose) polymerase-deficient mice are protected from streptozotocin-induced diabetes. Proc. Natl. Acad. Sci. (USA) (1999), 96(6), 3059-64.
Phillips M. K., Kell D. B. A novel inhibitor of NADH dehydrogenase in Paracoccus denitrificans. FEBS Lett. (1982), 140(2):248-50.
Posner B. L., Faure R, Burgess J. W., Bevan A. P., Lachance D., Zhang-Sun G., Fantus LG., Ng J. B., Hall D. A., Lum B. S., Shaver A. Peroxovanadium compounds. A new class of potent phosphotyrosine phosphatase inhibitors which are insulin mimetics. Jour. Biol. Chem. (1994), 269(6), 4596-604.
Press E. M., Porter R., Cebra J. The isolation and properties of a proteolytic enzyme, cathepsin D, from bovine spleen. Biochem. Jour. (1960), 74, 501-14.
Puius Y. A., Zhao Y., Sullivan M, Lawrence D. S., Almo S. C., Zhang Z. Y. Identification of a second aryl phosphate-binding site in protein-tyrosine phosphatase 1B: a paradigm for inhibitor design Proc. Natl. Acad Sci. (1997), 94(25), 13420-5.
Quigley H. A. Ganglion cell death in glaucoma: Pathology recapitulates ontogeny. Aust NZ J. Ophthalmol. (1995), 23, 85-91.
Razzaboni B. L., Papastoitsis G., Koo E. H., Abraham C. R., A calcium-stimulated serine protease from monkey brain degrades the B-amyloid precursor protein. Brain Res. (1992), 589,207-16.
Reaven G. M. Role of insulin resistance in human disease (syndrome X): an expanded definition. Ann Rev. Med. (1993), 44, 121-31.
Reul, B. A., Amin S. S., Buchet, J. P., Ongemba L. N., Crans D. C., Brichard S. M. Effects of vanadium complexes with organic ligands on glucose metabolism: a comparison study in diabetic rats. Brit Jour. Pharmacol. (1999) January, 126(2):467-77.
Reusser F. Mode of action of streptozotocin. Jour. Bacteriol. (1971), 105(2), 580-8.
Rose C., Camus A, Schwartz J. C. A serine peptidase responsible for the inactivation of endogenous cholecystokinin in brain. Proc. Natl. Acad. Sci. (1988), 85(21), 8326-30.
Rose C., Camus A, Schwartz J. Protection by serine peptidase inhibitors of endogenous cholecystokinin released from brain slices. Neuroscience (1989), 29(3), 583-94.
Rosenkranz H. S., Carr H. S. Differences in the action of nitrosomethylurea and streptozotocin. Cancer Res. (1970), (1), 112-7.
Sadun, A Acquired mitochondrial impairment as a cause of optic nerve disease. Trans. Amer. Ophthalmol. Soc. (1998), 96:881-923.
Ross W. E., Block E. R., Chang R. Y. Paraquat-induced damage in mammalian cells. Biochem. Biophys. Res. Commun. (1979), 4, 1302-08.
Scanu A. M. Proapolipoprotein-converting enzymes and high density lipoprotein early events in biogenesis. Amer. Heart Jour. (1987), 113,527-33.
Schapira, A H. Evidence for mitochondrial dysfunction in Parkinson's disease—a critical appraisal. Movement Disorders (1994), March, 9(2):125-38.
Schapira, A H. Mitochondrial dysfunction in neurodegenerative disorders. Biochim. Biophys. Acta (1998), August 10, 1366(1-2):225-33.
Schmitz C; Axmacher B; Zunker U; Korr R Age-related changes of DNA repair and mitochondrial DNA synthesis in the mouse brain. Acta Neuropath (1999), January, 97(1):71-81.
Schmitz-Pfeiffer C., Browne C. L., Oakes N. D., Watkinson A., Chissholm D. J., Kraegen E. W., Biden T. J. Alterations in the expression and cellular localization of protein kinase c isozymes θ and ε are associated with insulin resistance in skeletal muscle of the high fit-fed rat Diabetes (1997), 46, 169-78.
Schroeder H. A. Cadmium, chromium, and cardiovascular disease. Circulation 1967 March, 35(3):570-82.
Schroeder H. A. Chromium deficiency as a factor in atherosclerosis. Jour. Chronic Dis. (1970), 23, 123-42.
Seghieri G., Gironi A, Mammini P., Alviggi L., De Giorgio L. A., Bartolomei G., Ignesti G., Franconi F. Erythrocyte spermidine levels in IDDM patients. Diab. Care. (1992), (4), 543-5.
Seghieri G., Gironi A., Niccolai A, Mammini P., Alviggi L., De Giorgio L. A., Caselli P., Bartolomei G. Serum spermidine oxidase activity in patients with insulin-dependent diabetes mellitus and microvascular complications. Acta Diabetol. Lat (1990), (4), 303-8.
Seidman M D; Khan M J; Bai U; Shirwany N; Quirk W S. Biologic activity of mitochondrial metabolites on aging and age-related hearing loss. Amer. Jour. Otol. (2000), March, 21(2):161-7.
Serradas P., Girox M. H., Saulnier C., Mitochondrial deoxyribonucleic acid content is specifically decreased in adult but not fetal pancreatic islets of the Goto-Kakiki rat, a genetic model of insulin-dependent diabetes. Endocrinology(1995), 136,5623-31.
Sherry A. D. U.S. Pat. No. 5,188,816 Using Polyazamacrocyclic Compounds for Intracellular Measurement of Metal Ions Using MRI.
Sherry A. D. et al U.S. Pat. No. 5,342,606. Polyazamacrocyclic Compounds for Complexation of Metal Ions
Sherry A. D. et al. U.S. Pat. No. 5,362,476 Alkyl Phosphonate Polyazamacrocyclic Chelates for MRI
Sherry A. D. et al U.S. Pat. No. 5,630,997. Phosphorylated Polyazamacrocyclic Compounds for Complexation of Metal Ions.
Shoffler, J M; Brown, M D; Torroni, A; Lott, M T; Cabell, M F; Mirra, S S; Beal, M F; Yang, C C; Gearing, M; Salvo, R; et al. Mitochondrial DNA variants observed in Alzheimer disease and Parkinson disease patients. Genomics (1993), July, 17(1):171-84.
Sjoholm A. Role of polyamines in the regulation of proliferation and hormone production by insulin-secreting cells. Am J. Physiol. (1993), 264(3 Pt 1), C501-18.
Smith M. A., Harris P. L., Sayre L. M., Perry G. Iron Accumulation in Alzheimer's disease is a source of redox-generated free radicals. Proc. Natl. Acad. ScL (1997), 94, 9866-9868.
Snyder R. D. Inhibition of X-ray-induced DNA strand break repair in polyamine depleted HeLa cells. Int Jour. Radiat. Biol. (1989), 15(5), 773-79.
Soong N. W., Hinton D. P., Cortopassi G., and Amlheim N. Mosaicism for a specific somatic mitochondrial DNA mutation in adult human brain Nat. Genet. (1992), December; 2(4):318-23.
Spinosa D. J., Kaufinann J. A., Hartwell G. D. Gadolinium chelates in angiography and interventional radiology: a useful alternative to iodinated contrast media for angiography. Radiology. 2002 223(2), 319-25; discussion 326-7.
Steinke J., Soeldner J. S. Metabolic effects of diazoxide in normal subjects, patients with diabetes mellitus, and patients with organic hypoglycemia Ann. New York Acad. Sci. (1968), 150 (2), 326-36.
Strout H. V., Vicario P. P., Biswas C., Saperstein R., Brady E. J., Pilch P. F., Berger J. Vanadate treatment of streptozotocin diabetic rats restores expression of the insulin responsive glucose transporter in skeletal muscle. Endocrin. (1990), 126, 2728-32.
Sucher N. J., Lipton S. A., Dreyer E. B., (1997), Molecular pathology of glutamate toxicity in retinal ganglion cells. Vision Res. (1997), 37, 3483-93.
Suzuki Y., Kadowaki H, Atsumi Y., A case of diabetic amyotrophy associated with 3243 mitochondiial tRNA (Leu UUR) mutation and successful therapy with coenzyme Q10. Endoc. Jour. (1995), 42,141-45.
Suzuki S; ltinokio Y. Komatu K; Ohtomo M; Onoda M; Hirai S; Hirai M; Hirai A; Chiba M; Kasuga S; Akai H; Takahashi T., Hiratani K, Kimura E., N2-Binding Mononuclear Ru(II) Tertiary Polyamine Complex, Chemistry Letters, (1993), 1329-1332.
Swoboda B E; Egawhary D N; Chen 3; Vince F P. Diabetic complications and the mechanism of the hyperglycaemia-induced damage to the mt DNA of cultured vascular endothelial cells: (II) The involvement of protein kinase C. Biochem. Soc. Trans. (1995), November, 23(4):519S.
Tagami U, Ikeda K., Yamagata K., Nara Y., Fujino H, Kubota A., Numano F., Yamori Y. Vitamin E prevents apoptosis in hippocampal neurons caused by cerebral ischemia and reperfusion in stroke-prone spontaneously hypertensive rats. Lab. Invest (1999), 79,609-15.
Tappia P. S., Sharma R. P., Sale G. J., Dephosphorylation of autophosphorylated insulin and epidermal growth factor receptors by two major subtypes of protein tyrosine phosphatase from human placenta. Biochem. Jour. (1991), 278, 69-74.
Tasker R. C., Sahota S. K., Cotter F. E., Williams S. R. Early post-ischemic dantrolene induced amelioration of poly(ADP-ribose) polymerase-related bioenergetic failure in neonatal rat brain slices. Jour. Cereb. Blood Flow Metab. (1998), 18, 1346-56.
Tonks N. K., Diltz C. D., Fischer E. H. Characterization of the major protein-tyrosine-phosphatases of human placenta. Jour. Biol. Chem. (1988), 263(14), 6731-7.
Tontonoz P., Nagy L., Alvarez J. G., Thomazy V. A., Evans RLL,. PPARgamma promotes monocyte/macrophage differentiation and uptake of oxidized LDL. Cell. (1998), 93(2), 241-52.
Toyota, T. Oxidative damage to mitochondrial DNA and its relationship to diabetic complications. Diabetes Res. Clin. Prac. (1999), September, 45(2-3):161-8.
Trachtman E, Futterweit S., Maesake J. Taurine ameliorates chronic streptozotocin-induced diabetic nephropathy in rats. Amer. Jour. Physiol. (1995), 269, F429-F438.
Trembleau S, Penna G, Gregori S, Magistrelli G, Isacchi A, Adorini L. Early Th1 response in unpnned nonobese diabetic mice to the tyrosine phosphatase-like insulinoma-associated protein 2, an autoantigen in type I diabetes. Jour. Immunol. (2000), 165(12), 6748-55.
Tsuji A, Sakurai H. Vanadyl ion suppresses ntric oxide production from peritoneal macrophages of streptozotocin-induced diabetic mice. Biochem Biophys. Res. Commua (1996), September 13, 226(2):506-11.
Uchigata Y., Yamamoto H., Kawamura A, Okamoto H. Protection by superoxide dismutase, catalase and poly (ADP-ribose) synthetase inhibitors against alloxan and streptozotocmi-minduced islet DNA strand breaks and against the inhibition of proinsulin biosynthesis. Jour. Biol. Chem. (1982), 257,6084-88.
Uemura, S; Matsushita, H; Li, W; Glassford, A J; Asagami, T; Lee, K H; Harrison, D G; Tsao, P S. Diabetes mellitus enhances vascular matrix metalloproteinase activity: role of oxidative stress Circul. Res. (2001), June 22, 88(12):1291-8.
Umeda S; Muta T; Ohsato T; Takamatsu C; Hamasaki N; Kang D. The loop structure of human mtDNA is destabilized directly by 1-methyl-4-phenylpyridinium ion (MPP+), a Parkinsonism-causing toxin. Eur. Jour. Biochem. (2000), January, 267(1):200-6.
Uriu-Hare J. Y., Stern, J. S., Keen C. L. The effect of diabetes on the molecular localization of maternal and fetal zinc and copper metalloprotein in the rat Biol. Trace Element Res. (1988), December, 18:71-9.
Van Alphen J. On aliphatic polyamines III. Jour. Rec. Trav. Chim. (1936), 55, 835-40.
Valera A., Rodriguez-Gil I. E., Bosch F. Vanadate treatment restores the expression of genes for key enzymes in the glucose and ketone bodies metabolism in the liver of diabetic rats. Jour. Clin. Invest. (1992), 92, 4-11.
Van Gaal L. F., Vansant G. A. De Leeuw I. H. Haemostasis and visceral fat. In Progress in Obesity Research 8, Guy-Grand B., Ailhaud G. (eds.) London: John Libbey & Co., 1999, 577-85.
Villringer A., Rosen B. R., Belliveau J. W., Ackerman J. L., Lauffer R. B., Buxton R. B., Chao Y. S., Wedeen V. J., Brady T. J. Dynamic imaging with lanthanide chelates in normal brain: contrast due to magnetic susceptibility effects. Magn. Reson Med. (1988), 6(2), 164-74.
Vicent D., Villaneuva-Penacarillo M. L., Valverde L, Malaisse W. J. enhancement of the insulinotropic action of glibenclamide by succinnic acid methyl esters in anaesthetized rats. Med Sci. Res. (1993), 21, 517-18.
Waiclii S., Colinge J., Hooft van Huijsduijnen R MetaBlasts: tracing protein tyrosine phosphatase gene family roots from Man to Drosophila melanogaster and Caenorhabditis elegans genomes. Gene. (2000), 253(2), 137-43.
Wallace, D. C. Mitochondrial genetics: a paradigm for aging and degenerative diseases? Science (1992), May 1, 256(5057):628-32.
Wang C. Mangafodipir trisodium (MnDPDP)-enhanced magnetic resonance imaging of the liver and pancreas. Acta Radiol. Suppl. (1998), 415:1-31.
Wei Y. H., Kao S. H. Mitochondrial DNA mutations and lipid peroxidation in human aging. In: Nutrients and Gene Expression. Berdanier C. D. ed. Boca Raton Fla.: CRC Press, (1996); pp 165-88.
Wen G., Zhang X. L., Chang R. M., Xia Q., Cang P., Zhang Y. Superparamagnetic iron oxide (Feridex)-enhanced MRI in diagnosis of focal hepatic lesions. Di Yi Jun Yi Da Xue Xue Bao. (2002), 22(5), 451-2.
Welsh N., Sjoholm A., Polyamines and insulin production in isolated mouse pancreatic islets. Biochem. Jour. 1988), 252(3), 701-7.
Welsh N. A role for polyamines in glucose-stimulated insulin gene expression. Biochemt Jour. (1990), 271, 393-97.
Whitaker J. N., Seyer J. Isolation and characterization on of bovine brain cathepsin D. Jour. Neurochem. (1979), 32, 325-33.
Wolf. G., Yuen V. G., Thompson K. H., McNeill J. H., Orvig C. Vanadyl-biguanide complexes as potential synergistic insulin mimics. Jour. Inorg Biochem (1999), 76(3-4), 251-57.
Wolf G. Adipocyte differentiation is regulated by a prostaglandin liganded to the nuclear peroxisome proliferator-activated receptor. Nutr Rev. (1996), 54(9), 290-2.
Wrobel J., Sredy J., Moxham C., Dietrich A., Li Z., Sawicki D. R., Seestaller L., Wu L., Katz A., Sullivan D., Tio C., Zhang Z. Y. PTP1B inhibition and antihyperglycemic activity in the ob/ob mouse model of novel 11-arylbenzo[b]naphtho[2,3A4furans and 111-aylbenzo[b]naphtho[2,34thiophenes. J Med Chem. 1999 Aug. 26; 42(17):3199-202.
Yamamoto H., Uchigata Y., kamoto H. Streptozotocin and alloxan induce DNA strand breaks and poly (ADP-ribose) synthetase in pancreatic islets. Nature (1981a), 294,284-86.
Yamamoto H., Uchigata Y., kamoto H DNA strand breaks in pancreatic islets by in vivo administration of aloxan or streptozotocin. Biochem. Biophys. Res. Commun. (1981b), 103, 1014-20.
Yang, J; Cherian, M G. Protective effects of metallothionein on streptozotocin-induced diabetes in rats. Life Sci. (1994), 55(1):43-51.
Yoshimoto, S; Sakamoto, K; Wakabayashi, I; Masui, H. Effect of chromium administration on glucose tolerance in stroke-prone spontaneously hypertensive rats with streptozotocin-induced diabetes. Metabolism: Clin Exper. (1992), June, 41(6):636-42
Yu Z. F., Bruce-Keller A. J., Goodman Y., Mattson M. P. Uric acid protects neurons against excitotoxic and metabolic insults in cell culture, and against focal ischemic brain injury in vivo. Jour. Neurosci. Res. (1998), 53, 613-25.
Zasloff M, Willis J I, Chen Q, Anderson M, Maeder T, Holroyd K, Jones S, Kinney W, Cheshire K, McLane M. A spermine-coupled cholesterol metabolite from the shark with potent appetite suppressant and antidiabetic properties. Int J Obes Relat Metab Disord (2001), 25(5), 689-97.
Zeviani, M, Mariotti, C; Antozi, C; Fratta, GM; Rustin, P; Prelle, A. OXPHOS defects and mitochondrial DNA mutations in cardiomyopathy. Muscle and Nerve (1995), 3, S1704.
Zeviani M, Amati P., Comi G., Fratta G., Mariotti C., Tiranti V. Searching for genes affecting the structural integrity of the mitochondrial genome. Biochim. Biophys. Acta (1995), 1271,153-58.
Zhang J., Pieper A., Snyder S. H. Poly (ADP-Ribose) Synthetase Activation: An early Indicator of neurotoxc DNA damage. Jour. Neurochem. (1995), 1411-14.
Zhang Z. Protein-tyrosine phosphatases: biological function, structural characteristics, and mechanism of catalysis. Crit Rev Biochem Mol. Biol. (1998), 33(1), 1-52.

1. A method of treating degenerative diseases due to
acquired mitochondrial DNA damage,
redox damage to mitochondrial macromolecules
and inherited mitochondrial genetic defects
said method comprising the steps of: selecting a composition from a group consisting of
predominantly linear tetraamines and polyamines linked by 1,3-propylene and/or ethylene groups, predominately branched tetraamines and polyamines linked by 1,3-propylene and/or ethylene groups, cyclic polyamines linked by 1,3-propylene and/or ethylene groups, combinations of linear, branched and cyclic polyamines linked by one or more 1,3-propylene and/or ethylene groups, substituted polyamines, polyamines derivatized to form tyrosine phosphatase inhibitor molecules with linear or branched is chains attached, polyamine derivatives of 2,2′-diaminobiphenyl with linear or branched chains attached;
synthesizing said composition; and
administering an effective dose of said composition to a mammal.

2. The method of claim 1 wherein said step of synthesizing comprises converting by treatment with an alkyl halide a compound taken from a group consisting of compounds having the formula: 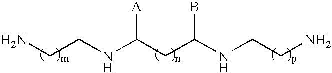
wherein A and B are hydrogen or alkyl and m, n, and p are the same or different, compounds having the formula: 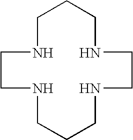
wherein:
M, n, and p may be the same or different and are bridging groups of variable length from 3-12 carbons.
X₁ and X₂ may be the same or different and are nitrogen, sulfur, phosporous and carbon.
, compounds having the formula: 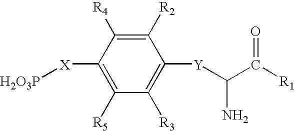
wherein
R₁-R₄ may be the same or different and are hydrogen, alkyl aryl, cycloalkyl, hydroxyl, thiol, amino acid, glutathione, phosphate, phosphonates, uric acid, ascorbic acid, taurine, estrogen, dehydroepiandrosterone, probucol, vitamin E, hydroxytoluene, carvidilol, α-lipoic acid, α-tocopherol, ubiquinone, phylloquinone, β-carotene, meanadione, succinate, acetyl-L-carnitine, co-enzyme Q, lazeroids, polyphenolic flavonoids, —(CH₂)ₙ[XCCH₂)ₙ]NH₂— wherin n=3-12 and X=nitrogen, sulfur, phosporous or carbon, or heterocycle wherin R₁ and R₂ taken together are —(CH₂XCH₂)ₙ— wherin n=3-12 and X=nitrogen, sulfur, phosphorous or carbon. R₅ and R₅ may be the same or different and are hydrogen, alkyl, aryl, cycloalkyl, hydroxyl, thiol, amino acid, glutathione, phosphate, phosphontes, uric acid, ascorbic acid, taurine, estrogen, dehydroepiandrosterone, probucol, vitamin E, hydroxytoluene, carvidilol, α-lipoic acid, α-tocopherol, ubiquinone, phylloquinone, β-carotene, meanadione, succinate, acetyl-L cartine, co-enzyme Q, lazeroids, polyphenolic flavonoids, or heterocycle wherin R₃ and R₄ taken together are —(CH₂XCH₂)ₙ— wherin n=3-12 and X=nitrogen, sulfur, phosphorous or carbon.
R₇ and % may be the same or different and are hydrogen, alkyl, aryl, cycloalkyl, hydroxyl, thiol, amino acid, glutathione, phosphate, phosphonate, uric acid, ascorbic acid, taurine, estrogen, dehydroepiandrosterone, probucol, vitamin E, hydroxytoluene, carvidilol, α-lipoic acid, α-tocopherol, ubiquinone, phylloquinone, β-carotene, meanadione, succinate, acetyl-L-carnitine, co-enzyme Q, lazeroids, polyphenolic flavonoids, —(CH₂)ₙ[CH₂)ₙ]NH₂— wherin n=3-12 and X=nitrogen, sulfur, phosporous or carbon, or heterocycle wherin R₅ and R₆ taken together are —(CH₂XCH₂)ₙ— wherin n=3-12 and X=nitrogen, sulfur, phosphorous or carbon.
R₉ is hydrogen, alkyl, aryl, cycloalkyl, hydroxyl, thiol, amino acid, glutathione, phosphate, phosphonate, uric acid, ascorbic acid, taurine, estrogen, dehydroepiandrosterone, probucol, vitamin E, hydroxytoluene, carvidilol, α-lipoic acid, α-tocopherol, ubiquinone, phylloquinone, β-carotene, meanadione, succinate, acetyl-L-carnitine, co-enzyme Q lazeroids, polyphenolic flavonoids, —(CH₂)ₙXCH₂)ₙ]NH₂— wherin n=3-12 and X=nitrogen, sulfur, phosporous or carbon, or heterocycle wherin R₅ and R₆ taken together are —(CH₂XCH₂)ₙ— wherin n=3-12 and X=nitrogen, sulfur, phosphorous or carbon.
X₁-X₄ may be the same or different and are nitrogen, sulfur, phosphorous or carbon.
and compounds having the formula: 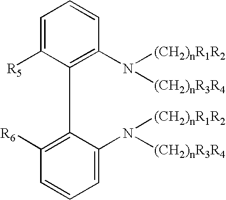
wherein
R₁-R₄ may be the same or different and are hydrogen, alkyl, aryl, cycloalkyl, hydroxyl, thiol, amino acid, pyridine, pyrazole, 3,5-dimethylpyrazole, imidazole, quinoline, phenol, 4-X-phenol and 5-X-phenol where X=chloro, bromo, nitro, methyl, ethyl, methoxy, amino, hydroxy; glutathione, phosphate, phosphonates, uric acid, ascorbic acid, taurine, estrogen, dehydroepiandrosterone, probucol, vitamin E, hydroxytoluene, carvidilol, α-lipoic acid, α-tocopherol, ubiquinone, phylloquinone, β-carotene, meanadione, succinate, acetyl-L-carmtie, co-enzyme Q, lazeroids, polyphenolic flavonoids, —(CH₂)ₙ[(CH₂)ₙ]NH₂— wherin n=34 and X=nitrogen, sulfur, phosporous or carbon, or heterocycle wherin R₁ and R₂ taken together are —(CH₂XCH₂)ₙ— wherin n=34 and X=nitrogen, sulfur, phosphorous or carbon. R₅ and R₅ may be the same or different and are hydrogen, alkyl, aryl, cycloalkyl, hydroxyl, thiol, amino acid, pyridine, pyrazole, 3,5-dimethylpyrazole, imidazole, quinoline, phenol, 4-X-phenol and 5-X-phenol where X=chloro, bromo, nitro, methyl, ethyl, methoxy, amino, hydroxy; glutathione, phosphate, phosphontes, uric acid, ascorbic acid, taurine, estrogen, dehydroepiandrosterone, probucol, vitamin E, hydroxytoluene, carvidilol, α-lipoic acid, α-tocopherol, ubiquinone, phylloquinone, β-carotene, meanadione, succinate, acetyl-L-carnitine, co-enzyme Q, lazeroids, polyphenolic flavonoids, or heterocycle wherin R₃ and R₄ taken together are —(CH₂XCH₂)ₙ— wherin n=34 and X=nitrogen, sulfur, phosphorous or carbon.
R₅-R₁₂ may be the same or different and are hydrogen, alkyl, aryl, cycloalkyl, hydroxyl, thiol, amino acid, pyridine, pyrazole, imidazole, quinoline, phenol, 4-X-phenol and 5-X-phenol where X=chloro, bromo, nitro, methyl, ethyl, methoxy, amino, hydroxy; glutathione, phosphate, phosphonate, uric acid, ascorbic acid, taurine, estrogen, dehydroepiandrosterone, probucol, vitamin E, hydroxytoluene, carvidilol, [symbol] -lipoic acid, [symbol] -tocopherol, ubiquinone, phylloquinone, [symbol] -carotene, meanadione, succinate, acetyl-L-carnitine, co-enzyme Q, lazeroids, polyphenolic flavonoids, —(CH₂)ₙ[XCH₂)ₙNH₂— wherin n=36 and X=nitrogen, sulfur, phosporous or carbon, or heterocycle wherin R₅ and R₆ taken together are —(CH₂XCH₂)ₙ— wherin n=3-6 and
X=nitrogen, sulfur, phosphorous or carbon.
N is an integer with values from 0-10.

3. The method of claim 2 wherein said composition is taken from a group consisting of those compositions having the formula: 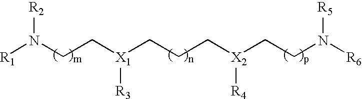
compositions having the formula: 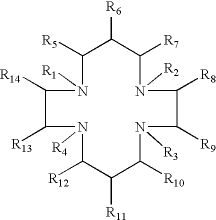
wherein:
R₁ and R₂ are taken from a group consisting of hydrogen, alkyl, aryl, cycloalkyl, amino acid, glutathione, uric acid, ascorbic acid, lane, estrogen, dehydroepiandrosterone, probucol, vitamin E, hydroxytoluene, carvidilol, α-lipoic acid, α-tocopherol, ubiquinone, phylloquinone, β-carotene, meanadione, glutamate, succinate, acetyl-L-carnitine, co-enzyme Q, lazeroids, polyphenolic flavonoids, homocysteine, menaquinone, idebenone, dantrolene, —(CH₂)ₙ[XCH₂)ₙ]NH₂— wherein n=3-6 and R₁ and R₂ taken together are —(CH₂XCH₂)ₙ— wherein n=3-6,
R₃ and R₄ are taken from a group consisting of hydrogen, alkyl, aryl, cycloalkyl, amino acid, glutathione, uric acid, ascorbic acid, taurine, estrogen, dehydroepiandrosterone, probucol, vitamin E, hydroxytoluene, carvidilol, α-lipoic acid, α-tocopherol, ubiquinone, phylloquinone, β-carotene, meanadione, glutamate, succinate, acetyl-L-carnitine, co-enzyme Q, lazeroids, polyphenolic flavonoids, homocysteine, menaquinone, idebenone, dantrolene or heterocycle and R₃ and R₄ taken together are —(CH₂XCH₂)ₙ— wherein n=3-6,
R₅ and R₆ are taken from a group consisting of hydrogen, alkyl, aryl, cycloalkyl, amino acid, glutathione, uric acid, ascorbic acid, taurine, estrogen, dehydroepiandrosterone, probucol, vitamin E, hydroxytoluene, carvidilol, α-lipoic acid, α-tocopherol, ubiquinone, phylloquinone, β-carotene, meanadione, glutamate, succinate, acetyl-L-carnitine, co-enzyme Q, lazeroids, polyphenolic flavonoids, homocysteine, menaquinone, idebenone, dantrolene —(CH₂)ₙ[XCH₂)ₙ]NH₂— wherein n=3-6, and R₅ and R₆ taken together are —( CH₂XCH₂)ₙ—
wherein n=3-6.
R₇, R₈, R₉, R₁₀, R₁₁, R₁₂, R₁₃, and R₁₄, may be the same or different and are hydrogen, alkyl, aryl cycloalkyl, amino acid, glutathione, uric acid, ascorbic acid, taurine, estrogen, dehydroepiandrosterone, probucol, vitamin E, hydroxytoluene, carvidilol, α-lipoic acid, α-tocopherol, ubiquinone, phylloquinone, β-carotene, meanadione, glutamate, succinate, acetyl-L-carnitine, co-enzyme Q, lazeroids, polyphenolic flavonoids, homocysteine, menaquinone, idebenone, dantrolene —CH₂)ₙ[XCH₂)ₙ]NH₂— wherein n=3-6 and X=nitrogen, sulfur, phosporous or carbon, or heterocycle wherein R₅ and R₆ taken together are —(CH₂XCH₂)ₙ— wherein n=3-6 and X=nitrogen, sulfur, phosporous or carbon.
M, n, and p may be the same or different and are bridging groups of variable length from 3-12 carbons.
X₁ and X₂ may be the same or different and are nitrogen, sulfur, phosporous and carbon.
, compositions having the formula: 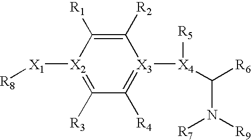
wherein
R₁-R₄ may be the same or different and are hydrogen, alkyl, aryl, cycloalkyl, hydroxyl, thiol, amino acid, glutathione, phosphate, phosphonates, uric acid, ascorbic acid, taurine, estrogen, dehydroepiandrosterone, probucol, vitamin E, hydroxytoluene, carvidilol, α-lipoic acid, α-tocopherol, ubiquinone, phylloquinone, β-carotene, meanadione, succinate, acetyl-L carnitine, co-enzyme Q, lazeroids, polyphenolic flavonoids, —(CH₂)ₙ[XCH₂)ₙ]NH₂— wherin n=3-12 and X=nitrogen, sulfur, phosporous or carbon, or heterocycle wherin R₁ and R₂ taken together are —(CH₂XCH₂)ₙ— wherin n=3-12 and X=nitrogen, sulfur, phosphorous or carbon.
R₅ and R₅ may be the same or different and are hydrogen, alkyl, aryl, cycloalkyl, hydroxyl, thiol, amino acid, glutathione, phosphate, phosphontes, uric acid, ascorbic acid, taurine, estrogen, dehydroepiandrosterone, probucol, vitamin E, hydroxytoluene, carvidilol, α-lipoic acid, α-tocopherol, ubiquinone, phylloquinone, β-carotene, meanadione, succinate, acetyl-L-carnitine, co-enzyme Q, lazeroids, polyphenolic flavonoids, or heterocycle wherin R₃ and R₄ taken together are —(CH₂XCH₂)ₙ— wherin n=3-12 and X=nitrogen, sulfur, phosphorous or carbon.
R₇ and R₈ may be the same or different and are hydrogen, alkyl, aryl, cycloalkyl, hydroxyl, thiol, amino acid, glutathione, phosphate, phosphonate, uric acid, ascorbic acid, taurine, estrogen, dehydroepiandrosterone, probucol, vitamin B, hydroxytoluene, carvidilol, α-lipoic acid, α-tocopherol, ubiquinone, phylloquinone, β-carotene, meanadione, succinate, acetyl-L-carnitine, co-enzyme Q, lazeroids, polyphenolic flavonoids, —(CH₂)ₙ[XCH₂)ₙ]NH₂— wherin n=3-12 and X=nitrogen, sulfur, phosporous or carbon, or heterocycle wherin R₅ and R₆ taken together are —(CH₂XCH₂)ₙ— wherin n=3-12 and X=nitrogen, sulfur, phosphorous or carbon.
R₉ is hydrogen, alkyl, aryl, cycloalkyl, hydroxyl, thiol amino acid, glutathione, phosphate, phosphonate, uric acid, ascorbic acid, taurine, estrogen, dehydroepiandrosterone, probucol, vitamin E, hydroxytoluene, carvidilol, α-lipoic acid, α-tocopherol, ubiquinone, phylloquinone, β-carotene, meanadione, succinate, acetyl-L-carnitine, co-enzyme Q, lazeroids, polyphenolic flavonoids, —(CH₂)ₙ[CH₂)ₙ]NH₂— wherin n=3-12 and X=nitrogen, sulfur, phosporous or carbon, or heterocycle wherin R₅ and R₆ taken together are —(CH₂XCH₂)ₙ— wherin n=3-12 and X=nitrogen, sulfur, phosphorous or carbon.
X₁-X₄ may be the same or different and are nitrogen, sulfur, phosphorous or carbon.
and compositions having the formula: 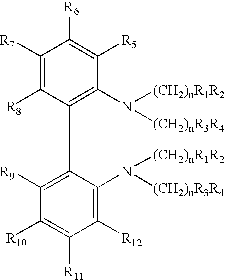
Wherein
R₁-R₄ may be the same or different and are hydrogen, alkyl, aryl, cycloalkyl, hydroxyl, thiol, amino acid, pyridine, pyrazole, 3,5-dimethylpyrazole, imidazole, quinoline, phenol, 4-X-phenol and 5-X-phenol where X=chloro, bromo, nitro, methyl, ethyl, methoxy, amino, hydroxy; glutathione, phosphate, phosphonates, uric acid, ascorbic acid, taurine, estrogen, dehydroepiandrosterone, probucol, vitamin E, hydroxytoluene, carvidilol, α-lipoic acid, α-tocopherol, ubiquinone, phylloquinone, β-carotene, meanadione, succinate, acetyl-L-carnitine, co-enzyme Q, lazeroids, polyphenolic flavonoids, —(CH₂)ₙ[XCH₂)ₙ]NH₂— wherin n=3-6 and X=nitrogen, sulfur, phosporous or carbon, or heterocycle wherin R₁ and R₂ taken together are —(CH₂XCH₂)ₙ— wherin n=3-6 and X=nitrogen, sulfur, phosphorous or carbon.
R₅ and R₆ may be the same or different and are hydrogen, alkyl, aryl, cycloalkyl, hydroxyl, thiol, amino acid, pyridine, pyrazole, 3,5dimethylpyrazole, imidazole, quinoline, phenol, 4-X-phenol and 5-X-phenol where X=chloro, bromo, nitro, methyl, ethyl, methoxy, amino, hydroxy, glutathione, phosphate, phosphontes, uric acid, ascorbic acid, taurine, estrogen, dehydroepiandrosterone, probucol, vitamin E, hydroxytoluene, carvidilol, α-lipoic acid, α-tocopherol, ubiquinone, phylloquinone, β-carotene, meanadione, succinate, acetyl-L-carnitine, co-enzyme Q, lazeroids, polyphenolic flavonoids, or heterocycle wherin R₃ and R₄ taken together are —(CH₂XCH₂)ₙ— wherin n=3-6 and X=nitrogen, sulfur, phosphorous or carbon.
R₅-R₁₂ may be the same or different and are hydrogen, alkyl, aryl, cycloalkyl, hydroxyl, thiol, amino acid, pyridine, pyrazole, imidazole, quinoline, phenol, 4-X-phenol and 5-X-phenol where X=chloro, bromo, nitro, methyl, ethyl, methoxy, amino, hydroxy; glutathione, phosphate, phosphonate, uric acid, ascorbic acid, taurine, estrogen, dehydroepiandrosterone, probucol, vitamin E, hydroxytoluene, carvidilol, α-lipoic acid, α-tocopherol, ubiquinone, phylloquinone, β-carotene, meanadione, succinate, acetyl-L-carnitine, co-enzyme Q, lazeroids, polyphenolic flavonoids, —(CH₂)ₙ[XCH₂)ₙ]NH₂— wherin n=3-6 and X=nitrogen, sulfur, phosporous or carbon, or heterocycle wherin R₅ and R₆ taken together are —(CH₂XCH₂)ₙ— wherin n=3-6 and
X=nitrogen, sulfur, phosphorous or carbon.
N is an integer with values from 0-10.

4. The method of claim 2 wherein said composition is taken from a group consisting of:
[2-methylethylamino)ethyl](3-{[2-methylamino)ethyl]amino}propyl)amine,
(2-piperidylethyl)-{3-[(2-p piperidylethyl)amino]propyl}amine,
(2-piperazinylethyl)-{3-[(2-piperazinylethyl)amino]propyl}anine,
[2-(bicyclo[3.3.-]non-3-ylamino)ethyl](3-{2-(bicyclo[3.3.1]non-3-ylamio)ethyl]amino}propyl)amine,
methyl(3-[methyl(2-pyridylmethyl)amino]propyl)(2-pyridylmethyl)amine,
1,4,8,11-tetraaza-1,4,8,11-tetra(2-piperidylethyl),
1,4,8,11-tetraaza-1,4,8,11-tetrabicyclo[3.3.1]non-3-ylcyclotetradecane,
N,N′-(2′-dinethylphosphioethyl)-propylenediamine,
3-3-(2-aminoethoxy)propoxy)propylamine,
Vanadyl 2,3,2-Tetrarine,
Chromium 2,3,2-Te ne,
Vanadyl (2-piperidylethyl){3-[(2-piperidylethyl)amino]propyl}amine,
Chromium (2-piperidylethyl)-{3-[(2-piperidylethyl)amino]propyl}amine,
Vanadyl 1,4,8,11-tetraza-1,4,8,11-tetrabicyclo[3.3.1]non-3-ylcyclotetradecane,
Chromium 1,4,8,11-tetraaza-1,4,8,11-tetrabicyclo[3.3.1]non-3-ylcyclotetradecane,
p-Phosphonomethyl)-DL-phenylalanine,
2-amino-N-2-{[3-(2-amino-3-(4-phosphonomethylphenyl)propanolylaminol ethyl}amino)propyl]aminoethyl-3-(4-phosphonomethylphenyl)propamide,
2,2′-amino(bis-N,N′-pyridylmethyl)-6,6′-dimethylbiphenyl,
2,2′-diamino (bis-N,N′-pyridinylmethyl)biphenyl,
[(3,5-dimethylpyrazolyl)methyl][2-2-{[(3,5dimethylpyrazinyl) methyl]amino}phenyl)phenyl]amine,
4-methyl-2-{[(2-{2-[(2-pyridylmethylamino]phenyl}-phenyl)amino]methyl}phenol,
3-nitro-2-{[(2-{2-[(2-pyridylmethylamino]phenyl}-phenyl)amino]methyl}phenol,
4-chloro-2-{[(2-{2-[(2-pyridylmethylamino]phenyl}-phenyl)amino]methyl}phenol,
2,amino-3-(-(4-phosphonomethylphenyl)-N-2-{-2-[benzylamino]phenyl}phenyl)propamide,
Manganese (2,2′-diamino (bis-N,N′-quinilylmethyl)biphenyl)(Cl)₂,
Iron (4-chloro-2-{[(2-{2-[(2-pyridylmethylamino]phenyl}-phenyl)amino]methyl}phenol)(Cl)₂]Cl,
Vanadium (2,2″-diamino(bis-N,N′-pyridylmethyl)biphenyl)Cl₂,
Gadolinium (2,2′-diamino(bis N,N′-pyridylmethyl)biphenyl)Cl₂]Cl, and
Chromium (2-({[2-2-{[2-hydroxyphenyl)methyl]amino}phenyl)phenyl]amino}methyl)phenol)Cl)₂]Cl,

5. The method of claim 4 wherein said diseases comprise:
Parkinson's disease, Alzheimer's disease, Lou Gelrig's disease, Binswanger's disease, Olivopontine Cerebellar Degeneration, Lewy Body disease, Stroke, Diabetes Mellitus, Diabetic Nephropathy, Obesity, Hyperinsulinism, Atherosclerosis, Myocardial Ischemia, Cardiomyopathy, Cardiac Failure, Nephropathy, Ischemia, Glaucoma, Presbycussis, Cancer, Osteoporosis and toxin induced disorders.

6. The method of claim 5 wherein said toxin induced disorders comprise paraquat, MPP⁺,
rotenone, diazoxide, streptozotocin and alloxan-induced disorders.

7. The method of claim 2 wherein said step of synthesizing further comprises the steps of:
admixing an element taken from a group consisting of 2,4 dibromopropane and 2,4 dibromopentane dissolved in absolute ethanol into 1,2-diaminoethane hydrate;
heating the resulting mixture to approximately 50° C. for about one hour;
adding potassium chloride;
continuing said heating for three hours;
filtering potassium bromide out of the mixture;
distilling the mixture at reduced pressure;
allowing the formation of top and bottom layers;
separating and distilling the top layer;
converting free amine in the distilled top layer to a tetrahydrochloride salt; and
converting said salt to a free amine by treatment with ammonium hydroxide.

8. The method of claim 7 wherein said steps of admixing, heating, adding and continuing said heating comprise:
forming a solution of 1,3dibromopropane and ethanol in a weight ratio of about 1 to 2.6;
slowly admixing 1,2-diaminoethane hydrate in a weight ratio of about 1 to 2.2 heating the solution to 50° C. for one about 1 hour;
admixing KCl in a weight ratio of 1 to 4; and continuing heating the solution for about 1 hour.

9. The method of claim 8 wherein said composition consists of 1,3-bis-[(2′-aminoethyl) amino]propane; and said steps of admixing, heating, adding and continuing said heating, comprise:
forming a solution of 1,34dibromopropane and ethanol in a weight ratio of about 1 to 2.6;
slowly admixing 1,2-diaminoethane hydrate in a weight ratio of about 1 to 2.2 heating the solution to 50° C. for one about 1 hour;
admixing KCl in a weight ratio of 1 to 4; and continuing heating the solution for about 1 hour.

10. The method of claim 2 wherein said step of selecting comprises:
ascertaining the heats of formation of a set of said compounds; and choosing said compound in consideration of its heat of formation compared to the heats of formation of other compounds in said set.

11. The method of claim 10 wherein: said step of ascertaining comprises: calculating the heats at the formation of said set of compounds from their respective constituent atoms.

12. The method of claim 11 wherein said step of choosing comprises determining the stabilities of said set of compounds as a function of their respective heats of formation;
wherein said stabilities are determined in inverse proportion to said respective heats of formation; and
whereby the relative stabilities of the set of compounds are deemed indicative of ability to yield the most stable complex when reacted with a group of metals.

13. The method of claim 12 wherein,
said group of metals includes copper, cobalt, iron, zinc, cadmium, manganese and chromium.

14. The method of claim 13 wherein said degenerative diseases comprise neurodegenerative diseases characterized by excess iron pools and said compound is selected from a group consisting of 2,2,2-piperidine and 2,3,2 adamantane.

15. The method of claim 13 wherein said degenerative diseases comprise ischemic damage and pump failure post myocardial infarction characterized by iron-induced toxic redox effects and depletion of tissue zinc stores; and said compound is selected from a group consisting of zinc cyclam methylated, zinc cyclam adamantane, cyclam methylated and cyclam adamantane.

16. The method of claim 13 wherein said degenerative diseases comprise neurodegenerative diseases and strokes; and said composition is selected from a group consisting of compositions having chain (open ring) metal binding molecules taken from a group consisting of compositions having copper binding molecules and manganese binding molecules.

17. The method of claim 16 wherein said compositions having copper-binding molecules include 2,3,2 isopropyl on N1/N4; and
said compositions having manganese-binding molecules include 3,3,3 tetrarine.

18. The method of claim 13 wherein said degenerative diseases comprise neurodegenerative disorders, stroke, glaucoma, atherosclerosis, cardiomyopathy, ischemia, optic neuropathy, peripheral neuropathy, presbycussis and cancer, and said composition is selected from derivatives of those compounds having the largest ring molecules.

19. The method of claim 18 wherein said compounds having the largest ring molecules includes 3,3,3 tetramine, cyclam adamantanes, cyclam 3,3,3 and compounds having alkyl substituted molecules.

20. The method of claim 13 wherein said degenerative diseases comprise Parkinson's, Lou Gehrig's, Binswanger's, and Lewy Body diseases, Olivopontine Cerebellar Degeneration, stroke, glaucoma and optic neuropathy; and
said composition is selected from a group of compositions having alkyl side chains.

21. The method of claim 13 wherein said degenerative diseases comprise neurodegenerative diseases, ischemia post myocardial infarction and atherosclerosis; and
said composition is selected from derivatives of compounds from a group consisting of piperidine, piperazine and adamantane.

22. The method of claim 3 wherein said degenerative diseases comprise stroke, diabetic neuropathy, peripheral neuropathy, Alzheimer's disease, atherosclerosis, ischemia, diabetes, presbycussis, cardiomyopathy and congestive heart failure; and said composition is derived from compounds having terminal nitrogen added molecule substitution with elements selected from a group consisting of glutathione, uric acid, ascorbic acid, taurine, estrogen, dehydroepiandrosterone, probucol, vitamin E, hydroxytoluene, carvidilol, a lipoic acid, tocopherols, ubiquinone, phylloquinone, carotenes, menadione, glutamate, succinate, acetyl-1-carnitine, co-enzyme Q, lazeroids, polyphenolic flavonoids, homocysteine, menaquinone, idebenone, dantrolene and phosporous.

23. The method of claim 22 wherein said degenerative disease comprises stroke; and said composition consists of uric acid polyamine.

24. The method of claim 22 wherein said degenerative disease comprises diabetes; and said composition is derived from compounds selected from a group consisting of phosphorous, taurine, CoEnzyme Q, a lipoic acid, tocopherol, succinate, glutamate and acetyl-1-carnitine polyamines.

25. The method of claim 22 wherein said degenerative disease comprises Alzheimer's disease and presbycussis; and
said composition is derived from compounds selected from a group consisting of a lipoic acid and acetyl-I-carnitine polyamines.

26. The method of claim 22 wherein said degenerative disease comprises atherosclerosis; and said composition selected from a group consisting of tocopherol polyamine and coenzyme Q polyamine.

27. The method of claim 22 wherein said degenerative disease comprises ischemia; and
said composition is selected from a group consisting of tocopherol polyamine and coenzme Q polyamine.

28. The method of claim 22 wherein said diseases comprise myocardial degeneration and congestive heart failure; and said composition consists of coenzyme Q polyamine.

29. The method of claim 22 wherein said degenerative diseases comprise cancer, and said composition is taken from a group consisting of cobalt di-homocysteine polyamines.

30. The method of claim 2 wherein said step of converting comprises adjusting the in vivo half life and pharmacokinetic properties of said composition by selective terminal nitrogen substitutions.

31. The method of claim 2 wherein said step of converting comprises adjusting the in vivo half life and pharmacokinetic properties of said composition by addition of side chains on amino or methylene groups.

32. The method of claim 2 wherein said step of selecting comprises:
finding the octanol I water coefficients of partition of a series of said compounds; and
picking said compound in consideration of its octanol/water coefficient compared to the octanol water coefficients of other compounds in said series.

33. The method of claim 32 wherein said step of picking comprises determining the abilities of said series of compounds to pass through the intestinal, blood brain and blood retinal barriers as a function of their respective octanol/water coefficients; wherein said abilities are determined according to a distribution curve centered about 2 and having a useful range extending towards 0.5 and 4, the numbers being log values.

34. The method of claim 2 wherein said step of selecting comprises;
measuring pKas of a list of said compounds; and
selecting said compound in consideration of its pKas compared to the pKa's of other compounds on the list.

35. The method of claim 34 wherein said step of selecting comprises;
selecting a composition with higher pKas in the treatment a disease characterized by lower tissue pH.

36. The method of claim 35 wherein said diseases include ischemia post myocardial infarction and diabetic ketoacidosis.

37. The method of claim 2 wherein said step of selecting comprises determining the respective likely efficiency of said compounds in consideration of the disease target to be treated and the route of administration.

38. The method of claim 20 wherein;
said compound consisting of pyridine tetramine.

39. The method of claim 20 wherein said degenerative disease consists of Alzheimer's disease; and
said compound comprises acetyl-1-carnitine polyamine.

40. The method of claim 22 wherein said degenerative disease consists of diabetes; and
said compounds are selected from a group consisting of 2,3,2 piperidine, glutamate polyamine, succinate polyamine, chromium tetramine and vanadyl tetramine and phosphorous polyamine.

41. The method of claim 2 wherein said degenerative diseases comprise peripheral and optic neuropathy; and
said compounds comprise taurine polyamine and a lipoic acid polyamines.

42. The method of claim 2 wherein said degenerative diseases comprise glaucoma;
and said compounds comprise adamantane 2,3,2 tetramine and adamantane cyclam.

43. The method of claim 3 wherein said degenerative disease comprise presbycussis; and said compounds comprise α lipoic acid polyamine and acetyl-1 carnitine polyamine.

44. The method of claim 4 wherein said composition consists of (2-aminoethyl){3-[(2-aminoethyl)amino]-1-methylbutyl}amine; and said step of admixing, heating adding and continuing said heating comprise forming a solution of 2,4-dibromopentane and ethanol in a weight ratio of about 1 to 17;
slowly admixing 1,2-diaminoethane hydrate in a ratio of about 1 to 35 heating the solution to 50° C. for about 1 hour;
admixing KC in a weight ratio of about 1 to 4; and
continuing heating the solution for about 30 minutes.

45. The method of claim 44 wherein said step of converting to a tetrahydrochloride salt comprises of adding hydrochloric acid.

46. The method of claim 2 wherein
said step of synthesizing further comprises; the steps of
admixing a solution of an element, taken from a group consisting of 1,3-diaminopropane and N,N-dimethyl-1,3-propanediamine and ethanol into 2-chloromethylpiperidine in water;
adjusting the pH of the resulting mixture to 9 by addition of 10% sodium hydroxide;
stirring the mixture at room temperature and maintaining the pH between 8 and 9 by addition of sodium hydroxide over 3 days;
allowing solvents to evaporate; and
extracting residues with CH₂Cl₂.

47. The method of claim 46 wherein said composition consists of (2-pyridylmethyl){3-[(2-pyridylmethyl)amino]propyl}amine;
said step of admixing comprises
forming a first solution of 1,3-diaminopropane and ethanol in a weight ratio of about 1 to 40;
forming a second solution of 2-chloromethyl piperidine and water in a weight ratio of about 1 to 5.5; and
mixing first and second solutions.

48. The method of claim 46 wherein said composition consists of methyl(3-[methyl(2-pyridylmethyl)amino]propyl}(2-pyridylmethyl)amine; and
said step of admixing comprises:
forming a first solution of N,N-dimethyl-1,3-propanediamine and ethanol in a weight ratio of about 1 to 40;
forming a second solution of 2-chloromethyl piperidine and water in a weight ratio of about 1 to 5.5; and
mixing first and second solutions.

49. The method of claim 2 wherein said composition consists of (2-piperidylethyl)-{3-[(2-piperidylethyl)amino]propyl}amine, and
said step of synthesizing comprises:
forming a first solution of 1,3-diaminopropane in ethanol in a weight ratio of about 1 to 80;
admixing NaOH in a weight ratio of about 1 to 25;
forming a second solution of 1-(2chloroethyl)piperidine in ethanol in a weight ratio of about 1 to 16;
adding said second solution to said first first solution dropwise in a weight ratio of about 1 to 1 over about 30 minutes;
stirring the combined solutions over about 25 hours;
evaporating solvents;
extracting residue with CH₂Cl₂ dried over Na₂SO₄;
evaporating to dryness;
converting to a hydrochloride salt by addition of HCl; and
converting back to free amine by treatment with NH₄OH.

50. The method of claim 2 wherein said composition consists of (2-piperazinylethyl)-{3-[(2-piperazinylethyl)aminolpropyl}amine; and
said step of synthesizing comprises:
forming a first solution of 1,3 diaminopropane in ethanol in a weight ratio of about 1 to 80;
admixing NaOH in a weight ratio of about 1 to 25;
forming a second solution of 1-2-chloroethyl)piperidine in ethanol in a weight ratio of about 1 to 16;
adding said second solution to said first first solution dropwise in a weight ratio of about 1 to 1 over about 30 minutes;
stirring the combined solutions over about 25 hours;
evaporating solvents;
extracting residue with CH₂Cl₂ dried over Na₂SO₄;
evaporating to dryness;
converting to a hydrochloride salt by addition of HCl; and
converting back to free amine by treatment with NH₄OH.

51. The method of claim 2 wherein said composition is taken from a group consisting of spermine, spermidine, 2,3,2-piperidine, 2,3,2-pyridine, 2,2,2-tetr ine, 2,3,2-tetramine, 2,3,2-diCH₃, cyclam adamantane, vanadium 2,3,2-piperidfine, 2,32 sulfur, vanadium cyclam adamantane and cyclam piperidine.

52. The method of claim 51 wherein said disease comprises;
paraquat-induced cell death, MPP⁺-induced cell death, rotenone-induced cell death, diazoxde induced cell death, streptozotocin-induced cell death and alloxan induced cell death.

53. The method of claim 52 wherein said disease consists of diazoxide-induced cell death, and said composition is taken from a group consisting of spermine, spermidine, 2,3,2-tetramine, 2,3,2-piperidine, 2,3,2-pyridine, cyclam, chromium 2,3,2-pyridine, 2,3,2-CH₃, 2,3,2-sulfur, cyclam adamantane, vanadium cyclam adamantane and cyclam piperidine.

54. The method of claim 2 wherein said composition consists of
[2-methylethylamino)ethyl](3-([2-methylamino)ethyl]amino}propyl)amine; and said step of synthesizing further comprises; preparing of first mixture of magnesium timings,
1,3-bis-[(2′-aminoethyl)-amino]propane, benzene and acetyl chloride respective approximate percentage of 0.6%, 8.5%, 8.45%, and 6.4% per weight;
cooling said first mixture;
separating the mixture into a liquid phase and a solid phase;
preparing a second mixture by mixing said solid phase with ether;
preparing a solution by pouring said second mixture over ice;
preparing a third mixture by adding said solution to said liquid phase;
washing said third mixture with sodium bicarbonate;
washing said third mixture with water.

55. The method of claim 2 wherein said step of synthesizing comprises converting the starting di- or tetramine component, at least one of said components in said compounds to the corresponding N-substituted compound by treatment with an allyl halide; and
purifying said composition by conversion to a salt through addition of hydrochloric acid.

56. The method of claim 2 wherein said composition consists of (2-aminoethyl){3-[(2-aminoethyl)methylamino]propyl}methylamine, and
said step of synthesizing further comprises:
preparing a first solution of N,N-dimethyl-1,3-propanediamine and ethanol in a ratio of approximately 1 to 50 per weight;
preparing a second solution of 2-chloroethylamine and ethanol in a ratio of approximately 1 to 17 per weight;
combining said first and second solutions into a third solution;
stirring said third solution at room temperature for approximately 20 hours;
evaporating solvents in said third solution; and
extracting residues in said solution with a volume of CH₂Cl₂.

57. The method of claim 2 wherein said composition consists of
[2-(bicyclo[3.3.1]non-3-ylamino)ethyl](3-{2-(bicyclo[3.3.1]non-3-ylamino)ethyl]amino}propyl)amine, and said step of synthesizing further comprises heating for approximately 6 hours at 215° C. a mixture of 1-bromoadamantane and 2,3,2-tetramine in a mol ratio of approximately 1 to 5;
admixing said mixture into a solution of 2NHCl and ether having a ratio of approximately 1.25 to 1 per weight, in a ratio of approximately 1 to 9 per weight;
separating the aqueous layer and alkali said layer in a volume of 50% aqueous NaOH;
extracting with ether;
drying the extract over K₂CO₃; and
evaporating to an oil.

58. The method of claim 2 wherein said composition consists of [2-(methylethylamino)ethyl](3 {[2-(methylamino)ethyl]amino}propyl)amine; and
said step of synthesizing further comprises;
methylating terminal nitrogens of 2,3,2 tetramine by refluxing in the presence of benzene and acetyl chloride.

59. The method of claim 58 wherein said step of synthesizing further comprises;
preparing a first mixture of magnesium turnings;
of 1,3-bis-[(2′-aminoethyl)-amino]propane, benzene and acetyl chloride respective approximate percentage of 0.6%, 8.5%, 8.45%, and 6.4% per weight;
cooling said first mixture;
separating the mixture into a liquid phase and a solid phase;
preparing a second mixture by mixing said solid phase with ether;
preparing a solution by pouring said second mixture over ice;
preparing a third mixture by adding said solution to said liquid phase;
washing said third mixture with sodium bicarbonate;
washing said third mixture with water;
drying said third mixture over CaCl₂;
filtering said third mixture;
preparing a fourth mixture of said third mixture sodium hydride and N,N, -dimethylformamide in a ratio of approximately 2.5, 1 and 37.5 respectively per weight;
heating said fourth mixture under N₂ at approximately 60° C. for about three hours;
treating said fourth mixture with approximately 14 its volume of iodomethane;
stirring said treated fourth mixture at 50° C. for approximately 24 hours;
quenching said treated fourth mixture with 95% ethanol;
removing volatiles at reduced pressure;
watering with addition of approximately ½ volume of water;
extracting organic products with approximately three ½ volumes of chloroform;
washing said organic products with water and NaCl;
drying said organic products over anhydrous sodium sulfate;
concentrating into an oil;
purifying said oil by flash chromatography with ¼ hexanes-ethyl acetate as eluent into an acetylated oil of said composition;
forming a solution of said acetylated oil, potassium hydroxide, methanol and water in respective proportions of 1, 3, 23 and 5 per weight respectively;
heating said solution under reflux for about 24 hours;
removing methanol at reduced pressure;
extracting into ether;
washing with NaCl;
drying over sodium sulfate;
concentrating under vacuum;
purifying by flash chromatography; and
evaporating solvents.

60. The method of claim 2 wherein said composition consists of [2-(dimethylamino)ethyl](3-{[2-dimethylamino)ethyl]methylamino}propyl)methylamine; and
said steps of synthesizing further comprises;
refluxing for about 20 hours a solution of 2,3,2 tetramine, formic acid and 37% formaldehyde and water in a weight proportions of approximately 1,10,10 and 1 respectively;
evaporating solvents from said solution;
making said solution basic by addition of NaOH; and
extracting residues with 3 times 1½ volume of CH₂Cl₂.

61. The method of claim 2 wherein said composition consists of 2-[3-(2-aminoethylthio)propylthio]ethylamine; and
said step of synthesizing further comprises:
preparing a first solution of 1,3-dimercaptopropane and water in a weight ration of about 1 to 50;
preparing a second solution of NaOH and water in a weight ratio of about 1.5 to 10;
forming a first mixture by mixing said first and second solutions in a weight ratio of about 5 to 1;
forming a third solution of 2chloroethylamine and ethanol in a weight ratio of about 8.5 to 1;
admixing said solution into said mixture in a ratio of about 1 to 3.8;
refluxing said mixture over approximately 8 hours;
evaporating solvents from said refluxed mixture;
extracting residues with CH₂Cl₂.

62. The method of claim 2 wherein said composition consists of:
1,4,8,11-tetraaza-1,4,8,11-tetramethylcyclotetradecane; and
said steps of synthesizing comprises:
refluxing for about 18 hours a solution of cyclam, formic acid, 37% formaldehyde and water in weight proportions of approximately 1, 5.3, 4.5 and 1 respectively;
adding water to said solution in a weight ratio of approximately 0.5 to 1;
cooling said solution to about 5° C.;
adjust the pH of said solution to above 12 with NaOH;
extracting the solution with CH₂Cl₂;

63. The method of claim 2 wherein said composition consists of 1,4,8,11-tetraaza-1,4,8,11-tetra(2-piperidylethyl)cyclotetradecane; and said step of synthesizing further comprises:
preparing a first solution of cyclam and CH₂Cl₂ in a weight ratio of approximately 1 to 50;
preparing a second solution of NaOH and water in a weight ratio of approximately 1 to 31;
preparing a mixture of said first and second solution in a weight ratio of approximately 1 to 1;
preparing a third solution of 1-2chloroethyl)piperidine and CH₂Cl₂ in a weight ratio of approximately 1 to 14;
adding said third solution dropwise into said mixture in a weight ratio of about 1 to 2;
string said mixture over about 24 hours;
evaporating solvents; and
extracting residues with CH₂Cl₂.

64. The method of claim 2 wherein said composition consists of 1,4,8,11-tetraaza-1,4,8,11-tetrabicyclo[3.3.1]non-3-ylcyclotetradecane; and
said step of synthesizing further comprises:
forming a first solution of cyclam and ethanol in a weight ratio of approximately 1 to 100;
forming a second solution of 1-bromoadamantane and ethanol in a weight ratio of 1 to 23;
forming a mixture by adding said second solution dropwise into said first solution in a weight ratio of about 1 to 1, over 30 minutes;
heating said mixture to reflux over about 20 hours;
evaporating said solution under reduced pressure; and
extracting residue from said solution with CH₂Cl₂;

65. The method of claim 2 wherein said composition consists of 1,4,8,11-tetraaza-1,4,8,11-tetraethylcyclotetradecane; and
said step of synthesizing further comprises:
forming a solution of cyclam and DMF in a weight ratio of approximately 1 to 50;
admixing under stirring small portions of NaH in a weight ratio of about 1 to 12.5;
heating said solution for about three hours at about 60° C.;
admixing iodoethane in a single portion into said solution in a weight ratio of about 1 to 17.5;
heating said solution at about 60° C. over about 18 hours;
quenching the solution with about 95% ethanol;
extracting residue with CH₂Cl₂.

66. The method of claim 2 wherein said composition consists of N,N′-(2′ dimethylphosphinomethyl)-propylenediamine; and the step of synthesizing further comprises:
incorporating phosphorus into a molecule of propylenediamine in place of two of its nitrogen atoms by addition and reduction reactions.

67. The method of claim 66 wherein said step of incorporating comprises:
preparing a first solution by dissolving propylenediamine into ethanol in a weight ratio of about 1 to 50;
admixing dimethylvinylphosphine sulfide into said solution in a weight ratio of about 1 to 22;
heating at reflux said solution for about 72 hours;
evaporating solvents under reduced pressure, leaving a residue.

68. The method of claim 67 wherein said step of incorporating further comprises:
dissolving said residue in chloroform;
washing said residue with NaOH; and
drying said residue over MgSO₄.

69. The method of claim 68 wherein said step of synthesizing further comprises:
removing solvents in said residue under reduced pressure to yield an oil, crystallizing said oil with ethyl acetate;
preparing a suspension of LiAlH₄ in dry dioxane in a weight ratio of about 1 to 100;
admixing said oil into said suspension;
to yield a mixture;
refluxing said mixture for about 36 hours;
cooling said mixture; and
adding a solution of dioxane in water and NaOH into said mixture.

70. The method of claim 2 wherein said diseases consist of diabetes and abnormal low density lipoprotein (LDL) to high density lipoprotein (HDL) ratio and said composition is selected from a group consisting of vanadyl 2,3,2-tetramine and chromium 2,3,2-tetramine; and
said step of synthesizing further comprises reacting a metallic salt with 2,3,2-tetramine in an ethanol solution.

71. The method of claim 70 wherein said step of reacting comprises:
forming a first solution of 2,3,2 tetramine in ethanol in a weight ratio of about 1 to 20;
forming a second solution of vanadyl acetylacetonate in ethanol in a weight ratio of about 1 to 275;
admixing said second solution into said first solution in a volume ratio of about 1 to 1; and
refluxing said solution for almost 30 minutes.

72. The method of claim 70 wherein said step of reacting further comprises:
preparing a first solution of 2,3,2-tetramine in ethanol in a weight ratio of about 1 to 20;
preparing a second solution of chromium (III) nitrate in ethanol in a weight ratio of about 1 to 80;
admixing said second solution into said first solution in a volume ratio of about 1 to 1; and
refluxing said solution for about 30 minutes.

73. The method of claim 55 wherein said step of converting comprises using amines to attach alkyl halide in a nucleophilic substitution of N atoms.

74. The method of claim 2 wherein said composition consists of 3-(3-(2-aminoethoxy)propoxy)propylamine; and
the step of synthesizing further comprises:
preparing a first solution by dissolving sodium into ethanol in a weight ratio of about 1 to 200;
after cessation of hydrogen evolution admixing and stirring for about one hour 1,3-propanediol;
into said first solution in a weight ratio of about 1 to 0.005;
preparing a second solution of chloroethylamine and ethanol in a weight ratio 1 to 40;
forming a third solution by admixing dropwise said second solution into said first solution over about 30 minutes;
refluxing said third solution over about 8 hours; and
evaporating solvents.

75. The method of claim 2 wherein said composition consists of vadadyl (2-piperidylethyl)-{3-[(2-piperidylethyl)amino]propyl}amine)(Cl)₂; and
the step of synthesizing further comprises:
forming a first solution of 2,3,2 and methanol in a weight ratio of about 1 to 100;
forming a second solution of V(II)Cl₂ and methanol in a weight ratio of about 1 to 145;
forming a third solution by mixing said first and second solutions;
heating said third solution over about 30 minutes;
cooling said third solution at room temperature.

76. The method of claim 2 wherein said composition consists of chromium (2-piperidylethyl)-{3-[(2-piperidylethyl)amino]propyl}amine (Cl)₂]Cl, and the step of synthesizing further comprises:
forming a first solution of 2,3,2-pip and methanol in a weight ratio of about 1 to 100;
forming a second solution of Cr(III)Cl₃ and methanol in a weight ratio of about 1 to 110 forming a third solution by mixing said first and second solutions;
heating said third solution over about 30 minutes;
cooling said third solution at room temperature; and
evaporating said third solution to two fifths of its original volume.

77. The method of claim 2 wherein said composition consists of: Vanadyl (1,4,8,11-tetraaza-1,4,8,11-tetrabicyclo[3.3.1]non-3-ylcyclotetra decane) (Cl)₂; and
said step of synthesizing further comprises:
preparing a first solution of cyclam adamantane and methanol in a weight ratio of about 1 to 66;
preparing a second solution of V(II)Cl₂ and methanol in a weight ratio of about 1 to 160;
forming a third solution by mixing said first and second solutions;
heating said third solution for about 30 minutes;
cooling said third solution to room temperature; and
evaporating said third solution to about one third of its original volume.

77. The method of claim 2 wherein said composition consists of; Chromium (1,4,8,11-tetraaza-1,4,8,11-tetrabicyclo[3.3.1]non-3-ylcyclotetradecane(Cl)₂]Cl and said step of synthesizing comprises:
preparing a first solution of cyclam adamantane and methanol in a weight ratio of about 1 to 66;
preparing a second solution of Cr(III)Cl₃ and methanol in a weight ratio of about 1 to 150;
forming a third solution by mixing said first and second solutions;
heating said third solution for about 30 minutes;
cooling said third solution at room temperature; and evaporating said third solution to about one fourth of its original volume.

78. The method of claim 2 wherein said composition consists of p-(Phosphonomethyl) DL-phenylalanine; and
said step of synthesizing comprises the steps of:
synthesizing a first compound consisting of diethyl(4-cyanobenzyl)acetamidomalonate by reaction of Na, diethylacetamidomalonate and p-eyanobenzyl bromide;
reacting a volume of said first compound with NaNO2 to obtain a second compound consisting of diethyl [4-Aminomethyl)benzyl]acetamidomalonate;
forming a solid diethyl [4-(Hydroxymethyl)benzyl]acetamidomalonate third compound by mixing a volume of said second compound with water, refluxing a volume of said compound with thionyl chloride in dichloromethane to obtain a third compound consisting of diethyl [4-(Chloromethyl)benzyl]acetamidomalonate;
dissolving a volume of said compound in triethyl phosphite to obtain a compound consisting of Diethyl [4[(Diethoxyphosphinyl)methyl]benzyl]acetamidomalonate;
mixing said fourth compound with methanol and HCl.

79. The method of claim 79 wherein
said step of synthesizing comprises:
forming a solution of Na, diethylacetoamidomalonate, p-cyanobenzyl bromide, and ethanol in a weight percentages of about 1%, 9%, 8%, and 83% respectively, refluxing said solution;
stirring said solution for about 17 hours at 110° C. admixing water in a weight ratio of about 2 to 1; and
filtering crystalline material from said solution;
said step of reacting comprises:
forming a hydrogenated solution of said first compound, ethanol, concentrated HCl and Pd/c in a weight percentages of 4.4%, 88%, 6.6% and 0.88% respectively;
keeping said solution at room temperature and under atmospheric pressure for about 22 hours;
filtrating the solution to a dry filtrate;
watering and then drying said filtrate.
said step of forming comprises:
forming a solution of said second compound, and water in a weight ratio of about 0.02 to 1;
heating said solution for about two hours at 110° C.;
drying said solution; and
extracting said solution with ethyl acetate;
washing said extract with a solution of 1 M HCl, water, 5% NaHCO₃, water and brine;
drying said extract over Na2SO4; and
filtering said extract.

81. The method of claim 79 which further comprises converting said composition into a zwittterionic form by treatment with an amine.

82. The method of claim 2 wherein said composition consists of 2,2′-diamino(bis-N,N′-pyridylmethyl)-6,6′ dimethylbiphenyl; and
said step of synthesizing comprises:
forming a first solution of 6,6′-dimethyl-2,2′-dinitrobiphenyl and ethanol in a weight ratio of about 1 to 10;
admixing palladium carbon in a weight ratio of about 1 to 66;
hydrogenating said solution on a Parr system for about 4 hours at 60 p.s.i;
filtering the solution and reducing to an oil under reduced pressure;
crystallizing said oil from ethanol into a first 2,2′-diamino-6,6′ dimethylbiphenyl compound;
forming a refluxed second solution of said first compound and ethanol in a weight ratio of about 0.047 to 1;
forming a third solution of 2-pyridinrcarboxaldehyde and ethanol in a weight ratio of about 0.075 to 1;
admixing said third solution into said second solution in a weight ratio of about 0.64 to 1 to form a fourth solution;
refluxing said fourth solution for 8 hours;
admixing NaBH₄ to said fourth solution once cooled in a weight ratio of about 0.02 to 1;
evaporating said fourth solution at reduced pressure to a residue;
dissolving said residue in water, extracting said residue with ether; and
crystallizing the residue fromethyl acetate/hexane.

83. The method of claim 2 wherein said composition consists of 2,2′-diamino (bis-N,N′-quinilylmethyl)biphenyl; and
said step of synthesizing comprises:
forming a first solution of 2,2′-diaminobiphenyl and ethanol in a weight ratio of about 0.025 to 1;
admixing 2quinolinecarboxaldehyde in a weight ratio of about 0.042 to 1;
refluxing said solution for about 30 minutes;
cooling to 0° C. to form crystals of a first 1-aza-1-(2-(2-(1-aza-2-(3-isoquinolyl)vinyl)phenyl)phenyl)-2-3-isoquinolyl)ethene compound;
forming a second solution of said compound in ethanol in a weight ratio of about 0.025 to 1;
admixing into said second solution NaBH₄ in a weight ratio of about 0.0049 to 1;
refluxing said second solution for about 30 minutes;
stirring said second solution at room temperature for about 30 minutes;
treating said second solution with HCl to acididty;
extracting said second solution with CH₂Cl₂;
drying said second solution on NaSO₄; and
evaporating said second solution under reduced pressure.

84. The method of claim 2 wherein said composition consists of 2-{[(2-{2-[(2-pyridylmethylamino]phenyl}-phenyl)amino]methyl}phenol; and
Said step of synthesizing comprises:
Forming a solution of 2,2′diaminobiphenyl and acetonitrile in a weight ratio of about 0.015 to 1;
admixing N-hydroxymethyl(3, 54dimethyl)pyrazole in a weight ratio of about 0.0197 to 1;
stirring said solution at room temperature for about 3 days;
drying said solution over MgSO₄;
filtering and evaporating said solution to dryness under reduced pressure to form an oil;
crystallizing said oil from ethyl acetate.

85. The method of claim 2 wherein said composition consists of 4-methyl-2-{[(2-{2-[(2-pyridylmethylamino]phenyl}-phenyl)amino]methyl}phenol; and
said step of synthesizing comprises:
forming a first solution of N-(2-pyridylmethyl)-2,2′-diaminobiphenyl in ethanol in a ratio of about 0.03 to 1;
admixing 2-hydroxy-5-methylbenzaldehyde in a weight ratio of about 0.0485 to 1;
refluxing said first solution for about 30 minutes;
cooling said first solution to room temperature;
evaporating said first solution to about one half of its volume;
cooling said first solution to 0° C. to yield a 2-(2-aza-2-(2-(2-pyridylmethyl)amino)phenyl)phenyl)vinyl)-4-methylphenol compound;
forming a second solution of said compound in ethanol in a weight ratio of about 0.05 to 1;
admixing NaBH₄ in a weight ratio of about 0.007 to 1;
stirring said second solution at room temperature for about 24 hours;
treating said second solution to acidity with concentrated HCl;
extracting said second solution with CH₂Cl₂;
drying and evaporating said second solution to an oil;
crystallizing said oil from methanol.

86. The method of claim 2 wherein said composition consists of 3-nitro-2-{[(2-{2-[(2-pyridylmethylamino]phenyl}-phenyl)amino]methyl}phenol and said step of synthesizing comprises:
forming a first solution of N-2-pyridylmethyl)-2,2′-diaminobiphenyl in ethanol in a weight ratio of about 0.075 to 1;
admixing 2-hydroxy-6-nitrobenzaldehyde in a weight ratio of 0.047 to 1;
refluxing said first solution for about 30 minutes; cololing said first solution to yield crystals of 2-2-aza-2-2-2-pyridylmethyl)amino)phenyl)phenyl)vinyl)₅-nphenol compound;
forming a second solution of said compound in ethanol in a weight ratio of about 0.009 to 1;
admixing in a second solution NaBH4 in a weight ratio of about 0.018 to 1;
stirring said second solution at rom temperature for about 24 hours;
treating said second solution with concentrated HCl to a cidity;
extracting said second solution with CH₂Cl₂;
drying and evaporating said second solution to an oil;
crystallizing said oil from methanol.

87. The method of claim 2 wherein said composition consists of 4-chloro-2-{[(2-{2-[(2-pyridylmethylamino]phenyl}-phenyl)amino]methyl}phenol; and the method of synthesizing comprises:
forming a first solution of N-2-pyridylmethyl)-2,2′-diaminobiphenyl in ethanol in a weight ratio of about 0.075 to 1;
admixing 5-chlorosalicaldehyde in a weight ratio of about 0.0395 to 1;
refluxing said first solution for about 30 minutes;
cooling said first solution to room temperature to yield crystals of 2-(2-aza-2-2-2-pyridylmethyl)amino)phenyl)phenyl)vinyl)₄-chlorophenol compound;
forming a second solution of said compound in ethanol in a weight ratio of about 0.00875 to 1;
admixing NaBH₄ in a weight ratio of about 0.0047 to 1;
stirring said second solution at room temperature for about 24 hours;
treating said second solution with concentrated HCl to acidity;
extracting said second solution with CH₂Cl₂;
drying and evaporating said second solution to yield an oil;
crystallizing said oil from ethyl acetate.

88. The method of claim 2 wherein said composition consists of 4-chloro-2-{[(2-{2-[(2-pyridylmethylamino]phenyl}-phenyl)amino]methyl}phenol; and said step of synthesizing comprises:
forming a first solution of N-2-pyridylmethyl)-2,2′-diaminobiphenyl in ethanol in a weight ratio of about 0.075 to 1;
admixing 5-chlorosalicylaldehyde in a weight ratio of about 0.0395 to 1;
refluxing said first solution for about 30 minutes;
cooling said first solution to room temperature to yield crystals of 2-(2-aza-2-(2-(2-pyridylmethyl)amino)phenyl)phenyl)vinyl)-4-chlorophenol compound;
forming a second solution of said compound in ethanol in a weight ratio of about 0.00875 to 1;
admixing NaBH₄ in a weight ratio of about 0.0047 to 1;
stirring said second solution at room temperature for about 24 hours;
treating said second solution with concentrated HCl to acidity;
extracting said second solution with CH₂Cl₂;
drying and evaporating said second solution to yield an oil;
crystallizing said oil from ethyl acetate.

89. The method of claim 2 where said composition consists of 2-amino-N-2-{[3-(2-[2-amino-3-4-phosphonomethylphenyl)propanolylamino]ethyl}amino)propyl]amino)ethyl)-3-4-phosphonomethylphenyl)propanamide; and said step of synthesizing comprises:
Preparing a first solution of dioxane, p-(phosphonomethyl)-DL-phenylalanine, triethylamine and di-ter-butyl dicarbonate in a weight percentage of about 52.6%, 21%, 8.6% and 17.7% respectively;
Stirring said first solution at room temperature for about 3 hours;
Evaporating and drying said first solution to obtain a first oil of the Boc-p-(phosphonomethyl)-DL-phenylalanine;
preparing a second solution of N-2-pyridylmethyl)-2,2′-diaminobiphenyl in DMF in a weight ratio of about 0.077 to 1;
admixing into said second solution a purified volume of said first oil under nitrogen atmosphere in a weight ratio of about 0.09 to 1;
preparing a third solution of DPPA, DMF and powered NaHCO₃ in weight percentages of about 0.95 to 1;
stirring said mixed second and third solutions with ethyl acetate;
washing said mixed solutions first with 1NHCl and then with NaHCO₃;
drying, filtering and evaporating said mixed solutions to obtain an oil of Boc-2-amino-3-4-phosphonomethylphenyl)-N-(2-{2-[benzylamino]phenyl)phenyl) propanamide;
forming a fourth solution of a purified volume of said oil, methylene chloride and tifluoracetic acid in weight percentages of about 4%, 80% and 16% respectively;
stirring said fourth solution at room temperature for about 20 minutes;
evaporating said fourth solution to obtain a trifluoretate salt; and
treating said salt with ammonia.

90. A composition for use in the treatment of degenerative diseases due to acquired mitochondrial DNA damage, redox damage to mitochondrial macromolecules and inherited mitochondrial genetic defects, said composition being selected from a group consisting of
predominantly linear tetraamines and polyamines linked by 1,3-propylene and/or ethylene groups, predominately branched tetraamines and polyamines linked by 1,3-propylene and/or ethylene groups, cyclic polyamines linked by 1,3-propylene and/or ethylene groups, combinations of linear, branched and cyclic polyamines linked by one or more 1,3-propylene and/or ethylene groups, substituted polyamines, and polyamine derivatives of 2,2′-diaminobiphenyl with linear or branched chains attached.

91. The composition of claim 90 wherein said composition is taken from a group consisting of those compositions having the formula: 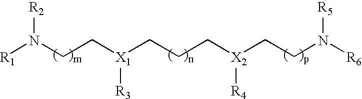
, compositions having the formula: 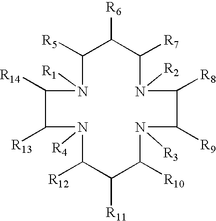
wherein:
R₁ and R₂ are taken from a group consisting of hydrogen, alkyl, aryl, cycloalkyl, amino acid, glutathione, uric acid, ascorbic acid, taurine, estrogen, dehydroepiandrosterone, probucol, vitamin E, hydroxytoluene, carvidilol, α-lipoic acid, α-tocopherol, ubiquinone, phylloquinone, β-carotene, meanadione, glutamate, succinate, acetyl-L-carnitine, co enzyme Q, lazeroids, polyphenolic flavonoids, homocysteine, menaquinone, idebenone, dantrolene, —(CH₂)ₙ[XCH₂)ₙ]NH₂— wherein n=3-6 and R₁ and R₂ taken together are —(CH₂XCH₂)ₙ— wherein n=3-6,
R₃ and R₄ are taken from a group consisting of hydrogen, alkyl, aryl, cycloalkyl, amino acid, glutathione, uric acid, ascorbic acid, taurine, estrogen, dehydroepiandrosterone, probucol, vitamin E, hydroxytoluene, carvidilol, α-lipoic acid, α-tocopherol, ubiquinone, phylloquinone, β-carotene, meanadione, glutamate, succinate, acetyl-L-carnitine, co-enzyme Q, lazeroids, polyphenolic flavonoids, homocysteine, menaquinone, idebenone, dantrolene or heterocycle and R₃ and R₄ taken together are —(CH₂XCH₂)ₙ— wherein n=3-6, R₅ and R₆ are taken from a group consisting of hydrogen, alkyl, aryl, cycloalkyl, amino acid, glutathione, uric acid, ascorbic acid, taurine, estrogen, dehydroepiandrosterone, probucol, vitamin E, hydroxytoluene, carvidilol, α-lipoic acid, α-tocopherol, ubiquinone, phylloquinone, β-carotene, meanadione, glutamate, succinate, acetyl-L-carnitine, coenne Q, lazeroids, polyphenolic flavonoids, homocysteine, menaquinone, idebenone, dantrolene —(CH₂)ₙ[XCH₂)ₙ]NH₂— wherein n=3-6, and R₅ and R₆ taken together are 4CH₂XCH₂)ₙ— wherein n=3-6.
R₇, R₈, R₉, R₁₀, R₁₁, R₁₂, R₁₃, and R₁₄, may be the same or different and are hydrogen, alkyl, aryl, cycloalkyl, amino acid, glutathione, uric acid, ascorbic acid, taurine, estrogen, dehydroepiandrosterone, probucol, vitamin E, hydroxytoluene, carvidilol, α-lipoic acid, α-tocopherol, ubiquinone, phylloquinone, β-carotene, meanadione, glutamate, succinate, acetyl-L-carnitine, co-enzyme Q, lazeroids, polyphenolic flavonoids, homocysteine, menaquinone, idebenone, dantrolene —(CH₂)ₙ[XCH₂)ₙ]NH₂— wherein n=3-6 and X=nitrogen, sulfur, phosphorous or carbon, or heterocycle wherein R₅ and R₆ taken together are —(CH₂XCH₂)ₙ— wherein n=3-6 and X=nitrogen, sulfur, phosphorous or carbon.
M, n, and p may be the same or different and are bridging groups of variable length from 3-12 carbons, and
X is taken from a group consisting of nitrogen, sulfur, phosphorous and carbon.
, compositions having the formula: 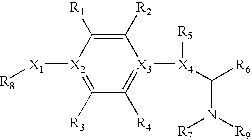
wherein
R₁-R₄ may be the same or different and are hydrogen, alkyl, aryl, cycloalkyl, hydroxyl, thiol, amino acid, glutathione, phosphate, phosphonates, uric acid, ascorbic acid, taurine, estrogen, dehydroepiandrosterone, probucol, vitamin E, hydroxytoluene, carridilol, α-lipoic acid, α-tocopherol, ubiquinone, phylloquinone, β-carotene, meanadione, succinate, acetyl-Lcarnitine, co-enzyme Q, lazeroids, polyphenolic flavonoids, —(CH₂)ₙ[XCH₂)ₙ]NH₂— wherin n=3-12 and X=nitrogen, sulfur, phosporous or carbon, or heterocycle wherin R₁ and R₂ taken together are —(CH₂XCH₂)ₙ— wherin n=3-12 and X=nitrogen, sulfur, phosphorous or carbon.
R₅ and R₅ may be the same or different and are hydrogen, alkyl, aryl, cycloalkyl, hydroxyl, thiol, amino acid, glutathione, phosphate, phosphontes, uric acid, ascorbic acid, tarine, estrogen, dehydroepiandrosterone, probucol, vitamin E, hydroxytoluene, carvidilol, α-lipoic acid, α-tocopherol, ubiquimone, phylloquinone, β-carotene, meanadione, succinate, acetyl-L-carnitine, co-enzyme Q, lazeroids, polyphenolic flavonoids, or heterocycle wherin R₃ and R₄ taken together are —(CH₂XCH₂)ₙ— wherin n=3-12 and X=nitrogen, sulfur, phosphorous or carbon.
R₇ and R₈ may be the same or different and are hydrogen, alkyl, aryl, cycloalkyl, hydroxyl, thiol, amino acid, glutathione, phosphate, phosphonate, uric acid, ascorbic acid, taurine, estrogen, dehydroepiandrosterone, probucol, vitamin E, hydroxytoluene, carvidilol, α-lipoic acid, α-tocopherol, ubiquinone, phylloquinone, β-carotene, meanadione, succinate, acetyl-L-carnitine, co-enzyme Q, lazeroids, polyphenolic flavonoids, —(CH₂)ₙ[XCH₂)ₙ]NH₂— wherin n=3-12 and X=nitrogen, sulfur, phosporous or carbon, or heterocycle wherin R₅ and R₆ taken together are —(CH₂XCH₂)ₙ— wherin n=3-12 and X=nitrogen, sulfur, phosphorous or carbon.
R₉ is hydrogen, alkyl, aryl, cycloalkyl, hydroxyl, thiol, amino acid, glutathione, phosphate, phosphonate, uric acid, ascorbic acid, taurine, estrogen, dehydroepiandrosterone, probucol, vitamin E, hydroxytoluene, carvidilol, α-lipoic acid, α-tocopherol, ubiquinone, phylloquinone, β-carotene, meanadione, succinate, acetyl-L-carnitine, co-enzyme Q, lazeroids, polyphenolic flavonoids, —(CH₂)ₙ[XCH₂)ₙ]NH₂— wherin n=3-12 and X=nitrogen, sulfur, phosporous or carbon, or heterocycle wherin R₅ and R₆ taken together are —(CH₂XCH₂)ₙ— wherin n=3-12 and X=nitrogen, sulfur, phosphorous or carbon.
X₁-X₄ may be the same or different and are nitrogen, sulfur, phosphorous or carbon.
and compositions having the formula: 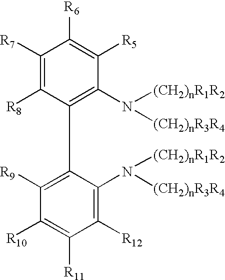
wherein
R₁-R₄ may be the same or different and are hydrogen, alkyl, aryl, cycloalkyl, hydroxyl, thiol, amino acid, pyridine, pyrazole, 3,5-dimethylpyrazole, imidazole, quinoline, phenol, 4-X-phenol and 5-X-phenol where X=chloro, bromo, nitro, methyl, ethyl, methoxy, amino, hydroxy; glutathione, phosphate, phosphonates, uric acid, ascorbic acid, taurine, estrogen, dehydroepiandrosterone, probucol, vitamin E, hydroxytoluene, carvidilol, α-lipoic acid, α-tocopherol, ubiquinone, phylloquinone, β-carotene, meanadione, succinate, acetyl-L-carnitine, co-enzyme Q, lazeroids, polyphenolic flavonoids, —(CH₂)ₙ[XCH₂)ₙ]NH₂— wherin n=36 and X=nitrogen, sulfur, phosphorous or carbon, or heterocycle wherin R₁ and R₂ taken together are —(CH₂XCH₂)ₙ— wherin n=36 and X=nitrogen, sulfur, phosphorous or carbon.
R₅ and R₆ may be the same or different and are hydrogen, alkyl, aryl, cycloalkyl, hydroxyl, thiol, amino acid, pyridine, pyrazole, 3,5-dimethylpyrazole, imidazole, quinoline, phenol, 4-X-phenol and 5-X-phenol where X=chloro, bromo, nitro, methyl, ethyl, methoxy, amino, hydroxy; glutathione, phosphate, phosphontes, uric acid, ascorbic acid, taurine, estrogen, dehydroepiandrosterone, probucol, vitamin E, hydroxytoluene, carvidilol, α-lipoic acid, α-tocopherol, ubiquinone, phylloquinone, β-carotene, meanadione, succinate, acetyl-L-carnitine, co-enzyme Q, lazeroids, polyphenolic flavonoids, or heterocycle wherin R₃ and R₄ taken together are —(CH₂XCH₂— wherin n=36 and X=nitrogen, sulfur, phosphorous or carbon R₅-R₁₂ may be the same or different and are hydrogen, alkyl, aryl, cycloalkyl, hydroxyl, thiol, amino acid, pyridine, pyrazole, imidazole, quinoline, phenol, 4-X-phenol and 5-X-phenol where X=chloro, bromo, nitro, methyl, ethyl, methoxy, amino, hydroxy; glutathione, phosphate, phosphonate, uric acid, ascorbic acid, taurine, estrogen, dehydroepiandrosterone, probucol, vitamin E, hydroxytoluene, carvidilol, α-lipoic acid, α-tocopherol, ubiquinone, phylloquinone, β-carotene, meanadione, succinate, acetyl-L-carnitine, co-enzyme Q, lazeroids, polyphenolic flavonoids, —(CH₂)ₙ[XCH₂)ₙ]NH₂— wherin n=36 and X=nitrogen, sulfur, phosporous or carbon, or heterocycle wherin R₅ and R₆ taken together are {CH₂XCH₂)ₙ— wherin n=36 and X=nitrogen, sulfur, phosphorous or carbon.
N is an integer with values from 0-10.

92. The composition of claim 91 which consists of 1,3-bis-[(2′-aminoethyl)-amino]propane.

93. The composition of claim 91 which consists of [2-methylethylamino)ethyl](3-([2-(methylamino)ethyl]amino}propyl)amine.

94. The composition of claim 91 which consists of (2-piperidylethyl)(3-[(2-piperidylethylaino]propyl}amine.

95. The composition of claim 91 which consists of (2-piperazinylethyl)-{3-[(2-piperazinylethyl)amino]propyl}amine.

96. The composition of claim 91 which consists of [2-bicyclo[3.3.1]non-3-ylamino)ethyl](3-{2-(bicyclo[3.3.1]non-3-ylamino)ethyl]amino)propyl)amine.

97. The composition of claim 91 which consists of methyl(3-[methyl(2-pyridylmethyl)amino]propyl}(2-pyridylmethyl)anine.

98. The composition of claim 91 which consists of 1,4,8,11-tetraaza-1,4,8,11-tetra(2-piperidylethyl)cyclotetradecane.

99. The composition of claim 91 which consists of 1,4,8,11-tetraaza-1,4,8,11-tetrabicyclo[3.3.1]non-3-ylcyclotetmadecane.

100. The composition of claim 91 which consists of N,N′-(2′-dimethylphosphinoethyl)-propylenediamine.

101. The composition of claim 91 which consists of 3-3-2-aminoethoxy)propoxy)propylamine.

102. The composition of claim 91 which consists of vanadyl 2,3,2-tetramine.

103. The composition of claim 91 which consists of chromium 2,3,2-tetramine.

104. The composition of claim 91 which consists of vanadyl (2-piperidylethyl){3-[(2-piperidylethyl)amino]propyl}amine)(Cl)₂.

105. The composition of claim 91 which consists of chromium (2-piperidylethyl)-{3-[(2-piperidylethyl)amino]propyl}amine (Cl)₂]Cl.

106. The composition of claim 91 which consists of vanadyl (1,4,8,11-tetraaa-1,4,8,11-tetrabicyclo[3.3.1]non-3-ylcyclotetra decane) (Cl)₂.

107. The composition of claim 91 which consists of (Chromium 1,4,8,11-tetraaza-1,4,8,11-tetrabicyclo[3.3.1]non-3-ylcyclotetradecane(Cl)₂]Cl.

108. The composition of claim 91 which consists of p-(Phosphonomethyl)-DL-phenylalanine.

109. The composition of claim 91 which consists of 2-amino-N-(2-{[3-(2-amino-3-4-phosphonomethylphenyl)propanolylamino]ethyl}amino)propyl]amino ethyl-3-(4 phosphono methylphenyl)propamide.

110. The composition of claim 91 which consists of 2,2′-diamino(bis-N,N′-pyridylmethyl)-6,6′ dimethylbiphenyl.

111. The composition of claim 91 which consists of 2,2′-diamino (bis-N,N′-quinilylmethyl)biphenyl.

112. The composition of claim 91 which consists of [(3,5-dimethylpyrazolyl)methyl][2-(2-{[(3,5-dimethylpyrazolyl) methyl]amino}phenyl)phenyl]amine.

113. The composition of claim 91 which consists of 4-methyl-2-{[(2-{2-[(2-pyridylmethylamino]phenyl}-phenyl)amino]methyl}phenol.

114. The composition of claim 91 which consists of 3-nitro-2-{[(2-{2-[(2-pyridylmethylamino]phenyl}-phenyl)amino]methyl}phenol.

115. The composition of claim 91 which consists of 4chloro-2-{[(2-{2-[(2-pyridylmethylamino]phenyl}-phenyl)amino]methyl}phenol.

116. The composition of claim 91 which consists of 2,amino-3-(-(4-phosphonomethylphenyl)-N-(2-{-2-[benzylamino]phenyl}phenyl)propamide.

117. The composition of claim 91 which consists of manganese (2,2′-diamino (bis N,N′-quinilylmethyl)biphenyl)(Cl)₂.

118. The composition of claim 91 which consists of iron (4-chloro-2-{[(2-{2-[(2-pyridylmethylamino]phenyl}-phenyl)amino]methyl}phenol)(Cl)₂]Cl.

119. The composition of claim 91 which consists of vanadium (2,2′-diamino(bis-N,N′-pyridylmethyl)biphenyl)Cl₂.

120. The composition of claim 91 which consists of gadolinium (2,2′-diamino(bis-N,N′-pyridylmethyl)biphenyl)Cl₂]Cl.

121. The composition of claim 91 which consists of chromium (2-({[2-2-{[2-hydroxyphenyl)methyl]amino}phenyl)phenyl]amino}methyl) phenol)Cl)₂]Cl.

122. The composition of claim 91 which consists of (2-aminoethyl){3-[(2-aminoethyl)methylamino]propyl}methylamine.

123. The composition of claim 91 which consists of (2-aminoethyl)(3-[(2-aminoethyl)amino]-1-methylbutyl) amine.

124. The composition of claim 91 which consists of (2-pyridylmethyl){3-[(2-pyridyl methyl)amino]propyl}amine.

125. The composition of claim 91 which consists of [2-dimethylamino)ethyl](3-{[2-(dimethylamino)ethyl]methylamino}propyl)methylamine.

126. The composition of claim 91 which consists of 2-[3-2-aminoetlhylthio)propylthio]ethylamine.

127. The composition of claim 91 which consists of 1,4,8,11-4tetraaza-1,4,8,11-tetra(2-piperidylethyl)cyclotetradecane.

128. The composition of claim 91 which consists of 1,4,8,11-tetraaza-1,4,8,11-tetraethylcyclotetadecane.

129. The composition of claim 91 which consists of 2,2′-diamino (bis-N,N′-pyridylmethyl)biphenyl.

130. The composition of claim 91 which consists of 2-{[(2-{2-[(2-pyridylmethylamino]phenyl}-phenyl)amino]methyl}phenol.

131. The composition of claim 91 which consists of 2-({[2-(2-{[2hydroxyphenyl)methyl]amino}phenyl)phenyl]amino}methyl)phenol.

132. A composition for use as RMI contrast medium which is taken from a group consisting of polyamine derivatives of 2,2′-diaminobiphenyl with linear or branched chains attached.

133. The composition of claim 132 which consists of manganese (2,2′-diamino (bis-N,N′-quinilylmethyl)biphenyl)(Cl)₂.

134. The composition of claim 132 which consists of [iron (4-chloro-2-{[(2-{2-[(2-pyridylmethylamino]phenyl}-phenyl)amino]methyl}phenol)(Cl)₂]Cl.

135. The composition of claim 132 which consists of [gadolinium (2,2′-diamino(bis-N,N′-pyridylmethyl)biphenyl)Cl₂]Cl.

136. The method of claim 2 wherein said composition consists of [(3,5-dimethylpyrazolyl)methyl][2-2-{[(3,5-dimethylpyrazolyl)methyl]amino}phenyl)phenyl]amine; and said step of synthesizing comprises:
forming a solution of 2,2′diaminobiphenyl and acetonitrile in a weight ratio of about 0.015 to 1;
admixing N-hydroxymethyl(3,5-dimethyl)pyrazole in a weight ratio of about 0.0197 to 1;
stirring said solution at room temperature for about 3 days;
drying said solution over MgSO₄;
filtering and evaporating said solution to dryness under reduced pressure to form an oil;
crystallizing said oil from ethyl acetate.

137. The method of claim 2 wherein said composition consists of 2-amino-N-(2-{[3-(2-[2-amino-3-(4-phosphonomethylphenyl) propanolylamino]ethyl}amino)propyl]amino}ethyl)-3-4 phosphonomethylphenyl)propanamide, and said step of synthesizing comprises:
preparing a first solution of diaxane, p-(phosphonomethyl)-DL-phenylalanine, triethylamine and di-tert-butyl dicarbonate in a weight percentages of about 52.6%, 21%, 8.5% and 17.7% respectively;
stirring said first solution at room temperature for about 3 hours;
evaporating and drying said first solution to obtain a first oil of Boc-p-(phosphonomethyl)-DL-phenylalanine;
preparing a second solution of 2,3,2-tetramine in DMF in a weight ratio of about 0.05 to 1;
admixing into said second solution a purified volume of said first oil in a weight ratio of about 0.2 to 1 under an atmosphere of nitrogen;
preparing a third solution of DPPA in DMF in a volume ratio of about 0.1 to 1;
admixing into said third solution powered NaHCO₃ in a weight ratio of about 0.03 to 1;
stirring said third solution for about 24 hours;
diluting said third solution with ethyl acetate;
washing said third solution first with 1NHCl and second with saturated NaHCO₃;
drying, filtering and evaporating said third solution to obtain a second oil of Boc-2-amino-N-(2-{[3-(2-[2-amino-3-(4-phosphonomethylphenyl)propanolylamino]ethyl}amino)propyl]aminolethyl)-3-(4 phosphonomethylphenyl)propamide;
preparing a fourth solution of a purified volume of said second oil, methylene chloride, and trifluoracetic acid in weight percentages of about 4%, 16% and 80% respectively; stirring said fourth solution at room temperature for about 30 minutes; evaporating said fourth solution to obtain a tifluoracetic acid salt;
treating said salt with ammonia.

138. The method of claim 52 wherein said disease consists of paraquat-induced cell death, and said composition is taken from a group consisting of spermine, 2,3,2-tetramine and cyclam.

139. The method of claim 52 wherein said disease consists of rotenone-induced cell death, and said composition is taken from a group consisting of 2,3,2-piperidine, 2,3,2-pyridine, chromium 2,3,2-pyridine, 2,2,2-tetramine, 2,3,2-diCH₃ and cyclam adamantane.

140. The method of claim 52 wherein said disease consists of MPP⁺-induced cell death, and said composition is taken from a group consisting of 2,3,2-piperidine, cyclam and cyclam adamantane.

141. The method of claim 52 wherein said disease consists of streptozotocin-induced cell death, and said composition is taken from a group consisting of spermidine, 2,3,2-piperidine, 2,3,2-pyridine, 2,3,2-diCH₃ and cyclam.

142. The method of claim 52 wherein said disease consists of alloxan-induced cell death, and said composition is taken from a group consisting of 2,3,2-adamantane, 2,3,2-pyridine, chromium 2,3,2-pyridine, 2,3,2-diCH₃ and cyclam adamantane.

143. The method of claim 2 wherein said idseases comprise Alpers Syndrome, Alzheimer's Disease, Atherosclerosis, Barth's Disease, Batten's Disease, Beta-Oxidation Disorders, Carnitine Deficiency, Cardiomyopathy, COX (Cytochrome C Oxidase Deficiency), Diabetes, Glaucoma, Glutaric Aciduria, Huntington's Disease, Kearns-Sayre/CPEO, Leigbhs Disease, leber's Optic Neuropathy/LHON, MELAS, Mitochondrial Cardiomyopathies, Mitochondrial Cytopathies, Mitochondrial Encephalomyopathies, Mitochondrial Myopathies, Optic Neuropatly, Parkinson's Disease, Peripheral Neuropathy, Presbycussis, Respiratory Chain disorders: Complexes I, II, III, IV and/or V, Seizures and Stroke; and
said composition is taken from a group consisting of spermine, spermidine, 2,3,2-piperidine, 2,3,2-pyridine, 2,2,2-etranine, 2,3,2-tetramine, 2,3,2-diCH₃, cyclam adamantane, vanadium 2,3,2-piperidine, 2,32 sulfur, vanadium cyclam adamantane and cyclam piperidine.

144. A method of treating osteoporosis, rheumatoid arthritis, inflammatory bowel disease and multiple sclerosis in a mammal, said method comprising:
administering an effective dose of a composition taken from a group of polyamine derived tyrosine phosphatase inhibitors or PPAR partial agonists/partial antagonists.

145. The method of claim 145 wherein said group consists essentially of vanadyl 2,3,2-tetramine, p-(Phosphonomethyl)-DL-phenylalanine, 2-amino-N-(2-{[3-(2-amino-3-(4-phosphonomethylphenyl)propanolylamino]ethyl}amino)propyl]amino}ethyl-3-(4-phosphono methylphenyl)propamide and 2,amino-3-(-(4-phosphonomethylphenyl)-N-(2-{-2-[benzylamino]phenyl}phenyl)propamide.
